(19) United States
(12) Patent Application Publication
(10) Pub. No.: US 2012/0009148 A1
(43) Pub. Date: Jan. 12, 2012

(54) METHODS OF PREDICTING RESPONSIVENESS TO INTERFERON TREATMENT AND TREATING HEPATITIS C INFECTION

(75) Inventor: Yoav Smith, Jerusalem (IL)

(73) Assignee: Yissum Research Development Company of the Hebrew University of Jerusalem, Ltd., Jerusalem (IL)

(21) Appl. No.: 13/142,610

(22) PCT Filed: Dec. 29, 2009
(86) PCT No.: PCT/IL09/01223

§ 371 Date: Sep. 25, 2011

Related U.S. Application Data
(60) Provisional application No. 61/141,048, filed on Dec. 29, 2008.
(60) Provisional application No. 61/146,150, filed on Jan. 21, 2009.
(60) Provisional application No. 61/184,910, filed on Jun. 8, 2009.
(60) Provisional application No. 61/253,703, filed on Oct. 21, 2009.

(51) Int. Cl. A61K 38/21; A61P 31/14; C12Q 1/68

(52) U.S. Cl. 424/85.4; 435/6.13

(57) ABSTRACT

Provided are methods of predicting responsiveness of a subject to interferon treatment, comprising comparing a level of expression in a cell of the subject of at least one gene selected from the group consisting KIR3DL3, KIR3DL2, KIR3DL1, KIR2DL1, KIR2DL2, KIR2DL3, KLRG1, KIR3DS1, CD160, HLA-A, HLA-B, HLA-C, HLA-F, HLA-G and IFI27 to a reference expression data of the at least one gene obtained from at least one interferon responder subject and/or at least one interferon non-responder subject. Also provided are methods and pharmaceutical compositions for treating a subject in need of interferon treatment.

### FIELD AND BACKGROUND OF THE INVENTION

The present invention, in some embodiments thereof, relates to methods of predicting responsiveness to interferon treatment in subjects infected with hepatitis C virus types 1, 2, 3 or 4 or patients diagnosed with multiple sclerosis, and methods of treating hepatitis c infection.

Interferon is widely used to treat a variety of diseases, in particular hepatitis C virus infection (HCV) and multiple sclerosis (MS). Interestingly, in both type 1 HCV and MS the success of the treatment is only about 50%, meaning that half of the population can not benefit from the treatment, while still suffering from its side effects. In HCV types 2, 3 and 4 the chances of interferon treatment success are about 80%. One approach of trying to understand the genetic scenario behind this reality is to look at the gene expression of people in these two groups, before and after the treatment using microarray technology.

Chen et al., 2005 compared the gene expression levels in liver specimens taken before treatment with interferon of 15 non-responders and 16 responders to Pegylated interferon (IFN-alpha) and identified 18 genes whose expression differed significantly between all responders and all non responders and concluded that up-regulation of a specific set of IFN-responsive genes predicts non response to exogenous therapy.

WO2007039906 discloses a method for selecting a set of genes whose expression is different in a first group (for example responders to a specific treatment) as compared to a second group (for example non-responders to a specific treatment) from a pre-determined set of genes such as the full genome.

Taylor M., et al., 2007 found that the induced levels of the OAS1 and 2, MX1, IRF-7 and TLR-7 is lower in poor-interferon response HCV patients than in marked or intermediate interferon response HCV patients.

Van Baarsen et al., 2008 show that the expression level of IFN response genes in the peripheral blood of multiple sclerosis patients prior to treatment can serve a role as a biomarker for the differential clinical response to interferon beta.

Zeremski M, et al., 2007 (J Acquir Immune Defic Syndr. 2007 Jul. 1; 45(3):262-8) showed that PEG-IFN-induced elevations in IP-10 are greater in virological responders than in nonresponders after the first PEG-IFN dose.

Tarantino et al., 2008, disclosed that serum levels of B-Lymphocytes stimulator (BLyS) have a potential role as a predictor of outcome in patients with acute hepatitis C.

Additional background art includes US Pat Appl. 20060177837 (Borozan Ivan et al.), Lopez-Vazquez et al., 2005 (JID 192: 162-165), Ahmad A and Alvarez F. 2004 (J. of Leukocyte Biology, 76:743-759), Parham P, 2004 (Science 305:786-787), Parham P, 2005 (Nature Reviews, Immunology 5:201-214), Zuniga J., et al., 2009 (Molecular Immunology 46:2723-2727), Rauch A., et al. 2007 (Tissue Antigens 69 Suppl 1:237-40), Paladino N., et al., 2007 (Tissue Antigens 69 Suppl 1:109-111); Giannini C, et al., 2008, (Blood. 112:4353-4); Querec T D et al., 2009 (Nat Immunol. 10:116-25. Epub 2008 Nov. 23), Khakoo S I., et al., 2004; Vidal-Casrineira J R., 2009; Rajagopalan S and Long E O. 2005; Gonzalez S, et al., 2005; Shah N., et al., 2009; Vitale M., et al. 2004.

### SUMMARY OF THE INVENTION

According to an aspect of some embodiments of the present invention there is provided a method of predicting responsiveness of a subject to interferon treatment, comprising comparing a level of expression in a cell of the subject of at least one gene selected from the group consisting KIR3DL3, KIR3DL2, KIR3DL1, KIR2DL1, KIR2DL2, KIR2DL3, KLRG1, KIR3DS1, CD160, HLA-A, HLA-B, HLA-C, HLA-F, HLA-G and IFI27 to a reference expression data of the at least one gene obtained from at least one interferon responder subject and/or at least one interferon non-responder subject, thereby predicting the responsiveness of the subject to interferon treatment.

According to an aspect of some embodiments of the present invention there is provided a method of predicting responsiveness of a subject to interferon treatment, comprising comparing a ratio determined between an expression level of TNFRSF17 gene in a cell of the subject following interferon treatment and an expression level of the gene in the cell of the subject prior to interferon treatment, or visa versa, to a reference ratio determined in a cell of at least one interferon responder subject and/or at least one interferon non-responder subject, the reference ratio is determined between an expression level of the gene following interferon treatment and an expression level of the gene prior to interferon treatment, or visa versa, thereby predicting the responsiveness to interferon treatment of a subject.

According to an aspect of some embodiments of the present invention there is provided a method of predicting responsiveness to interferon treatment of a subject diagnosed with multiple sclerosis or infected with HCV type 2, 3 or 4, comprising comparing a level of expression in a cell of the subject of IFI6, OAS2, ISG15, OAS3 and IFIT1 genes to a reference expression data of the genes obtained from at least one interferon responder subject and/or at least one interferon non-responder subject, thereby predicting the responsiveness of the subject to interferon treatment.

According to an aspect of some embodiments of the present invention there is provided a method of predicting responsiveness of a subject to interferon treatment, comprising comparing a ratio determined between an expression level of ISG15, IFI6, IFIT1, OAS2 and OAS3 genes in a cell of the subject following interferon treatment and an expression level of the genes in the cell of the subject prior to interferon treatment, or visa versa, to a reference ratio determined in a cell of at least one interferon responder subject and/or at least one interferon non-responder subject, the reference ratio is determined between an expression level of the genes following interferon treatment and an expression level of the genes prior to interferon treatment, or visa versa, thereby predicting the responsiveness to interferon treatment of a subject.

According to an aspect of some embodiments of the present invention there is provided a method of predicting responsiveness of a subject to interferon treatment, comprising comparing a ratio determined between an expression level of at least one gene selected from the group consisting of: TICAM1, MYD88, TLR7, TRAFD1 and IRF7 in a cell of the subject following interferon treatment and an expression level of the at least one gene in the cell of the subject prior to interferon treatment, or visa versa, to a reference ratio determined in a cell of at least one interferon responder subject and/or at least one interferon non-responder subject, the reference ratio is determined between an expression level of the gene following interferon treatment and an expression level of the gene prior to interferon treatment, or visa versa, thereby predicting the responsiveness to interferon treatment of a subject.

According to an aspect of some embodiments of the present invention there is provided a method of predicting responsiveness of a subject to interferon treatment, comprising comparing a ratio determined between an expression level of at least one gene selected from the group consisting of HERC5 and UBE2L6 in a liver cell of the subject following interferon treatment and an expression level of the at least one gene in the liver cell of the subject prior to interferon treatment, or visa versa, to a reference ratio determined in a cell of at least one interferon responder subject and/or at least one interferon non-responder subject, the reference ratio is determined between an expression level of the at least one gene following interferon treatment and an expression level of the at least one gene prior to interferon treatment, or visa versa, thereby predicting the responsiveness to interferon treatment of a subject.

According to an aspect of some embodiments of the present invention there is provided a method of predicting responsiveness of a subject to interferon treatment, comprising comparing a ratio determined between an expression level of at least one gene selected from the group consisting ISG15, IFI6, IFIT1, OAS2 and OAS3 in a liver cell of the subject following interferon treatment and an expression level of the at least one gene in the liver cell of the subject prior to interferon treatment, or visa versa, to a reference ratio determined in a liver cell of at least one interferon responder subject and/or at least one interferon non-responder subject, the reference ratio is determined between an expression level of the at least one gene following interferon treatment and an expression level of the at least one gene prior to interferon treatment, or visa versa, thereby predicting the responsiveness to interferon treatment of a subject.

According to an aspect of some embodiments of the present invention there is provided a method of treating of a subject in need of interferon treatment, the method comprising: (a) predicting the responsiveness of the subject to interferon treatment according to the method of the invention, and (b) selecting a treatment regimen based on the responsiveness; thereby treating the subject in need of interferon treatment.

According to an aspect of some embodiments of the present invention there is provided a method of treating a subject in need of interferon therapy, comprising co-administering to the subject interferon and an agent capable of downregulating HLA or KIR inhibitory receptor, thereby treating the subject in need of interferon therapy.

According to an aspect of some embodiments of the present invention there is provided a pharmaceutical composition comprising interferon, an agent capable of downregulating HLA or KIR inhibitory receptor, and a pharmaceutically acceptable carrier.

According to some embodiments of the invention, a decrease above a predetermined threshold in the level of expression of the at least one gene in the cell of the subject relative to the reference expression data of the at least one gene obtained from the at least one interferon non-responder subject predicts responsiveness of the subject to interferon treatment of the subject.

According to some embodiments of the invention, an increase above a predetermined threshold in the level of expression of the at least one gene in the cell of the subject relative to the reference expression data of the at least one gene obtained from the at least one interferon responder subject predicts lack of responsiveness of the subject to interferon treatment of the subject.

According to some embodiments of the invention, when a level of expression of the at least one gene in the cell of the subject is identical or changed below a predetermined threshold as compared to the reference expression data of the at least one gene obtained from the at least one interferon responder subject, then the subject is classified as responsive to interferon.

According to some embodiments of the invention, when a level of expression of the at least one gene in the cell of the subject is identical or changed below a predetermined threshold as compared to the reference expression data of the at least one gene obtained from the at least one interferon non-responder subject, then the subject is classified as a non-responsive to interferon.

According to some embodiments of the invention, an increase above a predetermined threshold in the ratio of the subject relative to the reference ratio of the at least one interferon non-responder subject predicts responsiveness of the subject to interferon treatment of the subject.

According to some embodiments of the invention, a decrease above a predetermined threshold in the ratio of the subject relative to the reference ratio of the at least one interferon responder subject predicts lack of responsiveness of the subject to interferon treatment of the subject.

According to some embodiments of the invention, when the ratio of the subject is identical or changed below a predetermined threshold as compared to the reference ratio of the at least one interferon responder subject, then the subject is classified as responsive to interferon.

According to some embodiments of the invention, when the ratio of the subject is identical or changed below a predetermined threshold as compared to the reference ratio of the at least one interferon non-responder subject, then the subject is classified as non-responsive to interferon.

According to some embodiments of the invention, the level of expression is determined prior to interferon treatment.

According to some embodiments of the invention, the cell is a blood cell.

According to some embodiments of the invention, the cell is a liver cell.

According to some embodiments of the invention, following interferon treatment is effected about 4 hours after interferon treatment.

According to some embodiments of the invention, following interferon treatment is effected about 24 hours after interferon treatment.

According to some embodiments of the invention, the subject is diagnosed with HCV infection type 1, 2, 3 or 4.

According to some embodiments of the invention, the method further comprising comparing a ratio determined between an expression level of at least one gene selected from the group consisting of CXCL10 and CD24 in a cell of the subject following interferon treatment and an expression level of the gene in the cell of the subject prior to interferon treatment, or visa versa, to a reference ratio determined in a cell of at least one interferon responder subject and/or at least one interferon non-responder subject, the reference ratio is determined between an expression level of the at least one gene following interferon treatment and an expression level of the at least one gene prior to interferon treatment, or visa versa, thereby predicting the responsiveness to interferon treatment of a subject.

According to some embodiments of the invention, the subject is diagnosed with multiple sclerosis the cell of the subject is a blood cell.

According to some embodiments of the invention, the subject is infected with HCV type 2, 3 or 4 the cell of the subject is a liver cell.

According to some embodiments of the invention, the cell is a blood cell.

According to some embodiments of the invention, further comprising comparing a ratio determined between an expression level of at least one gene selected from the group consisting of ISG15 and USP18 in a liver cell of the subject following interferon treatment and an expression level of the at least one gene in the liver cell of the subject prior to interferon treatment, or visa versa, to a reference ratio determined in a cell of at least one interferon responder subject and/or at least one interferon non-responder subject, the reference ratio is determined between an expression level of the at least one gene following interferon treatment and an expression level of the at least one gene prior to interferon treatment, or visa versa, thereby predicting the responsiveness to interferon treatment of a subject.

According to some embodiments of the invention, the co-administering is effected so as to enable a pharmacokinetic overlap between the interferon and the agent.

According to some embodiments of the invention, the subject is infected with HCV type 1.

According to some embodiments of the invention, the subject is infected with HCV type 2, 3 or 4

According to some embodiments of the invention, the subject is diagnosed with multiple sclerosis.

According to some embodiments of the invention, the level of expression is determined using an RNA detection method.

According to some embodiments of the invention, the level of expression is determined using a protein detection method.

According to some embodiments of the invention, the agent is selected from the group consisting of an antibody, an RNA silencing molecule, a ribozyme and a DNAzyme.

According to some embodiments of the invention, the antibody is an anti-KIR inhibitory receptor antibody.

According to some embodiments of the invention, the RNA silencing molecule is an siRNA directed against a KIR inhibitory receptor or a HLA.

According to some embodiments of the invention, the subject is a non-responder to interferon treatment.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E depict signature gene expression in liver tissue of HCV type 1 patients prior to interferon injection. Expression data was downloaded from the Gene Expression Omnibus Accession No. GSE11190. FIG. 1A—218400_at OAS3; FIG. 1B—204415_at IF1 6; FIG. 1C—205483_s_at ISG15; FIG. 1D—204972_at OAS2; FIG. 1E—203153_at IFIT1. Sequences of probes are provided in Table 2 (Example 2 of the Examples section which follows). Numbers on the “X” axis refer to subjects as follows: Subjects 1-2=healthy controls; subjects 3-6—non-responders to interferon; subjects 7-9—responders to interferon. Note that non responders (subjects 3-6) exhibit high expression of the tested probes (genes) while responders (subjects 7-9) and healthy controls (subjects 1-2) exhibit low expression of the tested probes (genes). Raw data is provided in Table 3 (Examples section which follows).

FIGS. 2A-E depict the logarithmic ratio between the expression level of the tested genes measured in liver tissues of type 1 HCV 4 hours after interferon treatment as compared to the expression level measured in the same tissues prior to interferon treatment. Expression data was downloaded from the Gene Expression Omnibus Accession No. GSE11190. FIG. 2A—ISG15; FIG. 2B—IFI6; FIG. 2C—IFIT1; FIG. 2D—OAS2; FIG. 2E—OAS3. Numbers on the “X” axis refer to subjects as follows: Subjects 1-4=non-responders to interferon; subjects 5-7—responders to interferon. Note that while in Type 1 HCV non responders (subjects 1-4) there is no change in the expression level of the tested genes 4 hours after in vivo injection of interferon as compared to the level prior to interferon injection, in interferon responders (subjects 5-7) there is a significant log 2 up-regulation in the expression level of the tested genes 4 hours after in vivo injection of interferon as compared to before injection. Raw data is provided in Table 4 (Examples section which follows).

FIG. 3 is a graph depicting the distribution of the ratio between the non-responders HCV type 1 base line and the expression level in responders of the 5-signature genes (ISG15, IFI6, IFIT1, OAS2, OAS3) among interferon responders as measured in liver tissues of naïve patients (i.e., time 0, before the first interferon injection). Each point in the graph depicts the percentage of interferon responders having the specific ratio between the non-responders base line and the level in responders. For example, while in 100% of the interferon responders the expression level is 1/1.17 than the non-responders baseline, in 7.6% of the responders the expression level is 1/35 than the non-responders baseline.

FIG. 4 is a volcano plot depicting the significance of changes in the expression levels of various genes in liver of HCV types 1-4 between interferon responders and non-responders as measured prior to interferon injection. Expression data was downloaded from the Gene Expression Omnibus Accession No. GSE11190. The “X” axis represents log 2 of ratio between responders to non responders where the vertical red lines on the right and left represent fold change of 2, meaning that points marked on the left of the left vertical line are up-regulated in non responders and points marked on the right of the right vertical red line are up-regulated more than 2 folds in the responders. The “Y” axis represents the p value assigned to the points. The horizontal red line corresponds to a p value of 0.05. Points appearing above the red horizontal line corresponds to p values lower than 0.05 (i.e., more significant). Note that in liver tissues of HCV types 1-4 interferon responders the 5 genes i.e., ISG15, IFIT1, IFI6, OAS2, OAS3 are down regulated as compared to the non-responders, i.e., the level in non-responders is significantly higher than in the responders.

FIG. 5 is a volcano plot depicting the significance of changes in expression levels of various genes in liver of HCV type 1 measured prior to interferon injection between interferon responders and non-responders. Expression data was downloaded from the Gene Expression Omnibus Accession No. GSE11190. The “X” and “Y” axes as well as the vertical and horizontal red lines are as described above with respect to FIG. 4. Note that in tissues of HCV type 1 the 5 genes i.e., ISG15, IFIT1, IFI6, OAS2, OAS3 (4 of them are marked) are down regulated in responders HCV type 1 before treatment as compared to non-responders.

FIGS. 6A-D depict the ratio on a logarithmic scale between the expression level of the HERC5, ISG15, USP18 and UBE2L6 genes involved in the ISGylated process in liver HCV type 1 biopsies before injection of interferon and 4 hours after in vivo injection of interferon. Expression data was downloaded from the Gene Expression Omnibus Accession No. GSE11190. FIG. 6A—HERC5; FIG. 6B—ISG15; FIG. 6C—USP18; FIG. 6D—UBE216; Numbers on the “x” axis refer to subjects as follows: Subjects 1-4=non-responders to interferon; subjects 5-7—responders to interferon. Sequences of probes are provided in Table 5 in the Examples section which follows. Note that while in tissues (liver biopsies) obtained from Type 1 HCV responders to interferon (subjects 5-7) there is a significant up-regulation of the genes involved in the ISGylated process 4 hours after interferon injection as compared to the level prior to interferon injection, in non-responders (subjects 1-4) there is no change in the expression level of these genes following interferon treatment. Raw data is provided in Table 6 in the Examples section which follows.

FIG. 7 is a schematic presentation of the genomic sequences of the signature genes OAS2, OAS3, IFIT1, IFI6 (G1P3) and ISG15 (G1P2) depicting analysis of the transcription factors and binding sites of the signature genes. Red—IRF7; Black—ISGf3; Blue—ISRE. Note that the regulatory sequences of all analyzed genes include the ISRE promoter where ISGF3 complex and IRF7 are the controlling elements.

FIGS. 8A-E depict the fold change gene expression levels of G1P2 (FIG. 8A), IFIT1 (FIG. 8B), OAS3 (FIG. 8C), G1P3 (FIG. 8D) and OAS2 (FIG. 8E) in PBMC of type 1 HCV patients detected 24 hours after interferon treatment compared to the level detected prior to interferon treatment. Numbers on the “X” axis refer to subjects as follows: Subjects 1-20—responders to interferon; subjects 21-37—non-responders to interferon. Numbers on the “y” axis refer to fold change in gene expression level. The t-test p-values were calculated on the average and standard variation between the 2 groups. RMA normalized data down loaded from GSE7123.

FIGS. 9A-E depict the fold change in gene expression level of key genes from the TLR-Mediated Type I IFN induction pathways in PBMC of type 1 HCV patients detected 24 hours after the interferon injection treatment as compared to the level detected before interferon treatment. FIG. 9A—TICAM1; FIG. 9B—TLR7; FIG. 9C—IRF7; FIG. 9D—MYD88; FIG. 9E—TRAFD1. Sequences of the probes and genes are provided in Table 7 in the Example section which follows. RMA normalized data down loaded from GSE7123. Numbers in the “x” axis refer to subjects as follows: Subjects 1-20—responders to interferon; subjects 21-37—non-responders to interferon. “Y” axis=numbers refer to fold change gene expression levels [fold change=2̂ log 2 level at 24−log 2 level at 0 on RMA data]. The t-test p-values were calculated on the average and standard variation between the 2 groups. Note that 24 hours following interferon treatment the key genes of the Tlr 9 mediated pathway show significant up-regulation as compared to before treatment in PBMC of responders, but not in non-responder.

FIG. 10 is a clustergram of the 5 signature genes IFI6, OAS2, ISG15, OAS3, IFIT1_avg, which were found to be switch response genes (for interferon response) in HCV (WO2007039906), using the expression level in a multiple sclerosis (MS) microarray data as downloaded from the Gene Expression Omnibus Accession No. GSE10655 [publicly available from Hypertext Transfer Protocol://World Wide Web (dot) ncbi (dot) nlm (dot) nih (dot) gov/projects/geo/; van Baarsen L G M, Vosslamber S, Tijssen M, Baggen J M C, van der Voort L F, et al. (2008) Pharmacogenomics of Interferon-β Therapy in Multiple Sclerosis: Baseline IFN Signature Determines Pharmacological Differences between Patients. PLoS ONE 3(4): e1927. doi:10.1371/journal.pone.0001927]. The expression level of IFI6, OAS2, ISG15, OAS3 and IFIT1 was determined in blood before interferon treatment in all MS patients (subjects 8, 16, 3, 14, 20, 10, 18, 21, 12, 2, 9, 22, 15, 11, 7 and 17). The color index of expression level is as follows: Green—low expression; black—middle expression; red—high expression. Note that MS patients numbers 7, 17 (non-responders to interferon as indicated by an increase in relapse rate from 3 relapses per year to 5 relapses per year) and MS patients 10 and 18 (responders to interferon as indicated by the switch from 3 relapses per year to 0 relapses per year) exhibit the same signature genes expression pattern as their parallel non responders and responders to interferon among HCV patients. For example, a very high expression of isg15 and ifi6 followed by a high expression of oas2 oas3 and ifit1 in non-responders (subjects 7 and 17) and the opposite (i.e., low expression) of these genes in the responders (subjects 10 and 18).

FIG. 11 is a volcano plot of the expression level of selected genes which compares the level of expression measured in PBMC of interferon responders versus non-responders HCV type 1 patients prior to interferon treatment (time 0). Data was up-loaded from the Gene Expression Omnibus Accession No. gse11190. The vertical red lines on the right and left represent fold change of 3.5, meaning that points marked on the left of the left vertical line are up-regulated in non responders and points marked on the right of the right vertical red line are up-regulated more than 3.5 folds in the responders; The horizontal red line corresponds to a p value of 0.05. Points appearing above the red horizontal line corresponds to p values lower than 0.05 (i.e., more significant). Note that the KIR2DL1, KIR2DL2, KIR2DL3, CD160, KLRG1, KIR3DL1, KIR3DL2, KIR3DL3, and KIR3DS1 are significantly down-regulated in interferon responders than in non-responders.

FIG. 12 is a volcano plot which compares the changes in gene expression in PBMC of type 1 HCV patients following interferon treatment between responders and non-responders to interferon. The changes in gene expression are calculated by the ratio between the expression level of a gene measured 4 hours after interferon injection and the expression level of the gene measured prior to interferon injection. “X” and “Y” axes and vertical and horizontal lines are as described with respect to FIG. 4. Data was up-loaded from the Gene Expression Omnibus Accession No. gse11190. Note that following interferon injection, the TNFRSF17, CXCL10 and CE24 are significantly up-regulated in interferon responders as compared to interferon non-responders.

FIG. 13 depicts the changes in the expression level of TNFRSF17 in blood of patients having type 1 HCV 4 hours after interferon treatment as compared to before interferon treatment. The changes in the expression level (Y axis) are presented in log 2 values of the expression level measured 4 hours after interferon treatment as compared to the expression level measured prior to interferon treatment. Numbers in the “x” axis refer to subjects as follows: Subjects 1-4—non-responders to interferon; subjects 5-7—responders to interferon. Note that the change in TNFRSF17 is highly persistent and significance.

FIG. 14 is a graph depicting the expression level of IFI27, HLA-A, HLA-B and HLA-C in HCV type 1 liver tissues before interferon treatment. Subjects include healthy individuals (Cont1 and Cont2), non-responders to interferon treatment (n_{—}15, n_{—}16, nr_{—}12 and nr_{—}14) and responders to interferon treatment (r_{—}10, r_{—}3 and r_{—}9). Data was up-loaded from the Gene Expression Omnibus Accession No. gse11190 (Affymetrix hu133 plus 2). Note that the IFI27, HLA-A, HLA-B and HLA-C exhibit the most similar (most correlated) expression pattern as the ISG15 gene; e.g., upregulated in non-responders and downregulated in interferon responders.

FIG. 15 is a volcano plot depicting the significance of changes in expression levels of various genes in liver of HCV type 1 measured in responders as compared to non responders prior to injection. “X” and “Y” axes and vertical and horizontal lines are as described with respect to FIG. 4. Data set was downloaded from the Gene Expression Omnibus Accession No. gse11190. Note that in tissues of HCV type 1 responder patients the HLA-B, HLA-F, HLA-C and HLA-G are significantly down-regulated in more than 2 fold change and with a p value lower than 0.05 as compared to responders, thus demonstrating a significant up-regulation of these genes in liver tissues of HCV type 1 non-responders.

FIG. 16 is a volcano plot depicting the significance of changes in expression levels of various genes in liver of HCV type 1 measured at time 0 in responders versus non responders. The vertical red lines on the right and left represent fold change of 4.6, meaning that points marked on the left of the left vertical line are up-regulated in non responders and points marked on the right of the right vertical red line are up-regulated more than 4.6 folds in the responders; The horizontal red line corresponds to a p value of 0.05. Points appearing above the red horizontal line corresponds to p values lower than 0.05 (i.e., more significant). Note that in parallel to the HLA genes the up-regulated switch genes (e.g., ISG15, IFIT1, USP18, OAS2, OAS3, and HERC6) from the Chen L. et al., 2005 set [Hepatic Gene Expression Discriminates Responders and Nonresponders in Treatment of Chronic Hepatitis C Viral Infection, Gastroenterology, Volume 128, Issue 5, Pages 1437-1444; Hypertext Transfer Protocol://142.150.56.35/˜LiverArrayProject/home (dot) html] are also up regulated in the gse11190 set with a higher fold change (i.e., at least 4.6 folds) as compared to the HLA genes (which is in the range of 2 folds, FIG. 15).

FIG. 17 is a volcano plot depicting the significance of changes in expression levels of various genes in PBMC of HCV type 1 patients as measured at time 0 between responders versus non-responders prior to interferon treatment. Data set was downloaded from the Gene Expression Omnibus Accession No. gse11190. The vertical red lines on the right and left represent fold change of 3.5, meaning that points marked on the left of the left vertical line are up-regulated in non responders and points marked on the right of the right vertical red line are up-regulated more than 3.5 folds in the responders; The horizontal red line corresponds to a p value of 0.05. Points appearing above the red horizontal line corresponds to p values lower than 0.05 (i.e., more significant). Note that in PBMC of type 1 HCV at time 0 (being naïve to interferon treatment, i.e., never received interferon) the KIR2D and KIR3D inhibitory natural killer (NK) receptors are up-regulated (at least 3.5 folds) in non responders compared to responders prior to Interferon injection.

FIG. 18 is a schematic presentation of the natural killer cell mediated cytotoxicity pathway in which genes which are significantly upregulated in liver tissues of non responders type 1 at time 0 are highlighted in yellow. The analysis was performed using the ontoexpress software (Intelligent Systems and Bioinformatics Laboratory, Computer Science Department, Wayne State University).

FIG. 19 is a schematic presentation of the natural killer cell mediated cytotoxicity pathway in which genes which are significantly upregulated in PBMC of non responders type 1 at time 0 are highlighted in yellow. The analysis was performed using the ontoexpress software (Intelligent Systems and Bioinformatics Laboratory, Computer Science Department, Wayne State University). Note that in the blood of non-responders the significant upregulation of the kir inhibitor NK receptors (e.g., KIR3D and KIR2D) matches the upregulation of the HLA genes in the liver as shown in FIG. 18.

FIGS. 20A-B are graphs depicting the expression level of HLA-G in a liver tissue and of his matched KIR2d4 in the blood of HCV type 1 subjects before interferon treatment. Subjects 1-3 (interferon responders); subjects 4-7 (interferon non-responders). The various colors in FIG. 20A indicate expression level using several HLA-G probes. FIG. 21A—HLA-G (in liver tissue); FIG. 21B—KIR2D4 (in PBMC).

FIGS. 21A-B are graphs depicting the expression level of HLA-B in a liver tissue and of his matched KIR3DL3 in the blood of HCV type 1 subjects before interferon treatment. Subjects 1-3 (interferon responders); subjects 4-7 (interferon non-responders). The various colors in FIG. 21A indicate expression level using several HLA-B probes. FIG. 21A—HLA-B (in liver tissue); FIG. 21B—KIR3DL3 (in PBMC).

FIGS. 22A-B are graphs depicting the expression level of HLA-C in a liver tissue and of his matched KIR3DL3 in the blood of HCV type 1 subjects before interferon treatment. Subjects 1-3 (interferon responders); subjects 4-7 (interferon non-responders). The various colors in FIG. 22A indicate expression level using several HLA-C probes. FIG. 22A—HLA-C (in liver tissue); FIG. 22B—KIR2DL3 (in PBMC).

FIG. 23 is a schematic presentation of the OAS2, HLA-A, HLA-B, OAS3, HLA-C, IFIT1, HLA-F, IFI6, IFI27 and ISG15 genes along with their regulatory sequences. Note that the ISRE (light blue solid bars) is a common promoter to all of these genes and is positioned within the 300 bp upstream sequence. Green empty bars=gene coding sequences; Blue empty bars=first exon.

FIG. 24 is a schematic presentation of the OAS2, HLA-A, HLA-B, OAS3, HLA-C, HLA-F, IFI6, IFI27 and ISG15 genes along with their upstream regulatory sequences. Note the 3 promoters ISRE (green squares), OCT1_{—}04 (pink solid squares) and FOXD3_{—}01 (light blue solid squares) are positioned in the 2000 bp upstream region. The ISRE sequence is closer than the two other promoters and appears already in the 300 bp upstream region.

FIG. 25 is a volcano plot depicting the significance of changes between responders and non-responders in expression levels of various genes in PBMC of HCV type 2-4 at time 0 (being naïve to interferon treatment). Data was downloaded from the Gene Expression Omnibus Accession No. GSE 11190. “X” and “Y” axes and vertical and horizontal lines are as described with respect to FIG. 4. Note the significant downregulation in expression level of the inhibitory KIR genes (e.g., KIR2DL5A, KIR2DL5B, KIR2DL3, KIR3DL1, KIR2DL1, KIR2DL2, KIR3DL3) in PBMC of responders HCV type 2-4 patients as compared to non-responders, similar to the expression pattern of these genes in subjects infected with HCV type 1.

FIG. 26 is a volcano plot depicting the significance of changes between responders and non-responders in expression levels of various genes in liver tissue of HCV type 2-4 at time 0 (being naïve to interferon treatment). Data was downloaded from the Gene Expression Omnibus Accession No. gse11190. “X” and “Y” axes and vertical and horizontal lines are as described with respect to FIG. 4. Note the significant downregulation in expression level of the HCV type 1 five switch genes i.e., IFI27, ISG15, IFIT1, OAS3 and OAS2 in responder HCV type 2-4 patients as compared to non-responders.

### DESCRIPTION OF SPECIFIC EMBODIMENTS OF THE INVENTION

The present invention, in some embodiments thereof, relates to methods of predicting responsiveness to interferon treatment and methods of treating hepatitis C infection.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventor has identified signature genes which can be used to predict responsiveness of a subject to interferon treatment.

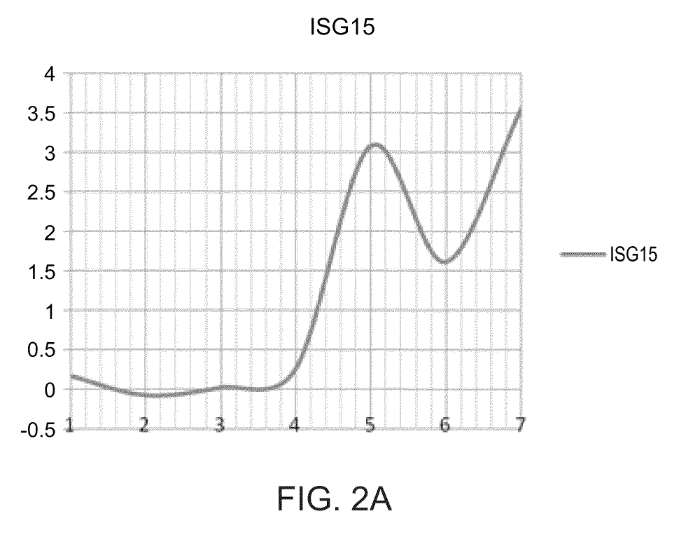

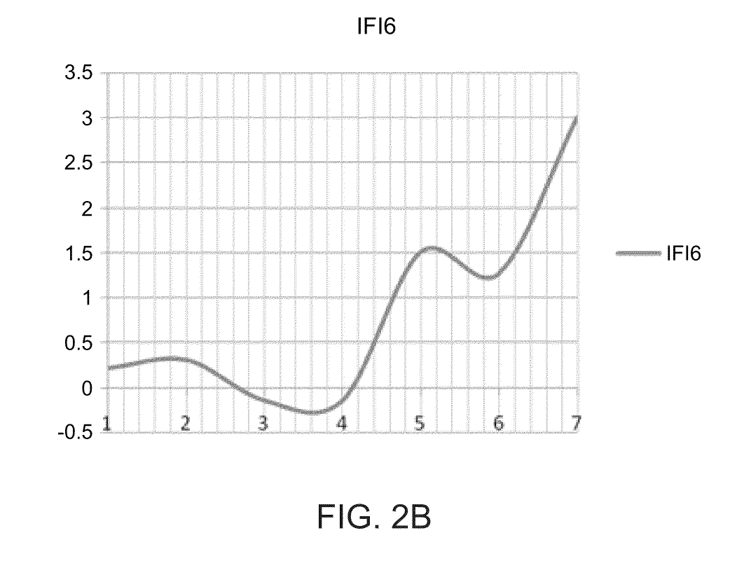

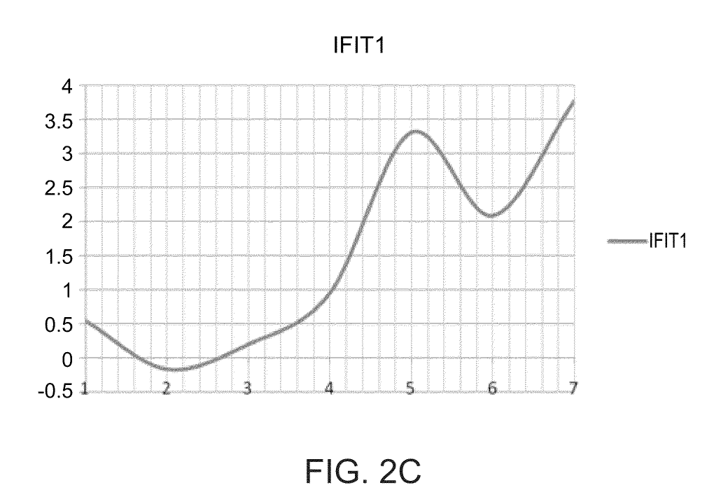

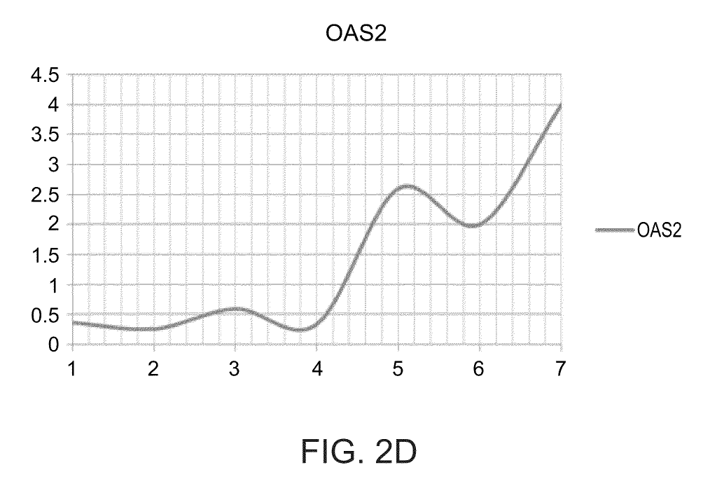

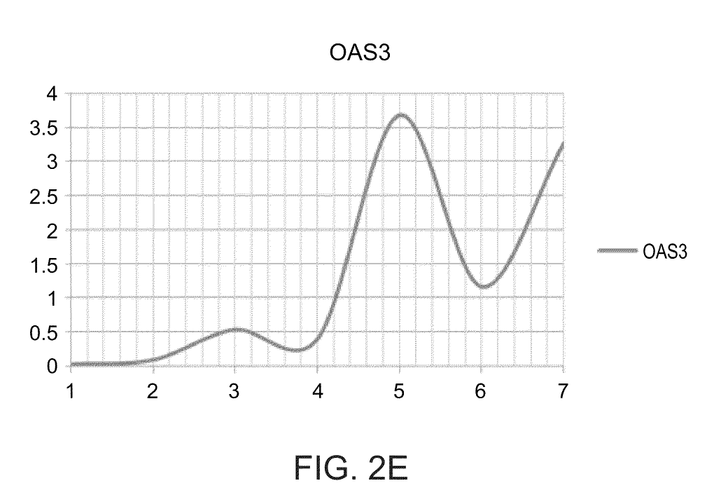

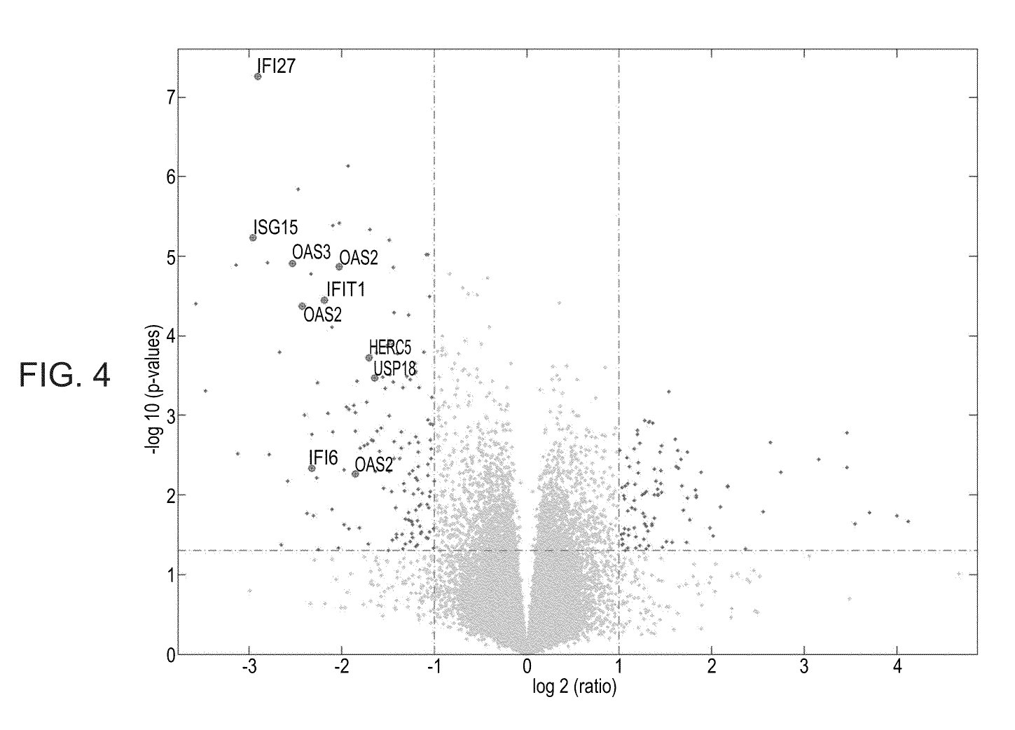

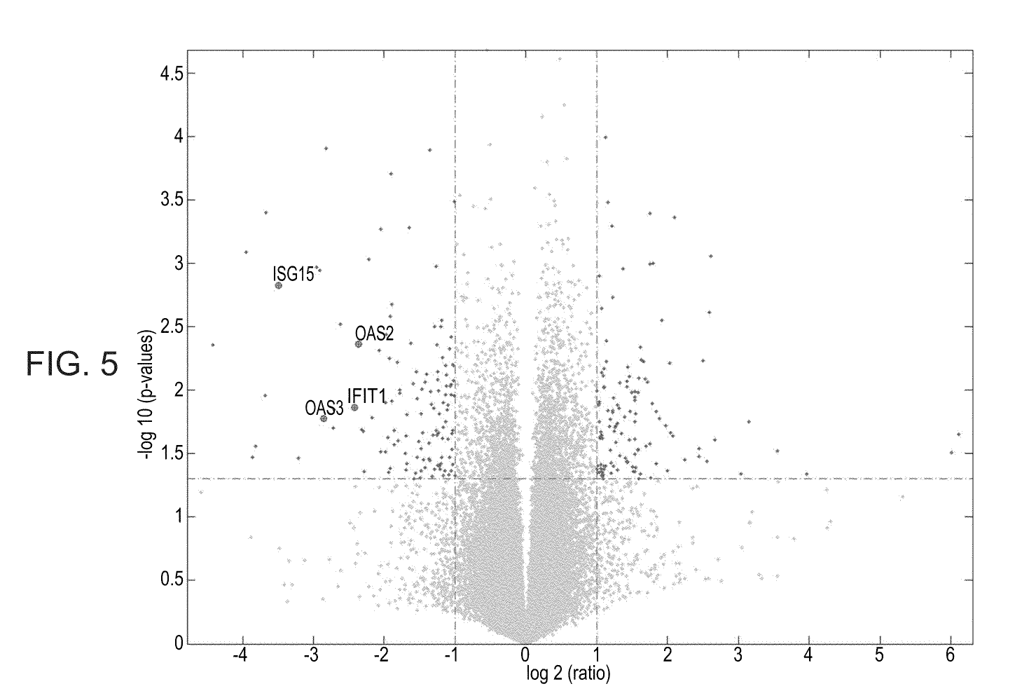

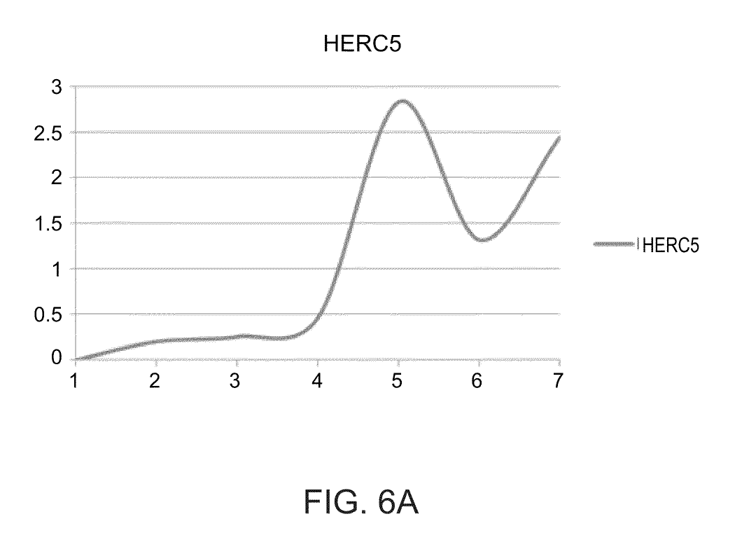

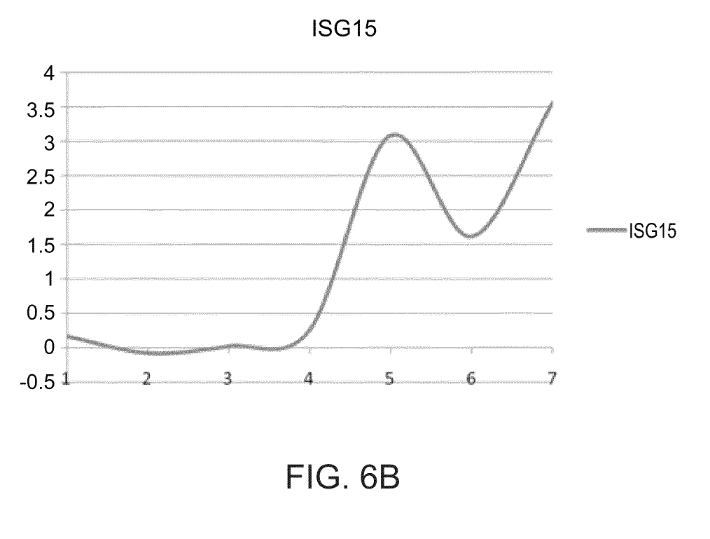

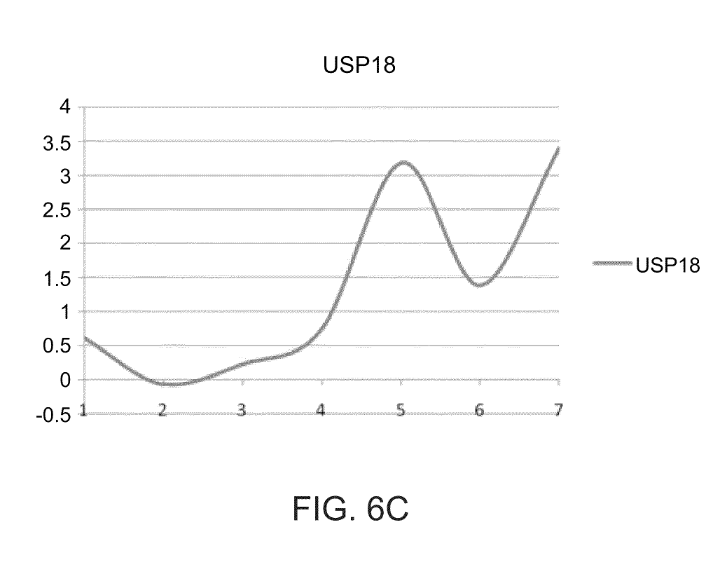

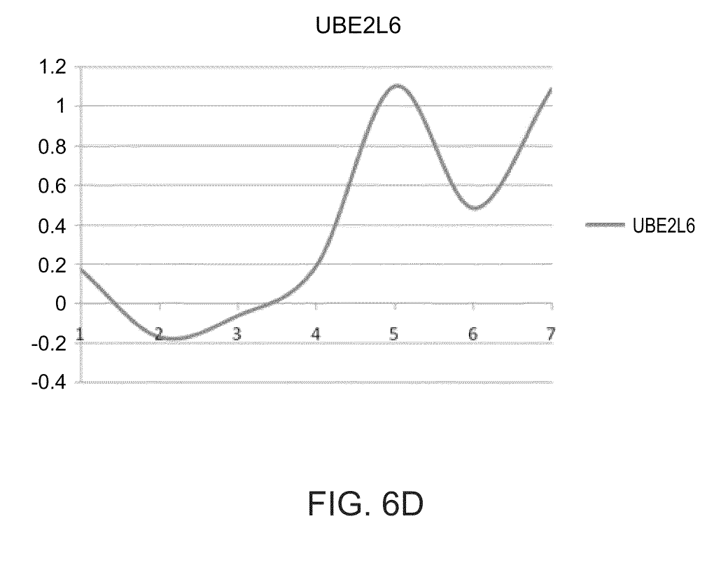

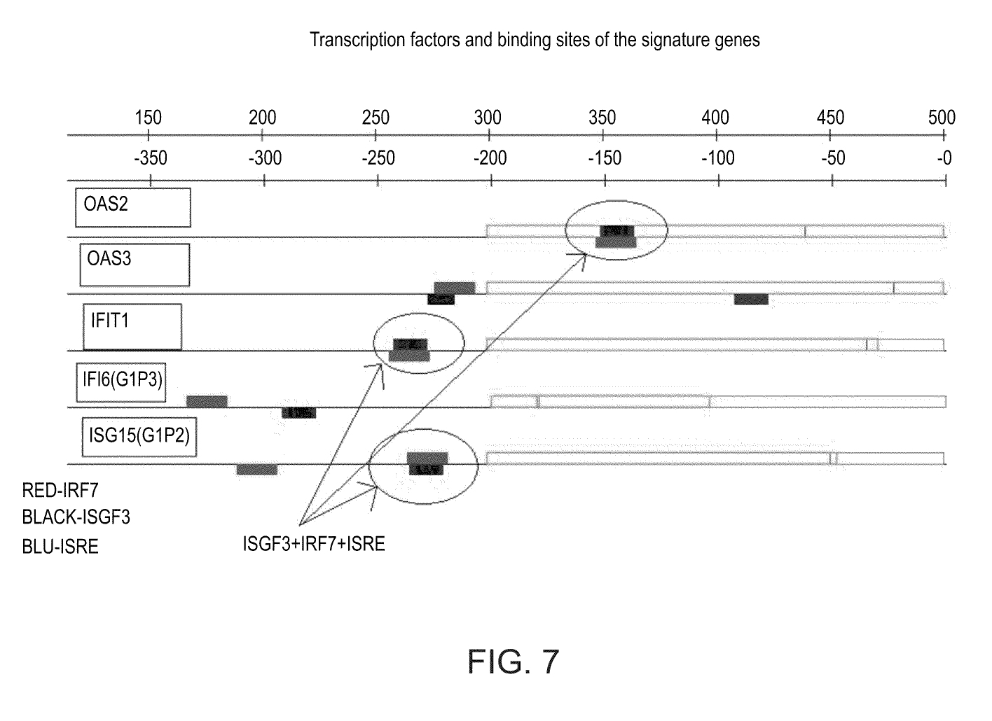

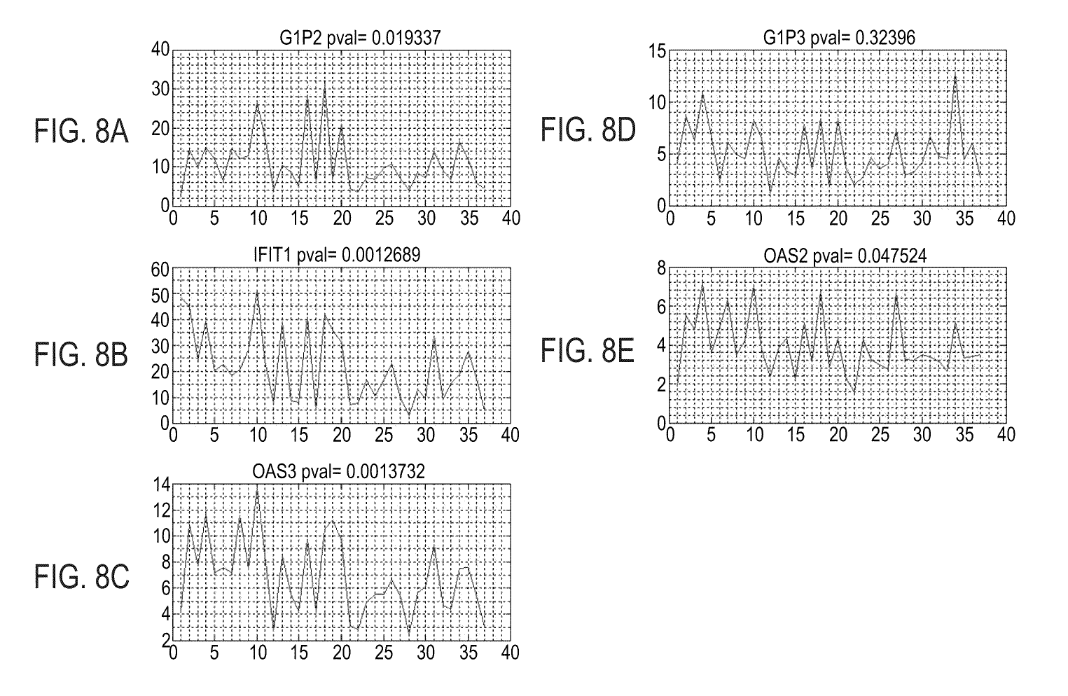

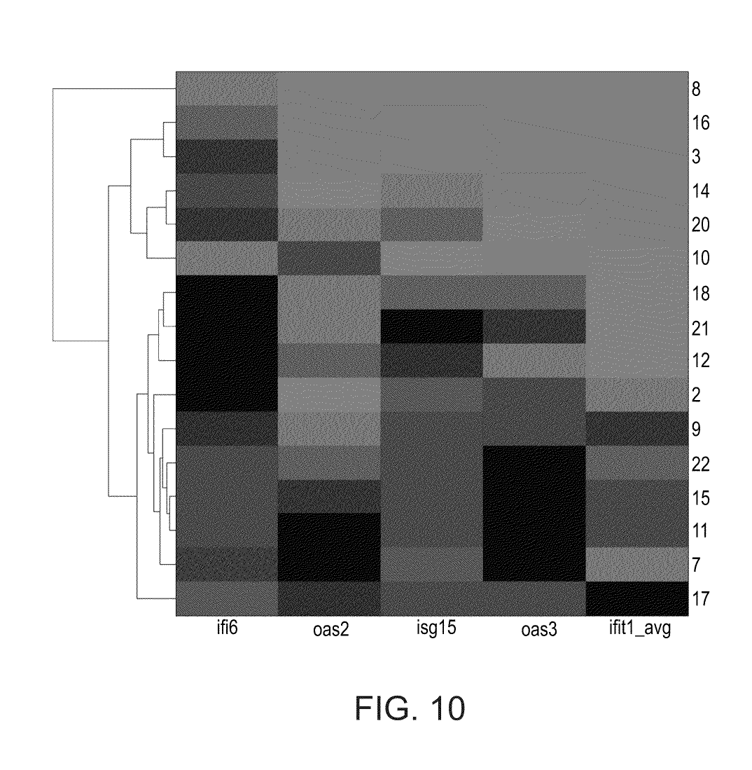

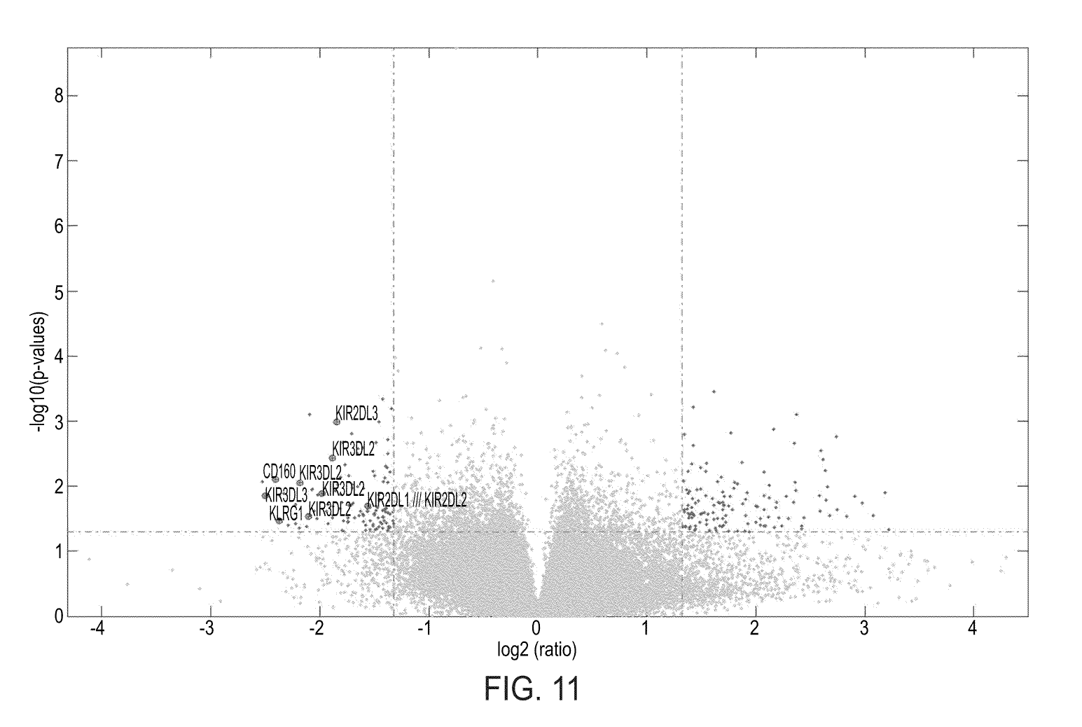

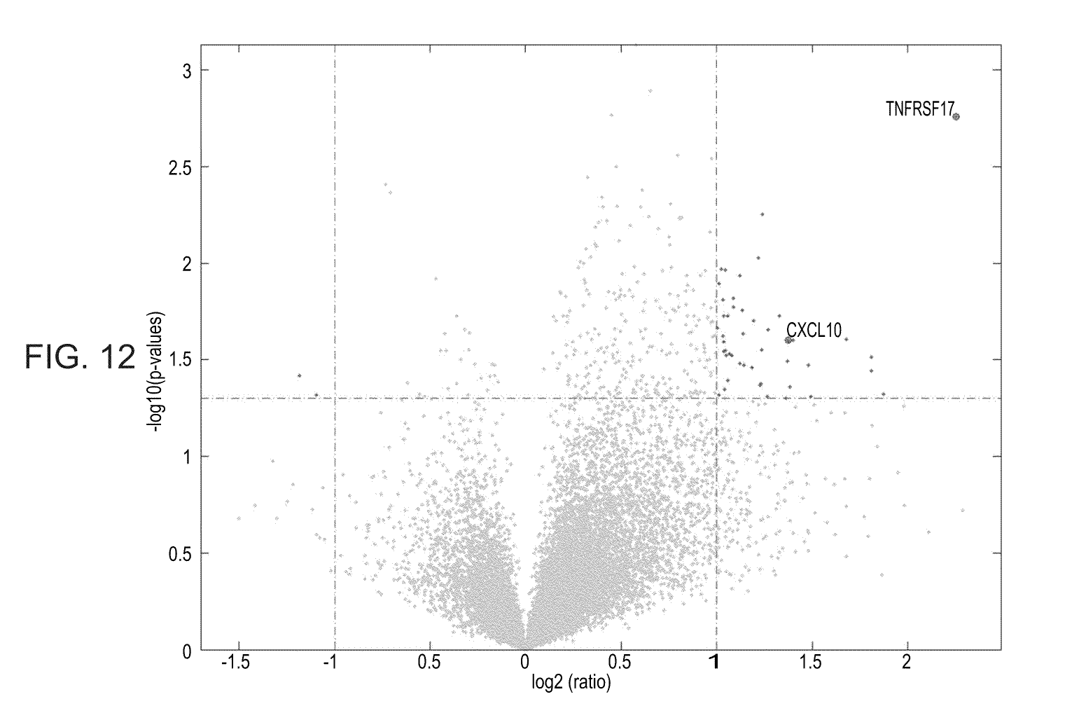

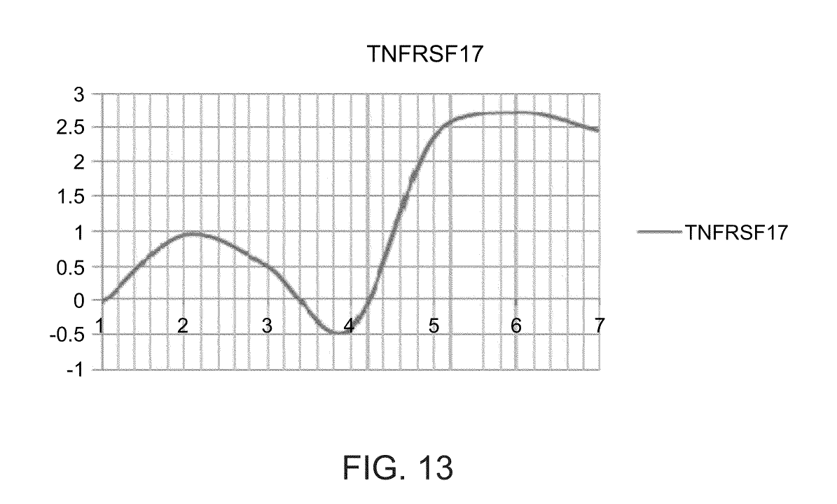

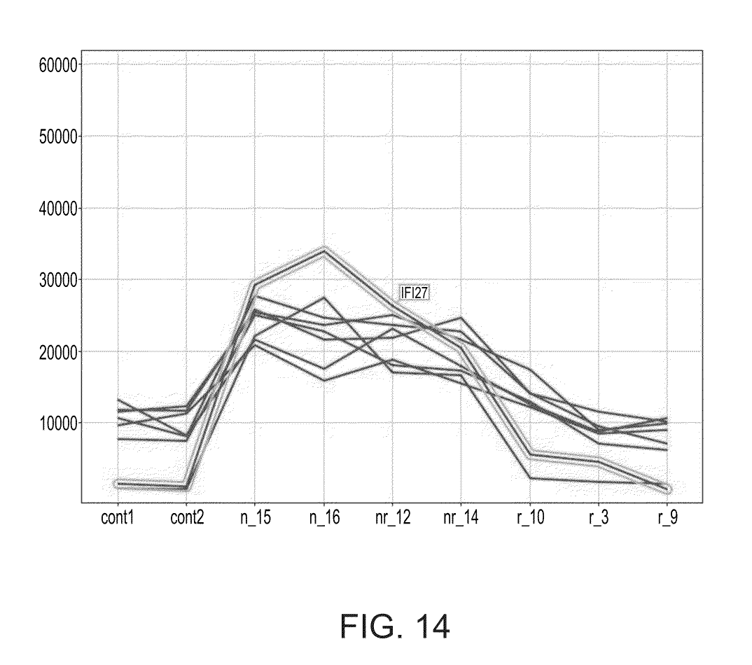

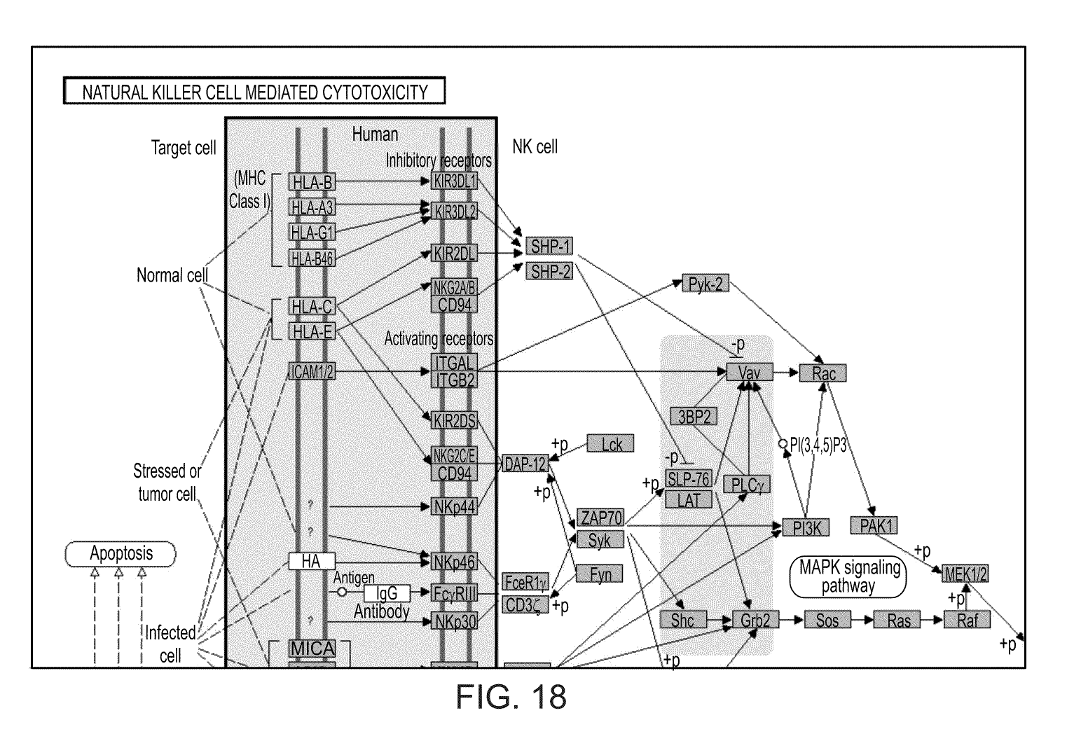

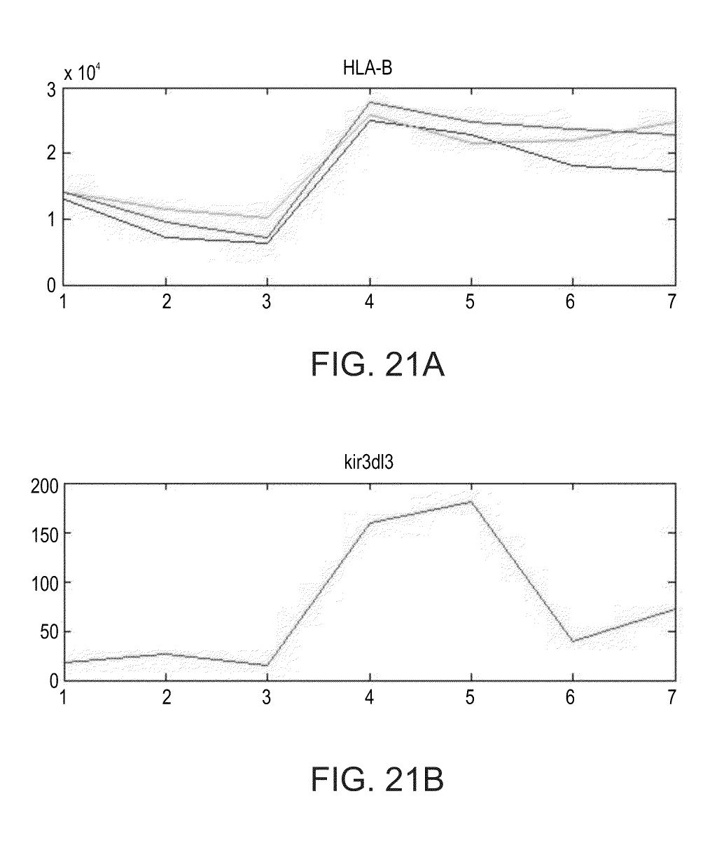

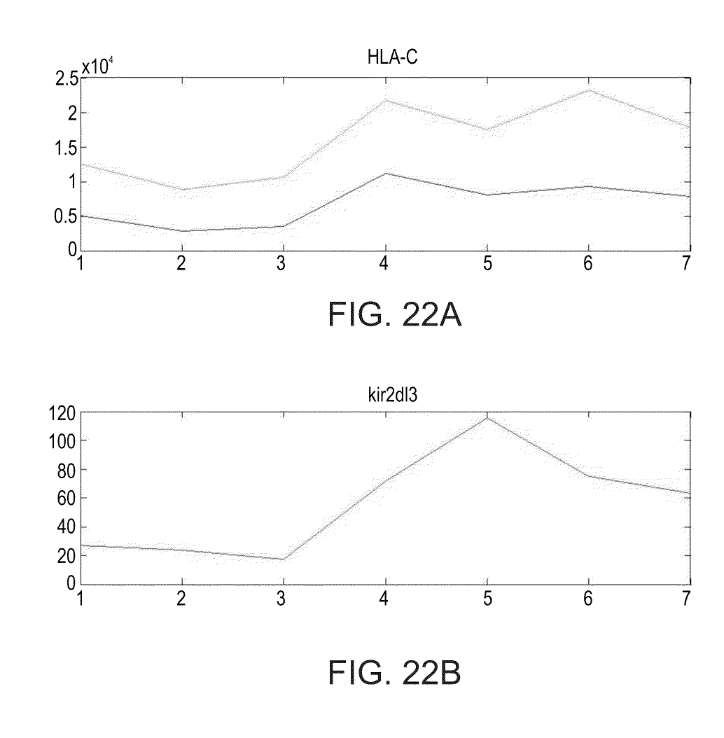

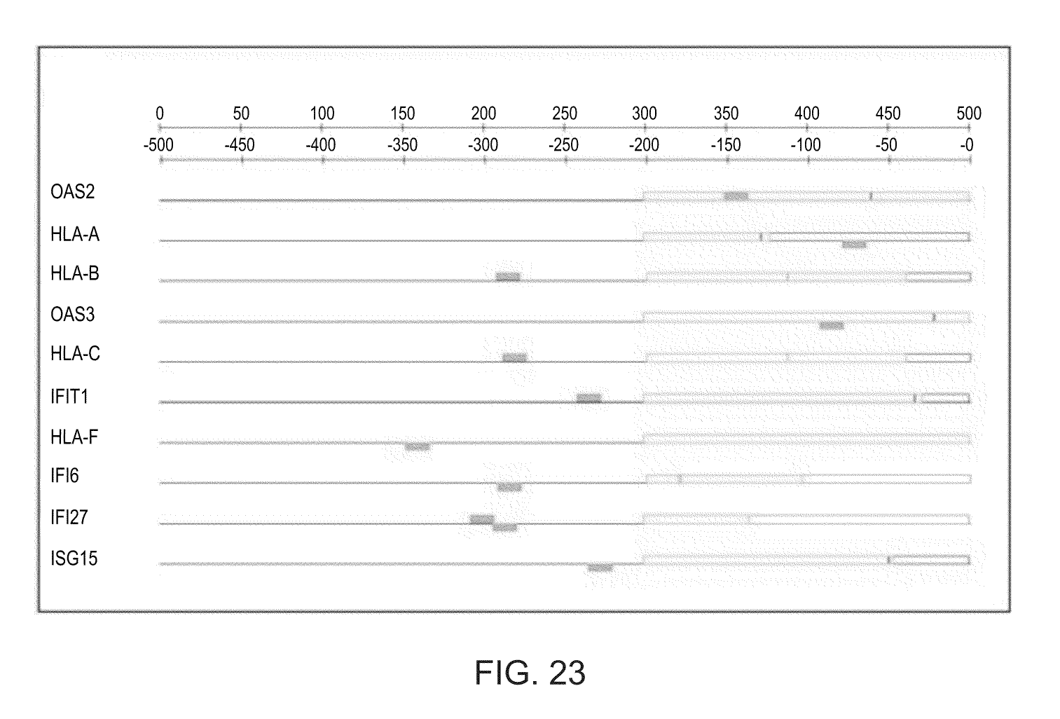

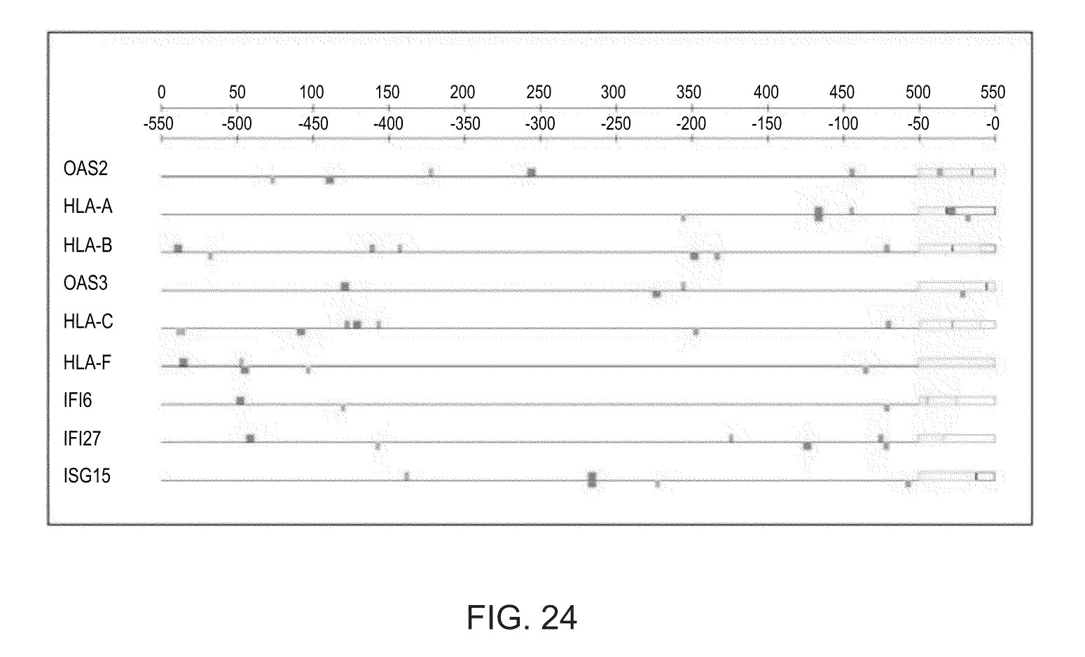

Thus, as shown in the Examples section which follows, the present inventor used a statistical scoring tool to identify genes which affect interferon response in a subject and found that the expression level of the 5-signature genes ISG15, IFIT1, IFI6, OAS2 and OAS3 in the liver can be used to predict response to interferon in all types of HCV virus infections, i.e., types 1-4, wherein upregulation of the level of expression before interferon treatment indicates that the subject will not response to interferon treatment (Example 4, FIGS. 4 and 5); that the ratio between the expression level of ISG15, IFI6, IFIT1, OAS2 and OAS3 as determined in liver biopsies following interferon treatment is significantly higher among interferon responders as compared to interferon non-responders of HCV type 1 subjects (Example 3, FIGS. 2A-E, Table 4); that upregulation of the HERC5, ISG15, USP18 and UBE2L6 genes of the ISGylation process in the liver following interferon treatment predicts responsiveness to interferon treatment (Example 5, FIGS. 6A-D, Table 6); that the signature genes IFI6, OAS2, ISG15, OAS3 and IFIT1 can predict the response to interferon treatment in subjects diagnosed with multiple sclerosis (Example 8 and FIG. 10); that the 5-signature genes (G1P2, G1P3, IFIT1, OAS2 and OSA3) and the TLR7-mediated pathway genes (TICAM1, MYD88, TLR7, TRAFD1, IRF7) are upregulated in blood of responders following the first interferon injection (Example 7, FIGS. 8A-E and 9A-E); and that the natural killer (NK) receptor genes KIR2DL1, KIR2DL2, KIR2DL3, KIR3DL1, KIR3DL2, KIR3DL3, KLRG1, KIR3DS1 and CD160 are upregulated in PBMC of HCV type 1 patients who are non-responders to interferon as compared to responders, thus, expression of these genes indicates poor prognosis of a subject infected with the HCV virus (Example 9, FIG. 11 and Tables 8 and 9). In addition, as shown in Example 11 (Tables 10-12, FIGS. 14, 15, 16) the expression pattern of the HLA family of genes (e.g., HLA-A, HLA-B, HLA-C, HLA-F, HLA-G) and of IFI27 was found to be similar to that of ISG15 in liver tissues of HCV type 1 patients prior to treatment. Moreover, the present inventor found that prior to treatment (in subjects naïve to interferon treatment) there is a coordinated upregulation of the HLA genes in the liver and the kir genes in the blood of HCV type 1 non-responders, as well as upregulation of the kir genes in blood samples of non-responders HCV types 2-4 patients prior to treatment (Example 12, FIGS. 18, 19, 20, 21, 22, 25, 26). Furthermore, the present inventor found that upregulation of the expression level of TNFRSF17, and optionally also of CXCL10 and CD24, following interferon treatment predicts the success of interferon treatment (Example 10, FIGS. 12 and 13). In addition, the present inventor uncovered that ISRE promoter is common to all of the signature genes (e.g., ISG15, IFI6, IFIT1, OAS2, OAS3, HLA-A, HLA-B, HLA-C and HLA-F) involved in determining the fate of interferon treatment (Example 6, FIG. 7; Example 13, FIG. 23, Tables 14 and 15).

Thus, according to an aspect of some embodiments of the invention there is provided a method of predicting responsiveness of a subject to interferon treatment. The method is effected by comparing a level of expression in a cell of the subject of at least one gene selected from the group consisting KIR3DL3, KIR3DL2, KIR3DL1, KIR2DL1, KIR2DL2, KIR2DL3, KLRG1, KIR3DS1, CD160, HLA-A, HLA-B, HLA-C, HLA-F, HLA-G and IFI27 to a reference expression data of the at least one gene obtained from at least one interferon responder subject and/or at least one interferon non-responder subject, thereby predicting the responsiveness of the subject to interferon treatment.

As used herein the term “interferon” or “IFN” which is interchangeably used herein, refers to a synthetic, recombinant or purified interferon, and encompasses interferon type I [in human include IFN-α (GenBank Accession No. NM_{—}024013 and NP_{—}076918; SEQ ID NOs:165 and 169 respectively), interferon alpha 2a (GenBank Accession No. NM_{—}000605 and NP_{—}000596; SEQ ID NO:173 and 174, respectively), IFN-β (GenBank Accession No. NM_{—}002176 and NP_{—}002167; SEQ ID NOs:166 and 170 respectively), interferon beta 1a [AVONEX (Biogen Idec); REBIF (EMD Serono)] or interferon beta 1b (BETASERON) and IFN-ω) (GenBank Accession No. NM_{—}002177 and NP_{—}002168; SEQ ID NOs:167 and 171 respectively)], which bind to the cell surface receptor complex IFN-a receptor (IFNAR) consisting of IFNAR1 and IFNAR2 chains; interferon type II [in human is IFN-γ (GenBank Accession No. NM_{—}000619 and NP_{—}000610; SEQ ID NOs:168 and 172 respectively)], which binds to the IFNGR receptor; and interferon type III, which bind to a receptor complex consisting of IL10R2 (also called CRF2-4) and IFNLR1 (also called CRF2-12).

As used herein the phrase “interferon treatment” refers to administration of interferon into a subject in need thereof. It should be noted that administration of interferon may comprise a single or multiple dosages, as well as a continuous administration, depending on the pathology to be treated and the subject receiving the treatment.

Interferon is used in the treatment of various pathologies such as autoimmune disorders (e.g., multiple sclerosis using e.g., interferon beta-1a and/or interferon beta-1b), various cancers (e.g., hematological malignancy, leukemia and lymphomas including hairy cell leukemia, chronic myeloid leukemia, nodular lymphoma, cutaneous T-cell lymphoma, recurrent melanomas, using e.g., recombinant IFN-α2b), and viral infections (e.g., hepatitis C virus infection, hepatitis B virus infection, viral respiratory diseases such as cold and flu).

Various modes of interferon administration are known in the art. These include, but are not limited to, injection (e.g., using a subcutaneous, intramuscular, intravenous, or intradermal injection), intranasal administration and oral administration.

According to some embodiments of the invention, interferon treatment is provided to the subject in doses matching his weight, at a frequency of once a week, for a period of up to 48 weeks.

According to some embodiments of the invention, the interferon treatment comprises type I interferon such as interferon alpha (SEQ ID NO:169), interferon alpha 2a (SEQ ID NO:174), interferon beta 1a or interferon beta 1b.

According to some embodiments of the invention, the interferon treatment comprises PEGylated interferon [i.e., conjugated to a polyethylene glycol (PEG) polymer].

According to some embodiments of the invention, the interferon treatment comprises interferon and ribavirin.

The term “subject” as used herein refers to a mammal, preferably a human being (male or female) at any age.

According to some embodiments of the invention, the subject is diagnosed with a pathology (disease, disorder or condition) which requires interferon treatment such as an autoimmune disease, a viral infection or cancer as described above.

According to some embodiments of the invention, the subject is diagnosed with hepatitis C virus (HCV) infection.

As used herein the term “HCV” refers to hepatitis C virus having genotype 1 (also known as HCV Type 1), genotype 2 (also known as HCV Type 2), genotype 3 (also known as HCV Type 3), genotype 4 (also known as HCV Type 4), genotype 5 (also known as HCV Type 5) or genotype 6 (also known as HCV Type 6).

The phrase “HCV infection” encompasses acute (refers to the first 6 months after infection) and chronic (refers to infection with hepatitis C virus which persists more than 6 month) infection with the hepatitis C virus.

According to some embodiments of the invention, the subject is diagnosed with chronic HCV infection.

According to some embodiments of the invention, the subject is infected with HCV type 1.

According to some embodiments of the invention, the subject is infected with HCV type 2, 3 or 4

According to some embodiments of the invention, the subject is diagnosed with multiple sclerosis.

As used herein the phrase “multiple sclerosis” refers to a pathology characterized by presence of at least two neurological attacks affecting the central nervous system (CNS) and accompanied by demyelinating lesions on brain magnetic resonance imaging (MRI). The disease course of patients diagnosed with multiple sclerosis can be a relapsing-remitting multiple sclerosis (RRMS) (occurring in 85% of the patients) or a progressive multiple sclerosis (occurring in 15% of the patients).

According to some embodiments of the invention, the subject is diagnosed with RRMS.

According to some embodiments of the invention, the subject is a healthy subject (e.g., not diagnosed with any disease which require interferon treatment). It should be noted that determining the responsiveness of a healthy subject to interferon treatment can be performed as part of a genetic testing of the healthy subject, which can be recorded in the subject's medical file for future use (e.g., in case the subject will be diagnosed with a disease requiring interferon treatment).

As used herein the phrase “predicting responsiveness of a subject to interferon treatment” refers to determining the likelihood that the subject will respond to interferon treatment, e.g., the success or failure of interferon treatment.

The term “response” to interferon treatment refers to an improvement in at least one relevant clinical parameter as compared to an untreated subject diagnosed with the same pathology (e.g., the same type, stage, degree and/or classification of the pathology), or as compared to the clinical parameters of the same subject prior to interferon treatment.

Typically only 50% of HCV type I and MS patients respond to interferon treatment. Therefore a “low probability of response to interferon” in connection with these diseases is a probability significantly lower than about 50%, e.g., a probability lower than about 40%, e.g., a probability lower than about 30%, e.g., a probability lower than about 20%, e.g., a probability lower than about 10% or 5%, and a “high probability of response to interferon” is a probability significantly higher than about 50%, e.g., a probability higher than about 60%, e.g., a probability higher than about 70%, e.g., a probability higher than about 80%, e.g., a probability higher than about 85%, e.g., a probability higher than about 90%, e.g., a probability higher than about 95%, e.g., a probability higher than about 99%.

In connection with HCV types 2, 3, 4 the typical rate of success of interferon treatment is about 80%, so a low probability of success is a probability lower than about 80%, e.g., a probability lower than about 70%, e.g., a probability lower than 60 about %, e.g., a probability lower than 50 about %, e.g., a probability lower than 40 about %, e.g., a probability lower than 30 about %, e.g., lower than 20 about %, e.g., a probability lower than about 10%, and a high probability is a probability higher than about 80%, e.g., a probability higher than about 90%, e.g., a probability higher than about 95%, e.g., a probability higher than about 99%.

For example, a successful interferon treatment in HCV patients can result in clearance of the virus from the subject's body (e.g., from the blood), decreased probability of liver damage and/or cirrhosis, and decreased probability of hepatocellular carcinoma. In addition, if HCV infection is diagnosed immediately after infection, interferon treatment can results in clearance of the virus from the body and prevention of chronic hepatitis C.

For example, a successful interferon treatment in multiple sclerosis patients can result in slowing disease progression and activity in relapsing-remitting multiple sclerosis and reducing attacks in secondary progressive multiple sclerosis.

In HCV infected subjects, the responsiveness to interferon can be evaluated by measuring virus load in blood, and presence and/or level of HCV RNA in liver cells or blood cells of the subject. Such tests can be done prior to interferon treatment, during interferon treatment and at a predetermined period after completion of the treatment course with interferon.

In multiple sclerosis subjects, the responsiveness to interferon can be evaluated using the Kurtzke Expanded Disability Status Scale (EDSS) of the subject which quantifies disability in MS by scoring eight Functional Systems (FS) (pyramidal, cerebellar, brainstem, sensory, bowel and bladder, visual, cerebral, and other), the number or relapses per year and/or the severity thereof.

According to some embodiments of the invention an HCV infected subject is considered an interferon responder when exhibiting a complete clearance of the hepatitis C virus from the subject's body (e.g., tissues, cells, body fluid) as determined within one about month after beginning of interferon treatment, e.g., within about 2 months, e.g., within about 3 months, e.g., within about 4 months, e.g., within about 5 months, e.g., within about 6 months, e.g., within about 7 months, e.g., within about 8 months, e.g., within about 9 months, e.g., within about 10 months, e.g., within about 11 months, e.g., within about 12 months, e.g., within about 3 months, e.g., within about 11 months, e.g., within about 12 months, e.g., within about 13 months, e.g., within about 14 months, e.g., within about 15 months, e.g., within about 16 months, e.g., within about 17 months, e.g., within about 18 months, e.g., within about 19 months, e.g., within about 20 months, e.g., within about 21 months, e.g., within about 22 months, e.g., within about 23 months, e.g., within about 24 after beginning of interferon treatment.

According to some embodiments of the invention an HCV infected subject is considered an interferon non-responder when exhibiting persistent levels [e.g., levels which are similar (±about 10-20%) or not significantly reduced as compared to before interferon treatment] of the hepatitis C virus in the subject's body (e.g., tissues, cells, body fluid) as determined at least about one month after beginning of interferon treatment, e.g., at least about 2 months, e.g., at least about 3 months, e.g., at least about 4 months, e.g., at least about 5 months, e.g., at least about 6 months, e.g., at least about 7 months, e.g., at least about 8 months, e.g., at least about 9 months, e.g., at least about 10 months, e.g., at least about 11 months, e.g., at least about 12 months, e.g., at least about 13 months, e.g., at least about 14 months, e.g., at least about 15 months, e.g., at least about 16 months, e.g., at least about 17 months, e.g., at least about 18 months, e.g., at least about 19 months, e.g., at least about 20 months, e.g., at least about 21 months, e.g., at least about 22 months, e.g., at least about 23 months, e.g., at least about 24 months, or more after beginning of interferon treatment.

As mentioned, the method according to this aspect of the invention is effected by comparing a level of expression in a cell of the subject of at least one gene selected from the group consisting KIR3DL3, KIR3DL2, KIR3DL1, KIR2DL1, KIR2DL2, KIR2DL3, KLRG1, KIR3DS1, CD160, HLA-A, HLA-B, HLA-C, HLA-F, HLA-G and IFI27 to a reference expression data of the at least one gene obtained from at least one interferon responder subject and/or at least one interferon non-responder subject, thereby predicting the responsiveness of the subject to interferon treatment.

As used herein, the phrase “level of expression” refers to the degree of gene expression and/or gene product activity in a specific cell. For example, up-regulation or down-regulation of various genes can affect the level of the gene product (i.e., RNA and/or protein) in a specific cell.

Sequence information regarding gene products (i.e., RNA transcripts and polypeptide sequences) of KIR2DL1, KIR2DL2, KIR2DL3, KIR3DL1, KIR3DL2, KIR3DL3, KLRG1, KIR3DS1, CD160, HLA-A, HLA-B, HLA-C, HLA-F, HLA-G and IFI27, can be found in Table 16 in the Examples section which follows. In addition, probes which can be used to detect transcripts of these genes are provided in Table 16 (Examples section).

It should be noted that the level of expression can be determined in arbitrary absolute units, or in normalized units (relative to known expression levels of a control reference). For example, when using DNA chips, the expression levels are normalized according to the chips' internal controls or by using quantile normalization such as RMA.

As used herein the phrase “a cell of the subject” refers to any cell (e.g., an isolated cell), cell culture, cell content and/or cell secreted content which contains RNA and/or proteins of the subject. Examples include a blood cell, a cell obtained from any tissue biopsy [e.g., liver biopsy, cerebrospinal fluid, (CSF), brain biopsy], a bone marrow cell, body fluids such as plasma, serum, saliva, spinal fluid, lymph fluid, the external sections of the skin, respiratory, intestinal, and genitourinary tracts, tears, saliva, sputum and milk. According to an embodiment of the invention, the cell is a blood cell (e.g., white blood cells, macrophages, B- and T-lymphocytes, monocytes, neutrophiles, eosinophiles, and basophiles) which can be obtained using a syringe needle from a vein of the subject. It should be noted that the cell may be isolated from the subject (e.g., for in vitro detection) or may optionally comprise a cell that has not been physically removed from the subject (e.g., in vivo detection).

According to some embodiments of the invention, the white blood cell comprises peripheral blood mononuclear cells (PBMC). The phrase, “peripheral blood mononuclear cells (PBMCs)” as used herein, refers to a mixture of monocytes and lymphocytes. Several methods for isolating white blood cells are known in the art. For example, PBMCs can be isolated from whole blood samples using density gradient centrifugation procedures. Typically, anticoagulated whole blood is layered over the separating medium. At the end of the centrifugation step, the following layers are visually observed from top to bottom: plasma/platelets, PBMCs, separating medium and erythrocytes/granulocytes. The PBMC layer is then removed and washed to remove contaminants (e.g., red blood cells) prior to determining the expression level of the polynucleotide(s) therein.

According to some embodiments of the invention, the cell is a liver cell.

It should be noted that liver cells (hepatic cell) can be obtained by a liver biopsy (e.g., using a surgical tool or a needle).

According to some embodiments of the invention, the level of expression of the gene(s) of the invention is determined using an RNA or a protein detection method.

According to some embodiments of the invention, the RNA or protein molecules are extracted from the cell of the subject.

Methods of extracting RNA or protein molecules from cells of a subject are well known in the art. Once obtained, the RNA or protein molecules can be characterized for the expression and/or activity level of various RNA and/or protein molecules using methods known in the arts.

Non-limiting examples of methods of detecting RNA molecules in a cell sample include Northern blot analysis, RT-PCR, RNA in situ hybridization (using e.g., DNA or RNA probes to hybridize RNA molecules present in the cells or tissue sections), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface [e.g., a glass wafer) with addressable location, such as Affymetrix microarray (Affymetrix®, Santa Clara, Calif.)].

Non-limiting examples of methods of detecting the level and/or activity of specific protein molecules in a cell sample include Enzyme linked immunosorbent assay (ELISA), Western blot analysis, radio-immunoassay (RIA), Fluorescence activated cell sorting (FACS), immunohistochemical analysis, in situ activity assay (using e.g., a chromogenic substrate applied on the cells containing an active enzyme), in vitro activity assays (in which the activity of a particular enzyme is measured in a protein mixture extracted from the cells). For example, in case the detection of the expression level of a secreted protein is desired, ELISA assay may be performed on a sample of fluid obtained from the subject (e.g., serum), which contains cell-secreted content.

As used herein the phrase “reference expression data” refers to the expression level of the gene in a cell of a subject whose responsiveness to interferon is already known (e.g., a reference responder or non-responder subject). Such as an expression level can be known from the literature, from the database [e.g., using the Gene Expression Omnibus at Hypertext Transfer Protocol://World Wide Web (dot) ncbi (dot) nlm (dot) nih (dot) gov/projects/geo/], or from biological samples comprising RNA or protein molecules obtained from a reference responder or non-responder subject.

According to some embodiments of the invention, the reference expression data is obtained from at least one interferon responder subject (e.g., from one interferon responder subject), e.g., from at least 2, from at least 3, from at least 4, from at least 5, from at least 6, from at least 7, from at least 8, from at least 9, from at least 10, from at least 20, from at least 30, from at least 40, from at least 50, from at least 100 or more interferon responder subjects.

According to some embodiments of the invention, the reference expression data is obtained from at least one interferon non-responder subject (e.g., from one interferon non-responder subject), e.g., from at least 2, from at least 3, from at least 4, from at least 5, from at least 6, from at least 7, from at least 8, from at least 9, from at least 10, from at least 20, from at least 30, from at least 40, from at least 50, from at least 100 or more interferon non-responder subjects.

It should be noted that when more than one reference subjects (i.e., interferon responders or non-responders) is used, the reference expression data may comprise an average of the expression level of several or all subjects, and those of skills in the art are capable of averaging expression levels from 2 or more subject, using e.g., normalized expression values.

According to some embodiments of the invention, a decrease above a predetermined threshold in the level of expression of the at least one gene in the cell of the subject relative to the reference expression data of the at least one gene obtained from the at least one interferon non-responder subject predicts responsiveness of the subject to interferon treatment, e.g., classifies the subject as responsive to interferon treatment (e.g., indicates that the subject is an interferon responder).

As used herein the phrase “a decrease above a predetermined threshold” refers to a decrease in the level of expression in the cell of the subject relative to the reference expression data obtained from the at least one interferon non-responder subject which is higher than a predetermined threshold such as a about 10%, e.g., higher than about 20%, e.g., higher than about 30%, e.g., higher than about 40%, e.g., higher than about 50%, e.g., higher than about 60%, higher than about 70%, higher than about 80%, higher than about 90%, higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times, higher than about 350, higher than about 500 times, higher than about 1000 times, or more relative to the reference expression data obtained from the at least one interferon non-responder subject.

According to some embodiments of the invention, an increase above a predetermined threshold in the level of expression of the at least one gene in the cell of the subject relative to the reference expression data of the at least one gene obtained from the at least one interferon responder subject predicts lack of responsiveness of the subject to interferon treatment, e.g., classifies the subject as non-responsive to interferon treatment (e.g., indicates that the subject is an interferon non-responder).

As used herein the phrase “an increase above a predetermined threshold” refers to an increase in the level of expression in the cell of the subject relative to the reference expression data obtained from the at least one interferon responder subject which is higher than a predetermined threshold such as a about 10%, e.g., higher than about 20%, e.g., higher than about 30%, e.g., higher than about 40%, e.g., higher than about 50%, e.g., higher than about 60%, higher than about 70%, higher than about 80%, higher than about 90%, higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times, higher than about 350, higher than about 500 times, higher than about 1000 times, or more relative to the reference expression data obtained from the at least one interferon responder subject.

According to some embodiments of the invention, when a level of expression of the at least one gene in the cell of the subject is identical or changed below a predetermined threshold as compared to the reference expression data of the at least one gene obtained from the at least one interferon responder subject, then the subject is classified as responsive to interferon (e.g., indicates that the subject is an interferon responder).

As used herein the phrase “changed below a predetermined threshold” refers to an increase or a decrease in the level of expression in the cell of the subject relative to the reference expression data obtained from the at least one interferon responder subject which is lower than a predetermined threshold, such as lower than about 10 times, e.g., lower than about 9 times, e.g., lower than about 8 times, e.g., lower than about 7 times, e.g., lower than about 6 times, e.g., lower than about 5 times, e.g., lower than about 4 times, e.g., lower than about 3 times, e.g., lower than about 2 times, e.g., lower than about 90%, e.g., lower than about 80%, e.g., lower than about 70%, e.g., lower than about 60%, e.g., lower than about 50%, e.g., lower than about 40%, e.g., lower than about 30%, e.g., lower than about 20%, e.g., lower than about 10%, e.g., lower than about 9%, e.g., lower than about 8%, e.g., lower than about 7%, e.g., lower than about 6%, e.g., lower than about 5%, e.g., lower than about 4%, e.g., lower than about 3%, e.g., lower than about 2%, e.g., lower than about 1% relative to the reference expression data obtained from the at least one interferon responder subject.

According to some embodiments of the invention, when a level of expression of the at least one gene in the cell of the subject is identical or changed below a predetermined threshold as compared to the reference expression data of the at least one gene obtained from the at least one interferon non-responder subject, then the subject is classified as a non-responsive to interferon.

As used herein the phrase “changed below a predetermined threshold” refers to an increase or a decrease in the level of expression in the cell of the subject relative to the reference expression data obtained from the at least one interferon non-responder subject which is lower than a predetermined threshold, such as lower than about 10 times, e.g., lower than about 9 times, e.g., lower than about 8 times, e.g., lower than about 7 times, e.g., lower than about 6 times, e.g., lower than about 5 times, e.g., lower than about 4 times, e.g., lower than about 3 times, e.g., lower than about 2 times, e.g., lower than about 90%, e.g., lower than about 80%, e.g., lower than about 70%, e.g., lower than about 60%, e.g., lower than about 50%, e.g., lower than about 40%, e.g., lower than about 30%, e.g., lower than about 20%, e.g., lower than about 10%, e.g., lower than about 9%, e.g., lower than about 8%, e.g., lower than about 7%, e.g., lower than about 6%, e.g., lower than about 5%, e.g., lower than about 4%, e.g., lower than about 3%, e.g., lower than about 2%, e.g., lower than about 1% relative to the reference expression data obtained from the at least one interferon non-responder subject.

For example, as is shown in Table 8 (Example 7 of the Examples section), while the level of expression of the KIR3DL2 gene among interferon responders was on average of 31 arbitrary units, the level of expression of this gene among interferon non-responders was on average of 134 arbitrary units, which demonstrates an increase of more than about 4 times in cells of non-responders as compared to cells of responders. Similar findings with respect to additional genes are presented in Table 9 (Example 7 of the Examples section).

According to some embodiments of the invention the level of expression is determined prior to interferon treatment.

As used herein the phrase “prior to interferon treatment” refers to any time before the first administration of interferon to the subject. Thus, prior to interferon treatment the subject is considered naïve to interferon treatment.

According to some embodiments of the invention the at least one gene comprises at least two genes, wherein a first gene is selected from the group consisting of KIR3DL3, KIR3DL2, KIR3DL1, KIR2DL1, KIR2DL2, KIR2DL3, KLRG1, and CD160, and wherein a second gene is selected from the group consisting of HLA-A, HLA-B, HLA-C, HLA-F, HLA-G and IFI27.

According to some embodiments of the invention the level of expression of the first gene is determined in a blood cell.

According to some embodiments of the invention the level of expression of the second gene is determined in a liver cell.

According to some embodiments of the invention the level of expression of the at least one gene comprises at least two, at least 3, at least 4, at least 5, at least 6, at least 7, at least 8, at least 9, at least 10, at least 11, at least 12, at least 13, at least 14, at least 15 genes from the group of KIR3DL3, KIR3DL2, KIR3DL1, KIR2DL1, KIR2DL2, KIR2DL3, KLRG1, KIR3DS1, CD160, HLA-A, HLA-B, HLA-C, HLA-F, HLA-G and IFI27 genes.

According to an aspect of some embodiments of the invention there is provided a method of predicting responsiveness to interferon treatment of a subject diagnosed with multiple sclerosis or infected with HCV type 2, 3 or 4. The method is effected by comparing a level of expression in a cell of the subject of IFI6, OAS2, ISG15, OAS3 and IFIT1 genes to a reference expression data of the genes obtained from at least one interferon responder subject and/or at least one interferon non-responder subject, thereby predicting the responsiveness of the subject to interferon treatment.

According to some embodiments of the invention the cell is a blood cell.

According to some embodiments of the invention when the subject is diagnosed with multiple sclerosis the cell of the subject is a blood cell.

According to some embodiments of the invention when the subject is infected with HCV type 2, 3 or 4 the cell of the subject is a liver cell.

As mentioned above and further described in the Examples section which follows, the present inventor has uncovered that in addition to testing the expression level of genes prior to interferon treatment, there is a significant value, with a high predictive power, to test the expression of certain genes in a cell of a subject prior to interferon treatment and at a predetermined time period following interferon treatment, since the switch in gene expression immediately after interferon treatment is significant among interferon responders as compared to interferon non-responders.

According to an aspect of some embodiments of the invention there is provided a method of predicting responsiveness of a subject to interferon treatment. The method is effected by comparing a ratio determined between an expression level of TNFRSF17 gene in a cell of the subject following interferon treatment and an expression level of the gene in the cell of the subject prior to interferon treatment, or visa versa, namely, comparing a ratio determined between an expression level of TNFRSF17 gene in a cell of the subject prior to interferon treatment and an expression level of the gene in the cell of the subject following interferon treatment, to a reference ratio determined in a cell of at least one interferon responder subject and/or at least one interferon non-responder subject, the reference ratio is determined between an expression level of the gene following interferon treatment and an expression level of the gene prior to interferon treatment, or visa versa, namely, the reference ratio is determined between an expression level of the gene prior to interferon treatment and an expression level of the gene following interferon treatment, thereby predicting the responsiveness to interferon treatment of a subject.

According to some embodiments of the invention, the method further comprising comparing a ratio determined between an expression level of at least one gene selected from the group consisting of CXCL10 and CD24 in a cell of the subject following interferon treatment and an expression level of the gene in the cell of the subject prior to interferon treatment, or visa versa, namely, comparing a ratio determined between an expression level of the at least one gene selected from the group consisting of CXCL10 and CD24 in a cell of the subject prior to interferon treatment and an expression level of the gene in the cell of the subject following interferon treatment, to a reference ratio determined in a cell of at least one interferon responder subject and/or at least one interferon non-responder subject, the reference ratio is determined between an expression level of the at least one gene following interferon treatment and an expression level of the at least one gene prior to interferon treatment, or visa versa, namely, the reference ratio is determined between an expression level of the at least one gene prior to interferon treatment and an expression level of the at least one gene following interferon treatment, thereby predicting the responsiveness to interferon treatment of a subject.

Sequence information regarding the gene products (i.e., RNA transcripts and polypeptide sequences) of TNFRSF17, CXCL10 and CD24 which can be detected according to the method of some embodiments of the invention is provided in Table 16 in the Examples section which follows. In addition, sequence information of probes which can be used to detect the TNFRSF17, CXCL10 and CD24 RNA transcripts is provided in Table 16 in the Examples section which follows.

The phrase “following interferon treatment” refers to any time ranging from at least about 1 hour after interferon administration to about 1 week after interferon administration. For example, from about 1 hour to about 24-72 hours after administration, e.g., from about 4 hours to about 24 hours after interferon administration.

According to some embodiments of the invention, following interferon treatment is effected about 4 hours following interferon treatment.

According to some embodiments of the invention, following interferon treatment is effected about 24 hours following interferon treatment.

According to some embodiments of the invention, prior to interferon treatment is effected any time before the first interferon administration, such as a few minutes before interferon administration, a few hours before interferon administration or a few days, weeks, months or years before interferon administration.

According to some embodiments of the invention administration of interferon is carried out in vivo (i.e., to the subject in need of therapy).

According to some embodiments of the invention administration of interferon is carried out in vitro (e.g., in a cell culture).

According to some embodiments of the invention an increase above a predetermined threshold in the ratio of the subject relative to the reference ratio of the at least one interferon non-responder subject predicts responsiveness of the subject to interferon treatment of the subject.

According to some embodiments of the invention, the increase in the ratio of the subject is of at least about 2%, e.g., at least about 4%, at least about 6%, e.g., at least about 10%, at least about 20%, e.g., at least about 30%, at least about 40%, e.g., at least about 50%, at least about 60%, e.g., at least about 70%, at least about 80%, e.g., at least about 90%, e.g., at least about 2 times, e.g., at least about 3 times, e.g., at least about 4 times, e.g., at least about 5 times, e.g., at least about 6 times, e.g., at least about 7 times, e.g., at least about 8 times, e.g., at least about 9 times, e.g., at least about 10 times, e.g., at least about 20 times, e.g., at least about 30 times, e.g., at least about 40 times, e.g., at least about 50 times, e.g., at least about 60 times, or more relative to the reference ratio determined in the at least one interferon non-responder subject.

According to some embodiments of the invention a decrease above a predetermined threshold in the ratio of the subject relative to the reference ratio of the at least one interferon responder subject predicts lack of responsiveness of the subject to interferon treatment of the subject.

According to some embodiments of the invention, the decrease in the ratio of the subject is of at least about 2%, e.g., at least about 4%, at least about 6%, e.g., at least about 10%, at least about 20%, e.g., at least about 30%, at least about 40%, e.g., at least about 50%, at least about 60%, e.g., at least about 70%, at least about 80%, e.g., at least about 90%, e.g., at least about 2 times, e.g., at least about 3 times, e.g., at least about 4 times, e.g., at least about 5 times, e.g., at least about 6 times, e.g., at least about 7 times, e.g., at least about 8 times, e.g., at least about 9 times, e.g., at least about 10 times, e.g., at least about 20 times, e.g., at least about 30 times, e.g., at least about 40 times, e.g., at least about 50 times, e.g., at least about 60 times, or more relative to the reference ratio determined in the at least one interferon responder subject.

According to some embodiments of the invention, when the ratio of the subject is identical or changed below a predetermined threshold as compared to the reference ratio of the at least one interferon responder subject, then the subject is classified as responsive to interferon.

According to some embodiments of the invention, the change (increase or decrease) between the ratio of the subject and the reference ratio obtained from at least one interferon responder subject is below a predetermined threshold such as below about 10 times, e.g., below about 9 times, e.g., below about 8 times, e.g., below about 7 times, e.g., below about 6 times, e.g., below about 5 times, e.g., below about 4 times, e.g., below about 3 times, e.g., below about 2 times, e.g., below about 90%, e.g., below about 80%, e.g., below about 70%, e.g., below about 60%, e.g., below about 50%, e.g., below about 40%, e.g., below about 30%, e.g., below about 20%, e.g., below about 10%, e.g., below about 9%, e.g., below about 8%, e.g., below about 7%, e.g., below about 6%, e.g., below about 5%, e.g., below about 4%, e.g., below about 3%, e.g., below about 2%, e.g., below about 1% relative to the reference ratio of the at least one interferon responder subject.

According to some embodiments of the invention, when the ratio of the subject is identical or changed below a predetermined threshold as compared to the reference ratio of the at least one interferon non-responder subject, then the subject is classified as non-responsive to interferon.

According to some embodiments of the invention, the change (increase or decrease) between the ratio of the subject and the reference ratio obtained from at least one interferon non-responder subject is below a predetermined threshold such as below about 10 times, e.g., below about 9 times, e.g., below about 8 times, e.g., below about 7 times, e.g., below about 6 times, e.g., below about 5 times, e.g., below about 4 times, e.g., below about 3 times, e.g., below about 2 times, e.g., below about 90%, e.g., below about 80%, e.g., below about 70%, e.g., below about 60%, e.g., below about 50%, e.g., below about 40%, e.g., below about 30%, e.g., below about 20%, e.g., below about 10%, e.g., below about 9%, e.g., below about 8%, e.g., below about 7%, e.g., below about 6%, e.g., below about 5%, e.g., below about 4%, e.g., below about 3%, e.g., below about 2%, e.g., below about 1% relative to the reference expression data obtained from the at least one interferon non-responder subject.

According to an aspect of some embodiments of the invention, there is provided a method of predicting responsiveness of a subject to interferon treatment, comprising comparing a ratio determined between an expression level of ISG15, IFI6, IFIT1, OAS2 and OAS3 genes in a cell of the subject following interferon treatment and an expression level of the genes in the cell of the subject prior to interferon treatment, or visa versa, namely, comparing a ratio determined between an expression level of ISG15, IFI6, IFIT1, OAS2 and OAS3 genes in a cell of the subject prior to interferon treatment and an expression level of the genes in the cell of the subject following interferon treatment, to a reference ratio determined in a cell of at least one interferon responder subject and/or at least one interferon non-responder subject, the reference ratio is determined between an expression level of the genes following interferon treatment and an expression level of the genes prior to interferon treatment, or visa versa, namely, the reference ratio is determined between an expression level of the genes prior to interferon treatment and an expression level of the genes following interferon treatment, thereby predicting the responsiveness to interferon treatment of a subject.

Sequence information regarding the gene products (i.e., RNA transcripts and polypeptide sequences) of ISG15, IFI6, IFIT1, OAS2 and OAS3 which can be detected according to the method of some embodiments of the invention is provided in Table 2 in the Examples section which follows. In addition, sequence information of probes which can be used to detect the ISG15, IFI6, IFIT1, OAS2 and OAS3 RNA transcripts is provided in Table 2 in the Examples section which follows.

According to an aspect of some embodiments of the invention, there is provided a method of predicting responsiveness of a subject to interferon treatment, comprising comparing a ratio determined between an expression level of at least one gene selected from the group consisting of: TICAM1, MYD88, TLR7, TRAFD1 and IRF7 in a cell of the subject following interferon treatment and an expression level of the at least one gene in the cell of the subject prior to interferon treatment, or visa versa, namely, comparing a ratio determined between an expression level of at least one gene selected from the group consisting of: TICAM1, MYD88, TLR7, TRAFD1 and IRF7 in a cell of the subject prior to interferon treatment and an expression level of the at least one gene in the cell of the subject following interferon treatment, to a reference ratio determined in a cell of at least one interferon responder subject and/or at least one interferon non-responder subject, the reference ratio is determined between an expression level of the gene following interferon treatment and an expression level of the gene prior to interferon treatment, or visa versa, namely, the reference ratio is determined between an expression level of the gene prior to interferon treatment and an expression level of the gene following interferon treatment, thereby predicting the responsiveness to interferon treatment of a subject.

According to some embodiments of the invention the at least one gene comprises one gene, at least two genes, at least three genes, at least four genes or at least 5 genes from the group of TICAM1, MYD88, TLR7, TRAFD1 and IRF7 genes.

Sequence information regarding the gene products (i.e., RNA transcripts and polypeptide sequences) of TICAM1, MYD88, TLR7, TRAFD1 and IRF7 which can be detected according to the method of some embodiments of the invention is provided in Table 7 in the Examples section which follows. In addition, sequence information of probes which can be used to detect the TICAM1, MYD88, TLR7, TRAFD1 and IRF7 RNA transcripts is provided in Table 7 in the Examples section which follows.

According to an aspect of some embodiments of the invention, there is provided a method of predicting responsiveness of a subject to interferon treatment, comprising comparing a ratio determined between an expression level of at least one gene selected from the group consisting of HERC5 and UBE2L6 in a liver cell of the subject following interferon treatment and an expression level of the at least one gene in the liver cell of the subject prior to interferon treatment, or visa versa, namely, comparing a ratio determined between an expression level of at least one gene selected from the group consisting of HERC5 and UBE2L6 in a liver cell of the subject prior to interferon treatment and an expression level of the at least one gene in the liver cell of the subject following interferon treatment, to a reference ratio determined in a cell of at least one interferon responder subject and/or at least one interferon non-responder subject, the reference ratio is determined between an expression level of the at least one gene following interferon treatment and an expression level of the at least one gene prior to interferon treatment, or visa versa, namely, the reference ratio is determined between an expression level of the at least one gene prior to interferon treatment and an expression level of the at least one gene following interferon treatment, thereby predicting the responsiveness to interferon treatment of a subject.

For example, as shown in Table 6 (Examples section), while the average ratio between the expression level of HERC5 following interferon injection as compared to prior interferon injection was about 5 in interferon responders, the average ratio between the expression level of HERC5 following interferon injection as compared to prior interferon injection in interferon non-responders was about 1.18. Thus, there is an increase of about 5 times between the ratio in responders to the ratio in non-responder.

According to some embodiments of the invention the method further comprising comparing a ratio determined between an expression level of at least one gene selected from the group consisting of ISG15 and USP18 in a liver cell of the subject following interferon treatment and an expression level of the at least one gene in the liver cell of the subject prior to interferon treatment, or visa versa, namely, comparing a ratio determined between an expression level of at least one gene selected from the group consisting of ISG15 and USP18 in a liver cell of the subject prior to interferon treatment and an expression level of the at least one gene in the liver cell of the subject following interferon treatment, to a reference ratio determined in a cell of at least one interferon responder subject and/or at least one interferon non-responder subject, the reference ratio is determined between an expression level of the at least one gene following interferon treatment and an expression level of the at least one gene prior to interferon treatment, or visa versa, namely, the reference ratio is determined between an expression level of the at least one gene prior to interferon treatment and an expression level of the at least one gene following interferon treatment, thereby predicting the responsiveness to interferon treatment of a subject.

According to some embodiments of the invention the level of expression of the at least one gene comprises at least two, at least 3, at least 4 genes from the group of HERC5, UBE2L6, ISG15 and USP18 genes.

Sequence information regarding the gene products (i.e., RNA transcripts and polypeptide sequences) of HERC5, UBE2L6, ISG15 and USP18 which can be detected according to the method of some embodiments of the invention is provided in Table 5 in the Examples section which follows. In addition, sequence information of probes which can be used to detect the HERC5, UBE2L6, ISG15 and USP18 RNA transcripts is provided in Table 5 in the Examples section which follows.

According to an aspect of some embodiments of the invention, there is provided a method of predicting responsiveness of a subject to interferon treatment, comprising comparing a ratio determined between an expression level of at least one gene selected from the group consisting ISG15, IFI6, IFIT1, OAS2 and OAS3 in a liver cell of the subject following interferon treatment and an expression level of the at least one gene in the liver cell of the subject prior to interferon treatment, or visa versa, namely, comparing a ratio determined between an expression level of at least one gene selected from the group consisting ISG15, IFI6, IFIT1, OAS2 and OAS3 in a liver cell of the subject prior to interferon treatment and an expression level of the at least one gene in the liver cell of the subject following interferon treatment, to a reference ratio determined in a liver cell of at least one interferon responder subject and/or at least one interferon non-responder subject, the reference ratio is determined between an expression level of the at least one gene following interferon treatment and an expression level of the at least one gene prior to interferon treatment, or visa versa, namely, the reference ratio is determined between an expression level of the at least one gene prior to interferon treatment and an expression level of the at least one gene following interferon treatment, thereby predicting the responsiveness to interferon treatment of a subject.

According to some embodiments of the invention the at least one gene comprises one gene, at least two genes, at least three genes, at least four genes or at least 5 genes from the group of ISG15, IFI6, IFIT1, OAS2 and OAS3 genes.

It should be noted that for predication of responsiveness to interferon treatment several of the above methods may be used in combination for example combination of static (e.g., determination prior to interferon treatment), dynamic (e.g., comparing the level of expression before and after interferon treatment) and in vitro methods, combination of samples from blood or liver biopsy, combination of checking various sets of genes.

According to some embodiments of the invention, the method further comprising selecting the subject as an HCV infected subject (e.g., chronic HCV).

According to some embodiments of the invention, the method further comprising selecting the subject as a multiple sclerosis diagnosed subject.

The method of predicting the responsiveness of a subject to interferon treatment according to some embodiments of the invention enables the classification of a subject as an interferon responder or an interferon non-responder.

Since as mentioned above the responsiveness to interferon treatment may affect disease outcome, the teachings of the invention can be used to determine the prognosis of a subject in need of interferon treatment.

According to some embodiments of the invention, the method further comprising informing the subject of the predicted responsiveness to interferon treatment (e.g., responder or non-responder) and/or the predicted prognosis of the subject.

As used herein the phrase “informing the subject” refers to advising the subject that based on the predicted responsiveness to interferon treatment the subject should seek a suitable treatment regimen. For example, if the subject is predicted to respond to interferon treatment and is diagnosed or suffers from a pathology requiring interferon treatment that such a treatment is advisable.

Once the responsiveness to interferon treatment is determined, the results can be recorded in the subject's medical file, which may assist in selecting a treatment regimen and/or determining prognosis of the subject.

According to some embodiments of the invention, the method further comprising recording the responsiveness of the subject to interferon treatment in the subject's medical file.

As mentioned, the prediction of the responsiveness of a subject to interferon treatment can be used to select the treatment regimen of a subject and thereby treat the subject in need thereof.

Thus, according to an aspect of some embodiments of the invention, there is provided a method of treating of a subject in need of interferon treatment, the method comprising: (a) predicting the responsiveness of the subject to interferon treatment according to the method of the invention, and (b) selecting a treatment regimen based on the responsiveness; thereby treating the subject in need of interferon treatment.

As used herein the phrase “a subject in need of interferon treatment” refers to any subject who is diagnosed with or suffers from a pathology (e.g., condition, disease or disorder) requiring interferon treatment. Non-limiting examples of such pathologies include as autoimmune disorder (e.g., multiple sclerosis), cancer (e.g., hematological malignancy, leukemia and lymphomas including hairy cell leukemia, chronic myeloid leukemia, nodular lymphoma, cutaneous T-cell lymphoma, recurrent melanomas), and viral infection (e.g., hepatitis C virus infection, hepatitis B virus infection, viral respiratory diseases such as cold and flu).

According to some embodiments of the invention, a subject in need of interferon treatment is diagnosed with multiple sclerosis.

According to some embodiments of the invention, a subject in need of interferon treatment is infected with HCV (any type of HCV as described above).

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein the phrase “treatment regimen” refers to a treatment plan that specifies the type of treatment, dosage, schedule and/or duration of a treatment provided to a subject in need thereof (e.g., a subject diagnosed with multiple sclerosis or infected with HCV). The selected treatment regimen can be an aggressive one which is expected to result in the best clinical outcome (e.g., complete cure of the pathology), yet may be associated with some discomfort to the subject or adverse side effects (e.g., a damage to healthy cells or tissue); or a more moderate one which may relief symptoms of the pathology yet may results in incomplete cure of the pathology. The type of treatment, dosage, schedule and duration of treatment can vary, depending on the severity of pathology and the predicted responsiveness of the subject to the treatment, and those of skills in the art are capable of adjusting the type of treatment with the dosage, schedule and duration of treatment.

According to some embodiments of the invention, when the subject is classified as a responder to interferon treatment the treatment regimen comprises administration of interferon.

As mentioned above and described in the Examples section which follows, the present inventor has uncovered that in interferon responders there is a coordinated increase in the level of the KIR inhibitory receptor genes in the blood and of their matched HLA genes in the liver. Thus, the inventor uncovered that downregulation of the interaction between the HLA and the KIR inhibitory receptors would increase the responsiveness of a subject to interferon.

According to an aspect of some embodiments of the invention, there is provided a method of treating a subject in need of interferon therapy, comprising co-administering to the subject interferon and an agent capable of downregulating an expression level and/or activity of an HLA gene product or of a KR inhibitory receptor gene product, thereby treating the subject in need of interferon therapy.

According to some embodiments of the invention, co-administering is effected so as to enable a pharmacokinetic overlap between the interferon and the agent which is capable of downregulating HLA or KIR inhibitory receptor gene product(s).

As used herein the phrase “pharmacokinetic overlap” refers to a substantial overlap between the efficacy window of the agent capable of downregulating HLA or KIR inhibitory receptor gene products and the efficacy window of interferon.

As used herein, the phrase “efficacy window” describes a time frame during which an active agent exhibits a desired pharmacological effect, herein prevention of the interaction between HLA and the KIR inhibitory receptor by the agent capable of downregulating HLA or KR inhibitory receptor gene product(s); and clearance of HCV from the body by interferon. In other words, this phrase describes the time period at which the plasma concentration of an active agent is equal to or higher than a minimal pharmacologically effective concentration thereof.

According to some embodiments of the invention, the co-administering is effected concomitantly. In some embodiments, concomitant co-administration is effected such that both agents [i.e., interferon and agent which is capable of downregulating HLA or KIR inhibitory receptor gene product(s)] are administered at the same time, or such that the agents are co-formulated in a unit dosage form, as is detailed hereinafter.

According to some embodiments of the invention, the method further comprising administering ribavirin to the subject.

According to some embodiments of the invention, the subject is a non-responder to interferon treatment.

According to some embodiments of the invention, the agent which is capable of downregulating HLA or KIR inhibitory receptor gene product(s) can be an antibody, an RNA silencing molecule, a ribozyme or a DNAzyme.

According to some embodiments of the invention, the HLA gene product which expression level is downregulated by the agent is HLA-A, HLA-B, HLA-C, HLA-F and/or HLA-G.

According to some embodiments of the invention, the KIR inhibitory receptor gene product which expression level is downregulated by the agent is KIR3DL3, KIR3DL2, KIR3DL1, KIR2DL1, KIR2DL2, KIR2DL3, CD160 and/or KLRG1.

According to some embodiments of the invention, the antibody is an anti-KIR inhibitory receptor antibody.

According to some embodiments of the invention the anti-KIR inhibitory receptor antibody is the human monoclonal antibody 1-7F9 (Shah N., et al., 2009, Blood 114:2567-2568) which targets KIR2DL1, KIR2DL2 and KIR2DL3 on natural killer (NK) cells. This antibody activates NK cells by blocking the interaction between inhibitory KIRs and target cell HLA class I molecules.

According to some embodiments of the invention the anti-KIR inhibitory receptor antibody is the ECM41 monoclonal antibody (Vitale M., et al., Int Immunol. 2004 October; 16(10):1459-66) which is specific to KIR2DL3.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

The term “siRNA” refers to small inhibitory RNA duplexes (generally between 18-30 basepairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is theorized to result from providing Dicer with a substrate (27 mer) instead of a product (21 mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of the present invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

According to another embodiment the RNA silencing agent may be a miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target) The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Synthesis of RNA silencing agents suitable for use with the present invention can be effected as follows. First, the target mRNA sequence (e.g, HLA or KIR inhibitory receptors as described above) is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

According to some embodiments of the invention, the RNA silencing molecule is an siRNA directed against a KIR inhibitory receptor or a HLA.

Non-limiting examples of siRNA molecules which can be used according to the method of some embodiments of the invention include the sequences set forth by SEQ ID NOs:175-178 (directed against HLA-B); SEQ ID NOs:179-197 (directed against HLA-F); SEQ ID NOs:198-210 (directed against HLA-C); SEQ ID NOs:244-286 (directed against HLA-G); SEQ ID NOs:325-354 (directed against KIR3DL1); SEQ ID NOs:419-468 (directed against KIRG1); SEQ ID NOs:211-243 (directed against HLA-A); SEQ ID NOs:287-301 (directed against KIR3DL2); SEQ ID NOs:302-324 (directed against KIR3DL3); SEQ ID NOs:355-368 (directed against KIR2DL3); SEQ ID NOs:369-418 (directed against CD160); and/or the siRNA described in Sergio Gonzalez S, et al., 2005 (Molecular Therapy Vol. 11: 811-8. Amplification of RNAi—Targeting HLA mRNAs), which is incorporated herein by reference in its entirety.

It will be appreciated that the RNA silencing agent of the present invention need not be limited to those molecules containing only RNA, but further encompasses chemically-modified nucleotides and non-nucleotides.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of the present invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of the present invention preferably include, but are not limited to, penetratin, transportan, pIsl, TAT(48-60), pVEC, MTS, and MAP.

Another agent capable of downregulating HLA or KIR inhibitory receptor is a DNAzyme molecule capable of specifically cleaving an mRNA transcript or DNA sequence of the HLA or KIR inhibitory receptor. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

Downregulation of the HLA or KIR inhibitory receptor can also be effected using an antisense polynucleotide capable of specifically hybridizing with an mRNA transcript encoding the HLA or KIR inhibitory receptor.

Design of antisense molecules which can be used to efficiently downregulate a HLA or KIR inhibitory receptor must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. In addition, several approaches for designing and predicting efficiency of specific oligonucleotides using an in vitro system were also published (Matveeva et al., Nature Biotechnology 16: 1374-1375 (1998)].

Another agent capable of downregulating the HLA or KIR inhibitory receptor is a ribozyme molecule capable of specifically cleaving an mRNA transcript encoding HLA or KIR inhibitory receptor. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

The agents described hereinabove (e.g., interferon and/or an agent which is capable of downregulating the expression level and/or activity of the HLA or KIR inhibitory receptor as described above) of the present invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the interferon and/or an agent which is capable of downregulating the expression level and/or activity of the HLA or KIR inhibitory receptor as described above accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., multiple sclerosis or HCV infection) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue and/or blood levels of the active ingredient which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The agents described hereinabove (e.g., the oligonucleotides, probes, antibodies) for predicting responsiveness of a subject to interferon treatment may be included in a diagnostic kit/article of manufacture preferably along with appropriate instructions for use and labels indicating FDA approval for use in predicting responsiveness of a subject to interferon treatment and/or treating the subject.

Thus, according to an aspect of some embodiments of the invention there is provided a diagnostic kit. The kit comprises at least one oligonucleotide or antibody for specifically determining an expression level of at least one gene of the genes which are differentially regulated between interferon responders and interferon non-responders as described hereinabove and in the Examples section which follows.

The term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions.

According to some embodiments of the invention, the at least one oligonucleotide does not exceed 1000 oligonucleotides, e.g., does not exceed 500 oligonucleotides, e.g., does not exceed 400 oligonucleotides, e.g., does not exceed 300 oligonucleotides, e.g., does not exceed 200 oligonucleotides, e.g., does not exceed 50 oligonucleotides, e.g., does not exceed 40 oligonucleotides, e.g., does not exceed 30 oligonucleotides.

According to some embodiments of the invention, the at least one oligonucleotide or antibody is capable of determining the expression level of at least one gene selected from the group consisting of KIR3DL3, KIR3DL2, KIR3DL1, KIR2DL1, KIR2DL2, KIR2DL3, KLRG1, KIR3DS1, CD160, HLA-A, HLA-B, HLA-C, HLA-F, HLA-G and IFI27.

According to some embodiments of the invention, the at least one oligonucleotide or antibody is capable of determining the expression level of TNFRSF17 gene.

According to some embodiments of the invention, the at least one oligonucleotide or antibody is capable of determining the expression level of at least one gene selected from the group consisting of TNFRSF17, CXCL10 and CD24.

According to some embodiments of the invention, the at least one oligonucleotide or antibody is capable of determining the expression level of at least one gene selected from the group consisting of IFI6, OAS2, ISG15, OAS3 and IFIT1 genes.

According to some embodiments of the invention, the at least one oligonucleotide or antibody is capable of determining the expression level of at least one gene selected from the group consisting of HERC5 and UBE2L6.

According to some embodiments of the invention, the at least one oligonucleotide or antibody is capable of determining the expression level of at least one gene selected from the group consisting of HERC5, UBE2L6, ISG15 and USP18.

According to some embodiments of the invention, the at least one oligonucleotide or antibody is capable of determining the expression level of at least one gene selected from the group consisting of KIR3DL3, KIR3DL2, KIR3DL1, KIR2DL1, KIR2DL2, KIR2DL3, KLRG1, KIR3DS1, CD160, HLA-A, HLA-B, HLA-C, HLA-F, HLA-G, IFI27, TNFRSF17, CXCL10, CD24, IFI6, OAS2, ISG15, OAS3, IFIT1, HERC5, UBE2L6, ISG15 and USP18.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Identification of Genes which are Differentially Expressed Between Responders and Non-Responders to Interferon Treatment

### A Static Method

In order to get a high resolution of the relative importance of the dominate genes which determine the fate of interferon I treatment, the present inventor has developed a statistical scoring tool that is based on the quantitative and qualitative ranking of all the genes in all possible permutations. The assumption behind this process is that the most important genes determining the outcome of treatment are the ones most persistent in all possible combinations of patient comparison selection. In the case of the Chen et al., 2005 dataset [L. Chen, I. Borozan, J. Feld, J. Sun, L. Tannis, C. Coltescu, J. Heathcote, A. Edwards, I. Mcgilvray. “Hepatic Gene Expression Discriminates Responders and Nonresponders in Treatment of Chronic Hepatitis C Viral Infection”; Gastroenterology, 128:1437-1444; Hypertext Transfer Protocol://142.150.56.35/˜LiverArrayProject/home (dot) html] the present inventor has selected all possible 11 combinations out of 15 cases in each group of interferon responders and non responders (13651 possibilities) which yielded 1365² or 18563225 t-test competitions between the genes as they are expressed in the two groups. In each race, the names of the top 10 winning genes were accumulated along with their p value scores, since the place of the winning genes in the race and the relative distance to the other genes in the race (how far away each gene is from the one before him and from the one after him) are important. Based on the tabulation of the results (Table 1, below) the prediction of the critical genes for the success of pegylated interferon (IFNIα) treatment for HCV were found. These are the same five genes identified by the present inventor in WO2007039906. However using the current methods the present inventor was able to pinpoint at the two most important genes required for the prediction, and to indicate the exact nature of the difference between respondents and non-respondents

TABLE 1
Top 10 gene scores

| | | | Ratio of | | g1p2 score |
|---|---|---|---|---|---|
| | Gene | Score | best score | Divided by | divider |
| 1 | ISG15 | 9.49E+09 | 1 | 1 | g1p2/1 |
| | (g1p2) | | | | |
| 2 | IFI6 (g1p3) | 4.47E+09 | 0.471521 | 2.120794 | g1p2/2.12 |
| 3 | ifit1 | 3.79E+08 | 0.03999 | 25.00643 | g1p2/25 |
| 4 | oas2 | 2.97E+08 | 0.031293 | 31.95613 | g1p2/31 |
| 5 | oas3 | 17968405 | 0.001894 | 528.0912 | g1p2/528 |
| 6 | | 2829517 | 0.000298 | 3353.56 | g1p2/3353 |
| 7 | | 2450741 | 0.000258 | 3871.872 | |
| 8 | | 896905 | 9.45E−05 | 10579.67 | |
| 9 | | 852981 | 8.99E−05 | 11124.46 | |
| 10 | | 427878.8 | 4.51E−05 | 22176.74 | |

Table 1: Scoring results top 5 critical genes.

In the tabulation process the statistical score of the top up-regulated genes in non-responders compared to responders was much higher than the scores obtained by the top down-regulated genes (which are therefore not included in the Table) in the opposite direction. Thus, an additional significant improvement was added to teachings of WO2007039906, in which during the scoring process, instead of using the median p-value of each of the 10 top places, each gene was scored by his own p-values obtained in the Table of the top 10 places.

These results demonstrate that the level of expression of g1p2 (ISG15) can be used as a predictor for response to interferon treatment before treatment has began.

### Example 2

### Validation of the Predictive Power of the Signature Genes to Interferon Response in Independent Data Sets

Experimental Procedures

The expression levels of the genes-of-interest were obtained from publicly available data bases [Hypertext Transfer Protocol://World Wide Web (dot) ncbi (dot) nlm (dot) nih (dot) gov/projects/geo/] using the Gene Expression Omnibus Accession No. GSE11190. Analysis of data was performed by custom programs written in MATLAB.

Validation of results was performed by analysing RNA extracted from paraffin embedded liver biopsies which were obtained from of HCV type 1 patients before the first injection of interferon (i.e., in time 0, naïve patients).

Results

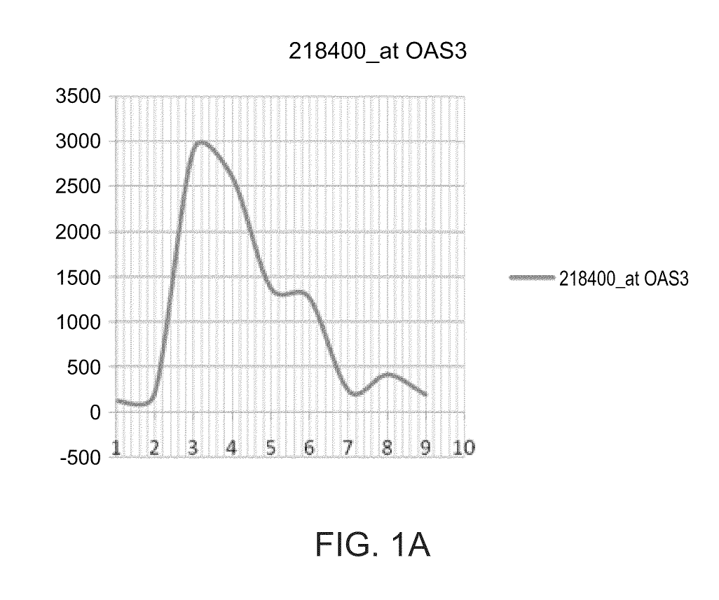

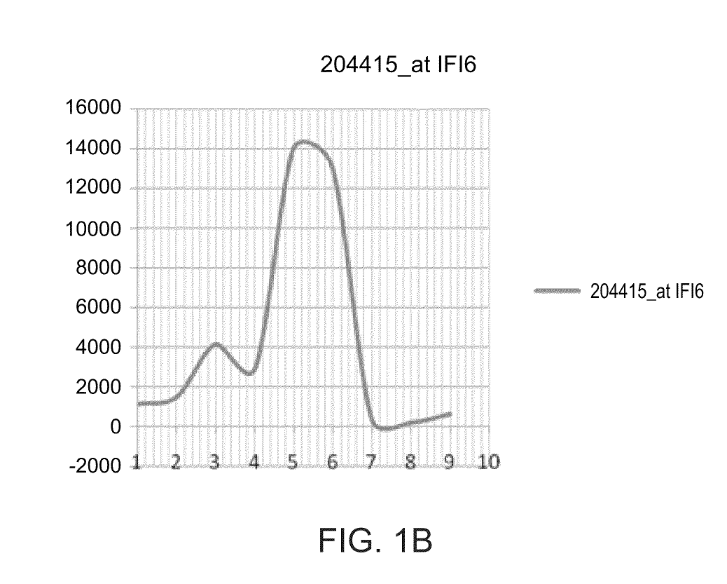

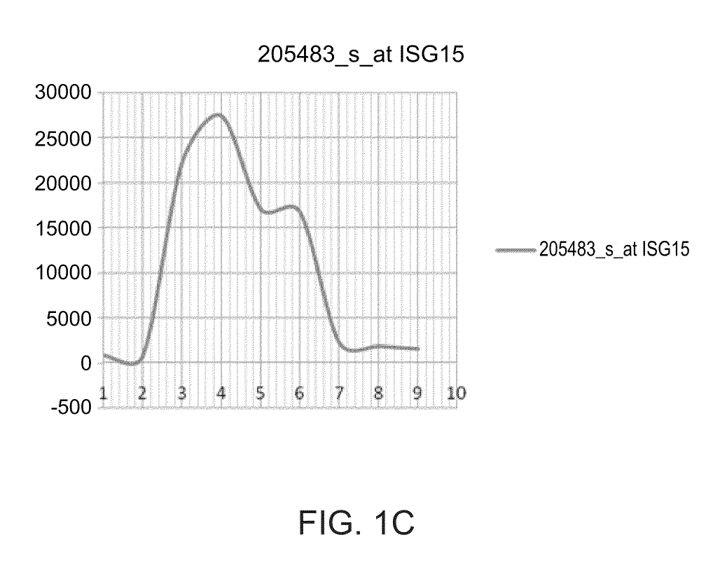

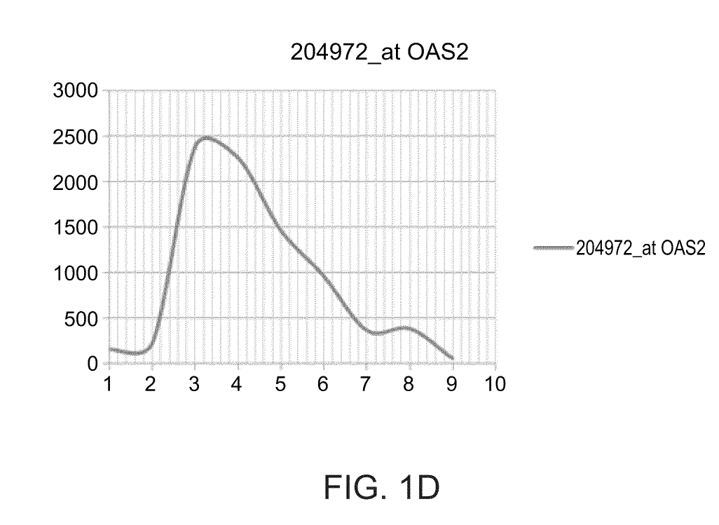

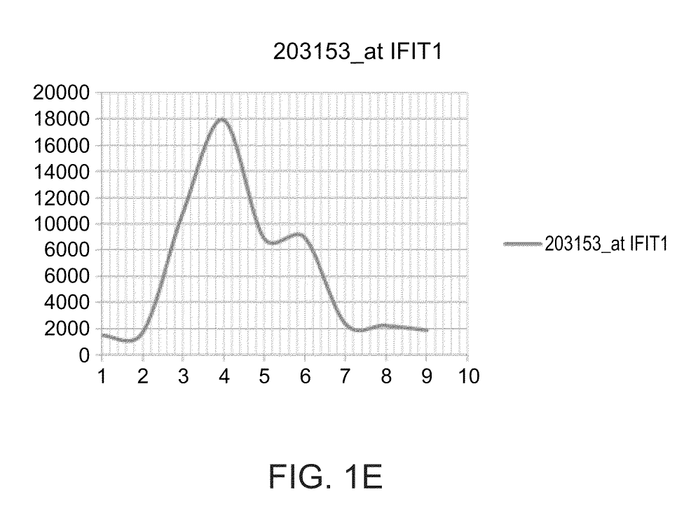

The OAS3, IF16, ISG15, OAS2 and IFIT1 gene signature is differentially expressed between interferon responder and non-responders—As shown in FIG. 1 and Table 3 below, the OAS3, IF16, ISG15, OAS2 and IFIT1 are up regulated in non-responders to interferon treatment as compared to responders or healthy controls. Sequence information of genes which were found to be differentially regulated in between responders and non-responders to interferon treatment is provided in Table 2, below.

TABLE 2
Sequence information

| | | | | RefSeq | | |
|---|---|---|---|---|---|---|
| | | | | Protein ID | RefSeq | |
| Probe Set | Gene | | Representative | (SEQ ID | Transcript ID | UniGene |
| ID | Symbol | Gene Title | Public ID | NO:) | (SEQ ID NO:) | ID |
| 204415_at | IFI6 | interferon, | NM_022873 | NP_002029 | NM_002038 | Hs.523847 |
| (SEQ ID | (G1P3) | alpha- | | (SEQ ID | (SEQ ID | |
| NO: 1); | | inducible | | NO: 6); | NO: 15); | |
| | | protein 6 | | NP_075010 | NM_022872 | |
| | | | | (SEQ ID | (SEQ ID | |
| | | | | NO: 7); | NO: 16); | |
| | | | | NP_075011 | NM_022873 | |
| | | | | (SEQ ID | (SEQ ID | |
| | | | | NO: 8); | NO: 17); | |
| 218400_at | OAS3 | 2′-5′- | NM_006187 | NP_006178 | NM_006187 | Hs.528634 |
| (SEQ ID | | oligoadenylate | | (SEQ ID | (SEQ ID | |
| NO: 2); | | synthetase 3, | | NO: 9); | NO: 18); | |
| | | 100 kDa | | | | |
| 205483_s_at | ISG15 | ISG15 | NM_005101 | NP_005092 | NM_005101 | Hs.458485 |
| (SEQ | (G1P2) | ubiquitin-like | | (SEQ ID | (SEQ ID | |
| ID NO: 3); | | modifier | | NO: 10); | NO: 19); | |
| 204972_at | OAS2 | 2′-5′- | NM_016817 | NP_001027903 | NM_001032731 | Hs.414332 |
| (SEQ ID | | oligoadenylate | | (SEQ ID | (SEQ ID | |
| NO: 4); | | synthetase 2, | | NO: 11); | NO: 20); | |
| | | 69/71 kDa | | NP_002526 | NM_002535 | |
| | | | | (SEQ ID | (SEQ ID | |
| | | | | NO: 12); | NO: 21); | |
| | | | | NP_058197 | NM_016817 | |
| | | | | (SEQ ID | (SEQ ID | |
| | | | | NO: 13); | NO: 22); | |
| 203153_at | IFIT1 | interferon- | NM_001548 | NP_001539 | NM_001548 | Hs.20315 |
| (SEQ ID | | induced | | (SEQ ID | (SEQ ID | |
| NO: 5); | | protein with | | NO: 14); | NO: 23); | |
| | | tetratricopeptide | | | | |
| | | repeats 1 | | | | |

Table 2: Probe sets ID refer to the GeneChip Array “Human Genome U133 Plus 2.0 Array” from Affimetrix (Affymetrix hu1333_plus2). Sequences from NCBI refer to Genome version March 2006 (NCBI Build 36.1).

TABLE 3
Expression analysis of signature genes in liver tissues in HCV type 1 patients

| probe | gene | c1 | c2 | nr_1 | nr_2 | nr_4 | nr_5 | r_1 | r_2 | r_3 |
|---|---|---|---|---|---|---|---|---|---|---|
| 218400_at | OAS3 | 126 | 193 | 2912 | 2601 | 1377 | 1275 | 236 | 413 | 196 |
| 205483_s_at | ISG15 | 842 | 775 | 22192 | 27471 | 17024 | 16703 | 2257 | 1806 | 1514 |
| 204415_at | IFI6 | 1105 | 1478 | 4118 | 2902 | 14001 | 13068 | 322 | 161 | 583 |
| 204972_at | OAS2 | 157 | 230 | 2385 | 2255 | 1469 | 964 | 362 | 382 | 64 |
| 203153_at | IFIT1 | 1475 | 1659 | 10685 | 17920 | 8919 | 8977 | 2374 | 2261 | 1889 |

Table 3. Provided are the raw data of the expression levels of various genes among 2 control subjects (c1, c2), 4 non-responders (nr_1, nr_2, nr_4 and nr_5) and 3 responders (r_1, r_2 and r_3) as measured in liver tissues of HCV type 1 (Results are presented in FIGS. 1A-E).

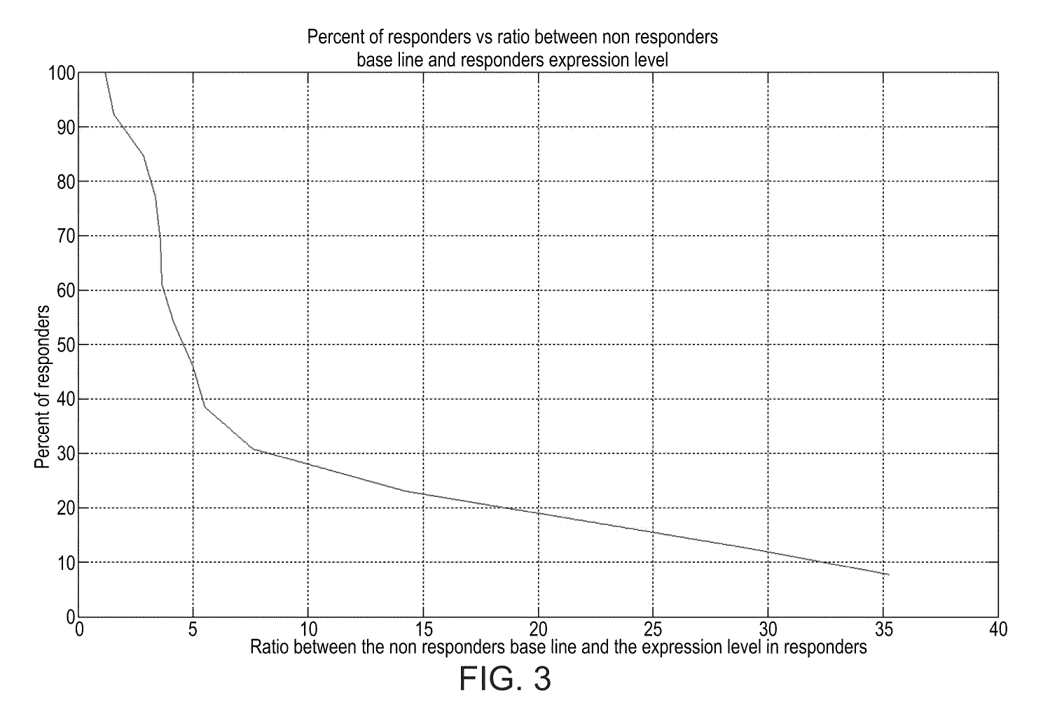

Determination of expression level of the 5-signature genes OAS3, IF16, ISG15, OAS2 and IFIT1 in naïve HCV type 1 patients—Archive liver tissues from 21 HCV type 1 patients were used to determine the level of the 5-signature genes before interferon treatment (i.e., at time 0, naïve patients) and the ratio between the base line expression level of the signature genes in interferon non-responders and the expression level in each of the interferon responders was determined (FIG. 3). The results show a high ratio between the expression level of non-responders base line and the expression level in responders, demonstrating that prior to interferon treatment the level of the 5-signature genes is low in patients which later on appear to be responders to interferon. These results confirm the previous results and demonstrate that the signature gene expression can be used to predict response to interferon.

### Example 3

### Identification of a Genetic Switch Immediately after Interferon Treatment as a Predictor for Response to Interferon Treatment

### A Dynamic Method

The expression levels of the genes-of-interest were obtained from publicly available data bases [Hypertext Transfer Protocol://World Wide Web (dot) ncbi (dot) nlm (dot) nih (dot) gov/projects/geo/] using the Gene Expression Omnibus Accession No. GSE11190. Analysis of data was performed by custom programs written in MATLAB.

Results

The expression levels of the OAS3, IF16, ISG15, OAS2 and IFIT1 gene signature is significantly upregulated among interferon responders following the first interferon treatment while being unchanged among non-responders—Based on the results presented in FIGS. 1A-E, Table 2 and FIG. 3, the present inventor has hypothesized that the gene signature reflects an on/off situation; thus injection of interferon to “on” genes at 0 time (i.e., before the first injection of interferon, naïve subjects with respect to interferon) can not upregulate them, while injection to “off” genes will upregulate them. To test this assumption the inventor used the data set described in Sarasin-Filipowicz M, et al., 2008, who analyzed a total of 78 samples of blood or liver biopsy (taken before and after interferon alpha treatment) using Affymetrix Human U133 Plus 2.0 Array. The original data set was divided to two groups of patient: HCV type 1 and HCV types 2, 3 and 4 (types 2-4 hereinafter). The hypothesis was tested in HCV type 1 patients using liver tissue biopsies taken 4 hours after injection of interferon and prior to injection with interferon. As shown in FIGS. 2A-E and Table 4 below, while in non-responders to interferon treatment (subjects 1-4 in FIGS. 2A-E) there is nearly no change in the expression of the signature genes following interferon injection, in interferon responders (subjects 5-7 in FIGS. 2A-E), the expression level of the signature genes is at least log 2 2.5 folds higher following interferon treatment. Thus, the ratio (in vivo log 2) between the expression level determined 4 hours after injection and the expression level determined before injection was significantly higher among interferon responders as compared to interferon non-responders. These results demonstrate a powerful validation and a new prediction power for response to treatment provided by this gene signature by testing the level of the signature genes before and after interferon injection in liver tissues of HCV type 1 subjects.

TABLE 4
Expression analysis of signature genes in liver of HCV type 1 patients

| Probe | Gene | Nr_1 | Nr_2 | Nr_4 | Nr_5 | R_1 | R_2 | R_3 |
|---|---|---|---|---|---|---|---|---|
| 205483_s_at | ISG15 | 0.161 | −0.081 | 0.019 | 0.243 | 3.078 | 1.609 | 3.557 |
| 203153_at | IFIT1 | 0.540 | −0.174 | 0.189 | 0.950 | 3.299 | 2.078 | 3.763 |
| 204972_at | OAS2 | 0.115 | −0.049 | 0.097 | 0.556 | 3.035 | 1.545 | 4.604 |
| 218400_at | OAS3 | 0.036 | 0.099 | 0.535 | 0.409 | 3.673 | 1.161 | 3.268 |
| 204415_at | IFI6 | 0.222 | 0.308 | −0.135 | −0.133 | 1.511 | 1.276 | 3.008 |

Table 4. Provided are the logarithmic ratios between the expression levels of various genes as measured in liver tissues of HCV type 1 patients 4 hours after interferon treatment and the level measured prior to interferon treatment among non-responders (Nr_1, Nr_2, Nr_4, and Nr_5) and responders (R_1, R_2, and R_3) to interferon treatment (Results are presented in FIGS. 2A-E).

These results demonstrate that in addition to testing the expression level of the 5 signature genes prior to interferon treatment, there is a significant value, with a high predictive power, to test the expression of the 5 signature genes in liver tissue (biopsies) taken prior to interferon treatment and 4 hours after interferon treatment, since the switch in gene expression immediately after interferon treatment is significant among interferon responders. On the other hand, in non-responders there is no change in the level of expression of these genes.

### Example 4

### The Signature Genes can Predict Response to Interferon Treatment in Types 1-4 HCV Patients

The expression levels of the genes-of-interest were obtained from publicly available data bases [Hypertext Transfer Protocol://World Wide Web (dot) ncbi (dot) nlm (dot) nih (dot) gov/projects/geo/] using the Gene Expression Omnibus Accession No. GSE11190. Analysis of data was performed by custom programs written in MATLAB.

Results

The 5 signature genes are upregulated in interferon non-responders as compared to responders in liver samples of HCV types 1-4 patients—To further substantiate the above results and in order to determine if the 5-signature genes can predict responsiveness to interferon also in other types of HCV infections, the present inventor has analyzed data from GSE11190 and compared the level of expression between responders and non-responders to interferon treatment. The results show that in liver tissues of HCV types 1-4 interferon non-responders the 5 genes i.e., ISG15, IFIT1, IFI6, OAS2, OAS3 are significantly up-regulated as compared to the interferon responders (FIG. 4).

Further analysis of the same data set showed that in HCV type 1 patients the ISG15, IFIT1, IFI6, OAS2 and OAS3 genes are significantly up-regulated in non-responders as compared to responders (FIG. 5).

These results demonstrate that the 5-signature genes (ISG15, IFIT1, IFI6, OAS2 and OAS3) can be used to predict response to interferon in all types of HCV virus infections, i.e., types 1-4, wherein upregulation of the level of expression before interferon treatment indicates that the patient will not response to a subsequent interferon treatment.

### Example 5

### Involvement of the ISGylated Process in Response to Interferon

### A Dynamic Method

The expression levels of the genes-of-interest were obtained from publicly available data bases [Hypertext Transfer Protocol://World Wide Web (dot) ncbi (dot) nlm (dot) nih (dot) gov/projects/geo/] using the Gene Expression Omnibus Accession No. GSE11190. Analysis of data was performed by custom programs written in MATLAB.

As shown in Example 1 hereinabove, the master gene of the predictive signature set is the ISG15 [interferon (IFN)-stimulated protein of 15 kDa]. G1P2/ISG15 is a ubiquitin-like protein that becomes conjugated to many cellular proteins upon activation by interferon-alpha (IFNA; MIM 147660) and interferon-beta (IFNB; MIM 147640) (Zhao et al., 2005 [PubMed16009940]). The ISGylated process is described in Anthony J. Sadler and Bryan R. G. Williams, 2008 [Nat Rev Immunol. 8(7):559-68. Review].

Table 5 below, provides sequence information of the probes/genes of the ISGylated process which were analyzed by the present inventor.

TABLE 5
Sequence information

| | | | | RefSeq | | |
|---|---|---|---|---|---|---|
| | | | | Protein ID | RefSeq | |
| Probe Set | Gene | | Representative | (SEQ ID | Transcript ID | UniGene |
| ID | Symbol | Gene Title | Public ID | NO:) | (SEQ ID NO:) | ID |
| 205483_s_at | ISG15 | ISG15 | NM_005101 | NP_005092 | NM_005101 | Hs.458485 |
| (SEQ | | ubiquitin-like | (SEQ ID | (SEQ ID | (SEQ ID | |
| ID NO: 3) | | modifier | NO: 19) | NO: 10) | NO: 19) | |
| 219211_at | USP18 | ubiquitin | NM_017414 | NP_059110 | NM_017414 | Hs.38260 |
| (SEQ ID | | specific | (SEQ ID | (SEQ ID | (SEQ ID | |
| NO: 24) | | peptidase 18 | NO: 27) | NO: 30) | NO: 27) | |
| 219863_at | HERC5 | hect domain | NM_016323 | NP_057407 | NM_016323 | Hs.26663 |
| (SEQ ID | | and RLD 5 | (SEQ ID | (SEQ ID | (SEQ ID | |
| NO: 25) | | | NO: 28) | NO: 31) | NO: 28) | |
| 201649_at | UBE2L6 | ubiquitin- | NM_004223 | NP_004214 | NM_004223 | Hs.425777 |
| (SEQ ID | | conjugating | (SEQ ID | (SEQ ID | (SEQ ID | |
| NO: 26) | | enzyme E2L6 | NO: 29) | NO: 32); | NO: 29); | |
| | | | | NP_937826 | NM_198183 | |
| | | | | (SEQ ID | (SEQ ID | |
| | | | | NO: 33) | NO: 34) | |

Table 5: Probe sets ID refer to the GeneChip Array “Human Genome U133 Plus 2.0 Array” from Affimetrix (Affymetrix hu1333_plus2). Sequences from NCBI refer to Genome version March 2006 (NCBI Build 36.1).

### Results

In order to determine the relevance of the genes involved in ISGylation in the response to interferon, the present inventor has analyzed the GSE11190 dataset for the expression level of the ISGylated genes. As shown in FIGS. 6A-D and Table 6 below, following interferon injection the genes belonging to the ISGylated process, ISG15, ISG15, HERC5, UBE2L6 are up-regulated in the responders (e.g., subjects 5, 6, 7 in FIGS. 6A-D) but not in the non-responders.

TABLE 6
Raw data analysis of expression analysis of genes involved in the
ISGylation process

| | nr1 | nr2 | nr3 | nr4 | r1 | r2 | r3 |
|---|---|---|---|---|---|---|---|
| HERC5 | 0.001 | 0.20 | 0.26 | 0.46 | 2.83 | 1.31 | 2.44 |
| USP18 | 0.62 | −0.07 | 0.22 | 0.74 | 3.18 | 1.38 | 3.40 |
| ISG15 | 0.16 | −0.08 | 0.02 | 0.24 | 3.08 | 1.61 | 3.56 |
| UBE2L6 | 0.17 | −0.17 | −0.06 | 0.19 | 1.10 | 0.48 | 1.09 |

Table 6: Provided are the changes in expression level between 4 hours after interferon treatment as compared to prior to interferon treatment on a log2 scale.
“Nr” = non-responder to interferon treatment;
“r” = responder to interferon treatment.

These results indicate that the ISGylation process is essential to the success of the interferon treatment and in cases where the ISGylation is not “switched on” (i.e., upregulated following the first injection as compared to the level before the first injection) the treatment of interferon is likely to fail.

The mechanism of action of ISG15 disabling in non responders—The expression of interferon (IFN)-stimulated protein of 15 kDa (ISG15), the E1-activating enzyme UBE1L or UBE2L6 shown here (E1-like ubiquitin-activating enzyme) and multiple E2-conjugating enzymes (shown here as an example is UBCH8) and E3-ligase enzymes [such as HERC5 (homologous to the E6-associated protein C terminus domain and RCC1-like domain containing protein 5)] is coordinately induced by type I IFNs through IFN-stimulated response elements (ISREs) in their respective gene promoter regions. E1, E2 and E3 proteins sequentially catalyse the conjugation of ISG15 to numerous protein substrates to modulate pleiotropic cellular responses to inhibit virus production. This process (known as ISGylation) is reversibly regulated by proteases [such as ubiquitin-specific protease 18 (USP18)]), which are also induced by IFNs Interferon-inducible antiviral effectors.

Analysis and Discussion

Altogether, these results demonstrate that the top 5 genes ISG15, IFIT1, IFI6, OAS2 and OAS3 are relevant genes for deciding the fate of the treatment among HCV types 1-4 patients. The scores of place 6 and (g1p2 score divide by 3353) and further down (as shown in Table 1, Example 1) are of no statistical importance. In addition, the 2 neighboring genes g1p2 (isg15) and g1p3 (ifi6) stand out as most important genes in deciding the fate of the treatment. If their expression level is up-regulated, a person would most probably not respond to the treatment. The next 3 genes are in concert with the top 2 but not as significant as the top 2 genes. IFIT1 and OAS2 add to the certainty of prediction, and with yet less significance, so does OAS3.

These results show that these genes act as a “fate switch”. If the switch is “ON” (genes up-regulated), then the interferon treatment can not yield any success as its defense scheme operates by up-regulating these genes. In contrast, if the switch is “OFF” (genes down-regulated), then the interferon treatment can up-regulate these key genes, and start the defense mechanism.

### Example 6

### Identification of Common Regulatory Sequences to the Interferon Response Switch Genes

The immune cells in the liver include hepatocytes, which are lined with biliary endothelial cells (EC) on the portal facet, and stellate cells (SC) in the space of Disse (SD). EC separate the SD from the blood flow. Dendritic cells of plasmacytoid (PDC) and myeloid (MDC) origin, monocytes (Mo), macrophages (Mf), and Kupffer cells (KC) are located in close proximity to EC.

The ISRE promoter is common to the 5-signature genes—The present inventor used Toucan 2 [workbench for regulatory sequence analysis; Hypertext Transfer Protocol://homes (dot) esat (dot) kuleuven (dot) be/˜saerts/software/toucan (dot) php] to search for common regulatory sequences of the signature genes. Thus, the 300 bp upstream sequence (relative to from exon 1) of each of the 5 signature genes was analyzed using a motif scanner with the TRANFAC Public 7.0 Verterbrates PWM Database and for Background using Human DBTSS Promoters(0) Database. As shown in FIG. 7, all gene expression controls are localized to the ISRE promoter where ISGF3 complex and IRF7 are the controlling elements.

### Example 7

### Activation of the TLR9 Pathway Genes in Blood of Responders HCV Patients Following Interferon Treatment

The TLR (Toll-like receptor)-mediated type I IFN pathway, in particular the MyD88 signaling pathway for IRF7 activation in pDCs, are in charge of robust type I gene induction.

Upon TLR7 or TLR9 (expressed in endosomes) stimulation, IRF7 interacting with MyD88 is activated by the IRAK4-IRAK1-IKK kinase cascade. Secreted type I IFNs enhance the expression of IRF7 gene, leading to further enhancement of type I IFN gene induction (Kenya Honda, Akinori Takaoka, and Tadatsugu Taniguchi. Type I Inteferon Gene Induction Review by the Interferon Regulatory Factor Family of Transcription Factors. Immunity 25, 349-360, September 2006). TLR receptors from the TLR9 subfamily (TLR7 and TLR9) which are expressed in the endosomes transmit downstream signals via the recruitment of TIR-containing adaptor protein, such as MyD88 and TICAM1. IRF7 also interacts with TRAF6, another adaptor molecule functioning downstream of MYD88.

The present inventor has tested the changes in expression level of genes in the TLR mediated type I IFN pathway following treatment with interferon in blood samples of HCV type 1 patients. For this purpose, the data set (Gene Expression Omnibus Accession No. Gse7123) published by Taylor M W, et al., 2007 (Virol. 81:3391-401. Changes in gene expression during pegylated interferon and ribavirin therapy of chronic hepatitis C virus distinguish responders from nonresponders to antiviral therapy) was analyzed. This data set includes gene expression data from RNA that was extracted from PBMC of type 1 HCV patients (responders and non-responders to interferon treatment) obtained prior to injection, 24 hours following the treatment, and on other time points along the end of the treatment. After RMA (Robust Multichip Average) normalization of all microarrays, the data was arranged to provide the fold change in a log 2 scale in the expression level determined 24 hours after interferon treatment as compared to prior to interferon treatment in both responders and non responders.

Results

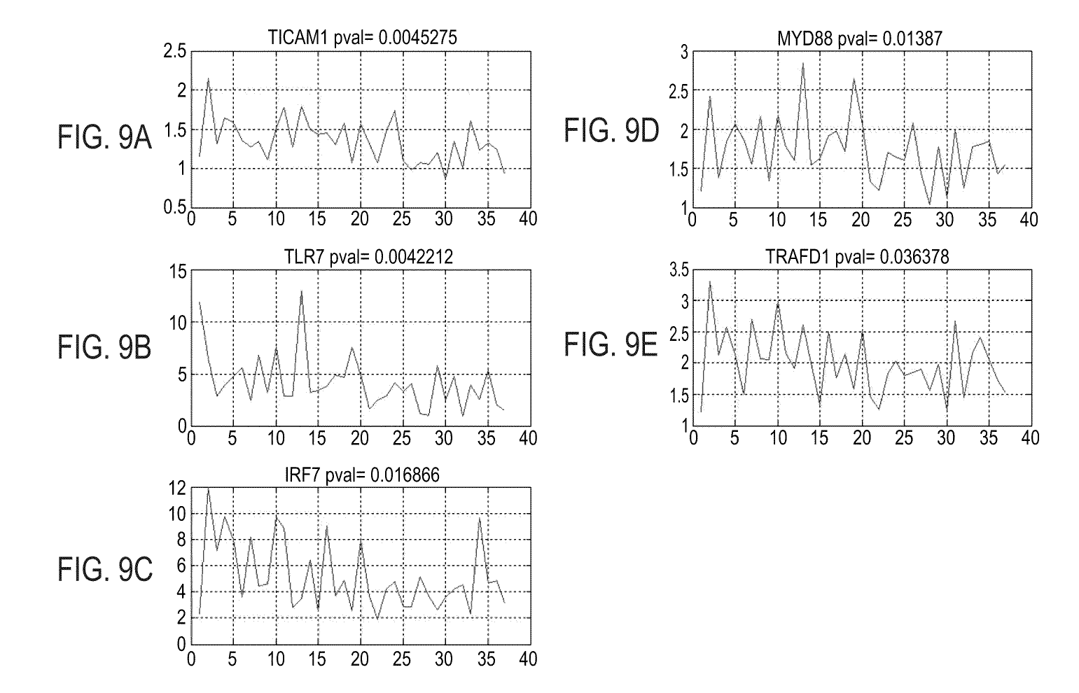

The 5-signature genes (G1P2, G1P3, IFIT1, OAS2 and OSA3) and the TLR7-mediated pathway genes (TICAM1, MYD88, TLR7, TRAFD1, IRF7) are upregulated in responders following the first interferon injection—The results of the analyses show that both the switch signature genes (FIGS. 8A-E) and the TLR7-mediated type I IFN pathway genes (FIGS. 9A-E) are up-regulated 24 hours following interferon treatment in responders but not in non-responders (p-value of less than 0.05). Thus, these results show that in responders, but not in non-responders to interferon treatment, there is a functionally “on” switch (i.e., upregulation of genes in response to interferon treatment) of the TLR signature genes.

Table 7 hereinbelow, provides sequence information of the TLR signature genes which were found to be upregulated in responders following treatment with interferon.

TABLE 7
Sequence information of the TLR7 pathway genes

| | | | | RefSeq | RefSeq | |
|---|---|---|---|---|---|---|
| | | | | Protein ID | Transcript ID | |
| Probe Set | Gene | | Transcript | (SEQ ID | (SEQ ID | UniGene |
| ID | Symbol | Gene Title | ID | NO:) | NO:) | ID |
| 209124_at | MYD88 | myeloid | AK097983; | NP_002459 | NM_002468 | Hs.82116 |
| (SEQ ID | | differentiation | AK124685; | (SEQ ID | (SEQ ID | |
| NO: 35) | | primary | BC013589; | NO: 40) | NO: 48) | |
| | | response | BX537602; | | | |
| | | gene (88) | ENST00000396334; | | | |
| | | | ENST00000415158; | | | |
| | | | ENST00000416282; | | | |
| | | | ENST00000417037; | | | |
| | | | ENST00000421516; | | | |
| | | | ENST00000421571; | | | |
| | | | ENST00000443433; | | | |
| | | | NM_002468; | | | |
| | | | U70451; | | | |
| | | | uc003chw.1; | | | |
| | | | uc003chx.1 | | | |
| 213191_at | TICAM1 | toll-like | AB086380; | NP_891549 | NM_182919 | Hs.29344 |
| (SEQ ID | | receptor | ENST00000248244; | (SEQ ID | (SEQ ID | |
| NO: 36) | | adaptor | NM_182919; | NO: 41) | NO: 49) | |
| | | molecule 1 | uc002mbh.1; | | | |
| | | | uc002mbi.1 | | | |
| 218400_at | OAS3 | 2′-5′- | AB044545; | NP_006178 | NM_006187 | Hs.528634 |
| (SEQ ID | | oligoadenylate | AF063613; | (SEQ ID | (SEQ ID | |
| NO: 2 | | synthetase | ENST00000228928; | NO: 9) | NO: 18) | |
| | | 3, 100 kDa | NM_006187; | | | |
| | | | uc001tug.1 | | | |
| 220146_at | TLR7 | toll-like | AF240467; | NP_057646 | NM_016562 | Hs.659215 |
| (SEQ ID | | receptor 7 | ENST00000380659; | (SEQ ID | (SEQ ID | |
| NO: 37 | | | NM_016562; | NO: 42) | NO: 50) | |
| | | | uc004cvc.1 | | | |
| 35254_at | TRAFD1 | TRAF- | AB007447; | NP_001137378 | NM_001143906 | Hs.5148 |
| (SEQ ID | | type zinc | BC003553; | (SEQ ID | (SEQ ID | |
| NO: 38) | | finger | ENST00000257604; | NO: 43); | NO: 51); | |
| | | domain | ENST00000412615; | NP_006691 | NM_006700 | |
| | | containing 1 | ENST00000432758; | (SEQ ID | (SEQ ID | |
| | | | NM_001143906; | NO: 44) | NO: 52) | |
| | | | NM_006700; | | | |
| | | | uc001tto.1; | | | |
| | | | uc001ttp.1 | | | |
| 208436_s_at | IRF7 | interferon | AF076494; | NP_001563 | NM_001572 | Hs.166120 |
| (SEQ | | regulatory | AK303752; | (SEQ ID | (SEQ ID | |
| ID NO: 39) | | factor 7 | BC136555; | NO: 45); | NO: 53); | |
| | | | ENST00000330243; | NP_004020 | NM_004029 | |
| | | | ENST00000348655; | (SEQ ID | (SEQ ID | |
| | | | ENST00000397562; | NO: 46); | NO: 54); | |
| | | | ENST00000397566; | NP_004022 | NM_004031 | |
| | | | ENST00000397570; | (SEQ ID | (SEQ ID | |
| | | | ENST00000397574; | NO: 47) | NO: 55) | |
| | | | GENSCAN00000065383; | | | |
| | | | NM_001572; | | | |
| | | | NM_004029; | | | |
| | | | NM_004031; | | | |
| | | | U53830; | | | |
| | | | U53831; | | | |
| | | | U53832; | | | |
| | | | U73036; | | | |
| | | | uc0011qf.1; | | | |
| | | | uc0011qg.1; | | | |
| | | | uc0011qh.1; | | | |
| | | | uc0011qi.1 | | | |

Table 7: Probe sets ID refer to the GeneChip Array “HG-U133A Array” from Affimetrix. Sequences from NCBI refer to Genome version March 2006 (NCBI Build 36.1). The genes were identified using the Gene Expression Omnibus Accession No. GSE7123 data set.

Thus, from the dynamics of the response in PBMC it can be seen that in positive responses the TLR pathway genes get activated (extra up regulation compared to non responders) resulting in the extra up regulation of the signature genes. From the static test (liver tissue of HCV patients before treatment) it can be seen that in non responders the 5 IFN signature genes are already up regulated prior to the treatment, meaning that the switch was pre-triggered to the “on” state, the genes reached their saturation level and can not increase their expression level as needed by the immune response to the interferon injection.

### Example 8

### The Signature Genes can Predict Response to Interferon in Multiple Sclerosis Patients

The differential expression of the 5 signature genes was validated in a data set of subjects receiving interferon I-β treatment for multiple sclerosis (MS). The microarray data set (Gene Expression Omnibus Accession No. GSE10655) was published by Baarsen et al 2008 [van Baarsen L G M, Vosslamber S, Tijssen M, Baggen J M C, van der Voort L F, et al. (2008) Pharmacogenomics of Interferon-β Therapy in Multiple Sclerosis: Baseline IFN Signature Determines Pharmacological Differences between Patients. PLoS ONE 3(4): e1927]. Interestingly only a third of the patients respond favorably to the treatment (e.g., by decreases of symptoms such as number of relapses per year), so there is a need to predict response to treatment in the MS group of patients as well. In this case the RNA was extracted from PBMC before treatment and at some points after treatment among interferon responders and non responders. Baarsen et al., 2008, found a switch behavior for a set of genes up regulated before treatment as indication for non responders.

Results

The 5-signature switch genes (G1P2, G1P3, IFIT1, OAS2 and OSA3) can also predict response to interferon treatment in multiple sclerosis (MS) patients—The present inventor has performed a clustergram analysis on the expression of the 5 signature genes using the MS microarray data (GSE10655). As shown in FIG. 10, in MS patients which do not respond to interferon (e.g., as determined by an increase in the number of relapses per year such as patients Nos. 7 and 17 in FIG. 10) the expression level of the signature genes is parallel to that of interferon non-responders among HCV patients. On the other hand, in MS patients which respond to interferon treatment (e.g., MS patients numbers 10 and 18 in FIG. 10, which switched from 3 relapses per year to 0 relapses per year), the expression level of the signature genes is in parallel to that of interferon responders among the HCV patients. Thus, these results demonstrate that the same expression pattern is observed in MS patients and HCV infected patients with respect to response to interferon.

These results suggest that the same genes act as a switch for the response to interferon among patients of two unrelated diseases, i.e., HCV (type 1, 2, 3 and 4) and multiple sclerosis.

Thus, it seems that the 5 signature genes (ISG15, IFI6, IF1T1, OAS2 and OAS3) act as a general static signature switch for any interferon I treatment, with the up state (genes up regulated) prior to treatment as an indication for a probable non response to the treatment.

As described, determination of the expression level of the 5 signature genes of type 1 HCV patients prior to injection can predict responders vs. non responders. In addition, the results of the dynamic approach (i.e., determining the level of expression in a liver biopsy before and after the first interferon injection) can be used to increase the predictability power of the method.

### Example 9

To test the involvement of natural killer inhibitory receptors in the response to interferon treatment the present inventor used the data set designated by Gene Expression Omnibus Accession No. gse11190.

Analysis of the expression pattern of genes in PBMC of HCV type 1 patients before interferon treatment (i.e., naïve to the treatment) revealed that the KIR2DL3, KIR3DL2, CD160, KLRG1, KIR3DL1, KIR3DL3, and KIR3DS1 are significantly down-regulated in interferon responders than in non-responders (FIG. 11).

Table 8 below provides the expression levels (in arbitrary expression units) of certain probes from the KIR3DL genes (KIR3DL1, KIR3DL2, and KIR3DL3) in PBMC of type 1 HCV patients prior to interferon treatment (time 0).

TABLE 8

| Probe | Gene | NR1_15 | NR1_14 | NR1_16 | NR1_12 | RR1_9 | RR1_10 | RR1_3 |
|---|---|---|---|---|---|---|---|---|
| 211687_x_at | KIR3DL1 | 54.34 | 99.89 | 154.84 | 45.13 | 29.58 | 15.70 | 17.87 |
| 207313_x_at | KIR3DL2 | 141.52 | 133.38 | 162.42 | 76.66 | 26.96 | 49.95 | 21.08 |
| 207314_x_at | KIR3DL2 | 123.76 | 178.36 | 421.33 | 97.30 | 65.26 | 54.63 | 23.61 |
| 211688_x_at | KIR3DL2 | 84.45 | 130.23 | 173.63 | 48.05 | 21.58 | 30.67 | 19.83 |
| 216907_x_at | KIR3DL2 | 91.87 | 135.88 | 148.08 | 81.13 | 29.06 | 41.32 | 22.43 |
| 216676_x_at | KIR3DL3 | 159.90 | 72.64 | 180.55 | 39.88 | 26.70 | 18.44 | 14.92 |

Table 8. Provided are the expression levels (arbitrary units) of the indicated KIR3DL genes in PBMC of type 1 HCV patients prior to interferon treatment.
NR1_15, NR1_14, NR1_16 and NT1_12 are non-responders to interferon;
RR1_9, RR1_10 and RR1_3 are responders to interferon.

Table 9 shows the fold change between the level of expression of the KIR inhibitory genes in non-responders as compared to responders in PBMC of HCV type 1 before interferon treatment (at time 0).

TABLE 9

| | Ratio non- | |
|---|---|---|
| | responders/ | |
| Gene name | responders | p-value |
| LOC730432 | 5.75848 | 0.006407 |
| KIR3DL3* | 5.65701 | 0.015599 |
| CD160* | 5.30257 | 0.004108 |
| KLRG1 | 5.17789 | 0.042744 |
| RAB8B | 5.16744 | 0.004787 |
| PLXDC1 | 5.02109 | 0.015832 |
| CLIC3 | 4.66021 | 0.001968 |
| KIR3DS1 | 4.65987 | 0.037145 |
| KIR3DL2* | 4.54073 | 0.009583 |
| KIR3DL2* | 4.28976 | 0.030791 |
| ETS1 | 4.25805 | 0.001047 |
| KIR3DL1* | 4.20611 | 0.014854 |
| KIR2DL1*///KIR2DL2*; | 4.19161 | 0.005581 |
| KIR2DL3*; KIR2DL5B*; | | |
| KIR2DS1*; KIR2DS2*; | | |
| KIR2DS3*; KIR2DS4*; | | |
| KIR2DS5*; KIR3DL1*; | | |
| KIR3DL2*; KIR3DL3*; | | |
| KIR3DS1* | | |
| APH1A | 4.14032 | 0.025299 |
| BAG2 | 4.07818 | 0.013842 |
| KIR3DL2* | 3.93383 | 0.004767 |
| KLRC3 | 3.93159 | 0.04951 |
| FLJ14213 | 3.81649 | 0.019087 |
| EDG8 | 3.79551 | 0.023646 |
| SLC16A3 | 3.79164 | 0.047258 |
| KIR3DL2* | 3.69283 | 0.002271 |
| SH2D1A | 3.68506 | 0.033533 |
| SENP7 | 3.67986 | 0.012762 |
| EDG8 | 3.67212 | 0.011557 |
| CCL4 | 3.63626 | 0.01551 |
| B3GNT2 | 3.58503 | 0.006146 |
| KIR2DL3* | 3.58336 | 0.001072 |
| ZNF146 | 3.53781 | 0.020323 |
| C1orf174 | 3.51223 | 0.044396 |
| SRR | 3.50893 | 0.014816 |

Table 9. Provided are the fold changes in expression levels of the indicated genes in non-responders versus responders to interferon treatment at time 0 (i.e., before the first interferon treatment). The data used for analysis is Gene Expression Omnibus Accession No. GSE11190. Genes marked with (*) are kir inhibitors.

The results presented in Table 9 above show that in type 1 HCV PBMC there are of 5 NK inhibitory receptors KIR3DL1, KIR3DL2, KIR3DL3, KLRG1 and CD160 which are upregulated in non-responders to interferon, thus indicating a poor prognosis to the subject infected with the HCV virus. Of them, CD160 exhibits a weak homology to KIR2DL4 and shows specific association with MHC class I molecule, and KLRG1 which belongs to the Natural Killer Receptor Family (KLR).

Altogether, these results show that in PBMC of non-responders the inhibitor KIR genes are significantly up regulated in comparison to the responders. Thus, upregulation of the KIR genes in HCV patients indicates probable failure of treatment by interferon. These results can explain the inability of the non responders to benefit from the support provided by the interferon injection.

### Example 10

### TNFRSF17 AND CXCL10 Can be Used in a Dynamic Method of Predicting Response to Interferon

In order to provide a further understanding and enhanced prediction power, the present inventor has analyzed the Gene Expression Omnibus Accession No. gse11190 data set by splitting into 2-groups (i.e., responders and non-responders), calculating log 2 ratios between the expression level in PBMC obtained 4 hours following interferon treatment and the expression level in PBMC before interferon treatment.

Results

Prediction power of the dynamic method using TNFRSF17 and CXCL10—As shown in FIGS. 12 and 13, using a Volcano graph analysis it is clear that the expression level of TNFRSF17, a receptor for the B cell growth factor BLyS-BAFF, by itself can predict the success (greater than 4 fold up-regulation) or failure (less than 2 fold up-regulation) of the IFN treatment.

In addition, as is further shown in FIG. 12, to further enhance and increase confidence of the prediction one can determine the expression level of CXCL10 or IP-10 (Interferon-inducible cytokine IP-10), which upregulation thereof 4 hours after interferon treatment as compared to prior to interferon treatment can predict success of interferon treatment.

Thus, the dynamic signature of both TNFRSF17 as a major indicator and IP-10 as a minor indicator, added to the previous KIR genes static expression prior to treatment and can provide a close to complete certainty prediction for type 1 HCV patients, taken from their PBMC samples.

### Example 11

### Identification of Genes which Exhibit the Most Correlated Expression Pattern to ISG15

The microarray used in the analyses described in Example 1 above, containing 14000 probes (Chen et al., 2005), revealed the exceptional consistency of ISG15 up-regulation in non responders compared to responders in type 1 HCV patients receiving pegylated IFN treatment. To identify additional genes which exhibit the most correlated expression pattern to ISG15 in liver tissues of HCV type 1 patients the present inventor analyzed the Gene Expression Omnibus Accession No. gse11190 data set (Affymetrix arrays u133 PLUS 2) which includes 47000 probe.

Results

As shown in Tables 10-12 below, the correlation between the expression pattern of various genes in non-responders and responders was compared to that of ISG15. It was found that the expression pattern of IFI27 is highly correlated with that of ISG15.

TABLE 10
Analysis of genes exhibiting a similar differential expression as ISG15 and IFI27 between
responders and non-responders to interferon

| Probe | Gen | | | | | | | | | |
|---|---|---|---|---|---|---|---|---|---|---|
| name | name | cont1 | cont2 | n_15 | n_16 | nr_12 | nr_14 | r_10 | r_3 | r_9 |
| 202411_at | IFI27 | 1468 | 1089 | 29259 | 33957 | 26342 | 20426 | 5577 | 4610 | 789 |
| 205483_s_at | ISG15 | 842 | 775 | 22192 | 27471 | 17024 | 16703 | 2257 | 1806 | 1514 |
| 211911_x_at | HLA-B | 10661 | 8206 | 27722 | 24707 | 23653 | 22816 | 14110 | 9484 | 7138 |
| 208729_x_at | HLA-B | 7812 | 7493 | 25039 | 22816 | 18026 | 17346 | 13035 | 7078 | 6264 |
| 215313_x_at | HLA-A | 11617 | 12395 | 25427 | 23653 | 25037 | 21670 | 17399 | 9009 | 9960 |
| 209140_x_at | HLA-B | 11882 | 11742 | 25831 | 21593 | 21928 | 24707 | 14074 | 11568 | 10117 |
| 214459_x_at | HLA-C | 13247 | 8304 | 21673 | 17499 | 23184 | 17873 | 12568 | 8840 | 10641 |
| 213932_x_at | HLA-A | 9655 | 11361 | 20901 | 15883 | 18819 | 15491 | 12174 | 8508 | 9090 |
| 214478_at | SPP2 | 8855 | 5969 | 18821 | 13627 | 16660 | 19769 | 11668 | 10096 | 7360 |
| 208812_x_at | HLA-C; | 16610 | 9282 | 19466 | 17824 | 23357 | 18076 | 11696 | 9655 | 11775 |
| | LOC732037 | | | | | | | | | |
| 206293_at | SULT2A1 | 4136 | 8292 | 17026 | 18132 | 11312 | 15666 | 12454 | 13345 | 6519 |
| 217757_at | A2M | 11914 | 13627 | 26660 | 25424 | 21359 | 19896 | 24807 | 7744 | 13175 |
| 208980_s_at | UBC | 9938 | 10703 | 16512 | 21359 | 13549 | 13695 | 13627 | 11964 | 11173 |
| 216526_x_at | HLA-C | 10434 | 10773 | 20486 | 13344 | 15624 | 15113 | 11052 | 9534 | 9219 |
| 206292_s_at | SULT2A1 | 5997 | 10618 | 18190 | 17973 | 11076 | 15193 | 13848 | 11775 | 9672 |
| 217933_s_at | LAP3 | 6364 | 7871 | 15842 | 16221 | 12075 | 8304 | 8746 | 7107 | 7255 |
| 211296_x_at | UBC | 14816 | 13035 | 18999 | 21438 | 16737 | 20761 | 16879 | 13280 | 12568 |
| 203153_at | IFIT1 | 1475 | 1659 | 10685 | 17920 | 8919 | 8977 | 2374 | 2261 | 1889 |
| 214328_s_at | HSP90AA1 | 11266 | 10272 | 14365 | 16275 | 15356 | 13549 | 12542 | 12846 | 8883 |
| 204533_at | CXCL10 | 261 | 697 | 14322 | 14364 | 10272 | 7097 | 9884 | 3145 | 1833 |
| 213738_s_at | ATP5A1 | 11853 | 14110 | 16416 | 20622 | 16082 | 19341 | 14542 | 13737 | 14699 |
| 224187_x_at | HSPA8 | 15024 | 11991 | 21595 | 19961 | 18248 | 16737 | 17697 | 18658 | 12454 |
| 209937_at | TM4SF4 | 7449 | 7549 | 12815 | 12937 | 13590 | 12568 | 8718 | 7838 | 10191 |
| 221891_x_at | HSPA8 | 10839 | 8780 | 18772 | 19278 | 14979 | 9906 | 14735 | 18998 | 12019 |
| 208687_x_at | HSPA8 | 12075 | 8152 | 15800 | 14320 | 14699 | 13590 | 12484 | 12783 | 11026 |
| 203382_s_at | APOE | 15883 | 12049 | 23359 | 17346 | 18366 | 14699 | 13411 | 18305 | 12938 |
| 217732_s_at | ITM2B | 13411 | 13445 | 15194 | 14857 | 18132 | 13411 | 11567 | 9549 | 10839 |
| 201553_s_at | LAMP1 | 7282 | 8584 | 13412 | 16703 | 10434 | 11076 | 11076 | 12543 | 9451 |
| 204532_x_at | UGT1A4 | 10389 | 12484 | 17401 | 16782 | 10250 | 16660 | 9250 | 14940 | 11441 |
| 205480_s_at | UGP2 | 13961 | 14901 | 18420 | 22443 | 17873 | 20691 | 16511 | 15955 | 16925 |

Table 10. Provided are the expression levels of the indicated genes among control subjects (cont1, cont2), non-responders (n_15, n_16, nr_12, nr_14) and responders (r_10, r_3, r_9).

TABLE 11
Continued analysis of genes exhibiting a similar differential expression as ISG15 and IFI27
between responders and non-responders to interferon

| | | | | Similarity | | | |
|---|---|---|---|---|---|---|---|
| | Similarity | Similarity | Similarity | to 1sg15 | | | |
| | to isg15 | to isg15 | to 1sg15 | corr | | | |
| Probe | euc | euc (rank) | corr | (rank) | avg_nr | avg_r | log2_nr_r |
| 202411_at | 14580.22 | 4.00 | 0.99 | 3.00 | 27495.63 | 3658.46 | 2.91 |
| 205483_s_at | 0.00 | 3.00 | 1.00 | 1.00 | 20847.43 | 1858.92 | 3.49 |
| 211911_x_at | 22410.78 | 11.00 | 0.94 | 43.00 | 24724.51 | 10243.84 | 1.27 |
| 208729_x_at | 17071.10 | 5.00 | 0.95 | 39.00 | 20806.84 | 8792.21 | 1.24 |
| 215313_x_at | 26786.77 | 42.00 | 0.90 | 131.00 | 23946.58 | 12122.75 | 0.98 |
| 209140_x_at | 26210.76 | 35.00 | 0.91 | 97.00 | 23514.59 | 11919.48 | 0.98 |
| 214459_x_at | 24262.36 | 20.00 | 0.83 | 401.00 | 20057.34 | 10682.67 | 0.91 |
| 213932_x_at | 23043.64 | 12.00 | 0.86 | 290.00 | 17773.48 | 9923.83 | 0.84 |
| 214478_at | 22253.19 | 10.00 | 0.79 | 663.00 | 17219.24 | 9708.23 | 0.83 |
| 208812_x_at | 26822.54 | 44.00 | 0.76 | 934.00 | 19680.85 | 11042.10 | 0.83 |
| 206293_at | 21843.55 | 9.00 | 0.79 | 626.00 | 15533.96 | 10772.65 | 0.53 |
| 217757_at | 31947.92 | 317.00 | 0.75 | 999.00 | 23334.58 | 15242.09 | 0.61 |
| 208980_s_at | 24449.96 | 22.00 | 0.88 | 185.00 | 16278.71 | 12254.79 | 0.41 |
| 216526_x_at | 24400.16 | 21.00 | 0.79 | 659.00 | 16141.86 | 9935.13 | 0.70 |
| 206292_s_at | 23825.51 | 17.00 | 0.79 | 670.00 | 15607.89 | 11765.17 | 0.41 |
| 217933_s_at | 21116.89 | 8.00 | 0.90 | 129.00 | 13110.50 | 7702.71 | 0.77 |
| 211296_x_at | 29603.65 | 126.00 | 0.88 | 206.00 | 19483.84 | 14242.53 | 0.45 |
| 203153_at | 18722.86 | 6.00 | 0.98 | 15.00 | 11625.39 | 2174.41 | 2.42 |
| 214328_s_at | 26079.73 | 34.00 | 0.85 | 301.00 | 14886.25 | 11423.97 | 0.38 |
| 204533_at | 20785.14 | 7.00 | 0.86 | 278.00 | 11513.50 | 4954.22 | 1.22 |
| 213738_s_at | 29228.83 | 105.00 | 0.86 | 266.00 | 18115.11 | 14326.35 | 0.34 |
| 224187_x_at | 32050.92 | 334.00 | 0.71 | 1336.00 | 19135.46 | 16269.75 | 0.23 |
| 209937_at | 23895.80 | 18.00 | 0.90 | 121.00 | 12977.32 | 8915.67 | 0.54 |
| 221891_x_at | 29237.70 | 106.00 | 0.50 | 5362.00 | 15733.82 | 15250.82 | 0.04 |
| 208687_x_at | 26927.02 | 46.00 | 0.77 | 835.00 | 14602.18 | 12097.82 | 0.27 |
| 203382_s_at | 31466.61 | 272.00 | 0.61 | 2842.00 | 18442.67 | 14884.66 | 0.31 |
| 217732_s_at | 27789.18 | 63.00 | 0.67 | 1849.00 | 15398.42 | 10651.75 | 0.53 |
| 201553_s_at | 25032.81 | 24.00 | 0.76 | 945.00 | 12906.13 | 11023.31 | 0.23 |
| 204532_x_at | 27042.20 | 52.00 | 0.66 | 2049.00 | 15273.05 | 11876.76 | 0.36 |
| 205480_s_at | 32690.47 | 418.00 | 0.88 | 203.00 | 19856.97 | 16463.68 | 0.27 |

Table 11. Provided is continued analysis of similarity of the expression of the indicated probes/genes to ISG15. isg15 euc (using Euclidean distance measurement); Similarity to isg15 euc (euc distance ranking distance rank); Similarity to 1sg15 corr (correlation measurement to isg15); Similarity to 1sg15 corr (ranking correlation rank); avg_nr (average value of non responders); avg_r (average value of responders); log2_nr_r (log2 ratio responders to non responders);

TABLE 12
Continued analysis of genes exhibiting a similar differential expression as ISG15 and IFI27
between responders and non-responders to interferon

| | | Similarity | | Similarity | | | | |
|---|---|---|---|---|---|---|---|---|
| | Similarity | to | Similarity | to | | | | |
| | to | Average | to | Average | | Similarity | | |
| | Average | (Edited) | Average | (Edited) | Similarity | to | Similarity | Similarity |
| | (Edited) | square | (Edited) | square* | to | 211799_x_at | to | to |
| Probe | square | (rank) | square*2 | 2 (rank) | 211799_x_at | (rank) | IFI27 | IFI27(rank) |
| 202411_at | 36952.58 | 54435.00 | 23456.36 | 159.00 | 38727.18 | 54514.00 | 0.00 | 1.00 |
| 205483_s_at | 23549.69 | 54257.00 | 11358.13 | 3.00 | 26451.97 | 54403.00 | 14580.22 | 2.00 |
| 211911_x_at | 36915.08 | 54433.00 | 25553.28 | 485.00 | 33978.02 | 54475.00 | 19310.99 | 3.00 |
| 208729_x_at | 29190.18 | 54353.00 | 19314.81 | 19.00 | 26082.72 | 54398.00 | 19836.98 | 4.00 |
| 215313_x_at | 38869.62 | 54460.00 | 28776.71 | 1355.00 | 34800.91 | 54483.00 | 24457.27 | 5.00 |
| 209140_x_at | 37552.37 | 54440.00 | 27522.52 | 942.00 | 33601.91 | 54469.00 | 25139.51 | 6.00 |
| 214459_x_at | 31306.57 | 54375.00 | 23040.75 | 133.00 | 26371.28 | 54402.00 | 26448.24 | 7.00 |
| 213932_x_at | 27339.65 | 54322.00 | 20758.71 | 38.00 | 21923.79 | 54353.00 | 27885.55 | 8.00 |
| 214478_at | 24523.09 | 54274.00 | 18138.98 | 16.00 | 20159.27 | 8643.00 | 28375.54 | 9.00 |
| 208812_x_at | 32862.79 | 54394.00 | 25436.27 | 455.00 | 27342.74 | 54410.00 | 29164.62 | 10.00 |
| 206293_at | 23941.72 | 54266.00 | 19687.37 | 24.00 | 20066.25 | 8218.00 | 29394.38 | 11.00 |
| 217757_at | 43143.76 | 54494.00 | 34465.50 | 54435.00 | 38186.94 | 54506.00 | 30067.75 | 12.00 |
| 208980_s_at | 28608.68 | 54340.00 | 23894.13 | 201.00 | 22966.72 | 54361.00 | 30348.44 | 13.00 |
| 216526_x_at | 25736.18 | 54290.00 | 20952.51 | 42.00 | 19466.05 | 6208.00 | 30730.96 | 14.00 |
| 206292_s_at | 26243.80 | 54303.00 | 22074.84 | 84.00 | 21094.42 | 28256.00 | 30796.30 | 15.00 |
| 217933_s_at | 18241.29 | 4296.00 | 17221.78 | 12.00 | 12543.33 | 888.00 | 31194.43 | 16.00 |
| 211296_x_at | 36412.45 | 54430.00 | 29699.13 | 1819.00 | 30625.46 | 54440.00 | 31888.64 | 17.00 |
| 203153_at | 8799.94 | 7.00 | 13110.90 | 4.00 | 12069.03 | 786.00 | 32466.44 | 18.00 |
| 214328_s_at | 26252.97 | 54304.00 | 22979.69 | 129.00 | 19760.38 | 7096.00 | 32628.46 | 19.00 |
| 204533_at | 11969.05 | 34.00 | 15047.21 | 5.00 | 11585.22 | 693.00 | 32654.10 | 20.00 |
| 213738_s_at | 34336.50 | 54411.00 | 28540.21 | 1255.00 | 28708.81 | 54423.00 | 32920.87 | 21.00 |
| 224187_x_at | 38361.15 | 54450.00 | 32152.70 | 4241.00 | 32084.17 | 54454.00 | 33416.61 | 22.00 |
| 209937_at | 18879.12 | 6303.00 | 18046.03 | 14.00 | 12778.13 | 944.00 | 33507.84 | 23.00 |
| 221891_x_at | 32407.07 | 54389.00 | 28549.63 | 1258.00 | 26184.39 | 54399.00 | 33603.24 | 24.00 |
| 208687_x_at | 26359.68 | 54307.00 | 23394.39 | 154.00 | 19425.69 | 6104.00 | 33626.30 | 25.00 |
| 203382_s_at | 36695.13 | 54431.00 | 30907.53 | 2670.00 | 30108.03 | 54436.00 | 33638.54 | 26.00 |
| 217732_s_at | 28051.87 | 54331.00 | 24413.91 | 284.00 | 21050.07 | 20970.00 | 33701.84 | 27.00 |
| 201553_s_at | 22667.66 | 54239.00 | 21696.97 | 64.00 | 16688.77 | 2525.00 | 33874.36 | 28.00 |
| 204532_x_at | 28327.05 | 54334.00 | 24835.40 | 359.00 | 22453.44 | 54356.00 | 33938.15 | 29.00 |
| 205480_s_at | 39619.05 | 54466.00 | 33024.61 | 6547.00 | 33908.87 | 54472.00 | 34312.25 | 30.00 |

Table 12. Continued analysis of similarity of the indicated probes/genes to ISG15. Similarity to Average (edited signal 250 250 1000 1000 1000 1000 500 500 500 euclidean distance) square; Similarity to Average (Edited) square (ranking of previous measured distance); Similarity to Average (Edited) square*2 (edited signal 700 700 17000 17000 17000 17000 1500 1500 1500); Similarity to Average (Edited) square*2 (ranking of the previous data); Similarity to 211799_x_at (Euclidean distance tO HLA-C); Similarity to 211799_x_at (ranking of the former); Similarity to IFI27 = (oclidean distance to the IFI27 profile); Similarity to IFI27(rank) (ranking of the previous);

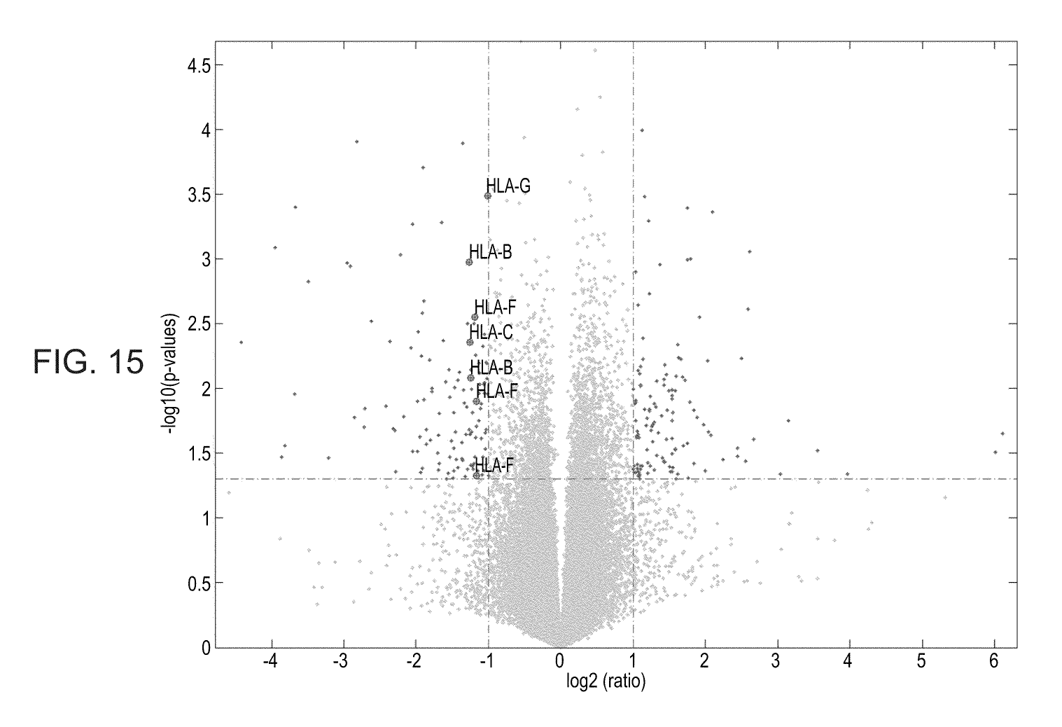

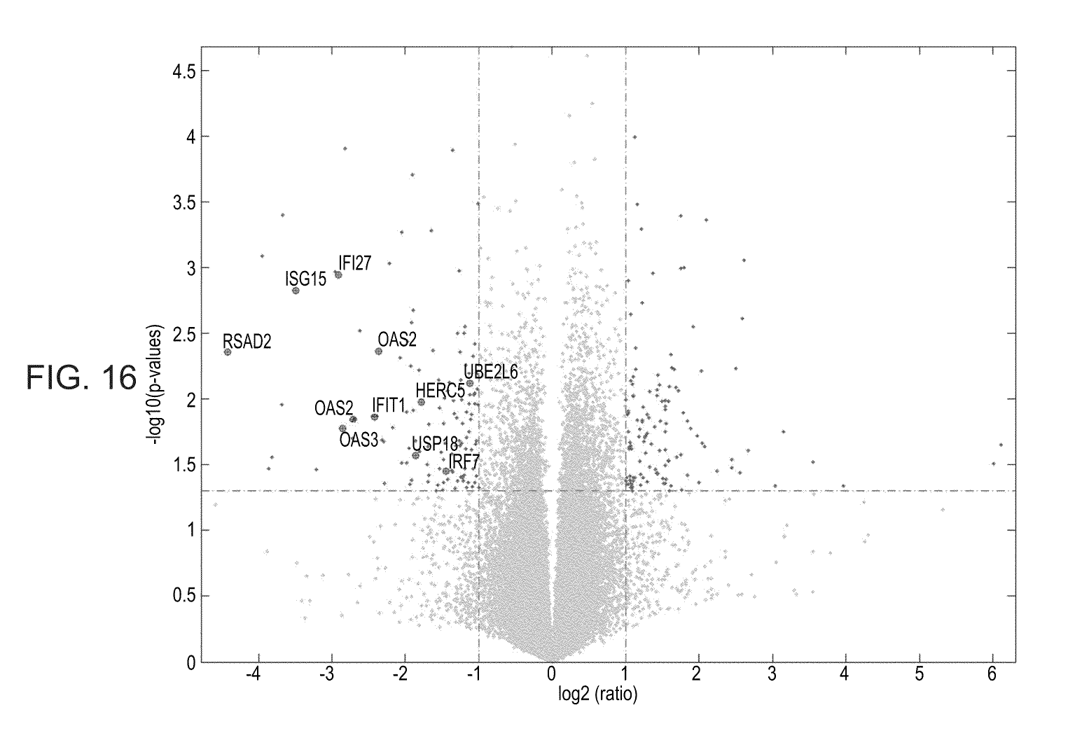

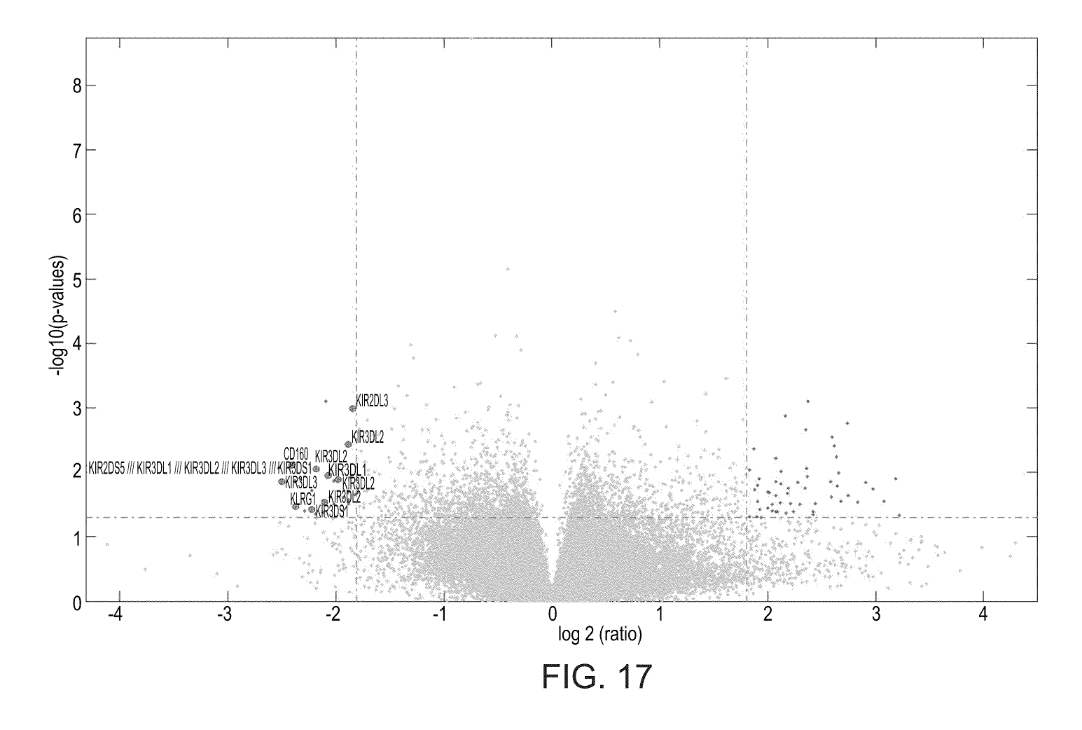

As shown in Tables 10-12 above and FIG. 14, the expression pattern of the HLA family of genes is similar to that of ISG15 in liver tissues of HCV type 1 patients. In addition, as shown in FIG. 15, in the liver of HCV type 1 patients the level of HLA-B, HLA-F, HLA-C and HLA-G before any injection of interferon is significantly upregulated (at least 2 fold change) in non-responders as compared to interferon responders. In parallel and even with a stronger up-regulation, the former described switch genes (Example 1) show similar behavior in non responders using the gse11190 data set. Thus, as shown in FIG. 16 the level of ISG15, IFIT1, USP18, OAS2, OAS3, and HERC6 in PBMC before any injection of interferon is significantly upregulated (at least 4.6 fold change) in non-responders HCV type 1 patients as compared to responders.

As described in Example 9 above, in PBMC of non responders type 1 HCV patients there is a significant up-regulation of the inhibitor KIR genes. Thus, one of the major actions of the HCV virus is deciphered here: In the liver tissue the virus succeeds in activating the HLA genes (HLA-A, HLA-B, HLA-C) which results in the appearance of their complementary inhibitory KIR (e.g., KIR3D, KIR2D) in the PBMC of these patients.

### Example 12

### Matching Expression Pattern of HLA Genes in Liver and kir Genes in Blood of Non-Responder HCV Types 1-4 Patients

Further analysis of the upregulated genes in various biological pathways (using the ontoexpress software (Intelligent Systems and Bioinformatics Laboratory, Computer Science Department, Wayne State University) revealed that the most statistically significant pathway for both expression in liver and blood of non-responders versus responder is the natural killer cell mediated cytotoxicity pathway as shown in FIGS. 18 and 19. These analyses show that while the MHC class 1 genes are up-regulated in the target cells (liver) of non-responders, there is a matching upregulation of the KIR inhibitory genes in the PBMC of non-responders, resulting in inhibition of NK cells and preventing their action against the liver cells hosting the HCV virus.

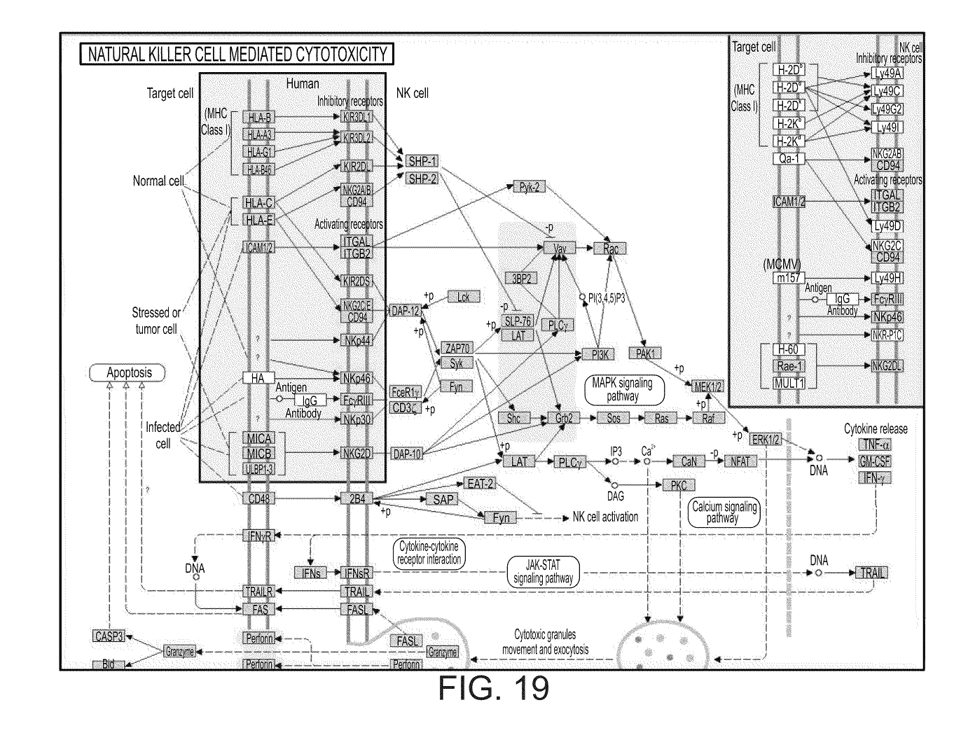

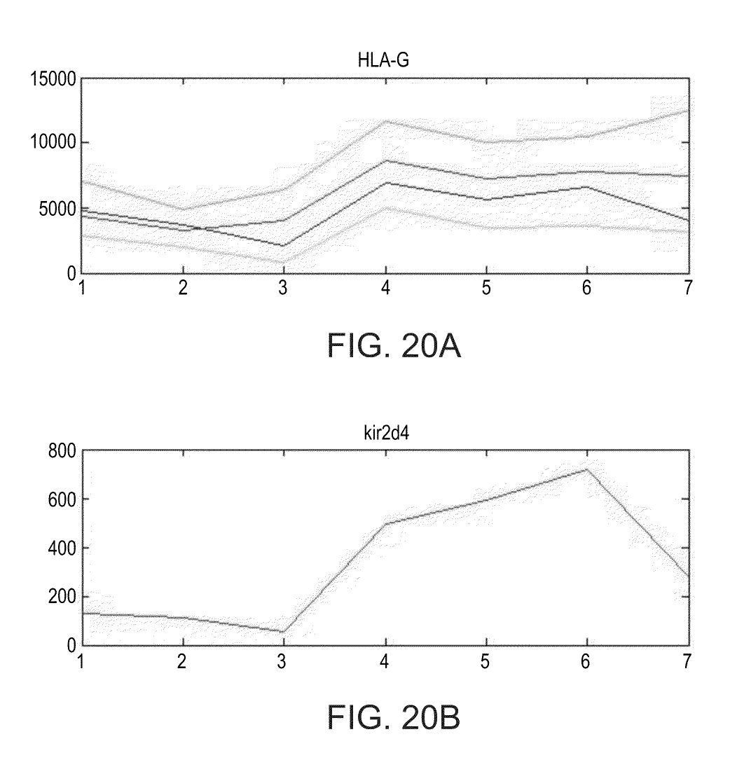

Coordinated upregulation of HLA genes in liver and kir genes in blood of non-responders HCV type 1 patients at time 0 (naïve to interferon)—FIGS. 20A-B, 21A-B and 22A-B demonstrate the expression of various HLA genes in liver tissues and of their matching kir genes in the blood of HCV type 1 patients. Hence in non responders to the IFN treatment the liver tissue shows up regulation of the HLA (MHC CLASS 1) genes while at the same time their corresponding inhibitory KIR genes are up regulated compared to the responders.

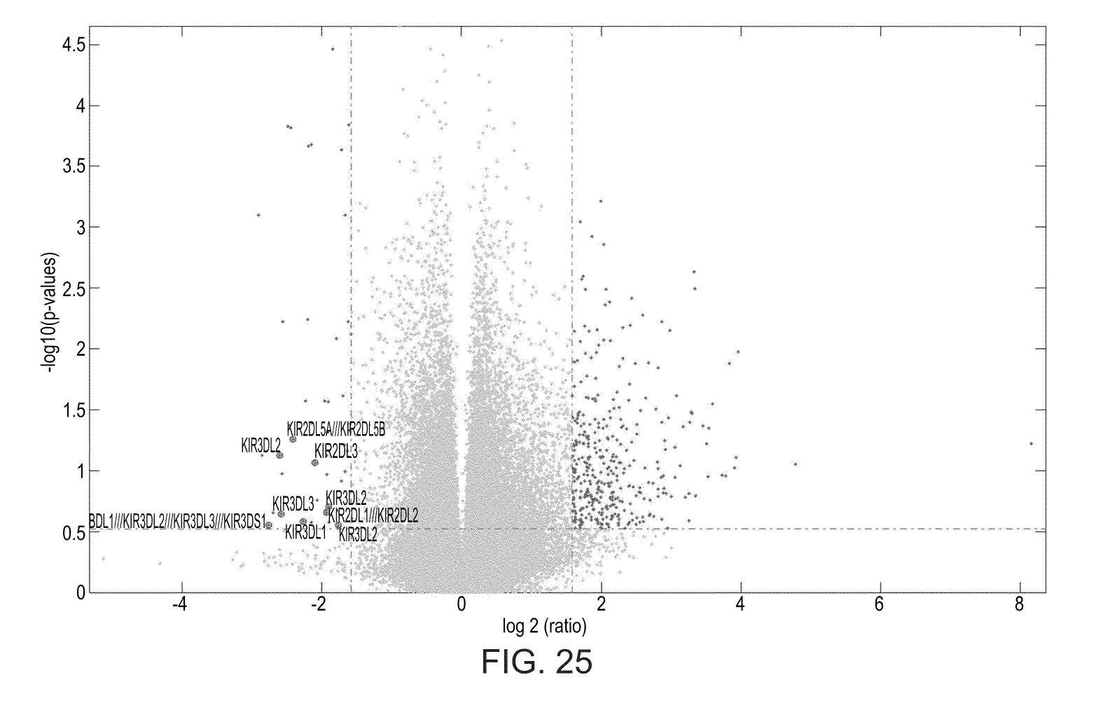

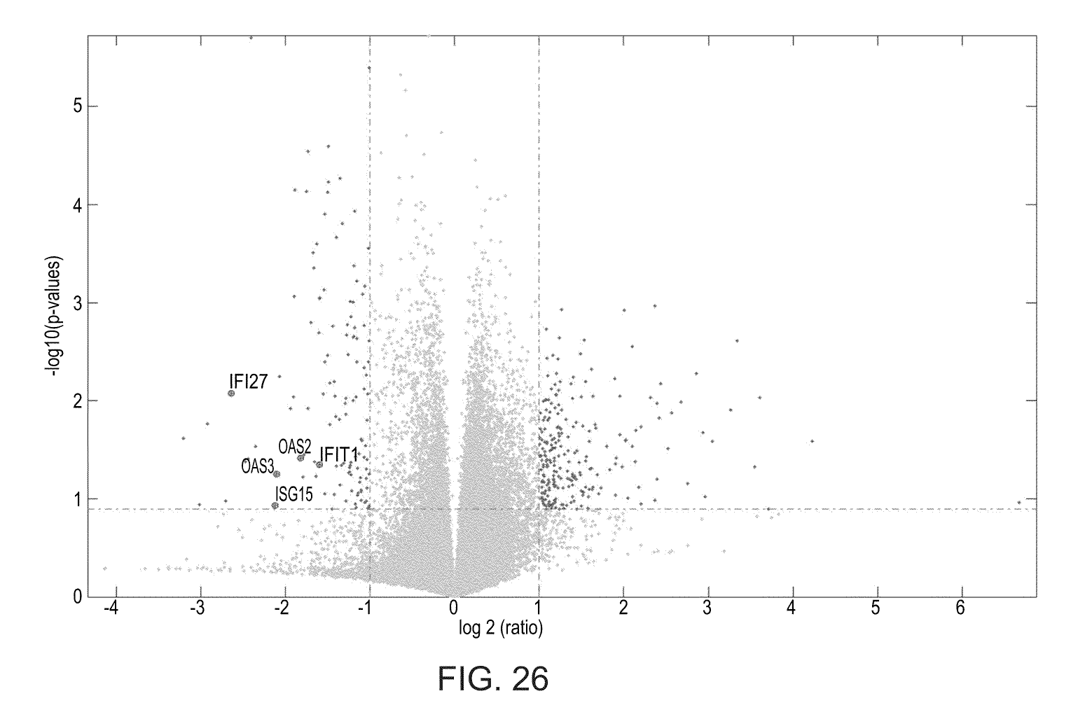

The kir genes are upregulated in blood samples of non-responders HCV types 2-4 patients at time 0 (naïve to interferon)—As shown in FIG. 25, in HCV types 2-4 patients the same kir genes (e.g., KIR2DL5A, KIR2DL5B, KIR2DL3, KIR3DL1, KIR2DL1, KIR2DL2, KIR3DL3) are upregulated in PBMC of non-responders as compared to responders, similar to the profile in blood of HCV type 1 non-responders. In addition, in liver tissues of non-responders HCV types 2-4, the same ISG15 pathway is activated in non-responders as compared to responders (FIG. 26), similar to the liver profile of HCV type 1 non-responders.

### Example 13

### Identification of a Common Promoter to the Genes which are Up-Regulated in Non-Responders to Interferon Treatment

The present inventor has surprisingly uncovered that the genes which are upregulated in non-responders to interferon (e.g., HLA-A, HLA-B, HLA-C, HLA-F, ISG15, IFI27, IFIT1, IFI6, OAS2, and OAS3) have the same promoter ISRE in the near 300 bp upstream region of the gene (FIG. 23). Tables 13 and 14 depict the frequency score [determined toucan workbench for regulatory sequence analysis; Hypertext Transfer Protocol://homes (dot) esat (dot) kuleuven (dot) be/˜saerts/software/toucan (dot) php] of promoters in the 300 bp (Table 14) and 2000 bp (Table 15) upstream region of the analyzed genes which are upregulated in liver of HCV type 1 non-responders. It should be noted that higher scores indicate higher probabilities of the promoter being active for these genes.

TABLE 14
Promoters in the 300 bp upstream region

| Promoter name | Score |
|---|---|
| M00302-V$NFAT_Q6 | 0.001097804 |
| M00258-V$ISRE_01 | 0.001097804 |
| M00062-V$IRF1_01 | 9.98E−04 |
| M00453-V$IRF7_01 | 9.98E−04 |
| M00063-V$IRF2_01 | 7.98E−04 |
| M00138-V$OCT1_04 | 6.99E−04 |
| M00148-V$SRY_01 | 6.99E−04 |
| M00380-V$PAX4_04 | 6.99E−04 |
| M00083-V$MZF1_01 | 5.99E−04 |
| M00141-V$LYF1_01 | 5.99E−04 |
| M00208-V$NFKB_C | 5.99E−04 |
| M00456-V$FAC1_01 | 5.99E−04 |
| M00194-V$NFKB_Q6 | 4.99E−04 |
| M00471-V$TBP_01 | 4.99E−04 |
| M00486-V$PAX2_02 | 4.99E−04 |
| M00145-V$BRN2_01 | 4.99E−04 |
| M00054-V$NFKAPPAB_01 | 4.99E−04 |
| M00051-V$NFKAPPAB50_01 | 4.99E−04 |
| M00130-V$FOXD3_01 | 4.99E−04 |

Table 14: Provided are the promoters found in the 300 bp upstream sequence of the analyzed genes (upregulated in liver tissue of HCV type 1 non-responders) along with the frequency score of each promoter.

TABLE 15
Promoters in the 2000 bp upstream region

| Promoter name | Score |
|---|---|
| M00138-V$OCT1_04 | 4.54E−04 |
| M00130-V$FOXD3_01 | 4.32E−04 |
| M00258-V$ISRE_01 | 3.86E−04 |
| M00096-V$PBX1_01 | 3.86E−04 |
| M00453-V$IRF7_01 | 3.86E−04 |
| M00380-V$PAX4_04 | 3.86E−04 |
| M00081-V$EVI1_04 | 3.63E−04 |
| M00456-V$FAC1_01 | 3.63E−04 |
| M00131-V$HNF3B_01 | 3.41E−04 |
| M00160-V$SRY_02 | 3.41E−04 |
| M00116-V$CEBPA_01 | 3.41E−04 |
| M00471-V$TBP_01 | 3.18E−04 |
| M00268-V$XFD2_01 | 3.18E−04 |
| FivePrimeUTR | 2.95E−04 |
| M00216-V$TATA_C | 2.95E−04 |
| M00302-V$NFAT_Q6 | 2.95E−04 |
| M00377-V$PAX4_02 | 2.95E−04 |
| exon | 2.95E−04 |
| M00472-V$FOXO4_01 | 2.95E−04 |
| M00473-V$FOXO1_01 | 2.73E−04 |
| M00289-V$HFH3_01 | 2.73E−04 |

Table 15: Provided are the promoters found in the 2000 bp upstream sequence of the analyzed genes (upregulated in liver tissue of HCV type 1 non-responders) along with the frequency score of each promoter.

Table 16 provides sequence information of identified genes according to some embodiments of the invention.

TABLE 16

| Probe Set | Gene | | RefSeq | RefSeq |
|---|---|---|---|---|
| ID | Symbol | Gene Title | Protein ID | Transcript ID |
| 202411_at | IFI27 | interferon alpha-inducible | NP_001123552 | NM_001130080 |
| (SEQ ID | | protein 27 | (SEQ ID NO: 91); | (SEQ ID NO: 123); |
| NO: 56) | | | NP_005523 (SEQ | NM_005532 (SEQ |
| | | | ID NO: 164) | ID NO: 124) |
| 204533_at | CXCL10 | chemokine (C-X-C motif) | NP_001556 (SEQ | NM_001565 (SEQ |
| (SEQ ID | | ligand 10 | ID NO: 92) | ID NO: 125) |
| NO: 57) | | | | |
| 206641_at | TNFRSF17 | tumor necrosis factor | NP_001183 (SEQ | NM_001192 (SEQ |
| (SEQ ID | | receptor superfamily, | ID NO: 93) | ID NO: 126) |
| NO: 58) | | member 17 | | |
| 207313_x_at | KIR3DL2; | killer cell immunoglobulin- | NP_006728 (SEQ | NM_006737 (SEQ |
| (SEQ ID | LOC727787 | like receptor, three | ID NO: 94); | ID NO: 127); |
| NO: 59) | | domains, long cytoplasmic | XP_001718677 | XM_001718625 |
| | | tail, 2; | (SEQ ID NO: 95) | (SEQ ID NO: 128) |
| 207314_x_at | KIR3DL2; | killer cell immunoglobulin- | NP_006728 (SEQ | NM_006737 (SEQ |
| (SEQ ID | LOC727787 | like receptor, three | ID NO: 94); | ID NO: 127); |
| NO: 60) | | domains, long cytoplasmic | XP_001718677 | XM_001718625 |
| | | tail, 2; | (SEQ ID NO: 95) | (SEQ ID NO: 128) |
| 211688_x_at | KIR3DL2; | killer cell immunoglobulin- | NP_006728 (SEQ | NM_006737 (SEQ |
| (SEQ ID | LOC727787 | like receptor, three | ID NO: 94); | ID NO: 127); |
| NO: 61) | | domains, long cytoplasmic | XP_001718677 | XM_001718625 |
| | | tail, 2; | (SEQ ID NO: 95) | (SEQ ID NO: 128) |
| 216907_x_at | KIR3DL2; | killer cell immunoglobulin- | NP_006728 (SEQ | NM_006737 (SEQ |
| (SEQ ID | LOC727787 | like receptor, three | ID NO: 94); | ID NO: 127); |
| NO: 62) | | domains, long cytoplasmic | XP_001718677 | XM_001718625 |
| | | tail, 2; | (SEQ ID NO: 95) | (SEQ ID NO: 128) |
| 211397_x_at | KIR2DL2 | killer cell immunoglobulin- | NP_055034 (SEQ | NM_014219 (SEQ |
| (SEQ ID | | like receptor, two domains, | ID NO: 96) | ID NO: 129) |
| NO: 63) | | long cytoplasmic tail, 2 | | |
| 207840_at | CD160 | CD160 molecule | NP_008984 (SEQ | NM_007053 (SEQ |
| (SEQ ID | | | ID NO: 97) | ID NO: 130) |
| NO: 64) | | | | |
| 208179_x_at | KIR2DL3 | killer cell immunoglobulin- | NP_055326 (SEQ | NM_014511 (SEQ |
| (SEQ ID | | like receptor, two domains, | ID NO: 98); | ID NO: 131); |
| NO: 65) | | long cytoplasmic tail, 3 | NP_056952 (SEQ | NM_015868 (SEQ |
| | | | ID NO: 99) | ID NO: 132) |
| 208426_x_at | KIR2DL3; | killer cell immunoglobulin- | NP_001074239 | NM_001080770 |
| (SEQ ID | KIR2DL4; | like receptor, two domains, | (SEQ ID NO: 100); | (SEQ ID NO: 133); |
| NO: 66) | KIR2DL5A | long cytoplasmic tail, 3 | NP_001074241 | NM_001080772 |
| | | (KIR2DL3); killer cell | (SEQ ID NO: 101); | (SEQ ID NO: 134); |
| | | immunoglobulin-like | NP_002246 (SEQ | NM_002255 (SEQ |
| | | receptor, two domains, long | ID NO: 102); | ID NO: 135); |
| | | cytoplasmic tail, 4 | NP_055326 (SEQ | NM_014511 (SEQ |
| | | (KIR2DL4); killer cell | ID NO: 98); | ID NO: 131); |
| | | immunoglobulin-like | NP_056952 (SEQ | NM_015868 (SEQ |
| | | receptor, two domains, long | ID NO: 99); | ID NO: 132); |
| | | cytoplasmic tail, 5A | NP_065396 (SEQ | NM_020535 (SEQ |
| | | (KIR2DL5A) | ID NO: 105) | ID NO: 136) |
| 208650_s_at | CD24 | CD24 molecule | NP_037362 (SEQ | NM_013230 (SEQ |
| (SEQ ID | | | ID NO: 106) | ID NO: 137) |
| NO: 67) | | | | |
| 208651_x_at | CD24 | CD24 molecule | NP_037362 (SEQ | NM_013230 (SEQ |
| (SEQ ID | | | ID NO: 106) | ID NO: 137) |
| NO: 68) | | | | |
| 216379_x_at | CD24 | CD24 molecule | NP_037362 (SEQ | NM_013230 (SEQ |
| (SEQ ID | | | ID NO: 106) | ID NO: 137) |
| NO: 69) | | | | |
| 209771_x_at | CD24 | CD24 molecule | NP_037362 (SEQ | NM_013230 (SEQ |
| (SEQ ID | | | ID NO: 106) | ID NO: 137) |
| NO: 70) | | | | |
| 209772_s_at | CD24 | CD24 molecule | NP_037362 (SEQ | NM_013230 (SEQ |
| (SEQ ID | | | ID NO: 106) | ID NO: 137) |
| NO: 71) | | | | |
| 266_s_at | CD24 | CD24 molecule | NP_037362 (SEQ | NM_013230 (SEQ |
| (SEQ ID | | | ID NO: 106) | ID NO: 137) |
| NO: 72) | | | | |
| 208729_x_at | HLA-B | major histocompatibility | NP_005505 (SEQ | NM_005514 (SEQ |
| (SEQ ID | | complex, class I, B | ID NO: 107) | ID NO: 138) |
| NO: 73) | | | | |
| 209140_x_at | HLA-B | major histocompatibility | NP_005505 (SEQ | NM_005514 (SEQ |
| (SEQ ID | | complex, class I, B | ID NO: 107) | ID NO: 138) |
| NO: 74) | | | | |
| 211911_x_at | HLA-B | major histocompatibility | NP_005505 (SEQ | NM_005514 (SEQ |
| (SEQ ID | | complex, class I, B | ID NO: 107) | ID NO: 138) |
| NO: 75) | | | | |
| 208812_x_at | HLA-C | major histocompatibility | NP_002108 (SEQ | NM_002117 (SEQ |
| (SEQ ID | | complex, class I, C | ID NO: 108) | ID NO: 139) |
| NO: 76) | | | | |
| 211146_at | HLA-C | Major histocompatibility | NP_002108 (SEQ | NM_002117 (SEQ |
| (SEQ ID | | complex, class I, C | ID NO: 108) | ID NO: 139) |
| NO: 77) | | | | |
| 211799_x_at | HLA-C | major histocompatibility | NP_002108 (SEQ | NM_002117 (SEQ |
| (SEQ ID | | complex, class I, C | ID NO: 108) | ID NO: 139) |
| NO: 78) | | | | |
| 214459_x_at | HLA-C | major histocompatibility | NP_002108 (SEQ | NM_002117 (SEQ |
| (SEQ ID | | complex, class I, C | ID NO: 108) | ID NO: 139) |
| NO: 79) | | | | |
| 216526_x_at | HLA-C | major histocompatibility | NP_002108 (SEQ | NM_002117 (SEQ |
| (SEQ ID | | complex, class I, C | ID NO: 108) | ID NO: 139 same as |
| NO: 80) | | | | above) |
| 210288_at | KLRG1 | killer cell lectin-like | NP_005801 (SEQ | NM_005810 (SEQ |
| (SEQ ID | | receptor subfamily G, | ID NO: 109) | ID NO: 140) |
| NO: 81) | | member 1 | | |
| 210514_x_at | HLA-G | major histocompatibility | NP_002118 (SEQ | NM_002127 (SEQ |
| (SEQ ID | | complex, class I, G | ID NO: 110) | ID NO: 141) |
| NO: 82) | | | | |
| 211528_x_at | HLA-G | major histocompatibility | NP_002118 (SEQ | NM_002127 (SEQ |
| (SEQ ID | | complex, class I, G | ID NO: 110) | ID NO: 141) |
| NO: 83) | | | | |
| 211529_x_at | HLA-G | major histocompatibility | NP_002118 (SEQ | NM_002127 (SEQ |
| (SEQ ID | | complex, class I, G | ID NO: 110) | ID NO: 141) |
| NO: 84) | | | | |
| 211530_x_at | HLA-G | major histocompatibility | NP_002118 (SEQ | NM_002127 (SEQ |
| (SEQ ID | | complex, class I, G | ID NO: 110) | ID NO: 141) |
| NO: 85) | | | | |
| 211389_x_at | KIR3DS1 | killer cell immunoglobulin- | NP_001077008 | NM_001083539 |
| (SEQ ID | | like receptor, three | (SEQ ID NO: 111) | (SEQ ID NO: 142) |
| NO: 86) | | domains, short cytoplasmic | | |
| | | tail, 1 | | |
| 211687_x_at | KIR3DL1 | killer cell immunoglobulin- | NP_037421 (SEQ | NM_013289 (SEQ |
| (SEQ ID | | like receptor, three | ID NO: 145) | ID NO: 143) |
| NO: 87) | | domains, long cytoplasmic | | |
| | | tail, 1 | | |
| 213932_x_at | HLA-A | major histocompatibility | NP_002107 (SEQ | NM_002116 (SEQ |
| (SEQ ID | | complex, class I, A | ID NO: 112) | ID NO: 144) |
| NO: 88) | | | | |
| 215313_x_at | HLA-A | major histocompatibility | NP_002107 (SEQ | NM_002116 (SEQ |
| (SEQ ID | | complex, class I, A | ID NO: 112) | ID NO: 144) |
| NO: 89) | | | | |
| 216676_x_at | KIR3DL3 | killer cell immunoglobulin- | NP_703144 (SEQ | NM_153443 (SEQ |
| (SEQ ID | | like receptor, three | ID NO: 113) | ID NO: 146) |
| NO: 90) | | domains, long cytoplasmic | | |
| | | tail, 3 | | |
| 217318_x_at | KIR2DL1; | killer cell immunoglobulin- | NP_001015070 | NM_001015070 |
| (SEQ ID | KIR2DL2; | like receptor, two domains, | (SEQ ID NO: 114); | (SEQ ID NO: 147); |
| NO: 160) | KIR2DL3; | long cytoplasmic tail, 1 | NP_001018091 | NM_001018081 |
| | KIR2DL5A; | (KIR2DL1); killer cell | (SEQ ID NO: 148); | (SEQ ID NO: 149); |
| | KIR2DL5B; | immunoglobulin-like | NP_006728 (SEQ | NM_006737 (SEQ |
| | KIR2DS1; | receptor, two domains, long | ID NO: 94); | ID NO: 127); |
| | KIR2DS2; | cytoplasmic tail, 2 | NP_036444 (SEQ | NM_012312 (SEQ |
| | KIR2DS3; | (KIR2DL2); killer cell | ID NO: 115); | ID NO: 150); |
| | KIR2DS4; | immunoglobulin-like | NP_036445 (SEQ | NM_012313 (SEQ |
| | KIR2DS5; | receptor, two domains, long | ID NO: 116); | ID NO: 151); |
| | KIR3DL2; | cytoplasmic tail 3, | NP_036446 (SEQ | NM_012314 (SEQ |
| | KIR3DL3; | (KIR2DL3); killer cell | ID NO: 117); | ID NO: 152); |
| | KIR3DP1; | immunoglobulin-like | NP_055033 (SEQ | NM_014218 (SEQ |
| | LOC727787 | receptor, two domains, long | ID NO: 118); | ID NO: 153); |
| | | cytoplasmic tail, 5A | NP_055034 (SEQ | NM_014219 (SEQ |
| | | (KIR2DL5A); killer cell | ID NO: 96); | ID NO: 129); |
| | | immunoglobulin-like | NP_055326 (SEQ | NM_014511 (SEQ |
| | | receptor, two domains, long | ID NO: 98); | ID NO: 131); |
| | | cytoplasmic tail, 5B | NP_055327 (SEQ | NM_014512 (SEQ |
| | | (KIR2DL5B); killer cell | ID NO: 119); | ID NO: 154); |
| | | immunoglobulin-like | NP_055328 (SEQ | NM_014513 (SEQ |
| | | receptor, two domains, | ID NO: 103); | ID NO: 155); |
| | | short cytoplasmic tail, 1 | NP_056952 (SEQ | NM_015868 (SEQ |
| | | (KIR2DS1); killer cell | ID NO: 99); | ID NO: 132); |
| | | immunoglobulin-like | NP_065396 (SEQ | NM_020535 (SEQ |
| | | receptor, two domains, | ID NO: 105); | ID NO: 136); |
| | | short cytoplasmic tail, 2 | NP_703144 (SEQ | NM_153443 (SEQ |
| | | (KIR2DS2); killer cell | ID NO: 113); | ID NO: 146); |
| | | immunoglobulin-like | XP_001718677 | XM_001718625 |
| | | receptor, two domains, | (SEQ ID NO: 95); | (SEQ ID NO: 128); |
| | | short cytoplasmic tail, 3 | XP_002346955 | XM_002346914 |
| | | (KIR2DS3); killer cell | (SEQ ID NO: 104) | (SEQ ID NO: 156) |
| | | immunoglobulin-like | | |
| | | receptor, two domains, | | |
| | | short cytoplasmic tail, 4 | | |
| | | (KIR2DS4); killer cell | | |
| | | immunoglobulin-like | | |
| | | receptor, two domains, | | |
| | | short cytoplasmic tail, 5 | | |
| | | (KIR2DS5); killer cell | | |
| | | immunoglobulin-like | | |
| | | receptor, three domains, | | |
| | | long cytoplasmic tail, 2 | | |
| | | (KIR3DL2); killer cell | | |
| | | immunoglobulin-like | | |
| | | receptor, three domains, | | |
| | | long cytoplasmic tail, 3 | | |
| | | (KIR3DL3); killer cell Ig- | | |
| | | like receptor; similar to | | |
| | | killer cell immunoglobulin- | | |
| | | like receptor 3DL2 | | |
| | | precursor (MHC class | | |
| | | I NK) | | |
| | | cell receptor) (KIR3DP1) | | |
| | | (Natural killer-associated | | |
| | | transcript 4) (NKAT-4) | | |
| | | (p70 natural killer cell | | |
| | | receptor clone CL-5) | | |
| | | (CD158k antigen) | | |
| 221875_x_at | HLA-F | major histocompatibility | NP_001091948 | NM_001098478 |
| (SEQ ID | | complex, class I, F | (SEQ ID NO: 120); | (SEQ ID NO: 157); |
| NO: 161) | | | NP_001091949 | NM_001098479 |
| | | | (SEQ ID NO: 121); | (SEQ ID NO: 158); |
| | | | NP_061823 (SEQ | NM_018950 (SEQ |
| | | | ID NO: 122) | ID NO: 159) |
| 221978_at | HLA-F | major histocompatibility | NP_001091948 | NM_001098478 |
| (SEQ ID | | complex, class I, F | (SEQ ID NO: 120 | (SEQ ID NO: 157); |
| NO: 162) | | | same as above); | NM_001098479 |
| | | | NP_001091949 | (SEQ ID NO: 158); |
| | | | (SEQ ID NO: 121); | NM_018950 (SEQ |
| | | | NP_061823 (SEQ | ID NO: 159) |
| | | | ID NO: 122) | |

### Example 14

### Antibodies Against Kir Receptors on Natural Killer Cells for Conversion of Non Responders to Responders of Interferon

The present inventor has uncovered that a potential solution for interferon non responders can be using a monoclonal antibody directed against KIR. For example, the 1-7F9, a human monoclonal antibody targeting KIRs on NK cells (Romagne F., et al., 2009, Blood 114:2667-2677, “Preclinical characterization of 1-7F9, a novel human anti-KIR receptor therapeutic antibody that augments natural killer—mediated killing of tumor cells”). This antibody activates NK cells by blocking the interaction between inhibitory KIRs and target cell HLA class I molecules.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

### Ahmad A and Alvarez F. 2004 (J. of Leukocyte Biology, 76:743-759)

Chen Limin, Borozan Ivan, Feld Jordan, Sun Jing, Tannis Laura-Lee, Coltescu Catalina, Heathcote Jenny, Edwards Aled M, And Mcgilvray Ian D. Hepatic Gene Expression Discriminates Responders and Nonresponders in Treatment of Chronic Hepatitis C Viral Infection. Gastroenterology 2005, 128:1437-1444.
Giannini C, et al., 2008, Blood. 112:4353-4. “Can BAFF promoter polymorphism be a predisposing condition for HCV-related mixed cryoglobulinemia?”
Gonzalez S, Castanotto D, Li H, Olivares S, Jensen M C, Forman S J, Rossi J J, Cooper L J. 2005, Molecular Therapy Vol. 11: 811-8. Amplification of RNAi—Targeting HLA mRNAs.
Khakoo S I., Thio C L., et al., 2004. Science 305:872-874. HLA and NK cell inhibitory receptor genes in resolving hepatitis C virus infection.
Kenya Honda, Akinori Takaoka, and Tadatsugu Taniguchi. Type I Inteferon Gene Induction by the Interferon Regulatory Factor, Family of Transcription Factors. Immunity 25, 349-360, 2006, Review.
Lopez-Vazquez et al., 2005 (JID 192: 162-165)
Magdalena Sarasin-Filipowicz*, Edward J. Oakeley†, Francois H. T. Duong*, Verena Christen*, Luigi Terracciano‡, Witold Filipowicz†, and Markus H. Heim. Chronic hepatitis C. PNAS_May 13, 2008_vol. 105_no. 19:7034-7039.
Paladino N., et al., 2007 (Tissue Antigens 69 Suppl 1:109-111)
Parham P, 2004 (Science 305:786-787)
Parham P, 2005 (Nature Reviews, Immunology 5:201-214)
Querec T D, Akondy R S, Lee E K, Cao W, Nakaya H I, Teuwen D, Pirani A, Gernert K, Deng J, Marzolf B, Kennedy K, Wu H, Bennouna S, Oluoch H, Miller J, Vencio R Z, Mulligan M, Aderem A, Ahmed R, Pulendran B. Nat. Immunol. 2009, 10:116-25
Rajagopalan S, and Long E O. 2005. JEM 201:1025-1029. Understanding how combination of HLA and KIR genes influence disease.
Rauch A., et al. 2007 (Tissue Antigens 69 Suppl 1:237-40)
Sarasin-Filipowicz Magdalena, Oakeley Edward J., Duong Francois H. T., Christen Verena, et al. Interferon signaling and treatment outcome in chronic hepatitis C. PNAS May 13, 2008, vol. 105 no. 19, Pages 7034-7039.
Shah N., et al., 2009, Blood 114:2567-2568
Stein Aerts, Peter Van Loo, Gert Thijs, Herbert Mayer, Rainer de Martin, Yves Moreau and Bart De Moor (2005) “TOUCAN 2: the all-inclusive open source workbench for regulatory sequence analysis”. Nucl Acids Res, vol. 33 (Web Server issue), W393-6.
Tarantino G. et al., 2008, SERUM BLYS/BAFF LEVELS IN ACUTE HEPATITIS C PREDICT CLINICAL OUTCOME. Digestive and Liver Disease 40:A1-A40)
Taylor, Milton W., Takuma Tsukahara, Leonid Brodsky, Joel Schaley, Corneliu Sanda, Matthew J. Stephens, Jeanette N. McClintick, Howard J. Edenberg, Lang Li, John E. Tavis, Charles Howell, and Steven H. Belle. Changes in Gene Expression during Pegylated Interferon and Ribavirin Therapy of Chronic Hepatitis C Virus Distinguish Responders from Nonresponders to Antiviral Therapy. Journal Of Virology, April 2007, p. 3391-3401.
van Baarsen L G M, Vosslamber S, Tijssen M, Baggen J M C, van der Voort L F, et al. (2008) Pharmacogenomics of Interferon-β Therapy in Multiple Sclerosis: Baseline IFN Signature Determines Pharmacological Differences between Patients. PLoS ONE 3(4): e1927. doi:10.1371/journal.pone.0001927
Vidal-Casrineira J R., et al., 2009, JVI online 21 Oct. 2009. Effect of killer immunoglobulin-like receptors (KIR) in the response to combined treatment in patients with chronic hepatitis C.
Vitale M., et al., Int Immunol. 2004 October; 16(10):1459-66.
Zeremski M, et al., 2007 (J Acquir Immune Defic Syndr. 2007 Jul. 1; 45(3):262-8)
Zuniga J., et al., 2009 (Molecular Immunology 46:2723-2727)

1. A method of predicting responsiveness of a subject to interferon treatment, comprising comparing a level of expression in a cell of the subject of at least one gene selected from the group consisting KIR3DL3, KIR3DL2, KIR3DL1, KIR2DL1, KIR2DL2, KIR2DL3, KLRG1, KIR3DS1, CD160, HLA-A, HLA-B, HLA-C, HLA-F, HLA-G and IFI27 to a reference expression data of said at least one gene obtained from at least one interferon responder subject and/or at least one interferon non-responder subject, thereby predicting the responsiveness of the subject to interferon treatment.

2. (canceled)

3. A method of predicting responsiveness to interferon treatment of a subject diagnosed with multiple sclerosis or infected with HCV type 2, 3 or 4, comprising comparing a level of expression in a cell of the subject of IFI6, OAS2, ISG15, OAS3 and IFIT1 genes to a reference expression data of said genes obtained from at least one interferon responder subject and/or at least one interferon non-responder subject, thereby predicting the responsiveness of the subject to interferon treatment.

4. A method of predicting responsiveness of a subject to interferon treatment, comprising comparing a ratio determined between an expression level of ISG15, IFI6, IFIT1, OAS2 and OAS3 genes in a cell of the subject following interferon treatment and an expression level of said genes in said cell of the subject prior to interferon treatment, or visa versa, to a reference ratio determined in a cell of at least one interferon responder subject and/or at least one interferon non-responder subject, said reference ratio is determined between an expression level of said genes following interferon treatment and an expression level of said genes prior to interferon treatment, or visa versa, thereby predicting the responsiveness to interferon treatment of a subject.

5-6. (canceled)

8. A method of treating of a subject in need of interferon treatment, the method comprising:
(a) predicting the responsiveness of the subject to interferon treatment according to the method of claim 1, and
(b) selecting a treatment regimen based on said responsiveness;
thereby treating the subject in need of interferon treatment.

9. A method of treating a subject in need of interferon therapy, comprising co-administering to the subject interferon and an agent capable of downregulating HLA or KIR inhibitory receptor, thereby treating the subject in need of interferon therapy.

10. The method of claim 1, wherein a decrease above a predetermined threshold in said level of expression of said at least one gene in said cell of the subject relative to said reference expression data of said at least one gene obtained from said at least one interferon non-responder subject predicts responsiveness of the subject to interferon treatment of the subject.

11. The method of claim 1, wherein an increase above a predetermined threshold in said level of expression of said at least one gene in said cell of the subject relative to said reference expression data of said at least one gene obtained from said at least one interferon responder subject predicts lack of responsiveness of the subject to interferon treatment of the subject.

12. The method of claim 1, wherein when a level of expression of said at least one gene in said cell of the subject is identical or changed below a predetermined threshold as compared to said reference expression data of said at least one gene obtained from said at least one interferon responder subject, then the subject is classified as responsive to interferon.

13. The method of claim 1, wherein when a level of expression of said at least one gene in said cell of the subject is identical or changed below a predetermined threshold as compared to said reference expression data of said at least one gene obtained from said at least one interferon non-responder subject, then the subject is classified as a non-responsive to interferon.

14. The method of claim 4, wherein an increase above a predetermined threshold in said ratio of the subject relative to said reference ratio of said at least one interferon non-responder subject predicts responsiveness of the subject to interferon treatment of the subject.

15. The method of claim 4, wherein a decrease above a predetermined threshold in said ratio of the subject relative to said reference ratio of said at least one interferon responder subject predicts lack of responsiveness of the subject to interferon treatment of the subject.

16. The method of claim 4, wherein when said ratio of the subject is identical or changed below a predetermined threshold as compared to said reference ratio of said at least one interferon responder subject, then the subject is classified as responsive to interferon.

17. The method of claim 4, wherein when said ratio of the subject is identical or changed below a predetermined threshold as compared to said reference ratio of said at least one interferon non-responder subject, then the subject is classified as non-responsive to interferon.

18. The method of claim 1, wherein said level of expression is determined prior to interferon treatment.

19. The method of claim 1, wherein said cell is a blood cell.

20. The method of claim 1, wherein said cell is a liver cell.

21. The method of claim 4, wherein said following interferon treatment is effected about 4 hours after interferon treatment.

22. The method of claim 4, wherein said following interferon treatment is effected about 24 hours after interferon treatment.

23. The method of claim 1, wherein said subject is diagnosed with HCV infection type 1, 2, 3 or 4.

24-29. (canceled)

30. A pharmaceutical composition comprising interferon, an agent capable of downregulating HLA or KIR inhibitory receptor, and a pharmaceutically acceptable carrier.

31-35. (canceled)

36. The method of claim 9, wherein said agent is selected from the group consisting of an antibody, an RNA silencing molecule, a ribozyme and a DNAzyme.

37. The method of claim 36, wherein said antibody is an anti-KIR inhibitory receptor antibody.

38. The method of claim 36, wherein said RNA silencing molecule is an siRNA directed against a KIR inhibitory receptor or a HLA.

39. The method of claim 9, wherein the subject is a non-responder to interferon treatment.


SEQUENCE LISTING (468 sequences)

SEQ ID NO: 1 - DNA, 528 bp, Homo sapiens
tgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaacttt

SEQ ID NO: 2 - DNA, 465 bp, Homo sapiens
ttggcatattggccccactgtaacttttgggggcttcccggtctagccacaccctcggatggaaagacttgactgcataaagatgtcagttctccctgagttgattgataggcttaatggtcaccctaaaaacacccacatatgcttttcgatggaaccagataagttgacgctaaagttcttatggaaaaatacacacgcaatagctaggaaaacacagggaaagaagagttctgagcagggcctagtcttagccaatattaaaacatactatgaagcctctgatacttaaacagcatggcgctggtacgtaaatagaccaatgcagttaggtggctctttccaagactctggggaaaaaagtagtaaaaagctaaatgcaatcaatcagcaattgaaagctaagtgagagagccagagggcctccttggtggtaaaagagggttgcatttcttgcagccagaa

SEQ ID NO: 3 - DNA, 432 bp, Homo sapiens
tggcgggcaacgaattccaggtgtccctgagcagctccatgtcggtgtcagagctgaaggcgcagatcacccagaagattggcgtgcacgccttccagcagcgtctggctgtccacccgagcggtgtggcgctgcaggacagggtcccccttgccagccagggcctgggccctggcagcacggtcctgctggtggtggacaaatgcgacgaacctctgagcatcctggtgaggaataacaagggccgcagcagcacctacgaggtccggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcc

SEQ ID NO: 4 - DNA, 555 bp, Homo sapiens
agactgcaaaccctttcataaagctgccttgctgaactcctctctgcaggagccctgcttaaaatagttgatgtcatcactttatgtgcatcttatttctgtcaacttgtatttttttttcttgtatttttccaattagctcctcctttttccttccagtctaaaaaaggaatcctctgtgtcttcaaagcaaagctctttactttccccttggttctcataactctgtgatcttgctctcggtgcttccaactcatccacgtcctgtctgtttcctctgtatacaaaaccctttctgcccctgctgacacagacatcctctatgccagcagccaggccaaccctttcattagaacttcaagctctccaaaggctcagattataactgttgtcatatttatatgaggctgttgtcttttccttctgagcctgcctttatccccccacccaggagtatcctcttgccaaagcaaaagactttttccttggctttagccttaaagatacttgaaggtctaggtgctttaacctcacataccctcacttaaactttta

SEQ ID NO: 5 - DNA, 509 bp, Homo sapiens
aaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttctactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctataaattggaaggaaatatgaatgaagccctggagtactatgagcgggccctgagactggctgctgactttgagaactctgtgagacaaggtccttaggcacccagatatcagccactttcacatttcatttcattttatgctaacatttactaatcatcttttctgcttac

SEQ ID NO: 6 - protein, 130 aa, Homo sapiens
MRQKAVSLFLCYLLLFTCSGVEAGKKKCSESSDSGSGFWKALTFMAVGGGLAVAGLPALGFTGAGIAANSVAASLMSWSAILNGGGVPAGGLVATLQSLGAGGSSVVIGNIGALMGYATHKYLDSEEDEE

SEQ ID NO: 7 - protein, 134 aa, Homo sapiens
MRQKAVSLFLCYLLLFTCSGVEAGENAGKKKCSESSDSGSGFWKALTFMAVGGGLAVAGLPALGFTGAGIAANSVAASLMSWSAILNGGGVPAGGLVATLQSLGAGGSSVVIGNIGALMGYATHKYLDSEEDEE

SEQ ID NO: 8 - protein, 138 aa, Homo sapiens
MRQKAVSLFLCYLLLFTCSGVEAGENAGKDAGKKKCSESSDSGSGFWKALTFMAVGGGLAVAGLPALGFTGAGIAANSVAASLMSWSAILNGGGVPAGGLVATLQSLGAGGSSVVIGNIGALMGYATHKYLDSEEDEE

SEQ ID NO: 9 - protein, 1087 aa, Homo sapiens
MDLYSTPAAALDRFVARRLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGPGLPRAGCSGLGHPIQLDPNQKTPENSKSLNAVYPRAGSKPPSCPAPGPTGAASIVPSVPGMALDLSQIPTKELDRFIQDHLKPSPQFQEQVKKAIDIILRCLHENCVHKASRVSKGGSFGRGTDLRDGCDVELIIFLNCFTDYKDQGPRRAEILDEMRAQLESWWQDQVPSLSLQFPEQNVPEALQFQLVSTALKSWTDVSLLPAFDAVGQLSSGTKPNPQVYSRLLTSGCQEGEHKACFAELRRNFMNIRPVKLKNLILLVKHWYRQVAAQNKGKGPAPASLPPAYALELLTIFAWEQGCRQDCFNMAQGFRTVLGLVQQHQQLCVYWTVNYSTEDPAMRMHLLGQLRKPRPLVLDPADPTWNVGHGSWELLAQEAAALGMQACFLSRDGTSVQPWDVMPALLYQTPAGDLDKFISEFLQPNRQFLAQVNKAVDTICSFLKENCFRNSPIKVIKVVKGGSSAKGTALRGRSDADLVVFLSCFSQFTEQGNKRAEIISEIRAQLEACQQERQFEVKFEVSKWENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGSLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVKAAV

SEQ ID NO: 10 - protein, 165 aa, Homo sapiens
MGWDLTVKMLAGNEFQVSLSSSMSVSELKAQITQKIGVHAFQQRLAVHPSGVALQDRVPLASQGLGPGSTVLLVVDKCDEPLSILVRNNKGRSSTYEVRLTQTVAHLKQQVSGLEGVQDDLFWLTFEGKPLEDQLPLGEYGLKPLSTVFMNLRLRGGGTEPGGRS

SEQ ID NO: 11 - protein, 172 aa, Homo sapiens
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWLKNNFEIQKSLDGFTIQVFTKNQRISFEVLAAFNALSKHCWVSGEKSQRSGCQTALCNL

SEQ ID NO: 12 - protein, 687 aa, Homo sapiens
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWLKNNFEIQKSLDGFTIQVFTKNQRISFEVLAAFNALSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEKLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPVKVI

SEQ ID NO: 13 - protein, 719 aa, Homo sapiens
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWLKNNFEIQKSLDGFTIQVFTKNQRISFEVLAAFNALSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEKLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPTMQTPGSCGARIHPIVNEMFSSRSHRILNNNSKRNF

SEQ ID NO: 14 - protein, 478 aa, Homo sapiens
MSTNGDDHQVKDSLEQLRCHFTWELSIDDDEMPDLENRVLDQIEFLDTKYSVGIHNLLAYVKHLKGQNEEALKSLKEAENLMQEEHDNQANVRSLVTWGNFAWMYYHMGRLAEAQTYLDKVENICKKLSNPFRYRMECPEIDCEEGWALLKCGGKNYERAKACFEKVLEVDPENPESSAGYAISAYRLDGFKLATKNHKPFSLLPLRQAVRLNPDNGYIKVLLALKLQDEGQEAEGEKYIEEALANMSSQTYVFRYAAKFYRRKGSVDKALELLKKALQETPTSVLLHHQIGLCYKAQMIQIKEATKGQPRGQNREKLDKMIRSAIFHFESAVEKKPTFEVAHLDLARMYIEAGNHRKAEENFQKLLCMKPVVEETMQDIHFHYGRFQEFQKKSDVNAIIHYLKAIKIEQASLTRDKSINSLKKLVLRKLRRKALDLESLSLLGFVYKLEGNMNEALEYYERALRLAADFENSVRQGP

SEQ ID NO: 15 - DNA, 836 bp, Homo sapiens
ccagccttcagccggagaaccgtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaacactt

SEQ ID NO: 16 - DNA, 848 bp, Homo sapiens
ccagccttcagccggagaaccgtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtgagaatgcgggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaacactt

SEQ ID NO: 17 - DNA, 860 bp, Homo sapiens
ccagccttcagccggagaaccgtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtgagaatgcgggtaaggatgcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaacactt

SEQ ID NO: 18 - DNA, 6646 bp, Homo sapiens
gttcggagagccgggcgggaaaacgaaaccagaaatccgaaggccgcgccagagccctgcttccccttgcacctgcgccgggcggccatggacttgtacagcaccccggccgctgcgctggacaggttcgtggccagaaggctgcagccgcggaaggagttcgtagagaaggcgcggcgcgctctgggcgccctggccgctgccctgagggagcgcgggggccgcctcggtgctgctgccccgcgggtgctgaaaactgtcaagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtcaggccggctccggcgtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgtgcctacaggggttgtggaaggagacgctgcccccggtctatgccctggaattgctgaccatcttcgcctgggagcagggctgtaagaaggatgctttcagcctagccgaaggcctccgaactgtcctgggcctgatccaacagcatcagcacctgtgtgttttctggactgtcaactatggcttcgaggaccctgcagttgggcagttcttgcagcggcagcttaagagacccaggcctgtgatcctggacccagctgaccccacatgggacctggggaatggggcagcctggcactgggatttgctagcccaggaggcagcatcctgctatgaccacccatgctttctgagggggatgggggacccagtgcagtcttggaaggggccgggccttccacgtgctggatgctcaggtttgggccaccccatccagctagaccctaaccagaagacccctgaaaacagcaagagcctcaatgctgtgtacccaagagcagggagcaaacctccctcatgcccagctcctggccccactggggcagccagcatcgtcccctctgtgccgggaatggccttggacctgtctcagatccccaccaaggagctggaccgcttcatccaggaccacctgaagccgagcccccagttccaggagcaggtgaaaaaggccatcgacatcatcttgcgctgcctccatgagaactgtgttcacaaggcctcaagagtcagtaaagggggctcatttggccggggcacagacctaagggatggctgtgatgttgaactcatcatcttcctcaactgcttcacggactacaaggaccaggggccccgccgcgcagagatccttgatgagatgcgagcgcagctagaatcctggtggcaggaccaggtgcccagcctgagccttcagtttcctgagcagaatgtgcctgaggctctgcagttccagctggtgtccacagccctgaagagctggacggatgttagcctgctgcctgccttcgatgctgtggggcagctcagttctggcaccaaaccaaatccccaggtctactcgaggctcctcaccagtggctgccaggagggcgagcataaggcctgcttcgcagagctgcggaggaacttcatgaacattcgccctgtcaagctgaagaacctgattctgctggtgaagcactggtaccgccaggttgcggctcagaacaaaggaaaaggaccagcccctgcctctctgcccccagcctatgccctggagctcctcaccatctttgcctgggagcagggctgcaggcaggattgtttcaacatggcccaaggcttccggacggtgctggggctcgtgcaacagcatcagcagctctgtgtctactggacggtcaactatagcactgaggacccagccatgagaatgcaccttcttggccagcttcgaaaacccagacccctggtcctggaccccgctgatcccacctggaacgtgggccacggtagctgggagctgttggcccaggaagcagcagcgctggggatgcaggcctgctttctgagtagagacgggacatctgtgcagccctgggatgtgatgccagccctcctttaccaaaccccagctggggaccttgacaagttcatcagtgaatttctccagcccaaccgccagttcctggcccaggtgaacaaggccgttgataccatctgttcatttttgaaggaaaactgcttccggaattctcccatcaaagtgatcaaggtggtcaagggtggctcttcagccaaaggcacagctctgcgaggccgctcagatgccgacctcgtggtgttcctcagctgcttcagccagttcactgagcagggcaacaagcgggccgagatcatctccgagatccgagcccagctggaggcatgtcaacaggagcggcagttcgaggtcaagtttgaagtctccaaatgggagaatccccgcgtgctgagcttctcactgacatcccagacgatgctggaccagagtgtggactttgatgtgctgccagcctttgacgccctaggccagctggtctctggctccaggcccagctctcaagtctacgtcgacctcatccacagctacagcaatgcgggcgagtactccacctgcttcacagagctacaacgggacttcatcatctctcgccctaccaagctgaagagcctgatccggctggtgaagcactggtaccagcagtgtaccaagatctccaaggggagaggctccctacccccacagcacgggctggaactcctgactgtgtatgcctgggagcagggcgggaaggactcccagttcaacatggctgagggcttccgcacggtcctggagctggtcacccagtaccgccagctctgtatctactggaccatcaactacaacgccaaggacaagactgttggagacttcctgaaacagcagcttcagaagcccaggcctatcatcctggatccggctgacccgacaggcaacctgggccacaatgcccgctgggacctgctggccaaggaagctgcagcctgcacatctgccctgtgctgcatgggacggaatggcatccccatccagccatggccagtgaaggctgctgtgtgaagttgagaaaatcagcggtcctactggatgaagagaagatggacaccagccctcagcatgaggaaattcagggtcccctaccagatgagagagattgtgtacatgtgtgtgtgagcacatgtgtgcatgtgtgtgcacacgtgtgcatgtgtgtgttttagtgaatctgctctcccagctcacacactcccctgcctcccatggcttacacactaggatccagactccatggtttgacaccagcctgcgtttgcagcttctctgtcacttccatgactctatcctcataccaccactgctgcttcccacccagctgagaatgccccctcctccctgactcctctctgcccatgcaaattagctcacatctttcctcctgctgcaatccatcccttcctcccattggcctctccttgccaaatctaaatagtttatatagggatggcagagagttcccatctcatctgtcagccacagtcatttggtactggctacctggagccttatcttctgaagggttttaaagaatggccaattagctgagaagaattatctaatcaattagtgatgtctgccatggatgcagtagaggaaagtggtggtacaagtgccatgattgattagcaatgtctgcactggatacggaaaaaagaaggtgcttgcaggtttacagtgtatatgtgggctattgaagagccctctgagctcggttgctagcaggagagcatgcccatattggcttactttgtctgccacagacacagacagagggagttgggacatgcatgctatggggaccctcttgttggacacctaattggatgcctcttcatgagaggcctccttttcttcaccttttatgctgcactcctcccctagtttacacatcttgatgctgtggctcagtttgccttcctgaatttttattgggtccctgttttctctcctaacatgctgagattctgcatccccacagcctaaactgagccagtggccaaacaaccgtgctcagcctgtttctctctgccctctagagcaaggcccaccaggtccatccaggaggctctcctgacctcaagtccaacaacagtgtccacactagtcaaggttcagcccagaaaacagaaagcactctaggaatcttaggcagaaagggattttatctaaatcactggaaaggctggaggagcagaaggcagaggccaccactggactattggtttcaatattagaccactgtagccgaatcagaggccagagagcagccactgctactgctaatgccaccactacccctgccatcactgccccacatggacaaaactggagtcgagacctaggttagattcctgcaaccacaaacatccatcagggatggccagctgccagagctgcgggaagacggatcccacctccctttcttagcagaatctaaattacagccagacctctggctgcagaggagtctgagacatgtatgattgaatgggtgccaagtgccagggggcggagtccccagcagatgcatcctggccatctgttgcgtggatgagggagtgggtctatctcagaggaaggaacaggaaacaaagaaaggaagccactgaacatcccttctctgctccacaggagtgccttagacagcctgactctccacaaaccactgttaaaacttacctgctaggaatgctagattgaatgggatgggaagagccttccctcattattgtcattcttggagagaggtgagcaaccaagggaagctcctctgattcacctagaacctgttctctgccgtctttggctcagcctacagagactagagtaggtgaagggacagaggacagggcttctaatacctgtgccatattgacagcctccatccctgtcccccatcttggtgctgaaccaacgctaagggcaccttcttagactcacctcatcgatactgcctggtaatccaaagctagaactctcaggaccccaaactccacctcttggattggccctggctgctgccacacacatatccaagagctcagggccagttctggtgggcagcagagacctgctctgccaagttgtccagcagcagagtggccctggcctgggcatcacaagccagtgatgctcctgggaagaccaggtggcaggtcgcagttgggtaccttccattcccaccacacagactctgggcctccccgcaaaatggctccagaattagagtaattatgagatggtgggaaccagagcaactcaggtgcatgatacaaggagaggttgtcatctgggtagggcagagaggagggcttgctcatctgaacaggggtgtatttcattccaggccctcagtctttggcaatggccaccctggtgttggcatattggccccactgtaacttttgggggcttcccggtctagccacaccctcggatggaaagacttgactgcataaagatgtcagttctccctgagttgattgataggcttaatggtcaccctaaaaacacccacatatgcttttcgatggaaccaggtaagttgacgctaaagttcttatggaaaaatacacacgcaatagctaggaaaacacagggaaagaagagttctgagcagggcctagtcttagccaatattaaaacatactatgaagcctctgatacttaaacagcatggcgctggtacgtaaatagaccaatgcagttaggtggctctttccaagactctggggaaaaaagtagtaaaaagctaaatgcaatcaatcagcaattgaaagctaagtgagagagccagagggcctccttggtggtaaaagagggttgcatttcttgcagccagaaggcagagaaagtgaagaccaagtccagaactgaatcctaagaaatgcaggactgcaaagaaattggtgtgtgtgtgtgtgtgtgtgtgtgtgtgtgtgtttaatttttaaaaagtttttattgagatacaagtcaataccataaagctctcacccttctaaagtgtacaattcagtggtgtgagtatattcataagatttatacttggtgtctattcataagacttatatccagcatattcataactagagccatatcacagatgcattcatcataataattccagacattttcatcaccctaaaaggaaaccctgaaacccattagcagtcattccccattcctccaacccattctctccctaatccctagaaaccaccaatctgctgtgtatttcatctattgccaacatttcatataaatggcatcatacaaaaaaaaaaaaaaaaaaaaa

SEQ ID NO: 19 - DNA, 685 bp, Homo sapiens
ataatagggccggtgctgcctgccgaagccggcggctgagaggcagcgaactcatctttgccagtacaggagcttgtgccgtggcccacagcccacagcccacagccatgggctgggacctgacggtgaagatgctggcgggcaacgaattccaggtgtccctgagcagctccatgtcggtgtcagagctgaaggcgcagatcacccagaagatcggcgtgcacgccttccagcagcgtctggctgtccacccgagcggtgtggcgctgcaggacagggtcccccttgccagccagggcctgggccccggcagcacggtcctgctggtggtggacaaatgcgacgaacctctgagcatcctggtgaggaataacaagggccgcagcagcacctacgaggtacggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcctgcggggaggcggcacagagcctggcgggcggagctaagggcctccaccagcatccgagcaggatcaagggccggaaataaaggctgttgtaaagagaaaaaaaaaaaaaaaaaaaaa

SEQ ID NO: 20 - DNA, 2123 bp, Homo sapiens
caagagttggtaagctcgctgcagtgggtggagagaggcctctagacttcagtttcagtttcctggctctgggcagcagcaagaattcctctgcctcccatcctaccattcactgtcttgccggcagccagctgagagcaatgggaaatggggagtcccagctgtcctcggtgcctgctcagaagctgggttggtttatccaggaatacctgaagccctacgaagaatgtcagacactgatcgacgagatggtgaacaccatctgtgacgtcctgcaggaacccgaacagttccccctggtgcagggagtggccataggtggctcctatggacggaaaacagtcttaagaggcaactccgatggtacccttgtcctcttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggttcaccatccaggtgttcacaaaaaatcagagaatctctttcgaggtgctggccgccttcaacgctctgagtaagcattgctgggtgtcaggagagaaaagccaaagaagcgggtgccagacagctctgtgcaacctctaggccatgagtgggatagataccactgctgctttaaaaaatgggagaccatagaccctcaggagagaagaatcccttctaccctggactcgctctcttctctggaactaacttctcccccataccctgattgtctttggagaaaatgttctggattctagaatctaaggcagagccttttaagccatactgtacacataaatcacctggaaccttgttaaaatgcagatcctgactcaggaggtctgagttagagcccaggatttcatatttctagccagctccatgatgagctgctggtccgcagatcatgcttgcaggttttgaccagagtcagtgttggttagagtaagaggatgaggcagacatctgggaaaagtccagctggggcaagcatttgaagtctgccttcctaccaggtcaaaatcaaggcaacgaccttccatagataactatcaaagcttgagggggtgccttgaacccaactcctaaatccctaagacctgcccacctcttgtgtctcctgtctcagcaaacattcccacactcttgcatattgttaaagtaacctctgcttaccaggcttctggtttaataaaagatggctagagtgactccatcttaaagcaagtagctaggcactcaaaaggaacctacaggcttaatacttgggtctgaaaatagccacagtctaagctgaccaccaattataattgcagaatatttaaggccatacaaaacatctcccactaagcctacaaaatgtccaggtgtcctaaaagttcagcccacttaaaggcagcattaatgagcaggtttaggttgaaggattaatggtcatcaataccactgttaagaagaaaattcttggccaaattgaatttaatggagtttaactgagcagacaattcacaaatctagaagcctcctgagccagagtaggttcagagagtcttgaacacagccacgtggtggaagaagatttatggacaggaaaaggaaaatgatgtactgaaaatgaaagtgaggtacagaaacagccagactggttatagctcagcattggccttatttgaacgagatttgaacagttggccacctttgattggccgaaactcagtgattggcacaagagtaggttgcagtctgtttacacatccttttaggttatagttcaccatgtacagagaaattttaggccaaacttaaaatatgtaaggaggcagctttaggctaaacttgatttaacagcaccaataccccctacctttagtgagcacatctgcacattccaattttaatgacagctccttagaatttcttatcaacgaagacactaacaaagaatggcgcattcctccttctcctttctgaggatgccctaccctgtaacaaagtcgtttctaataaatttgcttctttcaccataaaaaaaaaaaaaaaaaaaaaaaaaaaaaaaaaaaaaaa

SEQ ID NO: 21 - DNA, 3647 bp, Homo sapiens
caagagttggtaagctcgctgcagtgggtggagagaggcctctagacttcagtttcagtttcctggctctgggcagcagcaagaattcctctgcctcccatcctaccattcactgtcttgccggcagccagctgagagcaatgggaaatggggagtcccagctgtcctcggtgcctgctcagaagctgggttggtttatccaggaatacctgaagccctacgaagaatgtcagacactgatcgacgagatggtgaacaccatctgtgacgtcctgcaggaacccgaacagttccccctggtgcagggagtggccataggtggctcctatggacggaaaacagtcttaagaggcaactccgatggtacccttgtcctcttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggttcaccatccaggtgttcacaaaaaatcagagaatctctttcgaggtgctggccgccttcaacgctctgagcttaaatgataatcccagcccctggatctatcgagagctcaaaagatccttggataagacaaatgccagtcctggtgagtttgcagtctgcttcactgaactccagcagaagttttttgacaaccgtcctggaaaactaaaggatttgatcctcttgataaagcactggcatcaacagtgccagaaaaaaatcaaggatttaccctcgctgtctccgtatgccctggagctgcttacggtgtatgcctgggaacaggggtgcagaaaagacaactttgacattgctgaaggcgtcagaaccgtactggagctgatcaaatgccaggagaagctgtgtatctattggatggtcaactacaactttgaagatgagaccatcaggaacatcctgctgcaccagctccaatcagcgaggccagtaatcttggatccagttgacccaaccaataatgtgagtggagataaaatatgctggcaatggctgaaaaaagaagctcaaacctggttgacttctcccaacctggataatgagttacctgcaccatcttggaatgttctgcctgcaccactcttcacgaccccaggccaccttctggataagttcatcaaggagtttctccagcccaacaaatgcttcctagagcagattgacagtgctgttaacatcatccgtacattccttaaagaaaactgcttccgacaatcaacagccaagatccagattgtccggggaggatcaaccgccaaaggcacagctctgaagactggctctgatgccgatctcgtcgtgttccataactcacttaaaagctacacctcccaaaaaaacgagcggcacaaaatcgtcaaggaaatccatgaacagctgaaagccttttggagggagaaggaggaggagcttgaagtcagctttgagcctcccaagtggaaggctcccagggtgctgagcttctctctgaaatccaaagtcctcaacgaaagtgtcagctttgatgtgcttcctgcctttaatgcactgggtcagctgagttctggctccacacccagccccgaggtttatgcagggctcattgatctgtataaatcctcggacctcccgggaggagagttttctacctgtttcacagtcctgcagcgaaacttcattcgctcccggcccaccaaactaaaggatttaattcgcctggtgaagcactggtacaaagagtgtgaaaggaaactgaagccaaaggggtctttgcccccaaagtatgccttggagctgctcaccatctatgcctgggagcaggggagtggagtgccggattttgacactgcagaaggtttccggacagtcctggagctggtcacacaatatcagcagctctgcatcttctggaaggtcaattacaactttgaagatgagaccgtgaggaagtttctactgagccagttgcagaaaaccaggcctgtgatcttggacccagccgaacccacaggtgacgtgggtggaggggaccgttggtgttggcatcttctggcaaaagaagcaaaggaatggttatcctctccctgcttcaaggatgggactggaaacccaataccaccttggaaagtgccggtaaaagtcatctaaaggaggcgttgtctggaaatagccctgtaacaggcttgaatcaaagaacttctcctactgtagcaacctgaaattaactcagacacaaataaaggaaacccagctcacaggagcttaaacagctggtcagccccctaagcccccactacaagtgatcctcaggcaggtaaccccagattcatgcactgtagggtgctgcgcagcatccctagtctctacccagtagatgccactagccctcctctcccagtgacaaccaaaagtcttcagacattgtcaaacgttcccctgggttcacagatctttctgcctttggcttttggctccaccctctttagctgttaatttgagtacttatggccctgaaagcggccacggtgcctccagatggcaggtttgcaatccaagcaggaagaaggaaaagatacccaaaggtcaagaacacagtgattttattagaagtttcatccgcaaattttcttccatttcattgctcagaaatgtcatgtggctacctgtaacttgaaggtggctacaaagatgactgtggacgtgggttgcactggccacccaaggatgtctgccacacctctccaaagccctccctacctaccaagatatacctgatatattccaccaggatatcctccctccagatatacttggttctctccaccaggttctttctttaaagcaggatttctcaactttgatacttactcacatttggggctagacagttctttgtttggaggctctcttgtgcattgtaggatgttgagcagcatctctggcctgtacccagtagatgccacccagttgtgacaattaaaagtgtcttgagactttatcatgtgtcttctgccctaggtgagaacccttgcactagaggaaccctacaccccaaccctggggggaatgtagggaagaggtggccaagccaaccgtggggttagctctaattattaagatatgcattataaataaataccaaaaaattgtctctggcaatagttaccttcccagatacaggtccccccttttttcccctaactcttttaagcaatgattgtaactattaggagacattgctctcccacgtatgtttttctttttagacaatgcagacaccaggaagttgtggagctaggatccatcctattgtcaatgagatgttctcatccagaagccatagaatcctgaataataattctaaaagaaacttctagagatcatctggcaatcgcttttaaagactcggctcaccgtgagaaagagtcactcacatccattcttcccttgatggtccctattcctccttcccttgcttcttggacttcttgaaatcaatcaagactgcaaaccctttcataaagtcttgccttgctgaactccctctctgcaggcagcctgcctttaaaaatagttgctgtcatccacttt

SEQ ID NO: 22 - DNA, 3539 bp, Homo sapiens
caagagttggtaagctcgctgcagtgggtggagagaggcctctagacttcagtttcagtttcctggctctgggcagcagcaagaattcctctgcctcccatcctaccattcactgtcttgccggcagccagctgagagcaatgggaaatggggagtcccagctgtcctcggtgcctgctcagaagctgggttggtttatccaggaatacctgaagccctacgaagaatgtcagacactgatcgacgagatggtgaacaccatctgtgacgtcctgcaggaacccgaacagttccccctggtgcagggagtggccataggtggctcctatggacggaaaacagtcttaagaggcaactccgatggtacccttgtcctcttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggttcaccatccaggtgttcacaaaaaatcagagaatctctttcgaggtgctggccgccttcaacgctctgagcttaaatgataatcccagcccctggatctatcgagagctcaaaagatccttggataagacaaatgccagtcctggtgagtttgcagtctgcttcactgaactccagcagaagttttttgacaaccgtcctggaaaactaaaggatttgatcctcttgataaagcactggcatcaacagtgccagaaaaaaatcaaggatttaccctcgctgtctccgtatgccctggagctgcttacggtgtatgcctgggaacaggggtgcagaaaagacaactttgacattgctgaaggcgtcagaaccgtactggagctgatcaaatgccaggagaagctgtgtatctattggatggtcaactacaactttgaagatgagaccatcaggaacatcctgctgcaccagctccaatcagcgaggccagtaatcttggatccagttgacccaaccaataatgtgagtggagataaaatatgctggcaatggctgaaaaaagaagctcaaacctggttgacttctcccaacctggataatgagttacctgcaccatcttggaatgttctgcctgcaccactcttcacgaccccaggccaccttctggataagttcatcaaggagtttctccagcccaacaaatgcttcctagagcagattgacagtgctgttaacatcatccgtacattccttaaagaaaactgcttccgacaatcaacagccaagatccagattgtccggggaggatcaaccgccaaaggcacagctctgaagactggctctgatgccgatctcgtcgtgttccataactcacttaaaagctacacctcccaaaaaaacgagcggcacaaaatcgtcaaggaaatccatgaacagctgaaagccttttggagggagaaggaggaggagcttgaagtcagctttgagcctcccaagtggaaggctcccagggtgctgagcttctctctgaaatccaaagtcctcaacgaaagtgtcagctttgatgtgcttcctgcctttaatgcactgggtcagctgagttctggctccacacccagccccgaggtttatgcagggctcattgatctgtataaatcctcggacctcccgggaggagagttttctacctgtttcacagtcctgcagcgaaacttcattcgctcccggcccaccaaactaaaggatttaattcgcctggtgaagcactggtacaaagagtgtgaaaggaaactgaagccaaaggggtctttgcccccaaagtatgccttggagctgctcaccatctatgcctgggagcaggggagtggagtgccggattttgacactgcagaaggtttccggacagtcctggagctggtcacacaatatcagcagctctgcatcttctggaaggtcaattacaactttgaagatgagaccgtgaggaagtttctactgagccagttgcagaaaaccaggcctgtgatcttggacccagccgaacccacaggtgacgtgggtggaggggaccgttggtgttggcatcttctggcaaaagaagcaaaggaatggttatcctctccctgcttcaaggatgggactggaaacccaataccaccttggaaagtgccgacaatgcagacaccaggaagttgtggagctaggatccatcctattgtcaatgagatgttctcatccagaagccatagaatcctgaataataattctaaaagaaacttctagagatcatctggcaatcgcttttaaagactcggctcaccgtgagaaagagtcactcacatccattcttcccttgatggtccctattcctccttcccttgcttcttggacttcttgaaatcaatcaagactgcaaaccctttcataaagtcttgccttgctgaactccctctctgcaggcagcctgcctttaaaaatagttgctgtcatccactttatgtgcatcttatttctgtcaacttgtattttttttcttgtatttttccaattagctcctcctttttccttccagtctaaaaaaggaatcctctgtgtcttcaaagcaaagctctttactttccccttggttctcataactctgtgatcttgctctcggtgcttccaactcatccacgtcctgtctgtttcctctgtatacaaaaccctttctgcccctgctgacacagacatcctctatgccagcagccagccaaccctttcattagaacttcaagctctccaaaggctcagattataactgttgtcatatttatatgaggctgttgtcttttccttctgagcctgcctttctcccccccacccaggagtatcctcttgccaaatcaaaagactttttccttgggctttagccttaaagatacttgaaggtctaggtgctttaacctcacataccctcacttaaacttttatcactgttgcatataccagttgtgatacaataaagaatgtatctggattttgtgcctagttcctagcacacagcttcaaaaattctagagtttcctgataggagtgtcttttgtattcataacaagcccttttcacccatgcctgggtttatgctaacaaggttacccatggtgggcccttagtttcaaggaaggagttggccaagccagaaagaccaagcatgtggttaaagcattggaattttcagccccatcccacccccaatctccaaggaggtgatggggctggaaattgagttcaattttaacatggccagtgatttaagcaatgctgcctatgtaaagaaaccccaataaaaactctggacagtgaggcttggggagcttcctgattggcagacattccaatgtactaggaaggtagcgcatcttgattccacagggacaaaggctcctgagctctgggcccttccagtgcttgccaccctacatactctttgtctggctcttcatttgtattctttataataaaatggtgattgtaagtagagca

SEQ ID NO: 23 - DNA, 1876 bp, Homo sapiens
ttacaccattggctgctgtttagctcccttatataacactgtcttggggtttaaacgtaactgaaaatccacaagacagaatagccagatctcagaggagcctggctaagcaaaaccctgcagaacggctgcctaatttacagcaaccatgagtacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacagtgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaagaattatgaacgggccaaggcctgctttgaaaaggtgcttgaagtggaccctgaaaaccctgaatccagcgctgggtatgcgatctctgcctatcgcctggatggctttaaattagccacaaaaaatcacaagccattttctttgcttcccctaaggcaggctgtccgcttaaatccagacaatggatatattaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattaaaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttccactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctacaaattggaaggaaatatgaatgaagccctggagtactatgagcgggccctgagactggctgctgactttgagaactctgtgagacaaggtccttaggcacccagatatcagccactttcacatttcatttcattttatgctaacatttactaatcatcttttctgcttactgttttcagaaacattataattcactgtaatgatgtaattcttgaataataaatctgacaaaatattagttgtgttcaacaattagtgaaacagaatgtgtgtatgcatgtaagaaagagaaatcatttgtatgagtgctatgtagtagagaaaaaatgttagttaactttgtaggaaataaaacattggacttacactaaaaaaaaaaaaaaaaaa

SEQ ID NO: 24 - DNA, 532 bp, Homo sapiens
gaaatgcccaaaaccttcagagattgacacgctgtcattttccatttccgttcctggatctacggagtcttctaagagattttgcaatgaggagaagcattgttttcaaactatataactgaccttatttataattagggatattatcaaaatatgtaaccatgaggcccctcaggtcctgatcagtcagaatggatgctttcaccagcagacccggccatgtggctgctcggtcctgggtgctcgctgctgtgcaagacattagccctttagttatgagcctgtgggaacttcaggggttcccagtggggagagcagtggcagtgggaggcatctgggggccaaaggtcagtggcagggggtatttcagtattatacaactgctgtgaccagacttgtatactggctgaatatcagtgctgtttgtaatttttcactttgagaaccaacattaattccatatgaatcaagtgttttgtaactgctattcatttattcagcaaatatttattgagcatctcttctccataag

SEQ ID NO: 25 - DNA, 532 bp, Homo sapiens
acagttcacatcccaccatagtgatgttttggaaggctttccacaaattgactctggaagaaaagaaaaaattccttgtatttcttacaggaactgacagactacaaatgaaagatttaaataatatgaaaataacattttgctgtcctgaaagttggaatgaaagagaccctataagagcactgacatgtttcagtgtcctcttcctccctaaatattctacaatggaaacagttgaagaagcgcttcaagaagccatcaacaacaacagaggatttggctgaccagcttgcttgtccaacagccttattttgttgttgttatcgttgttgttgttgttgttgttgttgtttctctactttgttttgttttaggcttttagcagcctgaagccatggtttttcatttctgtctctagtgataagcaggaaagagggatgaagaagagggtttactggccggttagaacccgtgactgtattctctcccttggatacccctatgcctacatcatattccttacctcttttgg

SEQ ID NO: 26 - DNA, 511 bp, Homo sapiens
gtgtgttgtgtatggactcactcccaggttcacctggccacaggtgcacccttcccacaccctttacattccccagagccaagggagtttaagtttgcagttacaggccagttctccagctctccatcttagagagacaggtcaccttgcaggcctgcttgcaggaaatgaatccagcagccaactcgaatccccctagggctcaggcactgagggcctggggacagtggagcatatgggtgggagacagatggagggtaccctatttacaactgagtcagccaagccactgatgggaatatacagatttaggtgctaaaccgtttattttccacggatgagtcacaatctgaagaatcaaacttccatcctgaaaatctatatgtttcaaaaccacttgccatcctgttagattgccagttcctgggaccaggcctcagactgtgaagtatatatcctccagcattcagtccagggggagccacggaaaccatgttcttgcttaagccat

SEQ ID NO: 27 - DNA, 2037 bp, Homo sapiens
gggaagctcgggccggcagggtttccccgcacgctggcgcccagctcccggcgcggaggccgctgtaagtttcgctttccattcagtggaaaacgaaagctgggcggggtgccacgagcgcggggccagaccaaggcgggcccggagcggaacttcggtcccagctcggtccccggctcagtcccgacgtggaactcagcagcggaggctggacgcttgcatggcgcttgagagattccatcgtgcctggctcacataagcgcttcctggaagtgaagtcgtgctgtcctgaacgcgggccaggcagctgcggcctgggggttttggagtgatcacgaatgagcaaggcgtttgggctcctgaggcaaatctgtcagtccatcctggctgagtcctcgcagtccccggcagatcttgaagaaaagaaggaagaagacagcaacatgaagagagagcagcccagagagcgtcccagggcctgggactaccctcatggcctggttggtttacacaacattggacagacctgctgccttaactccttgattcaggtgttcgtaatgaatgtggacttcaccaggatattgaagaggatcacggtgcccaggggagctgacgagcagaggagaagcgtccctttccagatgcttctgctgctggagaagatgcaggacagccggcagaaagcagtgcggcccctggagctggcctactgcctgcagaagtgcaacgtgcccttgtttgtccaacatgatgctgcccaactgtacctcaaactctggaacctgattaaggaccagatcactgatgtgcacttggtggagagactgcaggccctgtatacgatccgggtgaaggactccttgatttgcgttgactgtgccatggagagtagcagaaacagcagcatgctcaccctcccactttctctttttgatgtggactcaaagcccctgaagacactggaggacgccctgcactgcttcttccagcccagggagttatcaagcaaaagcaagtgcttctgtgagaactgtgggaagaagacccgtgggaaacaggtcttgaagctgacccatttgccccagaccctgacaatccacctcatgcgattctccatcaggaattcacagacgagaaagatctgccactccctgtacttcccccagagcttggatttcagccagatccttccaatgaagcgagagtcttgtgatgctgaggagcagtctggagggcagtatgagctttttgctgtgattgcgcacgtgggaatggcagactccggtcattactgtgtctacatccggaatgctgtggatggaaaatggttctgcttcaatgactccaatatttgcttggtgtcctgggaagacatccagtgtacctacggaaatcctaactaccactggcaggaaactgcatatcttctggtttacatgaagatggagtgctaatggaaatgcccaaaaccttcagagattgacacgctgtcattttccatttccgttcctggatctacggagtcttctaagagattttgcaatgaggagaagcattgttttcaaactatataactgagccttatttataattagggatattatcaaaatatgtaaccatgaggcccctcaggtcctgatcagtcagaatggatgctttcaccagcagacccggccatgtggctgctcggtcctgggtgctcgctgctgtgcaagacattagccctttagttatgagcctgtgggaacttcaggggttcccagtggggagagcagtggcagtgggaggcatctgggggccaaaggtcagtggcagggggtatttcagtattatacaactgctgtgaccagacttgtatactggctgaatatcagtgctgtttgtaatttttcactttgagaaccaacattaattccatatgaatcaagtgttttgtaactgctattcatttattcagcaaatatttattgatcatctcttctccataagatagtgtgataaacacagtcatgaataaagttattttccacaaaa

SEQ ID NO: 28 - DNA, 3525 bp, Homo sapiens
tcagtagctgaggctgcggttccccgacgccacgcagctgcgcgcagctggttcccgctctgcagcgcaacgcctgaggcagtgggcgcgctcagtcccgggaccaggcgttctctcctctcgcctctgggcctgggaccccgcaaagcggcgatggagcggaggtcgcggaggaagtcgcggcgcaacgggcgctcgaccgcgggcaaggccgccgcgacccagcccgcgaagtctccgggcgcacagctctggctctttcccagcgccgcgggcctccaccgcgcgctgctccggagggtggaggtgacgcgccaactctgctgctcgccggggcgcctcgcggtcttggaacgcggcggggcgggcgtccaggttcaccagctgctcgccgggagcggcggcgcccggacgccgaaatgcattaaattaggaaaaaacatgaagatacattccgtggaccaaggagcagagcacatgctgattctctcatcagatggaaaaccatttgagtatgacaactatagcatgaaacatctaaggtttgaaagcattttacaagaaaaaaaaataattcagatcacatgtggagattaccattctcttgcactctcaaaaggtggtgagctttttgcctggggacagaacctgcatgggcagcttggagttggaaggaaatttccctcaaccaccacaccacagattgtggagcacctcgcaggagtacccttggctcagatttctgccggagaagcccacagcatggccttatccatgtctggcaacatttattcatggggaaaaaatgaatgtggacaactaggcctgggccacactgagagtaaagatgatccatcccttattgaaggactagacaatcagaaagttgaatttgtcgcttgtggtggctctcacagtgccctactcacacaggatgggctgctgtttactttcggtgctggaaaacatgggcaacttggtcataattcaacacagaatgagctaagaccctgtttggtggctgagcttgttgggtatagagtgactcagatagcatgtggaaggtggcacacacttgcctatgtttctgatttgggaaaggtcttttcctttggttctggaaaagatggacaactgggaaatggtggaacacgtgaccagctgatgccgcttccagtgaaagtatcatcaagtgaagaactcaaacttgaaagccatacctcagaaaaggagttaataatgattgctggagggaatcaaagcattttgctctggataaagaaagagaattcatatgttaatctgaagaggacaattcctactctgaatgaagggactgtaaagagatggattgctgatgtggagactaaacggtggcagagcacaaaaagggaaatccaagagatattttcatctcctgcttgtctaactggaagttttttaaggaaaagaagaactacagaaatgatgcctgtttatttggacttaaataaagcaagaaacatcttcaaggagttaacccaaaaggactggattactaacatgataaccacctgcctcaaagataatctgctcaaaagacttccatttcattctccaccccaagaagctttagaaattttcttccttctcccagaatgtcctatgatgcatatttccaacaactgggagagccttgtggttccatttgcaaaggttgtttgtaaaatgagtgaccagtcttcactggttctggaagagtattgggcaactctgcaagaatccactttcagcaaactggtccagatgtttaaaacagccgtcatatgccagttggattactgggatgaaagtgctgaggagaatggtaatgttcaagctctcctagaaatgttgaagaagctgcacagggtaaaccaggtgaaatgtcaactacctgaaagtattttccaagtagacgaactcttgcaccgtctcaatttttttgtagaagtatgcagaaggtacttgtggaaaatgactgtggacgcttcagaaaatgtacaatgctgcgtcatattcagtcactttccatttatctttaataatctgtcgaaaattaaactactacatacagacacacttttaaaaatagagagtaaaaaacataaagcttatcttaggtcggcagcaattgaggaagaaagagagtctgaattcgctttgaggcccacgtttgatctaacagtcagaaggaatcacttgattgaggatgttttgaatcagctaagtcaatttgagaatgaagacctgaggaaagagttatgggtttcatttagtggagaaattgggtatgacctcggaggagtcaagaaagagttcttctactgtctgtttgcagagatgatccagccggaatatgggatgttcatgtatcctgaaggggcttcctgcatgtggtttcctgtcaagcctaaatttgagaagaaaagatacttcttttttggggttctatgtggactttccctgttcaattgcaatgttgccaaccttcctttcccactggcactgtttaagaaacttttggaccaaatgccatcattggaagacttgaaagaactcagtcctgatttgggaaagaatttgcaaacacttctggatgatgaaggtgataactttgaggaagtattttacatccattttaatgtgcactgggacagaaacgacacaaacttaattcctaatggaagtagcataactgtcaaccagactaacaagagagactatgtttctaagtatatcaattacattttcaacgactctgtaaaggcggtttatgaagaatttcggagaggattttataaaatgtgcgacgaagacattatcaaattattccaccccgaagaactgaaggatgtgattgttggaaatacagattatgattggaaaacatttgaaaagaatgcacgttatgaaccaggatataacagttcacatcccaccatagtgatgttttggaaggctttccacaaattgactctggaagaaaagaaaaaattccttgtatttcttacaggaactgacagactacaaatgaaagatttaaataatatgaaaataacattttgctgtcctgaaagttggaatgaaagagaccctataagagcactgacatgtttcagtgtcctcttcctccctaaatattctacaatggaaacagttgaagaagcgcttcaagaagccatcaacaacaacagaggatttggctgaccagcttgcttgtccaacagccttattttgttgttgttatcgttgttgttgttgttgttgttgttgtttctctactttgttttgttttaggcttttagcagcctgaagccatggtttttcatttctgtctctagtgataagcaggaaagagggatgaagaagagggtttactggccggttagaacccgtgactgtattctctcccttggatacccctatgcctacatcatattccttacctcttttgggaaatatttttcaaaaataaaataaccgaaaaattaaaaaaaaaaaaaa

SEQ ID NO: 29 - DNA, 1260 bp, Homo sapiens
gggggtggggtccccggggcggggcggggcgcgctgtgtcgcgggtcggagctcggtcctgctggaggccacgggtgccacacactcggtcccgacatgatggcgagcatgcgagtggtgaaggagctggaggatcttcagaagaagcctcccccatacctgcggaacctgtccagcgatgatgccaatgtcctggtgtggcacgctctcctcctacccgaccaacctccctaccacctgaaagccttcaacctgcgcatcagcttcccgccggagtatccgttcaagcctcccatgatcaaattcacaaccaagatctaccaccccaacgtggacgagaacggacagatttgcctgcccatcatcagcagtgagaactggaagccttgcaccaagacttgccaagtcctggaggccctcaatgtgctggtgaatagaccgaatatcagggagcccctgcggatggacctcgctgacctgctgacacagaatccggagctgttcagaaagaatgccgaagagttcaccctccgattcggagtggaccggccctcctaactcatgttctgaccctctgtgcactggatcctcggcatagcggacggacacacctcatggactgaggccagagccccctgtggcccattccccattcatttttcccttcttaggttgttagtcattagtttgtgtgtgtgtgtggtggagggaagggagctatgagtgtgtgtgttgtgtatggactcactcccaggttcacctggccacaggtgcacccttcccacaccctttacattccccagagccaagggagtttaagtttgcagttacaggccagttctccagctctccatcttagagagacaggtcaccttgcaggcctgcttgcaggaaatgaatccagcagccaactcgaatccccctagggctcaggcactgagggcctggggacagtggagcatatgggtgggagacagatggagggtaccctatttacaactgagtcagccaagccactgatgggaatatacagatttaggtgctaaaccgtttattttccacggatgagtcacaatctgaagaatcaaacttccatcctgaaaatctatatgtttcaaaaccacttgccatcctgttagattgccagttcctgggaccaggcctcagactgtgaagtatatatcctccagcattcagtccagggggagccacggaaaccatgttcttgcttaagccattaaagtcagagatgaattctgg

SEQ ID NO: 30 - protein, 372 aa, Homo sapiens
MSKAFGLLRQICQSILAESSQSPADLEEKKEEDSNMKREQPRERPRAWDYPHGLVGLHNIGQTCCLNSLIQVFVMNVDFTRILKRITVPRGADEQRRSVPFQMLLLLEKMQDSRQKAVRPLELAYCLQKCNVPLFVQHDAAQLYLKLWNLIKDQITDVHLVERLQALYTIRVKDSLICVDCAMESSRNSSMLTLPLSLFDVDSKPLKTLEDALHCFFQPRELSSKSKCFCENCGKKTRGKQVLKLTHLPQTLTIHLMRFSIRNSQTRKICHSLYFPQSLDFSQILPMKRESCDAEEQSGGQYELFAVIAHVGMADSGHYCVYIRNAVDGKWFCFNDSNICLVSWEDIQCTYGNPNYHWQETAYLLVYMKMEC

SEQ ID NO: 31 - protein, 1024 aa, Homo sapiens
MERRSRRKSRRNGRSTAGKAAATQPAKSPGAQLWLFPSAAGLHRALLRRVEVTRQLCCSPGRLAVLERGGAGVQVHQLLAGSGGARTPKCIKLGKNMKIHSVDQGAEHMLILSSDGKPFEYDNYSMKHLRFESILQEKKIIQITCGDYHSLALSKGGELFAWGQNLHGQLGVGRKFPSTTTPQIVEHLAGVPLAQISAGEAHSMALSMSGNIYSWGKNECGQLGLGHTESKDDPSLIEGLDNQKVEFVACGGSHSALLTQDGLLFTFGAGKHGQLGHNSTQNELRPCLVAELVGYRVTQIACGRWHTLAYVSDLGKVFSFGSGKDGQLGNGGTRDQLMPLPVKVSSSEELKLESHTSEKELIMIAGGNQSILLWIKKENSYVNLKRTIPTLNEGTVKRWIADVETKRWQSTKREIQEIFSSPACLTGSFLRKRRTTEMMPVYLDLNKARNIFKELTQKDWITNMITTCLKDNLLKRLPFHSPPQEALEIFFLLPECPMMHISNNWESLVVPFAKVVCKMSDQSSLVLEEYWATLQESTFSKLVQMFKTAVICQLDYWDESAEENGNVQALLEMLKKLHRVNQVKCQLPESIFQVDELLHRLNFFVEVCRRYLWKMTVDASENVQCCVIFSHFPFIFNNLSKIKLLHTDTLLKIESKKHKAYLRSAAIEEERESEFALRPTFDLTVRRNHLIEDVLNQLSQFENEDLRKELWVSFSGEIGYDLGGVKKEFFYCLFAEMIQPEYGMFMYPEGASCMWFPVKPKFEKKRYFFFGVLCGLSLFNCNVANLPFPLALFKKLLDQMPSLEDLKELSPDLGKNLQTLLDDEGDNFEEVFYIHFNVHWDRNDTNLIPNGSSITVNQTNKRDYVSKYINYIFNDSVKAVYEEFRRGFYKMCDEDIIKLFHPEELKDVIVGNTDYDWKTFEKNARYEPGYNSSHPTIVMFWKAFHKLTLEEKKKFLVFLTGTDRLQMKDLNNMKITFCCPESWNERDPIRALTCFSVLFLPKYSTMETVEEALQEAINNNRGFG

SEQ ID NO: 32 - protein, 153 aa, Homo sapiens
MMASMRVVKELEDLQKKPPPYLRNLSSDDANVLVWHALLLPDQPPYHLKAFNLRISFPPEYPFKPPMIKFTTKIYHPNVDENGQICLPIISSENWKPCTKTCQVLEALNVLVNRPNIREPLRMDLADLLTQNPELFRKNAEEFTLRFGVDRPS

SEQ ID NO: 33 - protein, 87 aa, Homo sapiens
MIKFTTKIYHPNVDENGQICLPIISSENWKPCTKTCQVLEALNVLVNRPNIREPLRMDLADLLTQNPELFRKNAEEFTLRFGVDRPS

SEQ ID NO: 34 - DNA, 1269 bp, Homo sapiens
aagactggaacccgtatgagcgccccccagcgcccctgagcgctcgccgccggtgcacggcgcaccccgcgggaggcagggatcagcaaagccgtgcgccccgaggcccgcccccgtctccgcacaaagaccgagctggaggatcttcagaagaagcctcccccatacctgcggaacctgtccagcgatgatgccaatgtcctggtgtggcacgctctcctcctacccgaccaacctccctaccacctgaaagccttcaacctgcgcatcagcttcccgccggagtatccgttcaagcctcccatgatcaaattcacaaccaagatctaccaccccaacgtggacgagaacggacagatttgcctgcccatcatcagcagtgagaactggaagccttgcaccaagacttgccaagtcctggaggccctcaatgtgctggtgaatagaccgaatatcagggagcccctgcggatggacctcgctgacctgctgacacagaatccggagctgttcagaaagaatgccgaagagttcaccctccgattcggagtggaccggccctcctaactcatgttctgaccctctgtgcactggatcctcggcatagcggacggacacacctcatggactgaggccagagccccctgtggcccattccccattcatttttcccttcttaggttgttagtcattagtttgtgtgtgtgtgtggtggagggaagggagctatgagtgtgtgtgttgtgtatggactcactcccaggttcacctggccacaggtgcacccttcccacaccctttacattccccagagccaagggagtttaagtttgcagttacaggccagttctccagctctccatcttagagagacaggtcaccttgcaggcctgcttgcaggaaatgaatccagcagccaactcgaatccccctagggctcaggcactgagggcctggggacagtggagcatatgggtgggagacagatggagggtaccctatttacaactgagtcagccaagccactgatgggaatatacagatttaggtgctaaaccgtttattttccacggatgagtcacaatctgaagaatcaaacttccatcctgaaaatctatatgtttcaaaaccacttgccatcctgttagattgccagttcctgggaccaggcctcagactgtgaagtatatatcctccagcattcagtccagggggagccacggaaaccatgttcttgcttaagccattaaagtcagagatgaattctgg

SEQ ID NO: 35 - DNA, 507 bp, Homo sapiens
ttgtgtccctactcattgaaaccaaactctggaaaggacccaatgtaccagtatttatacctctaatgaagcacagagagaggaagagagctgcttaaactcacacaacaatgaactgcagacacagctgttctctccctctctccttcccagagcaatttatactttaccctcaggctgtcctctggggagaaggtgccatggtcttaggtgtctgtgccccaggacagaccctaggaccctaaatccaatagaaaatgcatatctttgctccactttcagccaggctggagcaaggtaccttttcttaggatcttgggagggaatggatgcccctctctgcatgatcttgttgaggcatttagctgccatgcacctgtccccctttaatactgggcattttaaagccatctcaagaggcatcttctacatgttttgtacgcattaaaataatttcaaagatatctgagaaaagccgatatttgccattcttcctatatcctggaa

SEQ ID NO: 36 - DNA, 491 bp, Homo sapiens
  misc_feature (35)..(35): n is a, c, g, or t
cctgcacgcatggcaggctggcacccccccaccgncctccccacagccagcagcctttccacagtcactgcccttcccgcagtccccagccttccntacggcctcacccgcaccccctcagagcccagggctgcaacccctcattatccaccacgcacagatggtacagctggggctgaacaaccacatgtggaaccagagagggtcccaggcgcccgaggacaagacgcaggaggcagaatgaccgcgtgtcnttgcctgaccacctggggaacacccctggacccaggcatcggccaggaccccatagagcaccccggtctgccctgtgccctgtggacagtggaagatgaggtcatctgccactttcaggacattgtccgggagcccttcatttaggacaaaacgggcgcgatgatgccctggctttcagggtggtcagaactggatacggtgtttacaattccaatctctctatttctgggtgaagg

SEQ ID NO: 37 - DNA, 522 bp, Homo sapiens
atctgcacacttgatacagcaacgttagatggttttgatggtaaaccctaaaggaggactccaagagtgtgtatttatttatagttttatcagagatgacaattatttgaatgccaattatatggattcctttcattttttgctggaggatgggagaagaaaccaaagtttatagaccttcacattgagaaagcttcagttttgaacttcagctatcagattcaaaaacaacagaaagaaccaagacattcttaagatgcctgtactttcagctgggtataaattcatgagttcaaagattgaaacctgaccaatttgctttatttcatggaagaagtgatctacaaaggtgtttgtgccatttggaaaacagcgtgcatgtgttcaagccttagattggcgatgtcgtattttcctcacgtgtggcaatgccaaaggctttactttacctgtgagtacacactatatgaattatttccaacgtacatttaatcaataagggtcacaaattcccaaatcaat

SEQ ID NO: 38 - DNA, 445 bp, Homo sapiens
  misc_feature (164)..(164): n is a, c, g, or t
cccttggtgctgtgtggtcccagtggaaggaggggaagattttggaaacctggtagccaccagtaaggtgattctctgccctgttggggcctaaatttgggggcttttgggcaacctctccgtgtactgcgtctgtccacactcgattgggccccaggtgtgtnatgnaggcgnctctggtaaggtgnctnnnnnnnnnnnnnnnnnnnnntcnagtaacgaggcttttgatgtgttgagctggaggtgagtggaccgggggctgtgttttaagctgcttccttggcatttggcatcactgccttctgttcccgggggagcatggatcttttgtcctcactgctttctaatggggagggctgagggctccctgtccccacagcaggtatgntnngctctgccccagccccacacttgctctgaaaaccaagtgtcagagcccctt

SEQ ID NO: 39 - DNA, 477 bp, Homo sapiens
cagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggag

SEQ ID NO: 40 - protein, 309 aa, Homo sapiens
MRPDRAEAPGPPAMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 41 - protein, 712 aa, Homo sapiens
MACTGPSLPSAFDILGAAGQDKLLYLKHKLKTPRPGCQGQDLLHAMVLLKLGQETEARISLEALKADAVARLVARQWAGVDSTEDPEEPPDVSWAVARLYHLLAEEKLCPASLRDVAYQEAVRTLSSRDDHRLGELQDEARNRCGWDIAGDPGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKEQDTRALREQSQHLDGERMQAAALNAAYSAYLQSYLSYQAQMEQLQVAFGSHMSFGTGAPYGARMPFGGQVPLGAPPPFPTWPGCPQPPPLHAWQAGTPPPPSPQPAAFPQSLPFPQSPAFPTASPAPPQSPGLQPLIIHHAQMVQLGLNNHMWNQRGSQAPEDKTQEAE

SEQ ID NO: 42 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 43 - protein, 582 aa, Homo sapiens
MAEFLDDQETRLCDNCKKEIPVFNFTIHEIHCQRNIGMCPTCKEPFPKSDMETHMAAEHCQVTCKCNKKLEKRLLKKHEETECPLRLAVCQHCDLELSILKLKEHEDYCGARTELCGNCGRNVLVKDLKTHPEVCGREGEEKRNEVAIPPNAYDESWGQDGIWIASQLLRQIEALDPPMRLPRRPLRAFESDVFHNRTTNQRNITAQVSIQNNLFEEQERQERNRGQQPPKEGGEESANLDFMLALSLQNEGQASSVAEQDFWRAVCEADQSHGGPRSLSDIKGAADEIMLPCEFCEELYPEELLIDHQTSCNPSRALPSLNTGSSSPRGVEEPDVIFQNFLQQAASNQLDSLMGLSNSHPVEESIIIPCEFCGVQLEEEVLFHHQDQCDQRPATATNHVTEGIPRLDSQPQETSPELPRRRVRHQGDLSSGYLDDTKQETANGPTSCLPPSRPINNMTATYNQLSRSTSGPRPGCQPSSPCVPKLSNSDSQDIQGRNRDSQNGAIAPGHVSVIRPPQNLYPENIVPSFSPGPSGRYGASGRSEGGRNSRVTPAAANYRSRTAKAKPSKQQGAGDAEEEEEE

SEQ ID NO: 44 - protein, 582 aa, Homo sapiens; identical to SEQ ID NO: 43

SEQ ID NO: 45 - protein, 503 aa, Homo sapiens
MALAPERAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 46 - protein, 474 aa, Homo sapiens
MALAPERAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 47 - protein, 516 aa, Homo sapiens
MPVPERPAAGPDSPRPGTRRAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 48 - DNA, 2862 bp, Homo sapiens
agattcctacttcttacgccccccacatcacccgcctcgagacctcaagggtagaggtgggcacccccgcctccgcacttttgctcggggctccagattgtagggcagggcggcgcttctcggaaagcgaaagccggcggggcggggcgggtgccgcaggagaaagaggaagcgctggcagacaatgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctgaagactgttctgaggccctgggtgtgtgtgtatctgtctgcctgtccatgtacttctgccctgcctcctcctttcgttgtaggaggaatctgtgctctacttacctctcaattcctggagatgccaacttcacagacacgtctgcagcagctggacatcacatttcatgtcctgcatggaaccagtggctgtgagtggcatgtccacttgctggattatcagccaggacactatagaacaggaccagctgagactaagaaggaccagcagagccagctcagctctgagccattcacacatcttcaccctcagtttcctcacttgaggagtgggatggggagaacagagagtagctgtgtttgaatccctgtaggaaatggtgaagcatagctctgggtctcctgggggagaccaggcttggctgcgggagagctggctgttgctggactacatgctggccactgctgtgaccacgacactgctggggcagcttcttccacagtgatgcctactgatgcttcagtgcctctgcacaccgcccattccacttcctccttccccacagggcaggtggggaagcagtttggcccagcccaaggagaccccaccttgagccttatttcctaatgggtccacctctcatctgcatctttcacacctcccagcttctgcccaaccttcagcagtgacaagtccccaagagactcgcctgagcagcttgggctgcttttcatttccacctgtcaggatgcctgtggtcatgctctcagctccacctggcatgagaagggatcctggcctctggcatattcatcaagtatgagttctggggatgagtcactgtaatgatgtgagcagggagccttcctccctgggccacctgcagagagctttcccaccaactttgtaccttgattgccttacaaagttatttgtttacaaacagcgaccatataaaagcctcctgccccaaagcttgtgggcacatgggcacatacagactcacatacagacacacacatatatgtacagacatgtactctcacacacacaggcaccagcatacacacgtttttctaggtacagctcccaggaacagctaggtgggaaagtcccatcactgagggagcctaaccatgtccctgaacaaaaattgggcactcatctattccttttctcttgtgtccctactcattgaaaccaaactctggaaaggacccaatgtaccagtatttatacctctaatgaagcacagagagaggaagagagctgcttaaactcacacaacaatgaactgcagacacagctgttctctccctctctccttcccagagcaatttatactttaccctcaggctgtcctctggggagaaggtgccatggtcttaggtgtctgtgccccaggacagaccctaggaccctaaatccaatagaaaatgcatatctttgctccactttcagccaggctggagcaaggtaccttttcttaggatcttgggagggaatggatgcccctctctgcatgatcttgttgaggcatttagctgccatgcacctgtccccctttaatactgggcattttaaagccatctcaagaggcatcttctacatgttttgtacgcattaaaataatttcaaagatatctgagaaaagccgatatttgccattcttcctatatcctggaatatatcttgcatcctgagtttataataataaataatattctaccttggaaaaaaaaaaaaaaa

SEQ ID NO: 49 - DNA, 2720 bp, Homo sapiens
gcggggtggagccagcgccctcagcgcgctacggtccgcgggcaactccgcagaagccccagcccccaggaccccaggacccagtggcgcagccggcagccccggatccctgatctgcttgggcagctcctgcagaacctggaacagtgaatgggtaggggacactgggcgtgcagaaggcggggggcagtgtggaacatgccttcaccacctccagcttctgctgccggaggctgcacccacctgtgcccatggcctgcacaggcccatcacttcctagcgccttcgacattctaggtgcagcaggccaggacaagctcttgtatctgaagcacaaactgaagaccccacgcccaggctgccaggggcaggacctcctgcatgccatggttctcctgaagctgggccaggaaactgaggccaggatctctctagaggcattgaaggccgatgcggtggcccggctggtggcccgccagtgggctggcgtggacagcaccgaggacccagaggagcccccagatgtgtcctgggctgtggcccgcttgtaccacctgctggctgaggagaagctgtgccccgcctcgctgcgggacgtggcctaccaggaagccgtccgcaccctcagctccagggacgaccaccggctgggggaacttcaggatgaggcccgaaaccggtgtgggtgggacattgctggggatccagggagcatccggacgctccagtccaatctgggctgcctcccaccatcctcggctttgccctctgggaccaggagcctcccacgccccattgacggtgtttcggactggagccaagggtgctccctgcgatccactggcagccctgcctccctggccagcaacttggaaatcagccagtcccctaccatgcccttcctcagcctgcaccgcagcccacatgggcccagcaagctctgtgacgacccccaggccagcttggtgcccgagcctgtccccggtggctgccaggagcctgaggagatgagctggccgccatcgggggagattgccagcccaccagagctgccaagcagcccacctcctgggcttcccgaagtggccccagatgcaacctccactggcctccctgatacccccgcagctccagaaaccagcaccaactacccagtggagtgcaccgaggggtctgcaggcccccagtctctccccttgcctattctggagccggtcaaaaacccctgctctgtcaaagaccagacgccactccaactttctgtagaagataccacctctccaaataccaagccgtgcccacctactcccaccaccccagaaacatcccctcctcctcctcctcctcctccttcatctactccttgttcagctcacctgaccccctcctccctgttcccttcctccctggaatcatcatcggaacagaaattctataactttgtgatcctccacgccagggcagacgaacacatcgccctgcgggttcgggagaagctggaggcccttggcgtgcccgacggggccaccttctgcgaggatttccaggtgccggggcgcggggagctgagctgcctgcaggacgccatagaccactcagctttcatcatcctacttctcacctccaacttcgactgtcgcctgagcctgcaccaggtgaaccaagccatgatgagcaacctcacgcgacaggggtcgccagactgtgtcatccccttcctgcccctggagagctccccggcccagctcagctccgacacggccagcctgctctccgggctggtgcggctggacgaacactcccagatcttcgccaggaaggtggccaacaccttcaagccccacaggcttcaggcccgaaaggccatgtggaggaaggaacaggacacccgagccctgcgggaacagagccaacacctggacggtgagcggatgcaggcggcggcactgaacgcagcctactcagcctacctccagagctacttgtcctaccaggcacagatggagcagctccaggtggcttttgggagccacatgtcatttgggactggggcgccctatggggctcgaatgccctttgggggccaggtgcccctgggagccccgccaccctttcccacttggccggggtgcccgcagccgccacccctgcacgcatggcaggctggcacccccccaccgccctccccacagccagcagcctttccacagtcactgcccttcccgcagtccccagccttccctacggcctcacccgcaccccctcagagcccagggctgcaacccctcattatccaccacgcacagatggtacagctggggctgaacaaccacatgtggaaccagagagggtcccaggcgcccgaggacaagacgcaggaggcagaatgaccgcgtgtccttgcctgaccacctggggaacacccctggacccaggcatcggccaggaccccatagagcaccccggtctgccctgtgccctgtggacagtggaagatgaggtcatctgccactttcaggacattgtccgggagcccttcatttaggacaaaacgggcgcgatgatgccctggctttcagggtggtcagaactggatacggtgtttacaattccaatctctctatttctgggtgaagggtcttggtggtgggggtattgctacggtcttttaattataataaatatttattgaatgcttccgcaaaaaaaaaaaaaaaaaa

SEQ ID NO: 50 - DNA, 4992 bp, Homo sapiens
gaagactccagatataggatcactccatgccatcaagaaagttgatgctattgggcccatctcaagctgatcttggcacctctcatgctctgctctcttcaaccagacctctacattccattttggaagaagactaaaaatggtgtttccaatgtggacactgaagagacaaattcttatcctttttaacataatcctaatttccaaactccttggggctagatggtttcctaaaactctgccctgtgatgtcactctggatgttccaaagaaccatgtgatcgtggactgcacagacaagcatttgacagaaattcctggaggtattcccacgaacaccacgaacctcaccctcaccattaaccacataccagacatctccccagcgtcctttcacagactggaccatctggtagagatcgatttcagatgcaactgtgtacctattccactggggtcaaaaaacaacatgtgcatcaagaggctgcagattaaacccagaagctttagtggactcacttatttaaaatccctttacctggatggaaaccagctactagagataccgcagggcctcccgcctagcttacagcttctcagccttgaggccaacaacatcttttccatcagaaaagagaatctaacagaactggccaacatagaaatactctacctgggccaaaactgttattatcgaaatccttgttatgtttcatattcaatagagaaagatgccttcctaaacttgacaaagttaaaagtgctctccctgaaagataacaatgtcacagccgtccctactgttttgccatctactttaacagaactatatctctacaacaacatgattgcaaaaatccaagaagatgattttaataacctcaaccaattacaaattcttgacctaagtggaaattgccctcgttgttataatgccccatttccttgtgcgccgtgtaaaaataattctcccctacagatccctgtaaatgcttttgatgcgctgacagaattaaaagttttacgtctacacagtaactctcttcagcatgtgcccccaagatggtttaagaacatcaacaaactccaggaactggatctgtcccaaaacttcttggccaaagaaattggggatgctaaatttctgcattttctccccagcctcatccaattggatctgtctttcaattttgaacttcaggtctatcgtgcatctatgaatctatcacaagcattttcttcactgaaaagcctgaaaattctgcggatcagaggatatgtctttaaagagttgaaaagctttaacctctcgccattacataatcttcaaaatcttgaagttcttgatcttggcactaactttataaaaattgctaacctcagcatgtttaaacaatttaaaagactgaaagtcatagatctttcagtgaataaaatatcaccttcaggagattcaagtgaagttggcttctgctcaaatgccagaacttctgtagaaagttatgaaccccaggtcctggaacaattacattatttcagatatgataagtatgcaaggagttgcagattcaaaaacaaagaggcttctttcatgtctgttaatgaaagctgctacaagtatgggcagaccttggatctaagtaaaaatagtatattttttgtcaagtcctctgattttcagcatctttctttcctcaaatgcctgaatctgtcaggaaatctcattagccaaactcttaatggcagtgaattccaacctttagcagagctgagatatttggacttctccaacaaccggcttgatttactccattcaacagcatttgaagagcttcacaaactggaagttctggatataagcagtaatagccattattttcaatcagaaggaattactcatatgctaaactttaccaagaacctaaaggttctgcagaaactgatgatgaacgacaatgacatctcttcctccaccagcaggaccatggagagtgagtctcttagaactctggaattcagaggaaatcacttagatgttttatggagagaaggtgataacagatacttacaattattcaagaatctgctaaaattagaggaattagacatctctaaaaattccctaagtttcttgccttctggagtttttgatggtatgcctccaaatctaaagaatctctctttggccaaaaatgggctcaaatctttcagttggaagaaactccagtgtctaaagaacctggaaactttggacctcagccacaaccaactgaccactgtccctgagagattatccaactgttccagaagcctcaagaatctgattcttaagaataatcaaatcaggagtctgacgaagtattttctacaagatgccttccagttgcgatatctggatctcagctcaaataaaatccagatgatccaaaagaccagcttcccagaaaatgtcctcaacaatctgaagatgttgcttttgcatcataatcggtttctgtgcacctgtgatgctgtgtggtttgtctggtgggttaaccatacggaggtgactattccttacctggccacagatgtgacttgtgtggggccaggagcacacaagggccaaagtgtgatctccctggatctgtacacctgtgagttagatctgactaacctgattctgttctcactttccatatctgtatctctctttctcatggtgatgatgacagcaagtcacctctatttctgggatgtgtggtatatttaccatttctgtaaggccaagataaaggggtatcagcgtctaatatcaccagactgttgctatgatgcttttattgtgtatgacactaaagacccagctgtgaccgagtgggttttggctgagctggtggccaaactggaagacccaagagagaaacattttaatttatgtctcgaggaaagggactggttaccagggcagccagttctggaaaacctttcccagagcatacagcttagcaaaaagacagtgtttgtgatgacagacaagtatgcaaagactgaaaattttaagatagcattttacttgtcccatcagaggctcatggatgaaaaagttgatgtgattatcttgatatttcttgagaagccctttcagaagtccaagttcctccagctccggaaaaggctctgtgggagttctgtccttgagtggccaacaaacccgcaagctcacccatacttctggcagtgtctaaagaacgccctggccacagacaatcatgtggcctatagtcaggtgttcaaggaaacggtctagcccttctttgcaaaacacaactgcctagtttaccaaggagaggcctggctgtttaaattgttttcatatatatcacaccaaaagcgtgttttgaaattcttcaagaaatgagattgcccatatttcaggggagccaccaacgtctgtcacaggagttggaaagatggggtttatataatgcatcaagtcttctttcttatctctctgtgtctctatttgcacttgagtctctcacctcagctcctgtaaaagagtggcaagtaaaaaacatggggctctgattctcctgtaattgtgataattaaatatacacacaatcatgacattgagaagaactgcatttctacccttaaaaagtactggtatatacagaaatagggttaaaaaaaactcaagctctctctatatgagaccaaaatgtactagagttagtttagtgaaataaaaaaccagtcagctggccgggcatggtggctcatgcttgtaatcccagcactttgggaggccgaggcaggtggatcacgaggtcaggagtttgagaccagtctggccaacatggtgaaaccccgtctgtactaaaaatacaaaaattagctgggcgtggtggtgggtgcctgtaatcccagctacttgggaggctgaggcaggagaatcgcttgaacccgggaggtggaggtggcagtgagccgagatcacgccactgcaatgcagcccgggcaacagagctagactgtctcaaaagaacaaaaaaaaaaaaacacaaaaaaactcagtcagcttcttaaccaattgcttccgtgtcatccagggccccattctgtgcagattgagtgtgggcaccacacaggtggttgctgcttcagtgcttcctgctctttttccttgggcctgcttctgggttccatagggaaacagtaagaaagaaagacacatccttaccataaatgcatatggtccacctacaaatagaaaaatatttaaatgatctgcctttatacaaagtgatattctctacctttgataatttacctgcttaaatgtttttatctgcactgcaaagtactgtatccaaagtaaaatttcctcatccaatatctttcaaactgttttgttaactaatgccatatatttgtaagtatctgcacacttgatacagcaacgttagatggttttgatggtaaaccctaaaggaggactccaagagtgtgtatttatttatagttttatcagagatgacaattatttgaatgccaattatatggattcctttcattttttgctggaggatgggagaagaaaccaaagtttatagaccttcacattgagaaagcttcagttttgaacttcagctatcagattcaaaaacaacagaaagaaccaagacattcttaagatgcctgtactttcagctgggtataaattcatgagttcaaagattgaaacctgaccaatttgctttatttcatggaagaagtgatctacaaaggtgtttgtgccatttggaaaacagcgtgcatgtgttcaagccttagattggcgatgtcgtattttcctcacgtgtggcaatgccaaaggctttactttacctgtgagtacacactatatgaattatttccaacgtacatttaatcaataagggtcacaaattcccaaatcaatctctggaataaatagagaggtaattaaattgctggagccaacta

SEQ ID NO: 51 - DNA, 3196 bp, Homo sapiens
agtgcggggtctgacagaggaggctccgtgtctgcagctagtgtgtcaactcagcgtttctcctctcgtccctggtgaggtgtagcggcggcacgcggctggagatcccctgtggcctccagtttaggaagggtccagcatcccaagggaggggtgtgtgggcgaggggtctctgggcccggggtcgcggctgtgaggagaggatgcccgcgcggcggcatctcaggcacctggaggaggccgcgctttctcctcagggaaccggcgccttggcagcccccggcgacgccgcccccttcgcggcctaggttggtctggtgagccgggaagcgggcgtcgttcgcagcgccgctgtgaccaccgcgtcccgggcggagctgggctcagtgccggcctgggcctagagtccgagcctcgagctgccggcgtggggggtcgcgagtggcctaatgcggcctcgaagccgaaggacccgagtccgagctcgcactccgacccgctggtgctgtggaaaactcaggtggccttccgctttcgtagcctctaaagtggggaccaagactttcacctcttaggattgtagtcgggattaaaagattttcccggaagagctaaagatggctgaatttctagatgaccaggaaactcgactgtgtgacaactgcaaaaaagaaattcctgtgtttaactttaccatccatgagatccactgtcaaaggaacattggtatgtgtcctacctgtaaggaaccatttcccaaatctgacatggagactcacatggctgcagaacactgtcaggtgacctgcaaatgtaacaagaagttggagaagaggctgttaaagaagcatgaggagactgagtgccctttgcggcttgctgtctgccagcactgtgatttagaactttccattctcaaactgaaggaacatgaagattattgtggtgcccggacggaactatgtggcaactgtggtcgcaatgtccttgtgaaagatctgaagactcaccctgaagtttgtgggagagagggggaggaaaagagaaatgaggttgccatacctcctaatgcatatgatgaatcttggggtcaggatggaatctggattgcatcccaactcctcagacaaattgaggctctggacccacccatgaggctgccgcgaaggcccctgagagcctttgaatcagatgttttccacaatagaactaccaaccaaaggaacattacagcccaggtttcaattcagaataatctgtttgaagaacaagagaggcaggaaaggaatagaggccaacagccccccaaagagggtggtgaagagagtgcaaacttggacttcatgttggccctaagtctgcaaaatgaaggccaagcctccagtgtggcagagcaggacttctggagggccgtatgtgaggccgaccagtctcatggcggtcccaggtctctcagtgacataaagggtgcagctgacgagatcatgttgccttgtgaattttgtgaggagctctacccagaggaactgctgattgaccatcagacaagctgtaacccttcacgtgccttaccttcactcaatactggcagctcttcccccagaggggtggaggaacctgatgtcatcttccagaacttcttgcaacaggctgcaagtaaccagttagactctttgatgggcctgagcaattcacaccctgtggaggagagcatcattatcccatgtgaattctgtggggtacagctggaagaggaggtgctgttccatcaccaggaccagtgtgaccaacgcccagccactgcaaccaaccatgtgacagaggggattcctagactggattcccagcctcaagagacctcaccagagctgcccaggaggcgtgtcagacaccagggagacctgtcttctggttacctggatgatactaagcaggaaacagctaatgggcccacctcctgtctgcctcccagccgacccattaacaatatgacagctacctataaccagctatcgagatcaacatcaggccccagacctgggtgccagcccagctctccttgtgtgccgaagctcagcaactcagacagccaggacatccaggggcggaatcgagacagccagaatggggccatagcccctgggcacgtttcagtgattcgccctcctcaaaatctctacccagaaaacattgtgccctctttctcccctgggccttcagggagatacggagctagtggtaggagtgaaggtggcaggaattcccgggtcacccctgcagctgccaactaccgcagcagaactgcaaaggcaaagccttccaagcaacagggagctggggatgcagaagaggaagaggaggagtaatggtgtctccagagactttacatcggttcctgtcttctgtgcacagcagcacttgccgctgtgcaggcccacctctttggctctttgggtgggagagtttttccagattttagatttttctaggttatggccattttgtgtcttttgaggttgtgctgtgggggtttgggtttgagggaagggagcagggtggcggttgaggaacgcttcagccttagctgctacctttcggcagcagtgaaatacaagctgcagcctcggctgccagggctcccttttgacttattgtcgccactgccccttggtgctgtgtggtcccagtggaaggaggggaagattttggaaacctggtagccaccagtaaggtgattctctgccctgttggggcctaaatttgggggcttttgggcaacctctccgtgtactgcgtctgtccacactcgattgggccccaggtgtgtatgaggcgctctggtaaggtgctcaggccagttgcaatgtctgtcagtaacgaggcttttgatgtgttgagctggaggtgagtggaccgggggctgtgttttaagctgcttccttggcatttggcatcactgccttctgttcccgggggagcatggatcttttgtcctcactgctttctaatggggagggctgagggctccctgtccccacagcaggtatgttgggctctgccccagccccacacttgctctgaaaaccaagtgtcagagccccttccccttgtttttattttactgttataataattattaacttccttgtaatagaaataaagtttgtacttggagttcaaaaaaaaaaaaaaaaaa

SEQ ID NO: 52 - DNA, 2665 bp, Homo sapiens
agtgcggggtctgacagaggaggctccgtgtctgcagctagtgtgtcaactcagcgtttctcctctcgtccctggaagagctaaagatggctgaatttctagatgaccaggaaactcgactgtgtgacaactgcaaaaaagaaattcctgtgtttaactttaccatccatgagatccactgtcaaaggaacattggtatgtgtcctacctgtaaggaaccatttcccaaatctgacatggagactcacatggctgcagaacactgtcaggtgacctgcaaatgtaacaagaagttggagaagaggctgttaaagaagcatgaggagactgagtgccctttgcggcttgctgtctgccagcactgtgatttagaactttccattctcaaactgaaggaacatgaagattattgtggtgcccggacggaactatgtggcaactgtggtcgcaatgtccttgtgaaagatctgaagactcaccctgaagtttgtgggagagagggggaggaaaagagaaatgaggttgccatacctcctaatgcatatgatgaatcttggggtcaggatggaatctggattgcatcccaactcctcagacaaattgaggctctggacccacccatgaggctgccgcgaaggcccctgagagcctttgaatcagatgttttccacaatagaactaccaaccaaaggaacattacagcccaggtttcaattcagaataatctgtttgaagaacaagagaggcaggaaaggaatagaggccaacagccccccaaagagggtggtgaagagagtgcaaacttggacttcatgttggccctaagtctgcaaaatgaaggccaagcctccagtgtggcagagcaggacttctggagggccgtatgtgaggccgaccagtctcatggcggtcccaggtctctcagtgacataaagggtgcagctgacgagatcatgttgccttgtgaattttgtgaggagctctacccagaggaactgctgattgaccatcagacaagctgtaacccttcacgtgccttaccttcactcaatactggcagctcttcccccagaggggtggaggaacctgatgtcatcttccagaacttcttgcaacaggctgcaagtaaccagttagactctttgatgggcctgagcaattcacaccctgtggaggagagcatcattatcccatgtgaattctgtggggtacagctggaagaggaggtgctgttccatcaccaggaccagtgtgaccaacgcccagccactgcaaccaaccatgtgacagaggggattcctagactggattcccagcctcaagagacctcaccagagctgcccaggaggcgtgtcagacaccagggagacctgtcttctggttacctggatgatactaagcaggaaacagctaatgggcccacctcctgtctgcctcccagccgacccattaacaatatgacagctacctataaccagctatcgagatcaacatcaggccccagacctgggtgccagcccagctctccttgtgtgccgaagctcagcaactcagacagccaggacatccaggggcggaatcgagacagccagaatggggccatagcccctgggcacgtttcagtgattcgccctcctcaaaatctctacccagaaaacattgtgccctctttctcccctgggccttcagggagatacggagctagtggtaggagtgaaggtggcaggaattcccgggtcacccctgcagctgccaactaccgcagcagaactgcaaaggcaaagccttccaagcaacagggagctggggatgcagaagaggaagaggaggagtaatggtgtctccagagactttacatcggttcctgtcttctgtgcacagcagcacttgccgctgtgcaggcccacctctttggctctttgggtgggagagtttttccagattttagatttttctaggttatggccattttgtgtcttttgaggttgtgctgtgggggtttgggtttgagggaagggagcagggtggcggttgaggaacgcttcagccttagctgctacctttcggcagcagtgaaatacaagctgcagcctcggctgccagggctcccttttgacttattgtcgccactgccccttggtgctgtgtggtcccagtggaaggaggggaagattttggaaacctggtagccaccagtaaggtgattctctgccctgttggggcctaaatttgggggcttttgggcaacctctccgtgtactgcgtctgtccacactcgattgggccccaggtgtgtatgaggcgctctggtaaggtgctcaggccagttgcaatgtctgtcagtaacgaggcttttgatgtgttgagctggaggtgagtggaccgggggctgtgttttaagctgcttccttggcatttggcatcactgccttctgttcccgggggagcatggatcttttgtcctcactgctttctaatggggagggctgagggctccctgtccccacagcaggtatgttgggctctgccccagccccacacttgctctgaaaaccaagtgtcagagccccttccccttgtttttattttactgttataataattattaacttccttgtaatagaaataaagtttgtacttggagttcaaaaaaaaaaaaaaaaaa

SEQ ID NO: 53 - DNA, 1972 bp, Homo sapiens
gaaactcccgcctggccaccataaaagcgccggccctccgcttccccgcgagacgaaacttcccgtcccggcggctctggcacccagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 54 - DNA, 1885 bp, Homo sapiens
gaaactcccgcctggccaccataaaagcgccggccctccgcttccccgcgagacgaaacttcccgtcccggcggctctggcacccagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 55 - DNA, 2051 bp, Homo sapiens
agggtgcgaagcgccactgtttaggtttcgctttcccgggagcctgacccgcccctgacgtcgcctttcccgtctccgcagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagaggtaagagcccggcccaccctctccagatgccagtccccgagcgccctgcagccggccctgactctccgcggccgggcacccgcagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 56 - DNA, 515 bp, Homo sapiens
gttgaggatctcttactctctaagccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggccatggcggctgtgcccatggtgctcagtgccatgggcttcactgcggcgggaatcgcctcgtcctccatagcagccaagatgatgtccgcggcggccattgccaatgggggtggagttgcctcgggcagccttgtgggtactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctg

SEQ ID NO: 57 - DNA, 544 bp, Homo sapiens
taactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtataagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaataaaaatgaggtactctcctggaaatatt

SEQ ID NO: 58 - DNA, 482 bp, Homo sapiens
atttctttggcagttttcgtgctaatgtttttgctaaggaagataagctctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactg

SEQ ID NO: 59 - DNA, 534 bp, Homo sapiens
gaacattccaggcagactttcctctgggccctgccacccacggagggacctacagatgcttcggctctttccgtgccctgccctgcgtgtggtcaaactcaagtgacccactgcttgtttctgtcacaggaaacccttcaagtagttggccttcacccacagaaccaagctccaaatctggtatctgcagacacctgcatgttctgattgggacctcagtggtcatcttcctcttcatcctcctcctcttctttctcctttatcgctggtgctccaacaaaaagaatgctgctgtaatggaccaagagcctgcgggggacagaacagtgaataggcaggactctgatgaacaagaccctcaggaggtgacgtacgcacagttggatcactgcgttttcatacagagaaaaatcagtcgcccttctcagaggcccaagacacccctaacagataccagcgtgtacacggaacttccaaatgctgagcccagatccaaagttgtctcctgcccacgagcaccacagtcaggtcttg

SEQ ID NO: 60 - DNA, 534 bp, Homo sapiens
ggaacttccaaatgctgagcccagatccaaagttgtctcctgcccacgagcaccacagtcaggtcttgagggggttttctagggagacaacagccctgtctcaaaaccaggttgccagatccaatgaaccagcagctggaatctgaaggcatcagtctgcatcttaggggatcgctcttcctcacaccacgaatctgaacatgcctctctcttgcttacaaatgcctaaggtcgccactgcctgctgcagagaaaacacactcctttgcttagcccacaaggtatctatttcacttgacccctgcccacctctccaacctaactggcttacttcctagtcctacttgaggctgcaatcacactgaggaactcacaattccaaacatacaagaggctccctcttaacacggcacttacacacttgctgttccaccttccctcatgctgttccacctcccctcagactatctttcagccttctgtcatcagtaaaatttataaattttttttataacttcagtgtagctctctcct

SEQ ID NO: 61 - DNA, 456 bp, Homo sapiens
taatggaccaagagcctgcgggggacagaacagtgaataggcaggactctgatgaacaagaccctcaggaggtgacgtacgcacagttggatcactgcgttttcatacagagaaaaatcagtcgcccttctcagaggcccaagacacccctaacagataccagcgtgtacacggaacttccaaatgctgagcccagatccaaagttgtctcctgcccacgagcaccacagtcaggtcttgagggggttttctagggagacaacagccctgtctcaaaaccaggttgccagatccaatgaaccagcagctggaatctgaaggcatcagtctgcatcttaggggatcgctcttcctcacaccacgaatctgaacatgcctctctcttgcttacaaatgcctaaggtcgccactgcctgctgcagagaaaacacactcctttgcttagcccacaagtat

SEQ ID NO: 62 - DNA, 547 bp, Homo sapiens
acctgcatgttctgattgggacctcagtggtcatcttcctcttcatcctcctcctcttctttctcctttatcgctggtgctccaacaaaaagaatgctgctgtaatggaccaagagcctgcgggggacagaacagtgaataggcaggactctgatgaacaagaccctcaggaggtgacgtacgcacagttggatcactgcgttttcatacagagaaaaatcagtcgcccttctcagaggcccaagacacccctaacagataccagcgtgtacacggaacttccaaatgctgagcccagatccaaagttgtctcctgcccacgagcaccacagtcaggtcttgagggggttttctagggagacaacagccctgtctcaaaaccaggttgccagatccaatgaaccagcagctggaatctgaaggcatcagtctgcatcttaggggatcgctcttcctcacaccacgaatctgaacatgcctctctcttgcttacaaatgcctaaggtcgccactgcctgctgcagagaaaacacactcctttgcttag

SEQ ID NO: 63 - DNA, 488 bp, Homo sapiens
gaacagcgaacaggcaggactctgatgaacaagaccctcaggaggtgacatacacacagttgaatcactgcgttttcacacagagaaaaatcactcgcccttctcagaggcccaagacacccccaacagatatcatcgtgtacgcggaacttccaaatgctgagtccagatccaaagttgtctcctgcccatgagcaccacagtcaggccttgagggcatcttctagggagacaacagccctgtctcaaaaccgggttgccagctcccatgtaccagcagctggaatctgaaggcatgagtctgcatcttagggcatcgctcttcctcacaccacaaatctgaatgtgcctctcacttgcttacaaatgtctaaggtccccactgcctgctggagaaaaaacacactcctttgcttagcccacagttctccatttcacttgacccctgcccacctctccaacctaactggcttacttcctagtctact

SEQ ID NO: 64 - DNA, 475 bp, Homo sapiens
tcagtgtaatccttgactttgctcctcaccatcagggcaaacttgccttcttccctcctaagctccagtaaataaacagaacagctttcaccaaagtgggtagtatagtcctcaaatatcggataaatatatgcgtttttgtaccccagaaaaacttttcctccctcttcatcaacatagtaaaataagtcaaacaaaatgagaacaccaaattttgggggaataaatttttatttaacactgcaaaggaaagagagagaaaacaagcaaagataggtaggacagaaaggaagacagccagatccagtgattgacttggcatgaaaatgagaaaatgcagacagacctcaacattcaacattcaacaacatccatacagcactgctggaggaagaggaagatttgtgcagaccaagagcaccacagactacaactgcccagcttcatctaaatacttgttaacctctttggtcat

SEQ ID NO: 65 - DNA, 526 bp, Homo sapiens
tgcagggcccaaggtcaacggaacattccaggccgactttcctctgggccctgccacccacggagggacctacagatgcttcggctctttccgtgactctccatacgagtggtcaaactcgagtgacccactgcttgtttctgtcacaggaaacccttcaaatagttggccttcacccactgaaccaagctccgaaaccggtaaccccagacacctgcatgttctgattgggacctcagtggtcatcatcctcttcatcctcctcctcttctttctccttcatcgctggtgctgcaacaaaaaaaatgctgttgtaatggaccaagagcctgcagggaacagaacagtgaacagggaggactctgatgaacaagaccctcaggaggtgacatatgcacagttgaatcactgcgttttcacacagagaaaaatcactcacccttctcagaggcccaagacacccccaacagatatcatcgtgtacacggaacttccaaatgctgagccctgatccaaagttgtctcc

SEQ ID NO: 66 - DNA, 542 bp, Homo sapiens
actggcccttctcagaggagcaagagaccctcaacagataccagcgtgtgtatagaacttccaaatgctgagcccagagcgttatctcctgcgcatgagcaccacagtcaggccttgatgggatcttctagggagacaacagccctgtctcaaacccagcttgccagctcccatgtaccagcagctggaatctgaaggcgtgagtctccatcttagagcatcactcttcctcacaccacaaatctggtgcctgtctcttgcttaccaatgtctaaggtccccactgcctgctgcagagaaaacacactcctttgcttagcccacaattctctatttcacttgacccctgcccacctctccaacctaactggcttacttcctagtctacttgaggctgcaatcacactgaggaactcacaattccaaacatacaagaggctctctcttaacacggcacttagacacgtgctgttccaccttccctcgtgctgttccacctttcctcagactatttttcagccttctggcatcagcaaacctta

SEQ ID NO: 67 - DNA, 225 bp, Homo sapiens
aatctactaattccacaccttttattgacacagaaaatgttgagaatcccaaatttgattgatttgaagaacatgtgagaggtttgactagatgatggatgccaatattaaatctgctggagtttcatgtacaagatgaaggagaggcaacatccaaaatagttaagacatgatttccttgaatgtggcttgagaaatatggacacttaatactaccttgaaaat

SEQ ID NO: 68 - DNA, 388 bp, Homo sapiens
ctgggattacaggcttgagcccccgcgcccagccatcaaaatgctttttatttctgcatatgtttgaatactttttacaatttaaaaaaatgatctgttttgaaggcaaaattgcaaatcttgaaattaagaaggcaaaatgtaaaggagtcaaactataaatcaagtatttgggaagtgaagactggaagctaatttgcataaattcacaaacttttatactctttctgtatatacattttttttctttaaaaaacaactatggatcagaatagccacatttagaacactttttgttatcagtcaatatttttagatagttagaacctggtcctaagcctaaaagtgggcttgattctgcagtaaatcttttacaactgcctcgaca

SEQ ID NO: 69 - DNA, 399 bp, Homo sapiens
tcacaaacttttatactctttctgtatatacattttttttctttaaaaaacaactatggatcagaatagccacatttagaacactttttgttatcagtcaatatttttagatagttagaacctggtcctaagcctaaaagtgggcttgattctgcagtaaatcttttacaactgcctcgacacacataaacctttttaaaaatagacactccccgaagtcttttgttcgcatggtcacacactgatgcttagatgttccagtaatctaatatggccacagtagtcttgatgaccaaagtcctttttttccatctttagaaaactacatgggaacaaacagatcgaacagttttgaagctactgtgtgtgtgaatgaacactcttgctttattccagaat

SEQ ID NO: 70 - DNA, 459 bp, Homo sapiens
  misc_feature (32)..(32): n is a, c, g, or t
tcacaaacttttatactctttctgtatatacnttttttttctttaaaaaacaactatggatcagaatagccacatttngaanactttttgttatcagtcaatatttttagatagttagaacctggtcctaagcctaaaagtgggcttgattctgcagtaaatcttttacaactgcctcgacacacataaacctttttaaaaatagacactccccgaagtcttttgttcgcatggtcacacanctgatgcttagatgttccagtaatctaatatggccacagtagtcttgatgaccaaagtcctttttttccatctttagaaaactacatgggaacaaacagatcgaacagttttgaagctactgtgtgtgtgaatgaacactcttgctttattccagaatgctgtacatctattttggattgtatattgtgtttgtgtatttacgctttgattcatagt

SEQ ID NO: 71 - DNA, 346 bp, Homo sapiens
cgcgcaccgacggaggggacatgggcagagcaatggtggccaggctcgggctggggctgctgctgctggcactgctcctacccacgcagatttattccagtgaaacaacaactggaacttcaagtaactcctcccagagtacttccaactctgggttggccccaaatccaactaatgccaccaccaaggtggctggtggtgccctgcagtcaacagccagtctcttcgtggtctcactctctcttctgcatctctactcttaagagactcaggccaagaaacgtcttctaaatttccccatcttctaaacccaatccaaatggcgtctggaagtccaatgtggcaa

SEQ ID NO: 72 - DNA, 478 bp, Homo sapiens
ttcacaaacttttatactctttctgtatatacattttttttctttaaaaaacaactatggatcagaatagcaacatttagaacactttttgttatcagtcaatatttttagatagttagaacctggtcctaagcctaaaagtgggcttgattctgcagtaaatcttttacaactgcctcgacacacataaacctttttaaaaatagacactccccgaagtcttttgtttgtatggtcacacactgatgcttagatgttccagtaatctaatatggccacagtagtcttgatgaccaaagtcctttttttccatctttagaaaactacatgggaacaaacagatcgaacagttttgaagctactgtgtgtgtgaatgaacactcttgctttattccagaatgctgtacatctattttggattgtatattgtggttgtgtatttacgctttgattcatagtaacttcttatggaattgat

SEQ ID NO: 73 - DNA, 517 bp, Homo sapiens
gtggcggagcagctgagagcctacctggagggcgagtgcgtggagtggctccgcagatacctggagaacgggaaggagacgctgcagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagctcaggtggaaaaggagggagctactctcaggctg

SEQ ID NO: 74 - DNA, 509 bp, Homo sapiens
gacctggcagcgggatggcgaggaccaaactcaggacaccgagcttgtggagaccagaccagcaggagacagaaccttccagaagtgggcagctgtggtggtgccttctggagaagagcagagatacacatgccatgtacagcatgaggggctgccgaagcccctcaccctgagatgggagccatcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtccagcgacagtgcccagggctctgatgtgtctctcacagcttgaaaagcctgagacagctgtcttgtgagggactgagatgcaggatttcttcacgcctcccctttgtgacttcaagagcctctggcatctctttctgcaaaggcacctgaatgtgtctgcgtccctgttagcataatgt

SEQ ID NO: 75 - DNA, 505 bp, Homo sapiens
ctgagagcctacctggagggcctgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagccatcttcccagtccaccatccccatcgtgggcattgttgctggcctggctgtcctagcagttgtggtcatcggagctgtggtcgctactgtgatgtgtaggaggaagagctcaggtggaaaaggagggagctactctcaggctg

SEQ ID NO: 76 - DNA, 425 bp, Homo sapiens
gctgtcctaggagctgtggtggctgttgttatgtgtaggaggaagagctcaggtggaaaaggagggagctgctctcaggctgcgtccagcaacagtgcccagggctctgatgagtctctcatcgcttgtaaagcctgagacagctgcctgtgtgggactgagatgcaggatttcttcacacctctcctttgtgacttcaagagcctctggcatctctttctgcaaaggcatctgaatgcgtctgcgttcctgttagcataatgtgaggaggtggagagacagcccacccccgtgtccaccgtgacccctgtccccacactgacctgtgttccctccccgatcatctttcctgttccagagaagtgggctggatgtctccatctctgtctcaactttacgtgtactgagctgcaacttcttacttc

SEQ ID NO: 77 - DNA, 230 bp, Homo sapiens
aggtctagtcagagcgtcgtacaccctgatggaaacacctagttgtgatggactcagcagcggccaggccatcttccaagagtccttatacacaagatttgtaaccgttgctttggggtcccaaaacgagttagcggcgggggcccagggacagatttcacactgaaaatcagcaggctggaagcgggggatgtcggggttcattaatacatgcgagatacacaatttcc

SEQ ID NO: 78 - DNA, 591 bp, Homo sapiens
gaggcggagcagctgagagcctacctggagggcgagtgcgtggagtggctccgcggatacctggagaacgggaaggagacgctgcagcgcgcggaacgcccaaagacacacgtgacccaccatcccgtctctgaccatgaggccaccctgaggtgctgggccctgggcttctaccctgcggagatcacactgacctggcagcgggatggggaggaccaaactcaggacaccgagcttgtggagaccaggccagcaggagatggaaccttccagaagtgggcagctgtggtggtgccttctggacaagaacagagatacacgtgccatgtgcagcacgaggggctgcaggagccctgcaccctgagatggaagccgtcttcccagcccaccatccccaacttgggcatcgtttctggcccagctgtcctggctgtcctggctgtcctggctgtcctagctgtcctaggagctgtggtcgctgctgtgatacataggaggaagagctcaggtggaaaaggagggagctgctctcaggctgcgtccagcaacagtgcccagggctctgatgagtctctcatcgcttgtaaagcc

SEQ ID NO: 79 - DNA, 524 bp, Homo sapiens
  misc_feature (425)..(427): n is a, c, g, or t
agctgtgatggtgccttctggagaagagcagagatacacgtgccatgtgcagcacgaggggctgccggagcccctcaccctgagatgggagccgtcttcccagcccaccatccccatcgtgggcatcgttgctggcctggctgtcctggctgtcctagctgtcctaggagctgtggtggctgttgtgatgtgtaggaggaagagctcaggtggaaaaggagggagctgctctcaggctgcgtccagcaacagtgcccagggctctgatgagtctctcatcgcttgtaaagcctgagacagctgcctgtgtgggactgagatgcaggatttcttcacacctctcctttgtgacttcaagagcctctggcatctctttctgcaaaggcatctgaatgtgtctgcgttcctgttagcataatgtgagnnngtggagagnacagcccacccccnnnnnnncaccgtgacccctgtccccaacacatgacctgtgttccctccccgatcatctttcctgttccagagaa

SEQ ID NO: 80 - DNA, 379 bp, Homo sapiens
ccatcatgggcatcgttgctggcctggctgtcctggttgtcctagctgtccttggagctgtggtcaccgctatgatgtgtaggaggaagagctcaggtggtgtgtgggactgagatgcaggatttcttcacacctctcctttgtgacttcaagagcctctggcatctctttctgcaaaggcgtctgaatgtgtctgcgttcctgttagcataatgtgaggaggtggagagacagcccacccccgtgtccaccgtgacccctgtccccacactgacctgtgttccctccccgatcatctttcctgttccagagaggtggggctggatgtctccatctctgtctcaaattcatggtgcactgagctgcaacttcttacttc

SEQ ID NO: 81 - DNA, 493 bp, Homo sapiens
tagctttggggcttctgactgcagttcttctgagtgtgctgctataccagtggatcctgtgccagggctccaactactccacttgtgccagctgtcctagctgcccagaccgctggatgaaatatggtaaccattgttattatttctcagtggaggaaaaggactggaattctagtctggaattctgcctagccagagactcacacctccttgtgataacggacaatcaggaaatgagcctgctccaagttttcctcagtgaggccttttgctggattggtctgaggaacaattctggctggaggtgggaagatggatcacctctaaacttctcaaggatttcttctaatagctttgtgcagacatgcggtgccatcaacaaaaatggtcttcaagcctcaagctgtgaagttcctttacactgggtgtgtaagaaggtcagactttgatagatgaccactctgtcctgaccctcagatctgtcatgtatccc

SEQ ID NO: 82 - DNA, 503 bp, Homo sapiens
tggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactgg

SEQ ID NO: 83 - DNA, 434 bp, Homo sapiens
caccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagt

SEQ ID NO: 84 - DNA, 482 bp, Homo sapiens
caccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaagcagtgacagtgcccagggctctaatgtgtctctcacggcttgtaaattgtgaaacagctgccctgtgtgggactgagt

SEQ ID NO: 85 - DNA, 469 bp, Homo sapiens
gctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgac

SEQ ID NO: 86 - DNA, 402 bp, Homo sapiens
ggagctcctatgacatgtaccatctatccagggaggggggagcccatgaacgtaggctccctgcagtgcgcaaggtcaacagaacattccaggcagatttccctctgggccctgccacccacggagggacctacagatgcttcggctctttccgtcactctccctacgagtggtcagacccgagtgacccactgcttgtttctgtcacaggaaacccttcaagtagttggccttcacccacagaaccaagctccaaatctggtaacctcagacacctgcacattctgattgggacctcagtggtcaaaatccctttcaccatcctcctcttctttctccttcatcgctggtgctccaacaaaaaaaaatgctgctgtaatggaccaagagcctgcagggaac

SEQ ID NO: 87 - DNA, 528 bp, Homo sapiens
tgggacctcagtggtcatcatcctcttcatcctcctcctcttctttctccttcatctctggtgctccaacaaaaaaaatgctgctgtaatggaccaagagcctgcagggaacagaacagccaacagcgaggactctgatgaacaagaccctcaggaggtgacatatgcacagttggatcactgcgttttcacacagagaaaaatcactcgcccttctcagaggcccaagacaccccctacagataccatcttgtacacggaacttccaaatgctaagcccagatccaaagttgtctcctgcccatgagcaccacagtcaggccttgagggcgtcttctagggagacaacagccctgtctcaaaaccgagttgccagctcccatgtaccagcagctggaatctgaaggcgtgagtcttcatcttagggcatcgctcctcctcacgccacaaatctggtgcctctctcttgcttacaaatgtctaggtccccactgcctgctggaaagaaaacacactcctttgcttagc

SEQ ID NO: 88 - DNA, 558 bp, Homo sapiens
  misc_feature (126)..(127): n is a, c, g, or t
gacagacctcaggagggctattggtccaggacccacacctgctttcttcatgtttcctgatcccgccctgggtctgcagtcacacatttctggaaacttctctggggtccaagactaggaggttcnnctnggaccttanggccntggntcntttctggtatctcacanggacattnncttctcacagatagaaaaggagggagttacactcaggctgcanncagtgacagtgcccaggctctgatgtgtcnctcacagcttgtaaagtgtgagacagctgccttgtgtgggactgagaggcaagagttgttcctgcccttccctttgtgacttgaagaaccctgactttgtttctgcaaaggcacctgcatgtgtctgtgttcgtgtaggcntaatgtgaggaggtggggagaccaccccaccccnatgtccaccatgaccctcttcccacgctgacctgtgctccctccccaatcatctttcctgttccagagaggtggggctgaggtgtctccatctctgtctcaacttcatggtgcactgagctgtaacttcttc

SEQ ID NO: 89 - DNA, 516 bp, Homo sapiens
  misc_feature (64)..(64): n is a, c, g, or t
tgagatgggagctgtcttcccagcccaccatccccatcgtgggcatcattgctggcctggttcnccttggagctgtgatcactggagctgtggtcgctgccgtgatgtggaggaggaagagctcaggtggagaaggggtgaaagatagaaaaggagggagttacactcaggctgcaagcagtgacagtgcccagggctctgatgtgtctctcacagcttgtaaagtgtgagacagctgccttgtgtgggactgagaggcaagagttgttcctgcccttccnnttgtgacttgaagaaccctgactttgtttctgcaaaggcncctgcatgtgtctgtgttcgtgtaggcataatgtgaggaggtggggagaccaccccaccccgatgtccaccatgaccctcttcccacgctgacctgtgctccctctccaatcatctttcctgttccagagaggtggggctgaggtgtctccatctctgtctcaacttcatggtgcactgagctgtaacttcttc

SEQ ID NO: 90 - DNA, 550 bp, Homo sapiens
ccttcatcgctggtgtgccaacaaaaagaatgctgttgtaatggaccaagagcctgcagggaacagaacagtgaacagggaggactctgatgaacaagaccctcaggaggtgacatacgcacagttgaatcactgcgttttcacacagagaaaaatcactcgcccttctcagaggcccaagacacccccaacagataccagcgtgtaacacggaacttccaaatgctgagcgcagatccaaagttgtcttctgtccactagcaccacagtcaggccttgatgggatcttctagggagacaatagccctgtctcaaaaccgggttgccagctcccatgtaccagcagctggactctgaaggcgtgagtctgcatcttagggcatcgctcttcctcacaccacgaatctgaacatgcctctctcttgcttacaaatgtctaaggtccccactgcctgctggagagaaaacacacttgcttagcccacaattctccatttcacttgacccctgcccacctctccaacctaactggcttacttcctagtctact

SEQ ID NO: 91 - protein, 122 aa, Homo sapiens
MEASALTSSAVTSVAKVVRVASGSAVVLPLARIATVVIGGVVAMAAVPMVLSAMGFTAAGIASSSIAAKMMSAAAIANGGGVASGSLVATLQSLGATGLSGLTKFILGSIGSAIAAVIARFY

SEQ ID NO: 92 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 93 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 94 - protein, 455 aa, Homo sapiens
MSLTVVSMACVGFFLLQGAWPLMGGQDKPFLSARPSTVVPRGGHVALQCHYRRGFNNFMLYKEDRSHVPIFHGRIFQESFIMGPVTPAHAGTYRCRGSRPHSLTGWSAPSNPLVIMVTGNHRKPSLLAHPGPLLKSGETVILQCWSDVMFEHFFLHREGISEDPSRLVGQIHDGVSKANFSIGPLMPVLAGTYRCYGSVPHSPYQLSAPSDPLDIVITGLYEKPSLSAQPGPTVQAGENVTLSCSSWSSYDIYHLSREGEAHERRLRAVPKVNRTFQADFPLGPATHGGTYRCFGSFRALPCVWSNSSDPLLVSVTGNPSSSWPSPTEPSSKSGICRHLHVLIGTSVVIFLFILLLFFLLYRWCSNKKNAAVMDQEPAGDRTVNRQDSDEQDPQEVTYAQLDHCVFIQRKISRPSQRPKTPLTDTSVYTELPNAEPRSKVVSCPRAPQSGLEGVF

SEQ ID NO: 95 - protein, 342 aa, Homo sapiens
MSLTVVSMACVGFFLLQGAWPLMGGQDKPFLSARPSTVVPRGGHVALQCHYRRGFNNFMLYKEDRSHVPIFHGRIFQESFIMGPVTPAHAGTYRCRGSRPHSLTGWSTPSNPLVIMVTGNHRKPSLLAHPGPLLKSGETVILQCWSDVMFEHFFLHREGISEDPSRLVGQIHDGVSKANFSIGPLMPVLAGTYRCYGSVPHSPYQLSAPSDPLDIVITGLYEKPSLSAQPGPTVQAGENVTLSCSSWSSYDIYHLSREGEAHERRLRAVPKVNRTFQADFPLGPATHGGTYRCFGSFRALPCVWSNSSDPLLVSVTGNPSSSWPSPTEPSSKSGETSYKLEE

SEQ ID NO: 96 - protein, 348 aa, Homo sapiens
MSLMVVSMACVGFFLLQGAWPHEGVHRKPSLLAHPGRLVKSEETVILQCWSDVRFEHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHECRFSAGPKVNGTFQADFPLGPATHGGTYRCFGSFRDSPYEWSNSSDPLLVSVTGNPSNSWPSPTEPSSKTGNPRHLHILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCP

SEQ ID NO: 97 - protein, 181 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQAL

SEQ ID NO: 98 - protein, 243 aa, Homo sapiens
MSLMVVSMVCVGFFLLQGAWPHEGVHRKPSLLAHPGPLVKSEETVILQCWSDVRFQHFLLHREGKFKDTLHLIGEHHDGVSKANFSIGPMMQDLAGTYRCYGSVTHSPYQLSAPSDPLLVSVTGNPSNSWPSPTEPSSETGNPRHLHVLIGTSVVIILFILLLFFLLHRWCCNKKNAVVMDQEPAGNRTVNREDSDEQDPQEVTYAQLNHCVFTQRKITRPSQRPKTPPTDIIVYTELPNAEP

SEQ ID NO: 99 - protein, 341 aa, Homo sapiens
MSLMVVSMVCVGFFLLQGAWPHEGVHRKPSLLAHPGPLVKSEETVILQCWSDVRFQHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHERRFSAGPKVNGTFQADFPLGPATHGGTYRCFGSFRDSPYEWSNSSDPLLVSVTGNPSNSWPSPTEPSSETGNPRHLHVLIGTSVVIILFILLLFFLLHRWCCNKKNAVVMDQEPAGNRTVNREDSDEQDPQEVTYAQLNHCVFTQRKITRPSQRPKTPPTDIIVYTELPNAEP

SEQ ID NO: 100 - protein, 342 aa, Homo sapiens
MSMSPTVIILACLGFFLDQSVWAHVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARPGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTDAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 101 - protein, 273 aa, Homo sapiens
MSMSPTVIILACLGFFLDQSVWAHVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARPGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTGIARHLHAVIRYSVAIILFTILPFFLLHRWCSKKKMLL

SEQ ID NO: 102 - protein, 377 aa, Homo sapiens
MSMSPTVIILACLGFFLDQSVWAHVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARPGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTGIARHLHAVIRYSVAIILFTILPFFLLHRWCSKKKNAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 103 - protein, 304 aa, Homo sapiens
MSLMVISMACVAFFLLQGAWPHEGFRRKPSLLAHPGPLVKSEETVILQCWSDVMFEHFLLHREGTFNHTLRLIGEHIDGVSKGNFSIGRMTQDLAGTYRCYGSVTHSPYQLSAPSDPLDIVITGLYEKPSLSAQPGPTVLAGESVTLSCSSRSSYDMYHLSREGEAHERRLPAGPKVNRTFQADFPLDPATHGGTYRCFGSFRDSPYEWSKSSDPLLVSVTGNSSNSWPSPTEPSSETGNPRHLHVLIGTSVVKLPFTILLFFLLHRWCSNKKNASVMDQGPAGNRTVNREDSDEQDHQEVSYA

SEQ ID NO: 104 - protein, 233 aa, Homo sapiens
GGQDKPLLSTWPSLVVPPEHVTLRCHSNLGFNNFSLYKDDGVPVPELYNRIFWKSLFMGPVTPSHTGTYRCRGSHTHSPSGGSAPSNPLVIVVTGFRRKPSLLAHPGRLVKSEETVILQCWSDVMFEHFLLHREGTFNDTLRLIGEHIDGVSKANFSIGRMRQDLAGTYRCYGSVPHSPYQFSAPSDPLDIVITGESVQTFFSLSFGHRVNDPGLGGPGGCKEDELGILMERD

SEQ ID NO: 105 - protein, 375 aa, Homo sapiens
MSLMVISMACVGFFLLQGAWTHEGGQDKPLLSAWPSAVVPRGGHVTLLCRSRLGFTIFSLYKEDGVPVPELYNKIFWKSILMGPVTPAHAGTYRCRGSHPRSPIEWSAPSNPLVIVVTGLFGKPSLSAQPGPTVRTGENVTLSCSSRSSFDMYHLSREGRAHEPRLPAVPSVNGTFQADFPLGPATHGGTYTCFGSLHDSPYEWSDPSDPLLVSVTGNSSSSSSSPTEPSSKTGIRRHLHILIGTSVAIILFIILFFFLLHCCCSNKKNAAVMDQEPAGDRTVNREDSDDQDPQEVTYAQLDHCVFTQTKITSPSQRPKTPPTDTTMYMELPNAKPRSLSPAHKHHSQALRGSSRETTALSQNRVASSHVPAAGI

SEQ ID NO: 106 - protein, 80 aa, Homo sapiens
MGRAMVARLGLGLLLLALLLPTQIYSSETTTGTSSNSSQSTSNSGLAPNPTNATTKAAGGALQSTASLFVVSLSLLHLYS

SEQ ID NO: 107 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 108 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 109 - protein, 189 aa, Homo sapiens
MTDSVIYSMLELPTATQAQNDYGPQQKSSSSRPSCSCLVAIALGLLTAVLLSVLLYQWILCQGSNYSTCASCPSCPDRWMKYGNHCYYFSVEEKDWNSSLEFCLARDSHLLVITDNQEMSLLQVFLSEAFCWIGLRNNSGWRWEDGSPLNFSRISSNSFVQTCGAINKNGLQASSCEVPLHWVCKKVRL

SEQ ID NO: 110 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 111 - protein, 382 aa, Homo sapiens
MLLMVVSMACVGLFLVQRAGPHMGGQDKPFLSAWPSAVVPRGGHVTLRCHYRHRFNNFMLYKEDRIHVPIFHGRIFQEGFNMSPVTTAHAGNYTCRGSHPHSPTGWSAPSNPMVIMVTGNHRKPSLLAHPGPLVKSGERVILQCWSDIMFEHFFLHKEWISKDPSRLVGQIHDGVSKANFSIGSMMRALAGTYRCYGSVTHTPYQLSAPSDPLDIVVTGLYEKPSLSAQPGPKVQAGESVTLSCSSRSSYDMYHLSREGGAHERRLPAVRKVNRTFQADFPLGPATHGGTYRCFGSFRHSPYEWSDPSDPLLVSVTGNPSSSWPSPTEPSSKSGNLRHLHILIGTSVVKIPFTILLFFLLHRWCSNKKKCCCNGPRACREQK

SEQ ID NO: 112 - protein, 365 aa, Homo sapiens
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 113 - protein, 410 aa, Homo sapiens
MSLMVVSMACVGFFLLEGPWPHVGGQDKPFLSAWPGTVVSEGQHVTLQCRSRLGFNEFSLSKEDGMPVPELYNRIFRNSFLMGPVTPAHAGTYRCCSSHPHSPTGWSAPSNPVVIMVTGVHRKPSLLAHPGPLVKSGETVILQCWSDVRFERFLLHREGITEDPLRLVGQLHDAGSQVNYSMGPMTPALAGTYRCFGSVTHLPYELSAPSDPLDIVVVGLYGKPSLSAQPGPTVQAGENVTLSCSSRSLFDIYHLSREAEAGELRLTAVLRVNGTFQANFPLGPVTHGGNYRCFGSFRALPHAWSDPSDPLPVSVTGNSRHLHVLIGTSVVIIPFAILLFFLLHRWCANKKNAVVMDQEPAGNRTVNREDSDEQDPQEVTYAQLNHCVFTQRKITRPSQRPKTPPTDTSV

SEQ ID NO: 114 - protein, 328 aa, Homo sapiens
MSLMVISMACVGFFLLQGAWTHEGGQDKPFLSAWPSPVVSEGEHVALQCRSRLGFNEFSLSKEDGMPVPELYNRVFRNTVFIGPVTPAHAGTYRCRGSHPHFLTGWSAPSNPLVIMVTGVHRKPSLLAHPGPLVKSEETVILQCWSDVMFEHFLLHREGKFNDTLRLTGELHDGVSKANFSIGRMTQDLAGTYRCYGSVPHSPYQLSAPSDPLDIVITGLCGKPSLSAQPRPMVKAGESVTLSCSSRSSYDIYHLSREGEAHELRFPAVPKVNGTFQANFPLGPATHGGTYRCFGSFRDSPYEWSDLSDPLLVSVTDSMKEKGKDVIL

SEQ ID NO: 115 - protein, 304 aa, Homo sapiens
MSLMVVSMACVGFFLLQGAWPHEGVHRKPSLLAHPGPLVKSEETVILQCWSDVRFEHFLLHREGKYKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHERRFSAGPKVNGTFQADFPLGPATHGGTYRCFGSFRDSPYEWSNSSDPLLVSVTGNPSNSWPSPTEPSSKTGNPRHLHVLIGTSVVKIPFTILLFFLLHRWCSNKKNAAVMDQEPAGNRTVNSEDSDEQDHQEVSYA

SEQ ID NO: 116 - protein, 304 aa, Homo sapiens
MSLMVISMACVGFFWLQGAWPHEGFRRKPSLLAHPGRLVKSEETVILQCWSDVMFEHFLLHREGTFNDTLRLIGEHIDGVSKANFSIGRMRQDLAGTYRCYGSVPHSPYQFSAPSDPLDIVITGLYEKPSLSAQPGPTVLAGESVTLSCSSWSSYDMYHLSTEGEAHERRFSAGPKVNGTFQADFPLGPATQGGTYRCFGSFHDSPYEWSKSSDPLLVSVTGNPSNSWPSPTEPSSKTGNPRHLHVLIGTSVVKLPFTILLFFLLHRWCSDKKNASVMDQGPAGNRTVNREDSDEQDHQEVSYA

SEQ ID NO: 117 - protein, 304 aa, Homo sapiens
MSLMVIIMACVGFFLLQGAWPQEGVHRKPSFLALPGHLVKSEETVILQCWSDVMFEHFLLHREGKFNNTLHLIGEHHDGVSKANFSIGPMMPVLAGTYRCYGSVPHSPYQLSAPSDPLDMVIIGLYEKPSLSAQPGPTVQAGENVTLSCSSRSSYDMYHLSREGEAHERRLPAVRSINGTFQADFPLGPATHGGTYRCFGSFRDAPYEWSNSSDPLLVSVTGNPSNSWPSPTEPSSKTGNPRHLHVLIGTSVVKIPFTILLFFLLHRWCSDKKNAAVMDQEPAGNRTVNSEDSDEQDHQEVSYA

SEQ ID NO: 118 - protein, 348 aa, Homo sapiens
MSLLVVSMACVGFFLLQGAWPHEGVHRKPSLLAHPGRLVKSEETVILQCWSDVMFEHFLLHREGMFNDTLRLIGEHHDGVSKANFSISRMTQDLAGTYRCYGSVTHSPYQVSAPSDPLDIVIIGLYEKPSLSAQLGPTVLAGENVTLSCSSRSSYDMYHLSREGEAHERRLPAGPKVNGTFQADFPLGPATHGGTYRCFGSFHDSPYEWSKSSDPLLVSVTGNPSNSWPSPTEPSSKTGNPRHLHILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCP

SEQ ID NO: 119 - protein, 304 aa, Homo sapiens
MSLTVVSMACVGFFLLQGAWPHEGVHRKPSLLAHPGRLVKSEETVILQCWSDVMFEHFLLHREGMFNDTLRLIGEHHDGVSKANFSISRMKQDLAGTYRCYGSVTHSPYQLSAPSDPLDIVIIGLYEKPSLSAQPGPTVLAGENVTLSCSSRSSYDMYHLSREGEAHERRLPAGTKVNGTFQANFPLGPATHGGTYRCFGSFRDSPYEWSKSSDPLLVSVTGNPSNSWPSPTEPSSETGNPRHLHVLIGTSVVKIPFTILLFFLLHRWCSDKKNAAVMDQEPAGNRTVNSEDSDEQDHQEVSYA

SEQ ID NO: 120 - protein, 254 aa, Homo sapiens
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRAEQSPQPTIPIVGIVAGLVVLGAVVTGAVVAAVMWRKKSSDRNRGSYSQAAV

SEQ ID NO: 121 - protein, 442 aa, Homo sapiens
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPPGEEQRYTCHVQHEGLPQPLILRWEQSPQPTIPIVGIVAGLVVLGAVVTGAVVAAVMWRKKSSDRNRGSYSQAAAYSVVSGNLMITWWSSLFLLGVLFQGYLGCLRSHSVLGRRKVGDMWILFFLWLWTSFNTAFLALQSLRFGFGFRRGRSFLLRSWHHLMKRVQIKIFD

SEQ ID NO: 122 - protein, 346 aa, Homo sapiens
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPPGEEQRYTCHVQHEGLPQPLILRWEQSPQPTIPIVGIVAGLVVLGAVVTGAVVAAVMWRKKSSDRNRGSYSQAAV

SEQ ID NO: 123 - DNA, 665 bp, Homo sapiens
gggaacacatccaagcttaagacggtgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggccatggcggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaaataagaaataaagatgaattgttgcaactcttcaaaa

SEQ ID NO: 124 - DNA, 656 bp, Homo sapiens
gggaacacatccaagcttaagacggtgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaaataagaaataaagatgaattgttgcaactcttcaaaa

SEQ ID NO: 125 - DNA, 1184 bp, Homo sapiens
gggggagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaaaaaaa

SEQ ID NO: 126 - DNA, 994 bp, Homo sapiens
aagactcaaacttagaaacttgaattagatgtggtattcaaatccttagctgccgcgaagacacagacagcccccgtaagaacccacgaagcaggcgaagttcattgttctcaacattctagctgctcttgctgcatttgctctggaattcttgtagagatattacttgtccttccaggctgttctttctgtagctcccttgttttctttttgtgatcatgttgcagatggctgggcagtgctcccaaaatgaatattttgacagtttgttgcatgcttgcataccttgtcaacttcgatgttcttctaatactcctcctctaacatgtcagcgttattgtaatgcaagtgtgaccaattcagtgaaaggaacgaatgcgattctctggacctgtttgggactgagcttaataatttctttggcagttttcgtgctaatgtttttgctaaggaagataaactctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactgtggaaactctttatgttagatatatttctctaggttactgttgggagcttaatggtagaaacttccttggtttcatgattaaactcttttttttcctga

SEQ ID NO: 127 - DNA, 1877 bp, Homo sapiens
ggggcgcggcctcctgtctgcaccggcagcaccatgtcgctcacggtcgtcagcatggcgtgcgttgggttcttcttgctgcagggggcctggccactcatgggtggtcaggacaaacccttcctgtctgcccggcccagcactgtggtgcctcgaggaggacacgtggctcttcagtgtcactatcgtcgtgggtttaacaatttcatgctgtacaaagaagacagaagccacgttcccatcttccacggcagaatattccaggagagcttcatcatgggccctgtgaccccagcacatgcagggacctacagatgtcggggttcacgcccacactccctcactgggtggtcggcacccagcaaccccctggtgatcatggtcacaggaaaccacagaaaaccttccctcctggcccacccagggcccctgctgaaatcaggagagacagtcatcctgcaatgttggtcagatgtcatgtttgagcacttctttctgcacagagaggggatctctgaggacccctcacgcctcgttggacagatccatgatggggtctccaaggccaacttctccatcggtcccttgatgcctgtccttgcaggaacctacagatgttatggttctgttcctcactccccctatcagttgtcagctcccagtgaccccctggacatcgtgatcacaggtctatatgagaaaccttctctctcagcccagccgggccccacggttcaggcaggagagaacgtgaccttgtcctgtagctcctggagctcctatgacatctaccatctgtccagggaaggggaggcccatgaacgtaggctccgtgcagtgcccaaggtcaacagaacattccaggcagactttcctctgggccctgccacccacggagggacctacagatgcttcggctctttccgtgccctgccctgcgtgtggtcaaactcaagtgacccactgcttgtttctgtcacaggaaacccttcaagtagttggccttcacccacagaaccaagctccaaatctggtatctgcagacacctgcatgttctgattgggacctcagtggtcatcttcctcttcatcctcctcctcttctttctcctttatcgctggtgctccaacaaaaagaatgctgctgtaatggaccaagagcctgcgggggacagaacagtgaataggcaggactctgatgaacaagaccctcaggaggtgacgtacgcacagttggatcactgcgttttcatacagagaaaaatcagtcgcccttctcagaggcccaagacacccctaacagataccagcgtgtacacggaacttccaaatgctgagcccagatccaaagttgtctcctgcccacgagcaccacagtcaggtcttgagggggttttctagggagacaacagccctgtctcaaaaccaggttgccagatccaatgaaccagcagctggaatctgaaggcatcagtctgcatcttaggggatcgctcttcctcacaccacgaatctgaacatgcctctctcttgcttacaaatgcctaaggtcgccactgcctgctgcagagaaaacacactcctttgcttagcccacaagtatctatttcacttgacccctgcccacctctccaacctaactggcttacttcctagtcctacttgaggctgcaatcacactgaggaactcacaattccaaacatacaagaggctccctcttaacacggcacttacacacttgctgttccaccttccctcatgctgttccacctcccctcagactatctttcagccttctgtcatcagtaaaatttataaattttttttataacttcagtgtagctctctcctcttcaaataaacatgtctgccctca

SEQ ID NO: 128 - DNA, 1029 bp, Homo sapiens
atgtcgctcactgtcgtcagcatggcgtgcgttgggttcttcttgctgcagggggcctggccactcatgggtggtcaggacaaacccttcctgtctgcccggcccagcactgtggtgcctcgaggaggacacgtggctcttcagtgtcactatcgtcgtgggtttaacaatttcatgctgtacaaagaagacagaagccacgttcccatcttccacggcagaatattccaggagagcttcatcatgggccctgtgaccccagcacatgcagggacctacagatgtcggggttcacgcccacactccctcactgggtggtcgacacccagcaaccccctggtgatcatggtcacaggaaaccacagaaaaccttccctcctggcccacccagggcccctgctgaaatcaggagagacagtcatcctgcaatgttggtcagatgtcatgtttgagcacttctttctgcacagagaggggatctctgaggacccctcacgcctcgttggacagatccatgatggggtctccaaggccaacttctccatcggtcccttgatgcctgtccttgcaggaacctacagatgttatggttctgttcctcactccccctatcagttgtcagctcccagtgaccccctggacatcgtgatcacaggtctatatgagaaaccttctctctcagcccagccgggccccacggttcaggcaggagagaacgtgaccttgtcctgtagctcctggagctcctatgacatctaccatctgtccagggaaggggaggcccatgaacgtaggctccgtgcagtgcccaaggtcaacagaacattccaggcagactttcctctgggccctgccacccacggagggacctacagatgcttcggctctttccgtgccctgccctgcgtgtggtcaaactcaagtgacccactgcttgtttctgtcacaggaaacccttcaagtagttggccttcacccacagaaccaagctccaaatctggtgagacctcctacaagctagaagaataa

SEQ ID NO: 129 - DNA, 1587 bp, Homo sapiens
cgcggccgcctgtctgcacagacagcaccatgtcgctcatggtcgtcagcatggcgtgtgttgggttcttcttgctgcagggggcctggccacatgagggagtccacagaaaaccttccctcctggcccacccaggtcgcctggtgaaatcagaagagacagtcatcctgcaatgttggtcagatgtcaggtttgagcacttccttctgcacagagaagggaagtttaaggacactttgcacctcattggagagcaccatgatggggtctccaaagccaacttctccatcggtcccatgatgcaagaccttgcagggacctacagatgctacggttctgttactcactccccctatcagttgtcagctcccagtgaccctctggacatcgtcatcacaggtctatatgagaaaccttctctctcagcccagccgggccccacggttctggcaggagagagcgtgaccttgtcctgcagctcccggagctcctatgacatgtaccatctatccagggagggggaggcccatgaatgtaggttctctgcagggcccaaggtcaacggaacattccaggccgactttcctctgggccctgccacccacggaggaacctacagatgcttcggctctttccgtgactctccatacgagtggtcaaactcgagtgacccactgcttgtttctgtcacaggaaacccttcaaatagttggccttcacccactgaaccaagctctaaaaccggtaacccccgacacctgcacattctgattgggacctcagtggtcatcatcctcttcatcctcctcttctttctccttcatcgctggtgctccaacaaaaaaaatgctgcggtaatggaccaagagtctgcagggaacagaacagcgaatagcgaggactctgatgaacaagaccctcaggaggtgacatacacacagttgaatcactgcgttttcacacagagaaaaatcactcgcccttctcagaggcccaagacacccccaacagatatcatcgtgtacacggaacttccaaatgctgagtccagatccaaagttgtctcctgcccatgagcaccacagtcaggccttgagggcgtcttctagggagacaacagccctgtctcaaaaccgggttgccagctcccatgtaccagcagctggaatctgaaggcatgagtctgcatcttagggcatcgctcttcctcacaccacaaatctgaatgtgcctctcacttgcttacaaatgtctaaggtccccactgcctgctggagaaaaaacacactcctttgcttagcccacagttctccatttcacttgacccctgcccacctctccaacctaactggcttacttcctagtctacttgaggctgcaatcacactgaggaactcacaattccaaacatacaagaggctccctcttaacgcagcacttagacacgtgttgttccaccttccctcatgctgttccacctcccctcagactagctttcagtcttctgtcagcagtaaaacttatatattttttaaaataacttcaatgtagttttccatccttcaaataaacatgtctgcccccatg

SEQ ID NO: 130 - DNA, 1559 bp, Homo sapiens
ccccagtctgagaacaagaaagaagaacttctgtctcgagggtctcactgtcaaccaggccagagtgcagtgaagatcatacctcactacatccgtgaactcccgggctcctcccacctaagtctcttgagtagctgggacttcaggagactgaagccaaggataccagcagagccaacatttgcttcaagttcctgggcctgctgacagcgtgcaggatgctgttggaacccggcagaggctgctgtgccctggccatcctgctggcaattgtggacatccagtctggtggatgcattaacatcaccagctcagcttcccaggaaggaacgcgactaaacttaatctgtactgtatggcataagaaagaagaggctgaggggtttgtagtgtttttgtgcaaggacaggtctggagactgttctcctgagaccagtttaaaacagctgagacttaaaagggatcctgggatagatggtgttggtgaaatatcatctcagttgatgttcaccataagccaagtcacaccgttgcacagtgggacctaccagtgttgtgccagaagccagaagtcaggtatccgccttcagggccattttttctccattctattcacagagacagggaactacacagtgacgggattgaaacaaagacaacaccttgagttcagccataatgaaggcactctcagttcaggcttcctacaagaaaaggtctgggtaatgctggtcaccagccttgtggcccttcaagctttgtaagccttgtgccaaaagaaacttttaaaacagctacagcaagatgagtctgactatggcttagtatctttctcattacaataggcacagagaagaatgcaacagggcacaggggaagagatgctaaatataccaagaatctgtggaaatataagctggggcaaatcagtgtaatccttgactttgctcctcaccatcagggcaaacttgccttcttccctcctaagctccagtaaataaacagaacagctttcaccaaagtgggtagtatagtcctcaaatatcggataaatatatgcgtttttgtaccccagaaaaacttttcctccctcttcatcaacatagtaaaataagtcaaacaaaatgagaacaccaaattttgggggaataaatttttatttaacactgcaaaggaaagagagagaaaacaagcaaagataggtaggacagaaaggaagacagccagatccagtgattgacttggcatgaaaatgagaaaatgcagacagacctcaacattcaacaacatccatacagcactgctggaggaagaggaagatttgtgcagaccaagagcaccacagactacaactgcccagcttcatctaaatacttgttaacctctttggtcatttctctttttaaataaatgcccatagcagtatttggagtattttcttttctcctaaatccacaaactctcttctttctctttggacagatgacctcttgtcatagttaagcagagagtgggcaggatattcctgataggaggaactacatgaataaaggggtaag

SEQ ID NO: 131 - DNA, 1302 bp, Homo sapiens
agctggggcgcggccgcctgtctgcacagacagcaccatgtcgctcatggtcgtcagcatggtgtgtgttgggttcttcttgctgcagggggcctggccacatgagggagtccacagaaaaccttccctcctggcccacccaggtcccctggtgaaatcagaagagacagtcatcctgcaatgttggtcagatgtcaggtttcagcacttccttctgcacagagaagggaagtttaaggacactttgcacctcattggagagcaccatgatggggtctccaaggccaacttctccatcggtcccatgatgcaagaccttgcagggacctacagatgctacggttctgttactcactccccctatcagttgtcagctccgagtgacccactgcttgtttctgtcacaggaaacccttcaaatagttggccttcacccactgaaccaagctccgaaaccggtaaccccagacacctgcatgttctgattgggacctcagtggtcatcatcctcttcatcctcctcctcttctttctccttcatcgctggtgctgcaacaaaaaaaatgctgttgtaatggaccaagagcctgcagggaacagaacagtgaacagggaggactctgatgaacaagaccctcaggaggtgacatatgcacagttgaatcactgcgttttcacacagagaaaaatcactcgcccttctcagaggcccaagacacccccaacagatatcatcgtgtacacggaacttccaaatgctgagccctgatccaaagttgtctcctgcccatgagcaccacagtcaggccttgaggggatcttctagggagacaacagccctgtctcaaaactgggttgccagctccaatgtaccagcagctggaatctgaaggcgtgagtctgcatcttagggcatcgctcttcctcacaccacaaatctgaacgtgcctctcccttgcttacaaatgtctaaggtccccactgcctgctggagagaaaacacactcctttgcttagcccacaattctccatttcacttgacccctgcccacctctccaacctaactggcttacttcctagtctacttgaggctgcaatcacactgaggaactcacaattccaaacatacaagaggctccctcttaacacggcacttagacacgtgctgttccaccttccctcatgctgttccacctcccctcagactagctttcagccttctgtcagcagtaaaacttatatattttttaaaataatttcaatgtagttttccctccttcaaataaacatgtctgccctca

SEQ ID NO: 132 - DNA, 1596 bp, Homo sapiens
agctggggcgcggccgcctgtctgcacagacagcaccatgtcgctcatggtcgtcagcatggtgtgtgttgggttcttcttgctgcagggggcctggccacatgagggagtccacagaaaaccttccctcctggcccacccaggtcccctggtgaaatcagaagagacagtcatcctgcaatgttggtcagatgtcaggtttcagcacttccttctgcacagagaagggaagtttaaggacactttgcacctcattggagagcaccatgatggggtctccaaggccaacttctccatcggtcccatgatgcaagaccttgcagggacctacagatgctacggttctgttactcactccccctatcagttgtcagctcccagtgaccctctggacatcgtcatcacaggtctatatgagaaaccttctctctcagcccagccgggccccacggttctggcaggagagagcgtgaccttgtcctgcagctcccggagctcctatgacatgtaccatctatccagggagggggaggcccatgaacgtaggttctctgcagggcccaaggtcaacggaacattccaggccgactttcctctgggccctgccacccacggaggaacctacagatgcttcggctctttccgtgactctccatacgagtggtcaaactcgagtgacccactgcttgtttctgtcacaggaaacccttcaaatagttggccttcacccactgaaccaagctccgaaaccggtaaccccagacacctgcatgttctgattgggacctcagtggtcatcatcctcttcatcctcctcctcttctttctccttcatcgctggtgctgcaacaaaaaaaatgctgttgtaatggaccaagagcctgcagggaacagaacagtgaacagggaggactctgatgaacaagaccctcaggaggtgacatatgcacagttgaatcactgcgttttcacacagagaaaaatcactcgcccttctcagaggcccaagacacccccaacagatatcatcgtgtacacggaacttccaaatgctgagccctgatccaaagttgtctcctgcccatgagcaccacagtcaggccttgaggggatcttctagggagacaacagccctgtctcaaaactgggttgccagctccaatgtaccagcagctggaatctgaaggcgtgagtctgcatcttagggcatcgctcttcctcacaccacaaatctgaacgtgcctctcccttgcttacaaatgtctaaggtccccactgcctgctggagagaaaacacactcctttgcttagcccacaattctccatttcacttgacccctgcccacctctccaacctaactggcttacttcctagtctacttgaggctgcaatcacactgaggaactcacaattccaaacatacaagaggctccctcttaacacggcacttagacacgtgctgttccaccttccctcatgctgttccacctcccctcagactagctttcagccttctgtcagcagtaaaacttatatattttttaaaataatttcaatgtagttttccctccttcaaataaacatgtctgccctca

SEQ ID NO: 133 - DNA, 1505 bp, Homo sapiens
tcgagccgagtcactgcgtcctggcagcagaagctgcaccatgtccatgtcacccacggtcatcatcctggcatgtcttgggttcttcttggaccagagtgtgtgggcacacgtgggtggtcaggacaagcccttctgctctgcctggcccagcgctgtggtgcctcaaggaggacacgtgactcttcggtgtcactatcgtcgtgggtttaacatcttcacgctgtacaagaaagatggggtccctgtccctgagctctacaacagaatattctggaacagtttcctcattagccctgtgaccccagcacacgcagggacctacagatgtcgaggttttcacccgcactcccccactgagtggtcggcacccagcaaccccctggtgatcatggtcacaggtctatatgagaaaccttcgcttacagcccggccgggccccacggttcgcgcaggagagaacgtgaccttgtcctgcagctcccagagctcctttgacatctaccatctatccagggagggggaagcccatgaacttaggctccctgcagtgcccagcatcaatggaacattccaggccgacttccctctgggtcctgccacccacggagagacctacagatgcttcggctctttccatggatctccctacgagtggtcagacccgagtgacccactgcctgtttctgtcacaggaaacccttctagtagttggccttcacccactgaaccaagcttcaaaactgatgctgctgtaatgaaccaagagcctgcgggacacagaacagtgaacagggaggactctgatgaacaagaccctcaggaggtgacatacgcacagttggatcactgcattttcacacagagaaaaatcactggcccttctcagaggagcaagagaccctcaacagataccagcgtgtgtatagaacttccaaatgctgagcccagagcgttgtctcctgcccatgagcaccacagtcaggccttgatgggatcttctagggagacaacagccctgtctcaaacccagcttgccagctctaatgtaccagcagctggaatctgaaggcgtgagtctccatcttagagcatcactcttcctcacaccacaaatctggtgcctgtctcttgcttaccaatgtctaaggtccccactgcctgctgcagagaaaacacactcctttgcttagcccacaattctctatttcacttgacccctgcccacctctccaacctaactggcttacttcctagtctacttgaggctgcaatcacactgaggaactcacaattccaaacatacaagaggctctctcttaacacggcacttagacacgtgctgttccaccttccctcgtgctgttccacctttcctcagactatttttcagccttctggcatcagcaaaccttataaaatttttttgatttcagtgtagttctctcctcttcaaataaacatgtctgccttcaaaaaaaaaaaaaaaaaaaaaaaaaaaaaaa

SEQ ID NO: 134 - DNA, 1609 bp, Homo sapiens
tcgagccgagtcactgcgtcctggcagcagaagctgcaccatgtccatgtcacccacggtcatcatcctggcatgtcttgggttcttcttggaccagagtgtgtgggcacacgtgggtggtcaggacaagcccttctgctctgcctggcccagcgctgtggtgcctcaaggaggacacgtgactcttcggtgtcactatcgtcgtgggtttaacatcttcacgctgtacaagaaagatggggtccctgtccctgagctctacaacagaatattctggaacagtttcctcattagccctgtgaccccagcacacgcagggacctacagatgtcgaggttttcacccgcactcccccactgagtggtcggcacccagcaaccccctggtgatcatggtcacaggtctatatgagaaaccttcgcttacagcccggccgggccccacggttcgcgcaggagagaacgtgaccttgtcctgcagctcccagagctcctttgacatctaccatctatccagggagggggaagcccatgaacttaggctccctgcagtgcccagcatcaatggaacattccaggccgacttccctctgggtcctgccacccacggagagacctacagatgcttcggctctttccatggatctccctacgagtggtcagacccgagtgacccactgcctgtttctgtcacaggaaacccttctagtagttggccttcacccactgaaccaagcttcaaaactggtatcgccagacacctgcatgctgtgattaggtactcagtggccatcatcctctttaccatccttcccttctttctccttcatcgctggtgctccaaaaaaaaaatgctgctgtaatgaaccaagagcctgcgggacacagaacagtgaacagggaggactctgatgaacaagaccctcaggaggtgacatacgcacagttggatcactgcattttcacacagagaaaaatcactggcccttctcagaggagcaagagaccctcaacagataccagcgtgtgtatagaacttccaaatgctgagcccagagcgttgtctcctgcccatgagcaccacagtcaggccttgatgggatcttctagggagacaacagccctgtctcaaacccagcttgccagctctaatgtaccagcagctggaatctgaaggcgtgagtctccatcttagagcatcactcttcctcacaccacaaatctggtgcctgtctcttgcttaccaatgtctaaggtccccactgcctgctgcagagaaaacacactcctttgcttagcccacaattctctatttcacttgacccctgcccacctctccaacctaactggcttacttcctagtctacttgaggctgcaatcacactgaggaactcacaattccaaacatacaagaggctctctcttaacacggcacttagacacgtgctgttccaccttccctcgtgctgttccacctttcctcagactatttttcagccttctggcatcagcaaaccttataaaatttttttgatttcagtgtagttctctcctcttcaaataaacatgtctgccttcaaaaaaaaaaaaaaaaaaaaaaaaaaaaaaa

SEQ ID NO: 135 - DNA, 1610 bp, Homo sapiens
tcgagccgagtcactgcgtcctggcagcagaagctgcaccatgtccatgtcacccacggtcatcatcctggcatgtcttgggttcttcttggaccagagtgtgtgggcacacgtgggtggtcaggacaagcccttctgctctgcctggcccagcgctgtggtgcctcaaggaggacacgtgactcttcggtgtcactatcgtcgtgggtttaacatcttcacgctgtacaagaaagatggggtccctgtccctgagctctacaacagaatattctggaacagtttcctcattagccctgtgaccccagcacacgcagggacctacagatgtcgaggttttcacccgcactcccccactgagtggtcggcacccagcaaccccctggtgatcatggtcacaggtctatatgagaaaccttcgcttacagcccggccgggccccacggttcgcgcaggagagaacgtgaccttgtcctgcagctcccagagctcctttgacatctaccatctatccagggagggggaagcccatgaacttaggctccctgcagtgcccagcatcaatggaacattccaggccgacttccctctgggtcctgccacccacggagagacctacagatgcttcggctctttccatggatctccctacgagtggtcagacccgagtgacccactgcctgtttctgtcacaggaaacccttctagtagttggccttcacccactgaaccaagcttcaaaactggtatcgccagacacctgcatgctgtgattaggtactcagtggccatcatcctctttaccatccttcccttctttctccttcatcgctggtgctccaaaaaaaaaaatgctgctgtaatgaaccaagagcctgcgggacacagaacagtgaacagggaggactctgatgaacaagaccctcaggaggtgacatacgcacagttggatcactgcattttcacacagagaaaaatcactggcccttctcagaggagcaagagaccctcaacagataccagcgtgtgtatagaacttccaaatgctgagcccagagcgttgtctcctgcccatgagcaccacagtcaggccttgatgggatcttctagggagacaacagccctgtctcaaacccagcttgccagctctaatgtaccagcagctggaatctgaaggcgtgagtctccatcttagagcatcactcttcctcacaccacaaatctggtgcctgtctcttgcttaccaatgtctaaggtccccactgcctgctgcagagaaaacacactcctttgcttagcccacaattctctatttcacttgacccctgcccacctctccaacctaactggcttacttcctagtctacttgaggctgcaatcacactgaggaactcacaattccaaacatacaagaggctctctcttaacacggcacttagacacgtgctgttccaccttccctcgtgctgttccacctttcctcagactatttttcagccttctggcatcagcaaaccttataaaatttttttgatttcagtgtagttctctcctcttcaaataaacatgtctgccttcaaaaaaaaaaaaaaaaaaaaaaaaaaaaaaa

SEQ ID NO: 136 - DNA, 1596 bp, Homo sapiens
ctgtgcgctgctgagctgagctggggcgcggccgcctgtctgcaccggcagcaccatgtcgctcatggtcatcagcatggcgtgtgttgggttcttcttgctgcagggggcctggacacatgagggtggtcaggacaagcccttgctgtctgcctggcccagcgctgtggtgcctcgaggaggacatgtgactcttctgtgtcgctctcgtcttgggtttaccatcttcagtctgtacaaagaagatggggtgcctgtccctgagctctacaacaaaatattctggaagagcatcctcatgggccctgtgacccctgcacacgcagggacctacagatgtcggggttcacacccacgctcccccattgagtggtcagcacccagcaaccccctggtgatcgtggtcacaggtctatttgggaaaccttcactctcagcccagccgggccccacggttcgcacaggagagaacgtgaccttgtcctgcagctccaggagctcatttgacatgtaccatctatccagggaggggagggcccatgaacctaggctccctgcagtgcccagcgtcaatggaacattccaggctgactttcctctgggccctgccacccacggagggacctacacatgcttcggctctctccatgactcaccctatgagtggtcagacccgagtgacccactgcttgtttctgtcacaggaaactcttcaagtagttcatcttcacccactgaaccaagctccaaaactggtatccgcagacacctgcacattctgattgggacctcagtggctatcatcctcttcatcatcctcttcttctttctccttcattgctgctgctccaacaaaaagaatgctgctgtaatggaccaagagcctgccggggacagaacagtgaacagggaggactctgatgatcaagaccctcaggaggtgacatatgcacagttggatcactgcgttttcacacagacaaaaatcacttccccttctcagaggcccaagacacctccaacagataccaccatgtacatggaacttccaaatgctaagccaagatcattgtctcctgcccataagcaccacagtcaggccttgaggggatcttctagggagacaacagccctgtctcaaaaccgggttgctagctcccatgtaccagcagctggaatctgaaggcatcagtcttcatcttaggggatcgctcttcctcacaccacaaatctgaacatgcctctctcttgcttacaaatgtctaaggtccccactgcctgctggagagaagacacactcctttgcttagcccacaattctctatttcacttgacccctgcccacctctccaactgaactggcttacttcctagtctacttgaggctgcaatcacactgaggaactcacaattccagacatacaagaggctccctcttaacatggcactgagacacgtgctgttccaccttccctcatgctgtttcacctttcctcagactattttccagccttctgtcagtcagcagtgaaacttataaaattttttgtgatttcaatgtagctgtctccttttcaaataaacatgtctgccctc

SEQ ID NO: 137 - DNA, 2194 bp, Homo sapiens
gggtctcgccggctcgccgcgctccccaccttgcctgcgcccgcccggagccagcggttctccaagcacccagcatcctgctagacgcgccgcgcaccgacggaggggacatgggcagagcaatggtggccaggctcgggctggggctgctgctgctggcactgctcctacccacgcagatttattccagtgaaacaacaactggaacttcaagtaactcctcccagagtacttccaactctgggttggccccaaatccaactaatgccaccaccaaggcggctggtggtgccctgcagtcaacagccagtctcttcgtggtctcactctctcttctgcatctctactcttaagagactcaggccaagaaacgtcttctaaatttccccatcttctaaacccaatccaaatggcgtctggaagtccaatgtggcaaggaaaaacaggtcttcatcgaatctactaattccacaccttttattgacacagaaaatgttgagaatcccaaatttgattgatttgaagaacatgtgagaggtttgactagatgatggatgccaatattaaatctgctggagtttcatgtacaagatgaaggagaggcaacatccaaaatagttaagacatgatttccttgaatgtggcttgagaaatatggacacttaatactaccttgaaaataagaatagaaataaaggatgggattgtggaatggagattcagttttcatttggttcattaattctataaggccataaaacaggtaatataaaaagcttccatgattctatttatatgtacatgagaaggaacttccaggtgttactgtaattcctcaacgtattgtttcgacagcactaatttaatgccgatatactctagatgaagttttacattgttgagctattgctgttctcttgggaactgaactcactttcctcctgaggctttggatttgacattgcatttgaccttttatgtagtaattgacatgtgccagggcaatgatgaatgagaatctacccccagatccaagcatcctgagcaactcttgattatccatattgagtcaaatggtaggcatttcctatcacctgtttccattcaacaagagcactacattcatttagctaaacggattccaaagagtagaattgcattgaccacgactaatttcaaaatgctttttattattattattttttagacagtctcactttgtcgcccaggccggagtgcagtggtgcgatctcagatcagtgtaccatttgcctcccgggctcaagcgattctcctgcctcagcctcccaagtagctgggattacaggcacctgccaccatgcccggctaatttttgtaattttagtagagacagggtttcaccatgttgcccaggctggtttcgaactcctgacctcaggtgatccacccgcctcggcctcccaaagtgctgggattacaggcttgagcccccgcgcccagccatcaaaatgctttttatttctgcatatgttgaatactttttacaatttaaaaaaatgatctgttttgaaggcaaaattgcaaatcttgaaattaagaaggcaaaaatgtaaaggagtcaaaactataaatcaagtatttgggaagtgaagactggaagctaatttgcattaaattcacaaacttttatactctttctgtatatacattttttttctttaaaaaacaactatggatcagaatagccacatttagaacactttttgttatcagtcaatatttttagatagttagaacctggtcctaagcctaaaagtgggcttgattctgcagtaaatcttttacaactgcctcgacacacataaacctttttaaaaatagacactccccgaagtcttttgttcgcatggtcacacactgatgcttagatgttccagtaatctaatatggccacagtagtcttgatgaccaaagtcctttttttccatctttagaaaactacatgggaacaaacagatcgaacagttttgaagctactgtgtgtgtgaatgaacactcttgctttattccagaatgctgtacatctattttggattgtatattgtgtttgtgtatttacgctttgattcatagtaacttcttatggaattgatttgcattgaacacaaactgtaaataaaaagaaatggctgaaagagcaaaaaaaaaaaaa

SEQ ID NO: 138 - DNA, 1578 bp, Homo sapiens
agttctaaagtccccacgcacccacccggactcagagtctcctcagacgccgagatgctggtcatggcgccccgaaccgtcctcctgctgctctcggcggccctggccctgaccgagacctgggccggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctactacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtgcagcgacagtgcccagggctctgatgtgtctctcacagcttgaaaagcctgagacagctgtcttgtgagggactgagatgcaggatttcttcacgcctcccctttgtgacttcaagagcctctggcatctctttctgcaaaggcacctgaatgtgtctgcgtccctgttagcataatgtgaggaggtggagagacagcccacccttgtgtccactgtgacccctgttcccatgctgacctgtgtttcctccccagtcatctttcttgttccagagaggtggggctggatgtctccatctctgtctcaactttacgtgcactgagctgcaacttcttacttccctactgaaaataagaatctgaatataaatttgttttctcaaatatttgctatgagaggttgatggattaattaaataagtcaattcctggaatttgagagagcaaataaagacctgagaaccttccagaaaaaaaa

SEQ ID NO: 139 - DNA, 1543 bp, Homo sapiens
ggccgagatgcgggtcatggcgccccgagccctcctcctgctgctctcgggaggcctggccctgaccgagacctgggcctgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacaggctgaccgagtgagcctgcggaacctgcgcggctactacaaccagagcgaggacgggtctcacaccctccagaggatgtctggctgcgacctggggcccgacgggcgcctcctccgcgggtatgaccagtccgcctacgacggcaaggattacatcgccctgaacgaggacctgcgctcctggaccgccgcggacaccgcggctcagatcacccagcgcaagttggaggcggcccgtgcggcggagcagctgagagcctacctggagggcacgtgcgtggagtggctccgcagatacctggagaacgggaaggagacgctgcagcgcgcagaacccccaaagacacacgtgacccaccaccccctctctgaccatgaggccaccctgaggtgctgggccctgggcttctaccctgcggagatcacactgacctggcagcgggatggggaggaccagacccaggacaccgagcttgtggagaccaggccagcaggagatggaaccttccagaagtgggcagctgtggtggtgccttctggacaagagcagagatacacgtgccatatgcagcacgaggggctgcaagagcccctcaccctgagctgggagccatcttcccagcccaccatccccatcatgggcatcgttgctggcctggctgtcctggttgtcctagctgtccttggagctgtggtcaccgctatgatgtgtaggaggaagagctcaggtggaaaaggagggagctgctctcaggctgcgtgcagcaacagtgcccagggctctgatgagtctctcatcacttgtaaagcctgagacagctgcctgtgtgggactgagatgcaggatttcttcacacctctcctttgtgacttcaagagcctctggcatctctttctgcaaaggcacctgaatgtgtctgcgttcctgttagcataatgtgaggaggtggagagacagcccacccccgtgtccaccgtgacccctgtccccacactgacctgtgttccctccccgatcatctttcctgttccagagaggtggggctggatgtctccatctctgtctcaaattcatggtgcactgagctgcaacttcttacttccctaatgaagttaagaacctgaatataaatttgtgttctcaaatatttgctatgaagcgttgatggattaattaaataagtcaattcctagaagttgagagagcaaataaagacctgagaaccttccaaaaaaaaaaaaaaaaaaa

SEQ ID NO: 140 - DNA, 1815 bp, Homo sapiens
cttagctgaagatgactgacagtgttatttattccatgttagagttgcctacggcaacccaagcccagaatgactatggaccacagcaaaaatcttcctcttccaggccttcttgttcttgccttgtggcaatagctttggggcttctgactgcagttcttctgagtgtgctgctataccagtggatcctgtgccagggctccaactactccacttgtgccagctgtcctagctgcccagaccgctggatgaaatatggtaaccattgttattatttctcagtggaggaaaaggactggaattctagtctggaattctgcctagccagagactcacacctccttgtgataacggacaatcaggaaatgagcctgctccaagttttcctcagtgaggccttttgctggattggtctgaggaacaattctggctggaggtgggaagatggatcacctctaaacttctcaaggatttcttctaatagctttgtgcagacatgcggtgccatcaacaaaaatggtcttcaagcctcaagctgtgaagttcctttacactgggtgtgtaagaaggtcagactttgatagatgaccactctgtcctgaccctcagatctgtcatgtatccctaaaaggagggagctggccactggctgttgggaaagccatgagtatatagttagcaaatactgaactttctcagatatggcattagatgcaagacaacctcctagggattgatgcctaactgatggattctctttgagactatttagatattatgtgagcaatttaaagaccagatctaagcaaattttgaaatagatgtttgttttttgtatttctcagtatggaaactaatgctgccactctcatccccgtcccaaccatctctgtcaaaaatatacctttttcatatgatattctgagctaatctgataaaatctatgccaatatatactattgcttgtgtactagagaagtacattattgctgtactcctctgtacattactgatccctgatggtatatttctatcctaacagtgtccctttgcagatcaagctttattctgaagaataaacctagctggcatgctggtgtgtacctgtagtcctagctatttgggaagctgaggtgggagggtcgcttgagcccaggagtttgaggctgcagtgagctatgattgtgccactgtactccagcctgggagatagagcaagactccatctctaaaaaaaaaaaaaaatgctaatgtgagaatataaattgtgggaaatgagtgagggcaaggtggtacttcctccttctgagctcttcacacgtaatgcaaaaacccggtcttaactgattttgttttttttctgagtatgcatatatgtggttgaatgaaccaatgtgtgattgtatcttttccattatgtgactgtttgacctgcatattaatttcaagatagcagtcaattcgataaggcattttcatagaggaaagtttacagaaacagtttatgtggttggatcaccaaattatcttaggtactaaggcctcaaaaataagaaaaactttattatttctcctcagtagagtttggacatacataaggagagaaggtacagtgatgaaggagaccataattctgtagtgttgatgatcctggattataatctttttctctttatctttcatagtttttttaaaaacatggactgtatcttatctaccactatatcccaaatacctaagatagtgcttacgttcagtgactattaaataaataaatggatgaattaaaaaaaaaaaaaaaaaaaaaaaaaaaaa

SEQ ID NO: 141 - DNA, 1578 bp, Homo sapiens
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 142 - DNA, 1741 bp, Homo sapiens
ccggcagcaccatgttgctcatggtcgtcagcatggcgtgtgttgggttgttcttggtccagagggccggtccacacatgggtggtcaggacaagcccttcctgtctgcctggcccagcgctgtggtgcctcgcggaggacacgtgactcttcggtgtcactatcgtcataggtttaacaatttcatgctatacaaagaagacagaatccacgttcccatcttccatggcagaatattccaggagggcttcaacatgagccctgtgaccacagcacatgcagggaactacacatgtcggggttcacacccacactcccccactgggtggtcggcacccagcaaccccatggtgatcatggtcacaggaaaccacagaaaaccttccctcctggcccacccaggtcccctggtgaaatcaggagagagagtcatcctgcaatgttggtcagatatcatgtttgagcacttctttctgcacaaagagtggatctctaaggacccctcacgcctcgttggacagatccatgatggggtctccaaggccaatttctccatcggttccatgatgcgtgcccttgcagggacctacagatgctacggttctgttactcacaccccctatcagttgtcagctcccagtgatcccctggacatcgtggtcacaggtctatatgagaaaccttctctctcagcccagccgggccccaaggttcaggcaggagagagcgtgaccttgtcctgtagctcccggagctcctatgacatgtaccatctatccagggaggggggagcccatgaacgtaggctccctgcagtgcgcaaggtcaacagaacattccaggcagatttccctctgggccctgccacccacggagggacctacagatgcttcggctctttccgtcactctccctacgagtggtcagacccgagtgacccactgcttgtttctgtcacaggaaacccttcaagtagttggccttcacccacagaaccaagctccaaatctggtaacctcagacacctgcacattctgattgggacctcagtggtcaaaatccctttcaccatcctcctcttctttctccttcatcgctggtgctccaacaaaaaaaaatgctgctgtaatggaccaagagcctgcagggaacagaagtgaacagcgaggattctgatgaacaagaccatcaggaggtgtcatacgcataattggaacactgtgttttcacacagagaaaaatcactcgcccttctcagaggcccaagacacccccaacagataccagcatgtacatagaacttccaaatgctgagcccagatccaaagttgtcttctgtccacgagcaccacagtcaggccttgaggggatcttctagggagacaacagccctgtctcaaaactgggttgccagctcccatgtaccagcagctggaatctgaaggcatcagtcttcatcttagggcatcgctcttcctcacaccacaaatctgaatgtgcctctcacttgcttacaaatgtctaaggtccccactgcctgctggagaaaaaacacactcctttgcttagcccacagttctccatttcacttgacccctgcccacctctccaacctaactggcttacttcctagtctacttgaggctgcaatcacactgaggaactcacaattccacacatacaagaggctccgtcttaacgcagcacttagacacgtgctgttccaccttccctcatgctg

SEQ ID NO: 143 - DNA, 1986 bp, Homo sapiens
ataacatcctgtgcgctgctgagctgagctggggcgcagccgcctgtctgcaccggcagcaccatgtcgctcatggtcgtcagcatggcgtgtgttgggttgttcttggtccagagggccggtccacacatgggtggtcaggacaaacccttcctgtctgcctggcccagcgctgtggtgcctcgaggaggacacgtgactcttcggtgtcactatcgtcataggtttaacaatttcatgctatacaaagaagacagaatccacattcccatcttccatggcagaatattccaggagagcttcaacatgagccctgtgaccacagcacatgcagggaactacacatgtcggggttcacacccacactcccccactgggtggtcggcacccagcaaccccgtggtgatcatggtcacaggaaaccacagaaaaccttccctcctggcccacccaggtcccctggtgaaatcaggagagagagtcatcctgcaatgttggtcagatatcatgtttgagcacttctttctgcacaaagaggggatctctaaggacccctcacgcctcgttggacagatccatgatggggtctccaaggccaatttctccatcggtcccatgatgcttgcccttgcagggacctacagatgctacggttctgttactcacaccccctatcagttgtcagctcccagtgatcccctggacatcgtggtcacaggtccatatgagaaaccttctctctcagcccagccgggccccaaggttcaggcaggagagagcgtgaccttgtcctgtagctcccggagctcctatgacatgtaccatctatccagggaggggggagcccatgaacgtaggctccctgcagtgcgcaaggtcaacagaacattccaggcagatttccctctgggccctgccacccacggagggacctacagatgcttcggctctttccgtcactctccctacgagtggtcagacccgagtgacccactgcttgtttctgtcacaggaaacccttcaagtagttggccttcacccacagaaccaagctccaaatctggtaaccccagacacctgcacattctgattgggacctcagtggtcatcatcctcttcatcctcctcctcttctttctccttcatctctggtgctccaacaaaaaaaatgctgctgtaatggaccaagagcctgcagggaacagaacagccaacagcgaggactctgatgaacaagaccctgaggaggtgacatacgcacagttggatcactgcgttttcacacagagaaaaatcactcgcccttctcagaggcccaagacaccccctacagataccatcttgtacacggaacttccaaatgctaagcccagatccaaagttgtctcctgcccatgagcaccacagtcaggccttgaggacgtcttctagggagacaacagccctgtctcaaaaccgagttgccagctcccatgtaccagcagctggaatctgaaggcgtgagtcttcatcttagggcatcgctcctcctcacgccacaaatctggtgcctctctcttgcttacaaatgtctaggtccccactgcctgctggaaagaaaacacactcctttgcttagcccacagttctccatttcacttgacccctgcccacctctccaacctaactggcttacttcctagtctacttgaggctgcaatcacactgaggaactcacaattccaaacatacaagaggctccctcttgacgtggcacttacccacgtgctgttccaccttccctcatgctgtttcacctttcttcggactattttccagccttctgtcagcagtgaaacttataaaattttttgtgatttcaatgtagctgtctcctcttcaaataaacatgtctgccctcaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 144 - DNA, 1574 bp, Homo sapiens
agattctccccagacgccgaggatggccgtcatggcgccccgaaccctcctcctgctactctcgggggccctggccctgacccagacctgggcgggctcccactccatgaggtatttcttcacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctactacaaccagagcgaggccggttctcacaccatccagataatgtatggctgcgacgtggggtcggacgggcgcttcctccgcgggtaccggcaggacgcctacgacggcaaggattacatcgccctgaacgaggacctgcgctcttggaccgcggcggacatggcggctcagatcaccaagcgcaagtgggaggcggcccatgaggcggagcagttgagagcctacctggatggcacgtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtccctcacagcttgtaaagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaattagaaccttagtataaatttactttctcaaattcttgccatgagaggttgatgagttaattaaaggagaagattcctaaaatttgagagacaaaataaatggaagacatgagaaccttccagagtccaaaaaaaaaaaaaaaaaaaaaaaaaa

SEQ ID NO: 145 - protein, 444 aa, Homo sapiens
MSLMVVSMACVGLFLVQRAGPHMGGQDKPFLSAWPSAVVPRGGHVTLRCHYRHRFNNFMLYKEDRIHIPIFHGRIFQESFNMSPVTTAHAGNYTCRGSHPHSPTGWSAPSNPVVIMVTGNHRKPSLLAHPGPLVKSGERVILQCWSDIMFEHFFLHKEGISKDPSRLVGQIHDGVSKANFSIGPMMLALAGTYRCYGSVTHTPYQLSAPSDPLDIVVTGPYEKPSLSAQPGPKVQAGESVTLSCSSRSSYDMYHLSREGGAHERRLPAVRKVNRTFQADFPLGPATHGGTYRCFGSFRHSPYEWSDPSDPLLVSVTGNPSSSWPSPTEPSSKSGNPRHLHILIGTSVVIILFILLLFFLLHLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 146 - DNA, 1691 bp, Homo sapiens
gtctgcaccggcagcaccatgtcgctcatggtcgtcagcatggcgtgtgttgggttcttcttgctggaggggccctggccacatgtgggtggtcaggacaagcccttcctctctgcctggcccggcactgtggtgtctgaaggacaacatgtgactcttcagtgtcgctctcgtcttgggtttaacgaattcagtctgtccaaagaagacgggatgcctgtccctgagctctacaacagaatattccggaacagctttctcatgggccctgtgaccccagcacatgcagggacctacagatgttgcagttcacacccacactcccccactgggtggtcggcacccagcaaccctgtggtgatcatggtcacaggagtccacagaaaaccttccctcctggcccacccaggtcccctggtgaaatcgggagagacggtcatcctgcaatgttggtcagatgtcaggtttgagcgcttccttctgcacagagaggggatcactgaggaccccttgcgcctcgttggacagctccacgatgcgggttcccaggtcaactattccatgggtcccatgacacctgcccttgcagggacctacagatgctttggttctgtcactcacttaccctatgagttgtcggctcccagtgaccctctggacatcgtggtcgtaggtctatatgggaaaccttctctctcagcccagccgggccccacggttcaggcaggagagaatgtgaccttgtcctgcagctcccggagcttgtttgacatttaccatctatccagggaggcagaggccggtgaacttaggctcactgcggtgctgagggtcaatggaacattccaggccaacttccctctgggccctgtgacccacggagggaactacagatgcttcggctctttccgtgccctgccccacgcgtggtcagacccgagtgacccactgcccgtttctgtcacaggtaactccagacacctgcacgttctgattgggacctcagtggtcatcatcccctttgctatcctcctcttctttctccttcatcgctggtgtgccaacaaaaagaatgctgttgtaatggaccaagagcctgcagggaacagaacagtgaacagggaggactctgatgaacaagaccctcaggaggtgacatacgcacagttgaatcactgcgttttcacacagagaaaaatcactcgcccttctcagaggcccaagacacccccaacagataccagcgtgtaacacggaacttccaaatgctgagcgcagatccaaagttgtcttctgtccactagcaccacagtcaggccttgatgggatcttctagggagacaatagccctgtctcaaaaccgggttgccagctcccatgtaccagcagctggactctgaaggcgtgagtctgcatcttagggcatcgctcttcctcacaccacgaatctgaacatgcctctctcttgcttacaaatgtctaaggtccccactgcctgctggagagaaaacacacttgcttagcccacaattctccatttcacttgacccctgcccacctctccaacctaactggcttacttcctagtctacttgaggctgcgatcacactgaggaactcacaattccaaacatataagaggctccctcttaacacggcacttagatacgtgctattccacctttcctcag

SEQ ID NO: 147 - DNA, 1294 bp, Homo sapiens
catgtcgctcatggtcatcagcatggcgtgtgttgggttcttcttgctgcagggggcctggacacatgagggtggtcaggacaagcccttcctctctgcctggcccagccctgtggtgtctgaaggagaacatgtggctcttcagtgtcgctctcgtcttgggtttaacgaattcagtctgtccaaagaagacgggatgcctgtccctgagctctacaacagagtattccgaaacaccgttttcataggccctgtgaccccagcacatgcagggacctacagatgtcggggttcacacccacacttcctcactgggtggtcagcacccagcaaccccctggtgatcatggtcacaggagtccacagaaaaccttccctcctggcccacccaggtcccctggtgaaatcagaagagacagtcatcctgcaatgttggtcagatgtcatgtttgagcacttccttctgcacagagaggggaagtttaatgacactttgcgcctcactggagagctccatgatggggtctccaaggccaacttctccatcggtcgcatgacgcaagaccttgcagggacctacagatgctacggttctgttcctcattccccctatcagttgtcagctcccagtgaccctctggacatcgtgattacaggtctatgtgggaaaccttctctctcagcccagccgcgccccatggttaaggcaggagagagcgtgaccttgtcctgcagctcccggagctcctatgacatctaccatctatcaagggagggggaggctcatgaacttaggttccctgcagtgcccaaggtcaatggaaccttccaggccaactttcctctgggccctgccacccacggagggacctacagatgcttcggctctttccgtgactctccctacgagtggtcagaccttagtgacccactgcttgtttctgtcacagattctatgaaggagaaaggaaaagatgtgatactgtaattttgctccatttgtctaaaatgagtaggctgcaactcctcttgaagtgataccttttctagctcttgttggaggtgtctcaggactcattacttcggggaacctgcaactgtgtcagtctggggaaactgcaaatattcttgtcttacatttgtctccagccaattgtgatggactccagtgacctgcaattgctgttattgcaggtaaaatgtacctgagtcaggccacagttctcctggactatgagcccctggccatgttcctgaggcaattctgttcatctaaatataataataataacac

SEQ ID NO: 148 - protein, 375 aa, Homo sapiens
MSLMVISMACVGFFLLQGAWTHEGGQDKPLLSAWPSAVVPRGGHVTLLCRSRLGFTIFSLYKEDGVPVPELYNKIFWKSILMGPVTPAHAGTYRCRGSHPRSPIEWSAPSNPLVIVVTGLFGKPSLSAQPGPTVRTGENVALSCSSRSSFDMYHLSREGRAHEPRLPAVPSVDGTFQADFPLGPATHGGTYTCFSSLHDSPYEWSDPSDPLLVSVTGNSSSSSSSPTEPSSKTGIRRHLHILIGTSVAIILFIILFFFLLHCCCSNKKNAAVMDQGPAGDRTVNREDSDDQDPQEVTYAQLDHCVFTQTKITSPSQRPKAPPTDTTMYMELPNAKPRSLSPAHKHHSQALRGSSRETTALSQNRVASSHVPAAGI

SEQ ID NO: 149 - DNA, 1632 bp, Homo sapiens
atgtcgctcatggtcatcagcatggcgtgtgttgggttcttcttgctgcagggggcctggacacatgagggtggacaggacaagcccttgctgtctgcctggcccagcgctgtggtgcctcgaggaggacatgtgactcttctgtgtcgctctcgtcttgggtttaccatcttcagtctgtacaaagaagatggggtgcctgtccctgagctctacaacaaaatattctggaagagcatcctcatgggccctgtgacccctgcacacgcagggacctacagatgtcggggttcacacccgcgctcccccattgagtggtcggcacccagcaaccccctggtgatcgtggtcacaggtctatttgggaaaccttcactctcagcccagccgggccccacggttcgcacaggagagaacgtggccttgtcctgcagctccaggagctcatttgacatgtaccatctatccagggaggggagggcccatgaacctaggctccctgcagtgcccagcgtcgatggaacattccaggctgactttcctctgggccctgccacccacggagggacctacacatgcttcagctctctccatgactcaccctatgagtggtcagacccgagtgacccactgcttgtttctgtcacaggaaactcttcaagtagttcatcttcacccactgaaccaagctccaaaactggtatccgcagacacctgcacattctgattgggacctcagtggctatcatcctcttcatcatcctcttcttctttctccttcattgctgctgctccaacaaaaagaatgctgctgtaatggaccaagggcctgccggggacagaacagtgaacagggaggactctgatgatcaagaccctcaggaggtgacatatgcacagttggatcactgcgttttcacacagacaaaaatcacttccccttctcagaggcccaaggcacctccaacagataccaccatgtacatggaacttccaaatgctaagccaagatcattgtctcctgcccataagcaccacagtcaggccttgaggggatcttctagggagacaacagccctgtctcaaaaccgggttgctagctcccatgtaccagcagctggaatctgaaggcatcagtcttcatcttaggggatcgctcttcctcacaccacaaatctgaacatgcctctctcttgcttacaaatgtctaaggtccccactgcctgctggagagaagacacacacctttgcttagcccacaattctctatttcacttgacccctgcccacctctccaactgaactggcttacttcctagtctacttgaggctgcaatcacactgaggaactcacaattccagacatacaagaggctccctcttaacatggcactgagacacgtgctgttccaccctccctcatgctgtttcacctttcctcagactattttccagccttctgtcagtcagcagtgaaacttataaaattttttgtgatttcaatgtagctgtctccttttcaaataaacatgtctgccctcattgctttaggtaatgtgacactattcgctgaaagaaaccgctgttatcattaccatgtccacataaccccatctgttatccactgggttc

SEQ ID NO: 150 - DNA, 1559 bp, Homo sapiens
tgtctgcacagacagcaccatgtcgctcatggtcgtcagcatggcgtgtgttgggttcttcttgctgcagggggcctggccacatgagggagtccacagaaaaccttccctcctggcccacccaggtcccctggtgaaatcagaagagacagtcatcctgcaatgttggtcagatgtcaggtttgagcacttccttctgcacagagaggggaagtataaggacactttgcacctcattggagagcaccatgatggggtctccaaggccaacttctccatcggtcccatgatgcaagaccttgcagggacctacagatgctacggttctgttactcactccccctatcagttgtcagctcccagtgaccctctggacatcgtcatcacaggtctatatgagaaaccttctctctcagcccagccgggccccacggttttggcaggagagagcgtgaccttgtcctgcagctcccggagctcctatgacatgtaccatctatccagggagggggaggcccatgaacgtaggttctctgcagggcccaaggtcaacggaacattccaggccgactttcctctgggccctgccacccacggaggaacctacagatgcttcggctctttccgtgactctccctatgagtggtcaaactcgagtgacccactgcttgtttctgtcacaggaaacccttcaaatagttggccttcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataattggatcactgtgttttcacacagagaaaaatcactcgcccttctgagaggcccaagacacccccaacagataccagcatgtacatagaacttccaaatgctgagcccagatccaaagttgtcttctgtccacgagcaccacagtcaggccttgaggggatcttctagggagacaacagccctgtctcaaaaccgggttgccagctcccatgtaccagcagctggaatctgaaggcatcagtcttcatcttagggcatcgctcttcctcacaccacgaatctgaacatgcctctctcttgcttacaaatgtctaaggtccccactgcctgctggagagaaaacacactcctttgcttagcccacaattctccatttcacttgacccctgcccacctctccaacctaactggcttacttcctagtctacctgaggctgcaatcacactgaggaactcacaattccaaacatacaagaggctgcctcttaacacagcacttagacacgtgctgttccacctcccttcagactatctttcagccttctgccagcagtaaaacttataaattttttaaataatttcaatgtagttttcccgccttcaaataaacatgtctgccctcatg

SEQ ID NO: 151 - DNA, 1113 bp, Homo sapiens
ccggcagcaccatgtcgctcatggtcatcagcatggcatgtgttgggttcttctggctgcagggggcctggccacatgagggattccgcagaaaaccttccctcctggcccacccaggtcgcctggtgaaatcagaagagacagtcatcctgcaatgttggtcagatgtcatgtttgagcacttccttctgcacagagaggggacgtttaacgacactttgcgcctcattggagagcacattgatggggtctccaaggccaacttctccatcggtcgcatgaggcaagacctggcagggacctacagatgctacggttctgttcctcactccccctatcagttttcagctcccagtgaccctctggacatcgtgatcacaggtctatatgagaaaccttctctctcagcccagccgggccccacggttctggcaggagagagcgtgaccttgtcctgcagctcctggagctcctatgacatgtaccatctatccacggagggggaggcccatgaacgtaggttctctgcagggcccaaggtcaacggaacattccaggccgactttcctctgggccctgccacccaaggaggaacctacagatgcttcggctctttccatgactctccctacgagtggtcaaagtcaagtgacccactgcttgtttctgtcacaggaaacccttcaaatagttggccttcacccactgaaccaagctccaaaaccggtaaccccagacacctacacgttctgattgggacctcagtggtcaaactccctttcaccatcctcctcttctttctccttcatcgctggtgctccgacaaaaaaaatgcatctgtaatggaccaagggcctgcggggaacagaacagtgaacagggaggattctgacgaacaggaccatcaggaggtgtcatacgcataattggatcactgtgttttcacacagagaaaaatcactcccccttctcagaggcccaagacacccccaacagatagcagcatgtacatagaacttccaaatgctgagtccagatccaaagctgtcttctgtccacgagcaccacagtcaggccttgaggggatcttctagggagacaacagccctgtct

SEQ ID NO: 152 - DNA, 1586 bp, Homo sapiens
gctgagctgagctggggcgcggccgcctgtctgcaccggcagcaccatgtcgctcatggtcatcatcatggcgtgtgttgggttcttcttgctgcagggggcctggccacaggagggagtccacagaaaaccttccttcctggccctcccaggtcacctggtgaaatcagaagagacagtcatcctgcaatgttggtcggatgtcatgtttgagcacttccttctgcacagagaggggaagtttaacaacactttgcacctcattggagagcaccatgatggggtttccaaggccaacttctccattggtcccatgatgcctgtccttgcaggaacctacagatgctacggttctgttcctcactccccctatcagttgtcagctcccagtgaccctctggacatggtgatcataggtctatatgagaaaccttctctctcagcccagccgggccccacggttcaggcaggagagaatgtgaccttgtcctgcagctcccggagctcctatgacatgtaccatctatccagggaaggggaggcccatgaacgtaggctccctgcagtgcgcagcatcaacggaacattccaggccgactttcctctgggccctgccacccacggagggacctacagatgcttcggctctttccgtgacgctccctacgagtggtcaaactcgagtgatccactgcttgtttccgtcacaggaaacccttcaaatagttggccttcacccactgaaccaagctccaaaaccggtaaccccagacacctacatgttctgattgggacctcagtggtcaaaatccctttcaccatcctcctcttctttctccttcatcgctggtgctccgacaaaaaaaatgctgctgtaatggaccaagagcctgcagggaacagaacagtgaacagcgaggattctgatgaacaagaccatcaggaggtgtcatacgcataattggatcactgtgttttcacacagagaaaaatcactcgcccttctgagaggcccaagacacccccaacagataccagcatgtacatagaacttccaaatgctgagcccagatccaaagttgtcttctgtccacgagcaccacagtcaggccttgaggggatcttctagggagacaacagccctgtctcaaaaccgggttgccagctcccatgtaccagcagctggaatctgaaggcatcagtcttcatcttagggcatcgctcttcctcacaccacgaatctgaacatgcctctctcttgcttacaaatgtctaaggtccccactgcctgctggagagaaaacacactcctttgcttagcccacaattctccatttcacttgacccctgcccacctctccaacctaactggcttacttcctagtctacctgaggctgcaatcacactgaggaactcacaattccaaacatacaagaggctgcctcttaacacagcacttagacacgtgctgttccacctcccttcagactatctttcagccttctgccagcagtaaaacttataaattttttaaataatttcaatgtagttttcccgccttcaaataaacatgtctgccctcatg

SEQ ID NO: 153 - DNA, 1598 bp, Homo sapiens
gagctcggtcgcggctgcctgtctgctccggcagcaccatgtcgctcttggtcgtcagcatggcgtgtgttgggttcttcttgctgcagggggcctggccacatgagggagtccacagaaaaccttccctcctggcccacccaggtcgcctggtgaaatcagaagagacagtcatcctgcagtgttggtcagatgtcatgtttgaacacttccttctgcacagagaggggatgtttaacgacactttgcgcctcattggagaacaccatgatggggtctccaaggccaacttctccatcagtcgcatgacgcaagacctggcagggacctacagatgctacggttctgttactcactccccctatcaggtgtcagctcccagtgaccctctggacatcgtgatcataggtctatatgagaaaccttctctctcagcccagctgggccccacggttctggcaggagagaatgtgaccttgtcctgcagctcccggagctcctatgacatgtaccatctatccagggaaggggaggcccatgaacgtaggctccctgcagggcccaaggtcaacggaacattccaggctgactttcctctgggccctgccacccacggagggacctacagatgcttcggctctttccatgactctccatacgagtggtcaaagtcaagtgacccactgcttgtttctgtcacaggaaacccttcaaatagttggccttcacccactgaaccaagctccaaaaccggtaacccccgacacctgcacattctgattgggacctcagtggtcatcatcctcttcatcctcctcttctttctccttcatcgctggtgctccaacaaaaaaaatgctgcggtaatggaccaagagtctgcaggaaacagaacagcgaatagcgaggactctgatgaacaagaccctcaggaggtgacatacacacagttgaatcactgcgttttcacacagagaaaaatcactcgcccttctcagaggcccaagacacccccaacagatatcatcgtgtacacggaacttccaaatgctgagtccagatccaaagttgtctcctgcccatgagcaccacagtcaggccttgagggcgtcttctagggagacaacagccctgtctcaaaaccgggttgccagctcccatgtaccagcagctggaatctgaaggcgtgagtctgcatcttagggcatcgatcttcctcacaccacaaatctgaatgtgcctctctcttgcttacaaatgtctaaggtccccactgcctgctggagaaaaaacacactcctttgcttaacccacagttctccatttcacttgacccctgcccacctctccaacctaactggcttacttcctagtctacttgaggctgcaatcacactgaggaactcacaattccaaacatacaagaggctccctcttaacgcagcacttagacacgtgttgttccaccttccctcatgctgttccacctcccctcagactagctttcagtcttctgtcagcagtaaaacttatatattttttaaaataacttcaatgtagttttccatccttcaaataaacatgtctgcccccatggt

SEQ ID NO: 154 - DNA, 1101 bp, Homo sapiens
caccggcagcaccatgtcgctcacggtcgtcagcatggcgtgtgttgggttcttcttgctgcagggggcctggccacatgagggagtccacagaaaaccttccctcctggcccacccaggtcgcctggtgaaatcagaagagacagtcatcctgcaatgttggtcagatgtcatgtttgaacacttccttctgcacagagaggggatgtttaacgacactttgcgcctcattggagaacaccatgatggggtctccaaggccaacttctccatcagtcgcatgaagcaagacctggcagggacctacagatgctacggttctgttactcactccccctatcagttgtcagctcccagtgaccctctggacatcgtgatcataggtctatatgagaaaccttctctctcagcccagccgggccccacggttctggcaggagagaatgtgaccttgtcctgcagctcccggagctcctatgacatgtaccatctatccagggaaggggaggcccatgaacgtaggctccctgcagggaccaaggtcaacggaacattccaggccaactttcctctgggccctgccacccatggagggacctacagatgcttcggctctttccgtgactctccatacgagtggtcaaagtcaagtgacccactgcttgtttctgtcacaggaaacccttcaaatagttggccttcacccactgaaccaagctccgaaaccggtaaccccagacacctacatgttctgattgggacctcagtggtcaaaatccctttcaccatcctcctcttctttctccttcatcgctggtgctccgacaaaaaaaatgctgctgtaatggaccaagagcctgcagggaacagaacagtgaacagcgaggattctgatgaacaagaccatcaggaggtgtcatacgcataattggatcactgtgttttcacacagagaaaaatcactcgcccttctgagaggcccaagacacccccaacagataccagcatgtacatagaacttccaaatgctgagcccagatccaaagttgtcttctgtccacgagcaccacagtcaggccttgaggggatcttctagggaga

SEQ ID NO: 155 - DNA, 1602 bp, Homo sapiens
aggggtgcggccgcctgtctgcaccggcagcaccatgtcgctcatggtcatcagcatggcgtgtgttgcgttcttcttgctgcagggggcctggccacatgagggattccgcagaaaaccttccctcctggcccacccaggtcccctggtgaaatcagaagagacagtcatcctgcaatgttggtcagatgtcatgtttgagcacttccttctgcacagagaggggacgtttaaccacactttgcgcctcattggagagcacattgatggggtctccaagggcaacttctccatcggtcgcatgacacaagacctggcagggacctacagatgctacggttctgttactcactccccctatcagttgtcagcgcccagtgaccctctggacatcgtgatcacaggtctatatgagaaaccttctctctcagcccagccgggccccacggttctggcaggagagagcgtgaccttgtcctgcagctcccggagctcctatgacatgtaccatctatccagggaaggggaggcccatgaacgtaggctccctgcagggcccaaggtcaacagaacattccaggccgactttcctctggaccctgccacccacggagggacctacagatgcttcggctctttccgtgactctccatacgagtggtcaaagtcaagtgacccactgcttgtttctgtcacaggaaactcttcaaatagttggccttcacccactgaaccaagctccgaaaccggtaaccccagacacctacacgttctgattgggacctcagtggtcaaactccctttcaccatcctcctcttctttctccttcatcgctggtgctccaacaaaaaaaatgcatctgtaatggaccaagggcctgcggggaacagaacagtgaacagggaggattctgatgaacaggaccatcaggaggtgtcatacgcataattggatcactgtgttttcacacagagaaaaatcactcccccttctcagaggcccaagacacccccaacagataccagcatgtacatagaacttccaaatgctgagtccagatccaaagctgtcttctgtccacgagcaccacagtcaggccttgaggggatcttctagggagacaacagccctgtctcaaaaccgggttgccagctcccatgtaccagcagctggaatctgaaggcatcagtcttcatcttaggggatcgctcttcctcaaaccacgaatctgaacatgcctctctcttgcttacaaatgtctaaggtccccactgcctgctggagagaaaacacactcctttgcttagcccacaattctccatttcacttgacccctgcccacctctccaacctaactggcttacttcctagtctacttgaggctgcaatcacactgaggaactcacaattccaaacatacaagaggctccctcttaacacagcacttagacacgtgctgttccaccttctctcatgcagttccacctcccctcagactatctttcagccttctgtcagcagtaaaacttataaattgtttttagtaatttcaatgtagttttccctccttcaaataaacatgtctgccctcatggtttcga

SEQ ID NO: 156 - DNA, 702 bp, Homo sapiens
ggtggtcaggacaagcccttgctttctacctggcccagccttgtggtgcctccagaacatgtgactcttcggtgtcactctaatcttgggtttaacaacttcagtctgtacaaggatgatggggtgcctgtccctgagctctacaacagaatattctggaaaagccttttcatgggccctgtgaccccgtcacacacagggacctatagatgccggggttcacacacacactcccccagtggggggtcggcacccagcaaccccctggtgatcgtggtcacaggattccgcagaaaaccttccctcctggcccacccaggtcgcctggtgaaatcagaagagacagtcatcctgcaatgttggtcagatgtcatgtttgagcacttccttctgcacagagaggggacgtttaacgacactttgcgcctcattggagagcacattgatggggtctccaaggccaacttctccatcggtcgcatgaggcaagacctggcagggacctacagatgctacggttctgttcctcactccccctatcagttttcagctcccagtgaccctctggacatcgtgatcacaggtgagagtgtccagacattcttctcattgtcattcggacacagagtgaatgatccaggacttggaggcccaggtggttgtaaggaagatgagcttggtattcttatggagagagactga

SEQ ID NO: 157 - DNA, 1025 bp, Homo sapiens
tttctcactcccattgggcgtcgcgtttctagagaagccaatcagtgtcgccgcagttcccaggttctaaagtcccacgcaccccgcgggactcatatttttcccagacgcggaggttggggtcatggcgccccgaagcctcctcctgctgctctcaggggccctggccctgaccgatacttgggcgggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtgtgagacagcttccttgtgtgggactgagaagcaagatatcaatgtagcagaattgcacttgtgcctcacgaacatacataaattttaaaaataaagaataaaaatatatctttttatagataaaaaaaaaaaaaaaaaa

SEQ ID NO: 158 - DNA, 1591 bp, Homo sapiens
tttctcactcccattgggcgtcgcgtttctagagaagccaatcagtgtcgccgcagttcccaggttctaaagtcccacgcaccccgcgggactcatatttttcccagacgcggaggttggggtcatggcgccccgaagcctcctcctgctgctctcaggggccctggccctgaccgatacttgggcgggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgcctcctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagcctactcagtggtcagcggaaacttgatgataacatggtggtcaagcttatttctcctgggggtgctcttccaaggatatttgggctgcctccggagtcacagtgtcttgggccgccggaaggtgggtgacatgtggatcttgttttttttgtggctgtggacatctttcaacactgccttcttggccttgcaaagccttcgctttggcttcggctttaggaggggcaggagcttccttcttcgttcttggcaccatcttatgaaaagggtccagattaagatttttgactgagtcattctaaagtaagttgcaagacccatgatactagaccactaaatacttcatcacacacctcctaagaataagaaccaacattatcacaccaaagaaaataaataattccataatattaaaaaaaaaaaaaaaaaa

SEQ ID NO: 159 - DNA, 1301 bp, Homo sapiens
tttctcactcccattgggcgtcgcgtttctagagaagccaatcagtgtcgccgcagttcccaggttctaaagtcccacgcaccccgcgggactcatatttttcccagacgcggaggttggggtcatggcgccccgaagcctcctcctgctgctctcaggggccctggccctgaccgatacttgggcgggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgcctcctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtgtgagacagcttccttgtgtgggactgagaagcaagatatcaatgtagcagaattgcacttgtgcctcacgaacatacataaattttaaaaataaagaataaaaatatatctttttatagataaaaaaaaaaaaaaaaaa

SEQ ID NO: 160 - DNA, 244 bp, Homo sapiens
  misc_feature (31)..(31): n is a, c, g, or t
ggcctggccacatgagggtggtcaggacaancccttgctgtctgcctggcccanccttgtggtgcctctnggacatgtcattcttcggtgtcactnttatcttgggtntaacaacttcagtctntanaangaaggtggggngccngtccctgagctcnacaacagaatantctggaacagccttttcatgggccctgtgacccccgcactacagggacatacagatgtcggggttcacacacac

SEQ ID NO: 161 - DNA, 429 bp, Homo sapiens
  misc_feature (129)..(129): n is a, c, g, or t
tctaccctgcggagatcacgctgacctggcagcgggatggggaggaacagacccaggacacagagcttgtggagaccaggcctgcaggggatggaaccttccagaagtgggccgctgtggtggtgcctnctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtgtgagacagcttccttgtgtgggactgagaagcaagatatcaatgtagcagaattgcacttgtgcctcacgaacata

SEQ ID NO: 162 - DNA, 392 bp, Homo sapiens
ggacattttcttcccatagatagaaacagagggagctactctcaggctgcaggtaagatgaaggaggctgatccctgagattgttgggatattgtggtcaggagcctatgagggagctcacccaccccacagttcctctagccacatctgtgggctctgaccaggtcctgtttttgttctaccccaatcactgacagtgcccagggctctggggtgtctctcacagctaataaaggtgacactccagggcaggggccctgatgtgagtggggtgttgggggggaacagaggggactcagctgtgctattgggtttctttgacttggatgtcttgagcatgaaatgggctatttagagtgttacctctcactgtgactgatacgaatttgttc

SEQ ID NO: 163 - DNA, 503 bp, Homo sapiens
cagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgccttctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtcactgacagtgcccagggctctggggtgtctctcacagctaataaagtgtgagacagcttccttgtgtgggac

SEQ ID NO: 164 - protein, 119 aa, Homo sapiens
MEASALTSSAVTSVAKVVRVASGSAVVLPLARIATVVIGGVVAVPMVLSAMGFTAAGIASSSIAAKMMSAAAIANGGGVASGSLVATLQSLGATGLSGLTKFILGSIGSAIAAVIARFY

SEQ ID NO: 165 - DNA, 876 bp, Homo sapiens
agaacctagagcccaaggttcagagtcacccatctcagcaagcccagaagtatctgcaatatctacgatggcctcgccctttgctttactgatggtcctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataacatctggtccaacatgaaaacaattcttattgactcatacaccaggtcacgctttcatgaattctgtcatttcaaagactctcacccctgctataactatgaccatgctgataaactgatttatctatttaaatatttatttaactattcataagatttaaattatttttgttcatataacgtcatgtgcacctttacactgtggttagtgtaataaaacatgttccttatatttactc

SEQ ID NO: 166 - DNA, 840 bp, Homo sapiens
acattctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtca

SEQ ID NO: 167 - DNA, 1514 bp, Homo sapiens
gatctggtaaacctgaagcaaatatagaaacctatagggcctgacttcctacataaagtaaggagggtaaaaatggaggctagaataagggttaaaattttgcttctagaacagagaaaatgatttttttcatatatatatgaatatatattatatatacacatatatacatatattcactatagtgtgtatacataaatatataatatatatattgttagtgtagtgtgtgtctgattatttacatgcatatagtatatacacttatgactttagtacccagacgtttttcatttgattaagcattcatttgtattgacacagctgaagtttactggagtttagctgaagtctaatgcaaaattaatagattgttgtcatcctcttaaggtcatagggagaacacacaaatgaaaacagtaaaagaaactgaaagtacagagaaatgttcagaaaatgaaaaccatgtgtttcctattaaaagccatgcatacaagcaatgtcttcagaaaacctagggtccaaggttaagccatatcccagctcagtaaagccaggagcatcctcatttcccaatggccctcctgttccctctactggcagccctagtgatgaccagctatagccctgttggatctctgggctgtgatctgcctcagaaccatggcctacttagcaggaacaccttggtgcttctgcaccaaatgaggagaatctcccctttcttgtgtctcaaggacagaagagacttcaggttcccccaggagatggtaaaagggagccagttgcagaaggcccatgtcatgtctgtcctccatgagatgctgcagcagatcttcagcctcttccacacagagcgctcctctgctgcctggaacatgaccctcctagaccaactccacactggacttcatcagcaactgcaacacctggagacctgcttgctgcaggtagtgggagaaggagaatctgctggggcaattagcagccctgcactgaccttgaggaggtacttccagggaatccgtgtctacctgaaagagaagaaatacagcgactgtgcctgggaagttgtcagaatggaaatcatgaaatccttgttcttatcaacaaacatgcaagaaagactgagaagtaaagatagagacctgggctcatcttgaaatgattctcattgattaatttgccatataacacttgcacatgtgactctggtcaattcaaaagactcttatttcggctttaatcacagaattgactgaattagttctgcaaatactttgtcggtatattaagccagtatatgttaaaaagacttaggttcaggggcatcagtccctaagatgttatttatttttactcatttatttattcttacattttatcatatttatactatttatattcttatataacaaatgtttgcctttacattgtattaagataacaaaacatgttcagctttccatttggttaaatattgtattttgttatttattaaattattttcaaac

SEQ ID NO: 168 - DNA, 1240 bp, Homo sapiens
cacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacaaaaaaaaaaaaaaaaaaaaaaaaaaaaaa

SEQ ID NO: 169 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 170 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 171 - protein, 195 aa, Homo sapiens
MALLFPLLAALVMTSYSPVGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 172 - protein, 164 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRA

SEQ ID NO: 173 - DNA, 1143 bp, Homo sapiens
gagaacctggagcctaaggtttaggctcacccatttcaaccagtctagcagcatctgcaacatctacaatggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgaaaactggttcaacatggaaatgattttcattgattcgtatgccagctcacctttttatgatctgccatttcaaagactcatgtttctgctatgaccatgacacgatttaaatcttttcaaatgtttttaggagtattaatcaacattgtattcagctcttaaggcactagtcccttacagaggaccatgctgactgatccattatctatttaaatatttttaaaatattatttatttaactatttataaaacaacttatttttgttcatattatgtcatgtgcacctttgcacagtggttaatgtaataaaatatgttctttgtatttggtaaatttattttgtgttgttcattgaacttttgctatggaaacttttgtacttgtttattctttaaaatgaaattccaagcctaattgtgcaacctgattacagaataactggtacacttcatttatccatcaatattatattcaagatataagtaaaaataaactttctgtaaacca

SEQ ID NO: 174 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 175 - DNA, 19 bp, Artificial sequence
  Exemplary siRNA target sequence
gggcattgttgctggcctg

SEQ ID NO: 176 - DNA, 19 bp, Artificial sequence
  Exemplary siRNA target sequence
catcgtgggcattgttgct

SEQ ID NO: 177 - DNA, 19 bp, Artificial sequence
  Exemplary siRNA target sequence
cattgttgctggcctggct

SEQ ID NO: 178 - DNA, 19 bp, Artificial sequence
  Exemplary siRNA target sequence
tgggcattgttgctggcct

SEQ ID NO: 179 - DNA, 19 bp, Artificial sequence
  Exemplary siRNA target sequence
tctatgaggcagaggaata

SEQ ID NO: 180 - DNA, 19 bp, Artificial sequence
  Exemplary siRNA target sequence
aggaatatgcagaggagtt

SEQ ID NO: 181 - DNA, 19 bp, Artificial sequence
  Exemplary siRNA target sequence
gctacatcgccgtggagta

SEQ ID NO: 182 - DNA, 19 bp, Artificial sequence
  Exemplary siRNA target sequence
ggaatgaatggctgcgaca

SEQ ID NO: 183 - DNA, 19 bp, Artificial sequence
  Exemplary siRNA target sequence
aggcagaggaatatgcaga

SEQ ID NO: 184 - DNA, 19 bp, Artificial sequence
  Exemplary siRNA target sequence
ccgcagatacttggagaat

SEQ ID NO: 185 - DNA, 19 bp, Artificial sequence
  Exemplary siRNA target sequence
gagctcagatagaaacaga

SEQ ID NO: 186 - DNA, 19 bp, Artificial sequence
  Exemplary siRNA target sequence
cagtattgggagtggacca

SEQ ID NO: 187 - DNA, 19 bp, Artificial sequence
  Exemplary siRNA target sequence
gcagaggagttcaggacct

SEQ ID NO: 188 - DNA, 19 bp, Artificial sequence
  Exemplary siRNA target sequence
gcagatacttggagaatgg

SEQ ID NO: 189 - DNA, 19 bp, Artificial sequence
  Exemplary siRNA target sequence
tcagatagaaacagaggga

SEQ ID NO: 190 - DNA, 19 bp, Artificial sequence
  Exemplary siRNA target sequence
agatagaaacagagggagc

SEQ ID NO: 191 - DNA, 19 bp, Artificial sequence
  Exemplary siRNA target sequence
cagggtacgccaaggccaa

SEQ ID NO: 192 - DNA, 19 bp, Artificial sequence
  Exemplary siRNA target sequence
cttctatgaggcagaggaa

SEQ ID NO: 193 - DNA, 19 bp, Artificial sequence
  Exemplary siRNA target sequence
gatacttggagaatgggaa

SEQ ID NO: 194 - DNA, 19 bp, Artificial sequence
  Exemplary siRNA target sequence
aggccaacgcacagactga

SEQ ID NO: 195 - DNA, 19 bp, Artificial sequence
  Exemplary siRNA target sequence
gtacgtagacgacacgcaa

SEQ ID NO: 196 - DNA, 19 bp, Artificial sequence
  Exemplary siRNA target sequence
acgtagacgacacgcaatt

SEQ ID NO: 197 - DNA, 19 bp, Artificial sequence
  Exemplary siRNA target sequence
cagaggagttcaggaccta

SEQ ID NO: 198 - DNA, 19 bp, Artificial sequence
  Exemplary siRNA target sequence
aggaagagctcaggtggaa

SEQ ID NO: 199 - DNA, 19 bp, Artificial sequence
  Exemplary siRNA target sequence
gaggaagagctcaggtgga

SEQ ID NO: 200 - DNA, 19 bp, Artificial sequence
  Exemplary siRNA target sequence
ggaagagctcaggtggaaa

SEQ ID NO: 201 - DNA, 19 bp, Artificial sequence
  Exemplary siRNA target sequence
ggaaaaggagggagctgct

SEQ ID NO: 202 - DNA, 19 bp, Artificial sequence
  Exemplary siRNA target sequence
cagcaggagatggaacctt

SEQ ID NO: 203 - DNA, 19 bp, Artificial sequence
  Exemplary siRNA target sequence
ccagggctctgatgagtct

SEQ ID NO: 204 - DNA, 19 bp, Artificial sequence
  Exemplary siRNA target sequence
ggccagcaggagatggaac

SEQ ID NO: 205 - DNA, 19 bp, Artificial sequence
  Exemplary siRNA target sequence
agcaggagatggaaccttc

SEQ ID NO: 206 - DNA, 19 bp, Artificial sequence
  Exemplary siRNA target sequence
aggaggaagagctcaggtg

SEQ ID NO: 207 - DNA, 19 bp, Artificial sequence
  Exemplary siRNA target sequence
ggaggaagagctcaggtgg

SEQ ID NO: 208 - DNA, 19 bp, Artificial sequence
  Exemplary siRNA target sequence
ccatcttcccagcccacca

SEQ ID NO: 209 - DNA, 19 bp, Artificial sequence
  Exemplary siRNA target sequence
gccagcaggagatggaacc

SEQ ID NO: 210 - DNA, 19 bp, Artificial sequence
  Exemplary siRNA target sequence
ccagcaggagatggaacct

SEQ ID NO: 211 - DNA, 19 bp, Artificial sequence
  Exemplary siRNA target sequence
ggaagagctcagatagaaa

SEQ ID NO: 212 - DNA, 19 bp, Artificial sequence
  Exemplary siRNA target sequence
gaggaagagctcagataga

SEQ ID NO: 213 - DNA, 19 bp, Artificial sequence
  Exemplary siRNA target sequence
gtgatgtggaggaggaaga

SEQ ID NO: 214 - DNA, 19 bp, Artificial sequence
  Exemplary siRNA target sequence
aggaggaagagctcagata

SEQ ID NO: 215 - DNA, 19 bp, Artificial sequence
  Exemplary siRNA target sequence
gcttcatcgccgtgggcta

SEQ ID NO: 216 - DNA, 19 bp, Artificial sequence
  Exemplary siRNA target sequence
ggaccaggagacacggaat

SEQ ID NO: 217 - DNA, 19 bp, Artificial sequence
  Exemplary siRNA target sequence
agatagaaaaggagggagt

SEQ ID NO: 218 - DNA, 19 bp, Artificial sequence
  Exemplary siRNA target sequence
aggaagagctcagatagaa

SEQ ID NO: 219 - DNA, 19 bp, Artificial sequence
  Exemplary siRNA target sequence
caggagacacggaatgtga

SEQ ID NO: 220 - DNA, 19 bp, Artificial sequence
  Exemplary siRNA target sequence
gagtattgggaccaggaga

SEQ ID NO: 221 - DNA, 19 bp, Artificial sequence
  Exemplary siRNA target sequence
ggacatggcggctcagatc

SEQ ID NO: 222 - DNA, 19 bp, Artificial sequence
  Exemplary siRNA target sequence
cccatgaggcggagcagtt

SEQ ID NO: 223 - DNA, 19 bp, Artificial sequence
  Exemplary siRNA target sequence
atagaaaaggagggagtta

SEQ ID NO: 224 - DNA, 19 bp, Artificial sequence
  Exemplary siRNA target sequence
agaaaaggagggagttaca

SEQ ID NO: 225 - DNA, 19 bp, Artificial sequence
  Exemplary siRNA target sequence
tcaccaagcgcaagtggga

SEQ ID NO: 226 - DNA, 19 bp, Artificial sequence
  Exemplary siRNA target sequence
gagcctacctggatggcac

SEQ ID NO: 227 - DNA, 19 bp, Artificial sequence
  Exemplary siRNA target sequence
gccgtgatgtggaggagga

SEQ ID NO: 228 - DNA, 19 bp, Artificial sequence
  Exemplary siRNA target sequence
tctcacaccatccagataa

SEQ ID NO: 229 - DNA, 19 bp, Artificial sequence
  Exemplary siRNA target sequence
cctcacagcttgtaaagtg

SEQ ID NO: 230 - DNA, 19 bp, Artificial sequence
  Exemplary siRNA target sequence
gggaccaggagacacggaa

SEQ ID NO: 231 - DNA, 19 bp, Artificial sequence
  Exemplary siRNA target sequence
ccaagacacatatgaccca

SEQ ID NO: 232 - DNA, 19 bp, Artificial sequence
  Exemplary siRNA target sequence
agactgaccgagtggacct

SEQ ID NO: 233 - DNA, 19 bp, Artificial sequence
  Exemplary siRNA target sequence
gaagagctcagatagaaaa

SEQ ID NO: 234 - DNA, 19 bp, Artificial sequence
  Exemplary siRNA target sequence
gagctcagatagaaaagga

SEQ ID NO: 235 - DNA, 19 bp, Artificial sequence
  Exemplary siRNA target sequence
gtattgggaccaggagaca

SEQ ID NO: 236 - DNA, 19 bp, Artificial sequence
  Exemplary siRNA target sequence
aggcggagcagttgagagc

SEQ ID NO: 237 - DNA, 19 bp, Artificial sequence
  Exemplary siRNA target sequence
atgtgcagcatgagggtct

SEQ ID NO: 238 - DNA, 19 bp, Artificial sequence
  Exemplary siRNA target sequence
ccgtgatgtggaggaggaa

SEQ ID NO: 239 - DNA, 19 bp, Artificial sequence
  Exemplary siRNA target sequence
cggagtattgggaccagga

SEQ ID NO: 240 - DNA, 19 bp, Artificial sequence
  Exemplary siRNA target sequence
ggagacacggaatgtgaag

SEQ ID NO: 241 - DNA, 19 bp, Artificial sequence
  Exemplary siRNA target sequence
cacggaatgtgaaggccca

SEQ ID NO: 242 - DNA, 19 bp, Artificial sequence
  Exemplary siRNA target sequence
cggacatggcggctcagat

SEQ ID NO: 243 - DNA, 19 bp, Artificial sequence
  Exemplary siRNA target sequence
gttgagagcctacctggat

SEQ ID NO: 244 - DNA, 19 bp, Artificial sequence
  Exemplary siRNA target sequence
agaaagaagagctcagatt

SEQ ID NO: 245 - DNA, 19 bp, Artificial sequence
  Exemplary siRNA target sequence
gcacagactgacagaatga

SEQ ID NO: 246 - DNA, 19 bp, Artificial sequence
  Exemplary siRNA target sequence
gggaagaggagacacggaa

SEQ ID NO: 247 - DNA, 19 bp, Artificial sequence
  Exemplary siRNA target sequence
cctacgatggcaaggatta

SEQ ID NO: 248 - DNA, 19 bp, Artificial sequence
  Exemplary siRNA target sequence
acgcacagactgacagaat

SEQ ID NO: 249 - DNA, 19 bp, Artificial sequence
  Exemplary siRNA target sequence
ggtatgaacagtatgccta

SEQ ID NO: 250 - DNA, 19 bp, Artificial sequence
  Exemplary siRNA target sequence
ctgctgtgctgtggagaaa

SEQ ID NO: 251 - DNA, 19 bp, Artificial sequence
  Exemplary siRNA target sequence
aaagaagagctcagattga

SEQ ID NO: 252 - DNA, 19 bp, Artificial sequence
  Exemplary siRNA target sequence
gcttcatcgccatgggcta

SEQ ID NO: 253 - DNA, 19 bp, Artificial sequence
  Exemplary siRNA target sequence
gaagaggagacacggaaca

SEQ ID NO: 254 - DNA, 19 bp, Artificial sequence
  Exemplary siRNA target sequence
gaatgaacctgcagaccct

SEQ ID NO: 255 - DNA, 19 bp, Artificial sequence
  Exemplary siRNA target sequence
gctcagatctccaagcgca

SEQ ID NO: 256 - DNA, 19 bp, Artificial sequence
  Exemplary siRNA target sequence
gctgtgctgtggagaaaga

SEQ ID NO: 257 - DNA, 19 bp, Artificial sequence
  Exemplary siRNA target sequence
tggagaacgggaaggagat

SEQ ID NO: 258 - DNA, 19 bp, Artificial sequence
  Exemplary siRNA target sequence
gctgctgtgctgtggagaa

SEQ ID NO: 259 - DNA, 19 bp, Artificial sequence
  Exemplary siRNA target sequence
acaccaaggcccacgcaca

SEQ ID NO: 260 - DNA, 19 bp, Artificial sequence
  Exemplary siRNA target sequence
tctccaagcgcaagtgtga

SEQ ID NO: 261 - DNA, 19 bp, Artificial sequence
  Exemplary siRNA target sequence
gtgctgtggagaaagaaga

SEQ ID NO: 262 - DNA, 19 bp, Artificial sequence
  Exemplary siRNA target sequence
gaggagacacggaacacca

SEQ ID NO: 263 - DNA, 19 bp, Artificial sequence
  Exemplary siRNA target sequence
aggattacctcgccctgaa

SEQ ID NO: 264 - DNA, 19 bp, Artificial sequence
  Exemplary siRNA target sequence
ccaagacacacgtgaccca

SEQ ID NO: 265 - DNA, 19 bp, Artificial sequence
  Exemplary siRNA target sequence
acgtggagctcgtggagac

SEQ ID NO: 266 - DNA, 19 bp, Artificial sequence
  Exemplary siRNA target sequence
cggagtattgggaagagga

SEQ ID NO: 267 - DNA, 19 bp, Artificial sequence
  Exemplary siRNA target sequence
ccacagatacctggagaac

SEQ ID NO: 268 - DNA, 19 bp, Artificial sequence
  Exemplary siRNA target sequence
acagatacctggagaacgg

SEQ ID NO: 269 - DNA, 19 bp, Artificial sequence
  Exemplary siRNA target sequence
ggaagaggagacacggaac

SEQ ID NO: 270 - DNA, 19 bp, Artificial sequence
  Exemplary siRNA target sequence
tgaacaaaggagagcctac

SEQ ID NO: 271 - DNA, 19 bp, Artificial sequence
  Exemplary siRNA target sequence
ccatgggctacgtggacga

SEQ ID NO: 272 - DNA, 19 bp, Artificial sequence
  Exemplary siRNA target sequence
gagaaagaagagctcagat

SEQ ID NO: 273 - DNA, 19 bp, Artificial sequence
  Exemplary siRNA target sequence
gagtattgggaagaggaga

SEQ ID NO: 274 - DNA, 19 bp, Artificial sequence
  Exemplary siRNA target sequence
gggtatgaacagtatgcct

SEQ ID NO: 275 - DNA, 19 bp, Artificial sequence
  Exemplary siRNA target sequence
ccaatgtggctgaacaaag

SEQ ID NO: 276 - DNA, 19 bp, Artificial sequence
  Exemplary siRNA target sequence
ccaccctgtctttgactat

SEQ ID NO: 277 - DNA, 19 bp, Artificial sequence
  Exemplary siRNA target sequence
ggttgtccttgcagctgta

SEQ ID NO: 278 - DNA, 19 bp, Artificial sequence
  Exemplary siRNA target sequence
gaaagaagagctcagattg

SEQ ID NO: 279 - DNA, 19 bp, Artificial sequence
  Exemplary siRNA target sequence
agtattgggaagaggagac

SEQ ID NO: 280 - DNA, 19 bp, Artificial sequence
  Exemplary siRNA target sequence
ctcagatctccaagcgcaa

SEQ ID NO: 281 - DNA, 19 bp, Artificial sequence
  Exemplary siRNA target sequence
tggagaaagaagagctcag

SEQ ID NO: 282 - DNA, 19 bp, Artificial sequence
  Exemplary siRNA target sequence
ggccggagtattgggaaga

SEQ ID NO: 283 - DNA, 19 bp, Artificial sequence
  Exemplary siRNA target sequence
ggagaaagaagagctcaga

SEQ ID NO: 284 - DNA, 19 bp, Artificial sequence
  Exemplary siRNA target sequence
gtattgggaagaggagaca

SEQ ID NO: 285 - DNA, 19 bp, Artificial sequence
  Exemplary siRNA target sequence
aggagacacggaacaccaa

SEQ ID NO: 286 - DNA, 19 bp, Artificial sequence
  Exemplary siRNA target sequence
aggcccacgcacagactga

SEQ ID NO: 287 - DNA, 19 bp, Artificial sequence
  Exemplary siRNA target sequence
cgtcgtgggtttaacaatt

SEQ ID NO: 288 - DNA, 19 bp, Artificial sequence
  Exemplary siRNA target sequence
gcaggaacctacagatgtt

SEQ ID NO: 289 - DNA, 19 bp, Artificial sequence
  Exemplary siRNA target sequence
ctacagatgttatggttct

SEQ ID NO: 290 - DNA, 19 bp, Artificial sequence
  Exemplary siRNA target sequence
gcaggactctgatgaacaa

SEQ ID NO: 291 - DNA, 19 bp, Artificial sequence
  Exemplary siRNA target sequence
gggacagaacagtgaatag

SEQ ID NO: 292 - DNA, 19 bp, Artificial sequence
  Exemplary siRNA target sequence
gaacattccaggcagactt

SEQ ID NO: 293 - DNA, 19 bp, Artificial sequence
  Exemplary siRNA target sequence
tgaaatcaggagagacagt

SEQ ID NO: 294 - DNA, 19 bp, Artificial sequence
  Exemplary siRNA target sequence
ggcaggactctgatgaaca

SEQ ID NO: 295 - DNA, 19 bp, Artificial sequence
  Exemplary siRNA target sequence
acagaacagtgaataggca

SEQ ID NO: 296 - DNA, 19 bp, Artificial sequence
  Exemplary siRNA target sequence
tcatgggtggtcaggacaa

SEQ ID NO: 297 - DNA, 19 bp, Artificial sequence
  Exemplary siRNA target sequence
catcttccacggcagaata

SEQ ID NO: 298 - DNA, 19 bp, Artificial sequence
  Exemplary siRNA target sequence
aaatcaggagagacagtca

SEQ ID NO: 299 - DNA, 19 bp, Artificial sequence
  Exemplary siRNA target sequence
tgaataggcaggactctga

SEQ ID NO: 300 - DNA, 19 bp, Artificial sequence
  Exemplary siRNA target sequence
tgcccaaggtcaacagaac

SEQ ID NO: 301 - DNA, 19 bp, Artificial sequence
  Exemplary siRNA target sequence
cccacgagcaccacagtca

SEQ ID NO: 302 - DNA, 19 bp, Artificial sequence
  Exemplary siRNA target sequence
ggagcttgtttgacattta

SEQ ID NO: 303 - DNA, 19 bp, Artificial sequence
  Exemplary siRNA target sequence
cgtaggtctatatgggaaa

SEQ ID NO: 304 - DNA, 19 bp, Artificial sequence
  Exemplary siRNA target sequence
tgacatacgcacagttgaa

SEQ ID NO: 305 - DNA, 19 bp, Artificial sequence
  Exemplary siRNA target sequence
gctgagggtcaatggaaca

SEQ ID NO: 306 - DNA, 19 bp, Artificial sequence
  Exemplary siRNA target sequence
gcagggacctacagatgtt

SEQ ID NO: 307 - DNA, 19 bp, Artificial sequence
  Exemplary siRNA target sequence
ctacagatgttgcagttca

SEQ ID NO: 308 - DNA, 19 bp, Artificial sequence
  Exemplary siRNA target sequence
catcgtggtcgtaggtcta

SEQ ID NO: 309 - DNA, 19 bp, Artificial sequence
  Exemplary siRNA target sequence
tcgtaggtctatatgggaa

SEQ ID NO: 310 - DNA, 19 bp, Artificial sequence
  Exemplary siRNA target sequence
ctacagatgctttggttct

SEQ ID NO: 311 - DNA, 19 bp, Artificial sequence
  Exemplary siRNA target sequence
ccagcaaccctgtggtgat

SEQ ID NO: 312 - DNA, 19 bp, Artificial sequence
  Exemplary siRNA target sequence
aaagaatgctgttgtaatg

SEQ ID NO: 313 - DNA, 19 bp, Artificial sequence
  Exemplary siRNA target sequence
gtgctgagggtcaatggaa

SEQ ID NO: 314 - DNA, 19 bp, Artificial sequence
  Exemplary siRNA target sequence
aacagataccagcgtgtaa

SEQ ID NO: 315 - DNA, 19 bp, Artificial sequence
  Exemplary siRNA target sequence
ccaacagataccagcgtgt

SEQ ID NO: 316 - DNA, 19 bp, Artificial sequence
  Exemplary siRNA target sequence
ggtctatatgggaaacctt

SEQ ID NO: 317 - DNA, 19 bp, Artificial sequence
  Exemplary siRNA target sequence
tcacttaccctatgagttg

SEQ ID NO: 318 - DNA, 19 bp, Artificial sequence
  Exemplary siRNA target sequence
catacgcacagttgaatca

SEQ ID NO: 319 - DNA, 19 bp, Artificial sequence
  Exemplary siRNA target sequence
acatcgtggtcgtaggtct

SEQ ID NO: 320 - DNA, 19 bp, Artificial sequence
  Exemplary siRNA target sequence
acctacagatgttgcagtt

SEQ ID NO: 321 - DNA, 19 bp, Artificial sequence
  Exemplary siRNA target sequence
tcccggagcttgtttgaca

SEQ ID NO: 322 - DNA, 19 bp, Artificial sequence
  Exemplary siRNA target sequence
tgaaggacaacatgtgact

SEQ ID NO: 323 - DNA, 19 bp, Artificial sequence
  Exemplary siRNA target sequence
aaggacaacatgtgactct

SEQ ID NO: 324 - DNA, 19 bp, Artificial sequence
  Exemplary siRNA target sequence
gcaaccctgtggtgatcat

SEQ ID NO: 325 - DNA, 19 bp, Artificial sequence
  Exemplary siRNA target sequence
cgtcataggtttaacaatt

SEQ ID NO: 326 - DNA, 19 bp, Artificial sequence
  Exemplary siRNA target sequence
ctgcaatgttggtcagata

SEQ ID NO: 327 - DNA, 19 bp, Artificial sequence
  Exemplary siRNA target sequence
ggtcagatatcatgtttga

SEQ ID NO: 328 - DNA, 19 bp, Artificial sequence
  Exemplary siRNA target sequence
gaacattccaggcagattt

SEQ ID NO: 329 - DNA, 19 bp, Artificial sequence
  Exemplary siRNA target sequence
gcgcaaggtcaacagaaca

SEQ ID NO: 330 - DNA, 19 bp, Artificial sequence
  Exemplary siRNA target sequence
ccacagcacatgcagggaa

SEQ ID NO: 331 - DNA, 19 bp, Artificial sequence
  Exemplary siRNA target sequence
cggtgtcactatcgtcata

SEQ ID NO: 332 - DNA, 19 bp, Artificial sequence
  Exemplary siRNA target sequence
gctatacaaagaagacaga

SEQ ID NO: 333 - DNA, 19 bp, Artificial sequence
  Exemplary siRNA target sequence
cagcacatgcagggaacta

SEQ ID NO: 334 - DNA, 19 bp, Artificial sequence
  Exemplary siRNA target sequence
atatcatgtttgagcactt

SEQ ID NO: 335 - DNA, 19 bp, Artificial sequence
  Exemplary siRNA target sequence
gcacatgcagggaactaca

SEQ ID NO: 336 - DNA, 19 bp, Artificial sequence
  Exemplary siRNA target sequence
cgcaaggtcaacagaacat

SEQ ID NO: 337 - DNA, 19 bp, Artificial sequence
  Exemplary siRNA target sequence
gcagggaacagaacagcca

SEQ ID NO: 338 - DNA, 19 bp, Artificial sequence
  Exemplary siRNA target sequence
ctggtgaaatcaggagaga

SEQ ID NO: 339 - DNA, 19 bp, Artificial sequence
  Exemplary siRNA target sequence
ggttcaggcaggagagagc

SEQ ID NO: 340 - DNA, 19 bp, Artificial sequence
  Exemplary siRNA target sequence
atacaaagaagacagaatc

SEQ ID NO: 341 - DNA, 19 bp, Artificial sequence
  Exemplary siRNA target sequence
ccaaggttcaggcaggaga

SEQ ID NO: 342 - DNA, 19 bp, Artificial sequence
  Exemplary siRNA target sequence
tgctaagcccagatccaaa

SEQ ID NO: 343 - DNA, 19 bp, Artificial sequence
  Exemplary siRNA target sequence
cagatatcatgtttgagca

SEQ ID NO: 344 - DNA, 19 bp, Artificial sequence
  Exemplary siRNA target sequence
caaagaagacagaatccac

SEQ ID NO: 345 - DNA, 19 bp, Artificial sequence
  Exemplary siRNA target sequence
caacatgagccctgtgacc

SEQ ID NO: 346 - DNA, 19 bp, Artificial sequence
  Exemplary siRNA target sequence
agcacttctttctgcacaa

SEQ ID NO: 347 - DNA, 19 bp, Artificial sequence
  Exemplary siRNA target sequence
gtgcgcaaggtcaacagaa

SEQ ID NO: 348 - DNA, 19 bp, Artificial sequence
  Exemplary siRNA target sequence
ccaaatgctaagcccagat

SEQ ID NO: 349 - DNA, 19 bp, Artificial sequence
  Exemplary siRNA target sequence
aatgctaagcccagatcca

SEQ ID NO: 350 - DNA, 19 bp, Artificial sequence
  Exemplary siRNA target sequence
gcaatgttggtcagatatc

SEQ ID NO: 351 - DNA, 19 bp, Artificial sequence
  Exemplary siRNA target sequence
gcacttctttctgcacaaa

SEQ ID NO: 352 - DNA, 19 bp, Artificial sequence
  Exemplary siRNA target sequence
agaacattccaggcagatt

SEQ ID NO: 353 - DNA, 19 bp, Artificial sequence
  Exemplary siRNA target sequence
gcagggaactacacatgtc

SEQ ID NO: 354 - DNA, 19 bp, Artificial sequence
  Exemplary siRNA target sequence
acaaagaagacagaatcca

SEQ ID NO: 355 - DNA, 19 bp, Artificial sequence
  Exemplary siRNA target sequence
caacagatatcatcgtgta

SEQ ID NO: 356 - DNA, 19 bp, Artificial sequence
  Exemplary siRNA target sequence
atgagggagtccacagaaa

SEQ ID NO: 357 - DNA, 19 bp, Artificial sequence
  Exemplary siRNA target sequence
catcgctggtgctgcaaca

SEQ ID NO: 358 - DNA, 19 bp, Artificial sequence
  Exemplary siRNA target sequence
cagatatcatcgtgtacac

SEQ ID NO: 359 - DNA, 19 bp, Artificial sequence
  Exemplary siRNA target sequence
tgagggagtccacagaaaa

SEQ ID NO: 360 - DNA, 19 bp, Artificial sequence
  Exemplary siRNA target sequence
tcgctggtgctgcaacaaa

SEQ ID NO: 361 - DNA, 19 bp, Artificial sequence
  Exemplary siRNA target sequence
cgctggtgctgcaacaaaa

SEQ ID NO: 362 - DNA, 19 bp, Artificial sequence
  Exemplary siRNA target sequence
ccagacacctgcatgttct

SEQ ID NO: 363 - DNA, 19 bp, Artificial sequence
  Exemplary siRNA target sequence
ccaacagatatcatcgtgt

SEQ ID NO: 364 - DNA, 19 bp, Artificial sequence
  Exemplary siRNA target sequence
acatgagggagtccacaga

SEQ ID NO: 365 - DNA, 19 bp, Artificial sequence
  Exemplary siRNA target sequence
catgagggagtccacagaa

SEQ ID NO: 366 - DNA, 19 bp, Artificial sequence
  Exemplary siRNA target sequence
cctggccacatgagggagt

SEQ ID NO: 367 - DNA, 19 bp, Artificial sequence
  Exemplary siRNA target sequence
acagatatcatcgtgtaca

SEQ ID NO: 368 - DNA, 19 bp, Artificial sequence
  Exemplary siRNA target sequence
ccacatgagggagtccaca

SEQ ID NO: 369 - DNA, 19 bp, Artificial sequence
  Exemplary siRNA target sequence
gatagatggtgttggtgaa

SEQ ID NO: 370 - DNA, 19 bp, Artificial sequence
  Exemplary siRNA target sequence
caagaaaaggtctgggtaa

SEQ ID NO: 371 - DNA, 19 bp, Artificial sequence
  Exemplary siRNA target sequence
cgactaaacttaatctgta

SEQ ID NO: 372 - DNA, 19 bp, Artificial sequence
  Exemplary siRNA target sequence
gtgacgggattgaaacaaa

SEQ ID NO: 373 - DNA, 19 bp, Artificial sequence
  Exemplary siRNA target sequence
caggaaggaacgcgactaa

SEQ ID NO: 374 - DNA, 19 bp, Artificial sequence
  Exemplary siRNA target sequence
ataagaaagaagaggctga

SEQ ID NO: 375 - DNA, 19 bp, Artificial sequence
  Exemplary siRNA target sequence
ggatagatggtgttggtga

SEQ ID NO: 376 - DNA, 19 bp, Artificial sequence
  Exemplary siRNA target sequence
gggattgaaacaaagacaa

SEQ ID NO: 377 - DNA, 19 bp, Artificial sequence
  Exemplary siRNA target sequence
aagaagaggctgaggggtt

SEQ ID NO: 378 - DNA, 19 bp, Artificial sequence
  Exemplary siRNA target sequence
cagttgatgttcaccataa

SEQ ID NO: 379 - DNA, 19 bp, Artificial sequence
  Exemplary siRNA target sequence
tcacagagacagggaacta

SEQ ID NO: 380 - DNA, 19 bp, Artificial sequence
  Exemplary siRNA target sequence
gcataagaaagaagaggct

SEQ ID NO: 381 - DNA, 19 bp, Artificial sequence
  Exemplary siRNA target sequence
ttaaaacagctgagactta

SEQ ID NO: 382 - DNA, 19 bp, Artificial sequence
  Exemplary siRNA target sequence
gaaacaaagacaacacctt

SEQ ID NO: 383 - DNA, 19 bp, Artificial sequence
  Exemplary siRNA target sequence
aagaaaaggtctgggtaat

SEQ ID NO: 384 - DNA, 19 bp, Artificial sequence
  Exemplary siRNA target sequence
cattctattcacagagaca

SEQ ID NO: 385 - DNA, 19 bp, Artificial sequence
  Exemplary siRNA target sequence
acagggaactacacagtga

SEQ ID NO: 386 - DNA, 19 bp, Artificial sequence
  Exemplary siRNA target sequence
cgggattgaaacaaagaca

SEQ ID NO: 387 - DNA, 19 bp, Artificial sequence
  Exemplary siRNA target sequence
ggattgaaacaaagacaac

SEQ ID NO: 388 - DNA, 19 bp, Artificial sequence
  Exemplary siRNA target sequence
ctaccagtgttgtgccaga

SEQ ID NO: 389 - DNA, 19 bp, Artificial sequence
  Exemplary siRNA target sequence
ggaaggaacgcgactaaac

SEQ ID NO: 390 - DNA, 19 bp, Artificial sequence
  Exemplary siRNA target sequence
aggaacgcgactaaactta

SEQ ID NO: 391 - DNA, 19 bp, Artificial sequence
  Exemplary siRNA target sequence
actacacagtgacgggatt

SEQ ID NO: 392 - DNA, 19 bp, Artificial sequence
  Exemplary siRNA target sequence
cagtctggtggatgcatta

SEQ ID NO: 393 - DNA, 19 bp, Artificial sequence
  Exemplary siRNA target sequence
ggaacgcgactaaacttaa

SEQ ID NO: 394 - DNA, 19 bp, Artificial sequence
  Exemplary siRNA target sequence
cagccataatgaaggcact

SEQ ID NO: 395 - DNA, 19 bp, Artificial sequence
  Exemplary siRNA target sequence
gttcaggcttcctacaaga

SEQ ID NO: 396 - DNA, 19 bp, Artificial sequence
  Exemplary siRNA target sequence
aatctgtactgtatggcat

SEQ ID NO: 397 - DNA, 19 bp, Artificial sequence
  Exemplary siRNA target sequence
cacagtgacgggattgaaa

SEQ ID NO: 398 - DNA, 19 bp, Artificial sequence
  Exemplary siRNA target sequence
cagtgacgggattgaaaca

SEQ ID NO: 399 - DNA, 19 bp, Artificial sequence
  Exemplary siRNA target sequence
gcaaggacaggtctggaga

SEQ ID NO: 400 - DNA, 19 bp, Artificial sequence
  Exemplary siRNA target sequence
tgggatagatggtgttggt

SEQ ID NO: 401 - DNA, 19 bp, Artificial sequence
  Exemplary siRNA target sequence
aagacaacaccttgagttc

SEQ ID NO: 402 - DNA, 19 bp, Artificial sequence
  Exemplary siRNA target sequence
gaacgcgactaaacttaat

SEQ ID NO: 403 - DNA, 19 bp, Artificial sequence
  Exemplary siRNA target sequence
agaagaggctgaggggttt

SEQ ID NO: 404 - DNA, 19 bp, Artificial sequence
  Exemplary siRNA target sequence
caggcttcctacaagaaaa

SEQ ID NO: 405 - DNA, 19 bp, Artificial sequence
  Exemplary siRNA target sequence
agacagggaactacacagt

SEQ ID NO: 406 - DNA, 19 bp, Artificial sequence
  Exemplary siRNA target sequence
gcgactaaacttaatctgt

SEQ ID NO: 407 - DNA, 19 bp, Artificial sequence
  Exemplary siRNA target sequence
gatgttcaccataagccaa

SEQ ID NO: 408 - DNA, 19 bp, Artificial sequence
  Exemplary siRNA target sequence
acagagacagggaactaca

SEQ ID NO: 409 - DNA, 19 bp, Artificial sequence
  Exemplary siRNA target sequence
ccaggaaggaacgcgacta

SEQ ID NO: 410 - DNA, 19 bp, Artificial sequence
  Exemplary siRNA target sequence
aggaaggaacgcgactaaa

SEQ ID NO: 411 - DNA, 19 bp, Artificial sequence
  Exemplary siRNA target sequence
gtatccgccttcagggcca

SEQ ID NO: 412 - DNA, 19 bp, Artificial sequence
  Exemplary siRNA target sequence
gcattaacatcaccagctc

SEQ ID NO: 413 - DNA, 19 bp, Artificial sequence
  Exemplary siRNA target sequence
gaagccagaagtcaggtat

SEQ ID NO: 414 - DNA, 19 bp, Artificial sequence
  Exemplary siRNA target sequence
attcacagagacagggaac

SEQ ID NO: 415 - DNA, 19 bp, Artificial sequence
  Exemplary siRNA target sequence
gactaaacttaatctgtac

SEQ ID NO: 416 - DNA, 19 bp, Artificial sequence
  Exemplary siRNA target sequence
gattgaaacaaagacaaca

SEQ ID NO: 417 - DNA, 19 bp, Artificial sequence
  Exemplary siRNA target sequence
gctgttggaacccggcaga

SEQ ID NO: 418 - DNA, 19 bp, Artificial sequence
  Exemplary siRNA target sequence
gccagaagccagaagtcag

SEQ ID NO: 419 - DNA, 19 bp, Artificial sequence
  Exemplary siRNA target sequence
ctaaacttctcaaggattt

SEQ ID NO: 420 - DNA, 19 bp, Artificial sequence
  Exemplary siRNA target sequence
tgtaagaaggtcagacttt

SEQ ID NO: 421 - DNA, 19 bp, Artificial sequence
  Exemplary siRNA target sequence
gaggaaaaggactggaatt

SEQ ID NO: 422 - DNA, 19 bp, Artificial sequence
  Exemplary siRNA target sequence
gaaaaggactggaattcta

SEQ ID NO: 423 - DNA, 19 bp, Artificial sequence
  Exemplary siRNA target sequence
gctggatgaaatatggtaa

SEQ ID NO: 424 - DNA, 19 bp, Artificial sequence
  Exemplary siRNA target sequence
ctgcctagccagagactca

SEQ ID NO: 425 - DNA, 19 bp, Artificial sequence
  Exemplary siRNA target sequence
tggattggtctgaggaaca

SEQ ID NO: 426 - DNA, 19 bp, Artificial sequence
  Exemplary siRNA target sequence
caacccaagcccagaatga

SEQ ID NO: 427 - DNA, 19 bp, Artificial sequence
  Exemplary siRNA target sequence
ccagaatgactatggacca

SEQ ID NO: 428 - DNA, 19 bp, Artificial sequence
  Exemplary siRNA target sequence
tgactatggaccacagcaa

SEQ ID NO: 429 - DNA, 19 bp, Artificial sequence
  Exemplary siRNA target sequence
taacggacaatcaggaaat

SEQ ID NO: 430 - DNA, 19 bp, Artificial sequence
  Exemplary siRNA target sequence
cctctaaacttctcaagga

SEQ ID NO: 431 - DNA, 19 bp, Artificial sequence
  Exemplary siRNA target sequence
ggaagatggatcacctcta

SEQ ID NO: 432 - DNA, 19 bp, Artificial sequence
  Exemplary siRNA target sequence
ctggaattctagtctggaa

SEQ ID NO: 433 - DNA, 19 bp, Artificial sequence
  Exemplary siRNA target sequence
ggattggtctgaggaacaa

SEQ ID NO: 434 - DNA, 19 bp, Artificial sequence
  Exemplary siRNA target sequence
ctgcagttcttctgagtgt

SEQ ID NO: 435 - DNA, 19 bp, Artificial sequence
  Exemplary siRNA target sequence
tctcaaggatttcttctaa

SEQ ID NO: 436 - DNA, 19 bp, Artificial sequence
  Exemplary siRNA target sequence
actatggaccacagcaaaa

SEQ ID NO: 437 - DNA, 19 bp, Artificial sequence
  Exemplary siRNA target sequence
ggcaatagctttggggctt

SEQ ID NO: 438 - DNA, 19 bp, Artificial sequence
  Exemplary siRNA target sequence
ataacggacaatcaggaaa

SEQ ID NO: 439 - DNA, 19 bp, Artificial sequence
  Exemplary siRNA target sequence
tctaatagctttgtgcaga

SEQ ID NO: 440 - DNA, 19 bp, Artificial sequence
  Exemplary siRNA target sequence
gactatggaccacagcaaa

SEQ ID NO: 441 - DNA, 19 bp, Artificial sequence
  Exemplary siRNA target sequence
gcttctgactgcagttctt

SEQ ID NO: 442 - DNA, 19 bp, Artificial sequence
  Exemplary siRNA target sequence
gctggattggtctgaggaa

SEQ ID NO: 443 - DNA, 19 bp, Artificial sequence
  Exemplary siRNA target sequence
gaggtgggaagatggatca

SEQ ID NO: 444 - DNA, 19 bp, Artificial sequence
  Exemplary siRNA target sequence
tctaaacttctcaaggatt

SEQ ID NO: 445 - DNA, 19 bp, Artificial sequence
  Exemplary siRNA target sequence
agacatgcggtgccatcaa

SEQ ID NO: 446 - DNA, 19 bp, Artificial sequence
  Exemplary siRNA target sequence
cagaccgctggatgaaata

SEQ ID NO: 447 - DNA, 19 bp, Artificial sequence
  Exemplary siRNA target sequence
ggaggtgggaagatggatc

SEQ ID NO: 448 - DNA, 19 bp, Artificial sequence
  Exemplary siRNA target sequence
ctctaaacttctcaaggat

SEQ ID NO: 449 - DNA, 19 bp, Artificial sequence
  Exemplary siRNA target sequence
cggcaacccaagcccagaa

SEQ ID NO: 450 - DNA, 19 bp, Artificial sequence
  Exemplary siRNA target sequence
gtgtaagaaggtcagactt

SEQ ID NO: 451 - DNA, 19 bp, Artificial sequence
  Exemplary siRNA target sequence
taagaaggtcagactttga

SEQ ID NO: 452 - DNA, 19 bp, Artificial sequence
  Exemplary siRNA target sequence
gaacaattctggctggagg

SEQ ID NO: 453 - DNA, 19 bp, Artificial sequence
  Exemplary siRNA target sequence
tgtgtaagaaggtcagact

SEQ ID NO: 454 - DNA, 19 bp, Artificial sequence
  Exemplary siRNA target sequence
ggaggaaaaggactggaat

SEQ ID NO: 455 - DNA, 19 bp, Artificial sequence
  Exemplary siRNA target sequence
gtgggaagatggatcacct

SEQ ID NO: 456 - DNA, 19 bp, Artificial sequence
  Exemplary siRNA target sequence
ctaatagctttgtgcagac

SEQ ID NO: 457 - DNA, 19 bp, Artificial sequence
  Exemplary siRNA target sequence
gtgcagacatgcggtgcca

SEQ ID NO: 458 - DNA, 19 bp, Artificial sequence
  Exemplary siRNA target sequence
atgcggtgccatcaacaaa

SEQ ID NO: 459 - DNA, 19 bp, Artificial sequence
  Exemplary siRNA target sequence
agcctcaagctgtgaagtt

SEQ ID NO: 460 - DNA, 19 bp, Artificial sequence
  Exemplary siRNA target sequence
ctggatgaaatatggtaac

SEQ ID NO: 461 - DNA, 19 bp, Artificial sequence
  Exemplary siRNA target sequence
gttattatttctcagtgga

SEQ ID NO: 462 - DNA, 19 bp, Artificial sequence
  Exemplary siRNA target sequence
aggaaaaggactggaattc

SEQ ID NO: 463 - DNA, 19 bp, Artificial sequence
  Exemplary siRNA target sequence
cagaatgactatggaccac

SEQ ID NO: 464 - DNA, 19 bp, Artificial sequence
  Exemplary siRNA target sequence
ctcaaggatttcttctaat

SEQ ID NO: 465 - DNA, 19 bp, Artificial sequence
  Exemplary siRNA target sequence
aagctgtgaagttccttta

SEQ ID NO: 466 - DNA, 19 bp, Artificial sequence
  Exemplary siRNA target sequence
gctgtgaagttcctttaca

SEQ ID NO: 467 - DNA, 19 bp, Artificial sequence
  Exemplary siRNA target sequence
tgggtgtgtaagaaggtca

SEQ ID NO: 468 - DNA, 19 bp, Artificial sequence
  Exemplary siRNA target sequence
tggcaatagctttggggct